(19) United States
(12) Patent Application Publication
(10) Pub. No.: US 2009/0075272 A1
(43) Pub. Date: Mar. 19, 2009

(54) Method to Identify CD40-Sensitive Cells Using Gene Expression

(76) Inventors: C. Annette Hollmann, Montreal (CA); Robert Sladek, Montreal (CA); Trevor Owens, Odense (DK); Thomas J. Hudson, Toronto (CA)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/087,854

(22) PCT Filed: Jan. 18, 2007
(86) PCT No.: PCT/CA2007/000073

§ 371 Date: Sep. 24, 2008

(51) Int. Cl. C12Q 1/68; C12Q 1/02; G01N 33/53; G01N 33/574

(52) U.S. Cl. 435/6; 435/29; 435/7.21; 435/7.23

(57) ABSTRACT

Gene expression patterns were analyzed in CD40-sensitive and CD40-resistant diffuse large-cell B-lymphoma (DLCBL) cell lines to identify signaling pathways which are involved in CD40-mediated apoptosis. CD40-resistant lines expressed pre-B cell markers including RAG and VPREB, whereas CD40-sensitive cells resembled mature B-cells and expressed higher levels of transcripts encoding several members of the CD40 signaling pathway including LCK and VAV. In addition, CD40 sensitive DLCBL cell lines also displayed constitutive activation of ERK and failed to undergo apoptosis when ERK phosphorylation was inhibited. In contrast, CD40 resistant lines showed no constitutive activation of ERK and no increase in ERK activity in response to CD40 stimulation. The invention includes methods to differentiate between CD40-sensitive and CD-40 resistant cells based on these differences in gene expression.

### RELATED APPLICATION

This application claims the benefit of U.S. Provisional Application No. 60/760,648, filed on Jan. 20, 2006. The entire teachings of the above application are incorporated herein by reference.

### BACKGROUND OF THE INVENTION

CD40 promotes survival, proliferation, and differentiation of normal B-cells, but can cause activation-induced cell death in malignant B-lymphocytes. CD40 ligand and anti-CD40 antibodies have been used successfully to induce apoptosis in lymphoma lines both in vitro and in xenograft tumor models. While this makes CD40 an attractive target for anti-tumor therapies, the response of malignant B-cells to CD40 signaling is variable and CD40 stimulation can enhance proliferation and increase chemoresistance in some cell lines.

CD40 is first expressed prior to the rearrangement of immunoglobulin heavy chain genes in early B-cell development; its expression is maintained through all subsequent stages of B-cell development and is not lost during malignant transformation (2). Malignant B-lymphocytes and other CD40-positive tumor cells differ from normal B-cells in that they undergo apoptosis following CD40 stimulation (3). CD40 stimulation shows promise as an anti-tumor therapy in murine models of B-cell lymphoma and breast cancer (4, 5). CD40 ligand has also been tested in phase I clinical trials (6); however, the use of CD40-directed therapy remains controversial because CD40 signaling can enhance cell proliferation and survival as well as induce resistance to chemotherapeutic agents in some B-cell malignancies (7, 8). Consequently, elucidation of the mechanisms involved in CD40-mediated apoptosis may help to identify markers for susceptibility to CD40-mediated cell death and reveal proteins that specifically control the apoptotic arm of CD40 signaling. It would be useful to have a method to use to predict whether a specific cell line or tumor will undergo apoptosis when stimulated with CD40, and to identify targets downstream of CD40 which affect only the apoptotic arm of CD40 signaling.

### SUMMARY OF THE INVENTION

In one embodiment, the invention is a method of determining whether first cells respond to CD40 stimulation by undergoing apoptosis, said method comprising testing said first cells for their profile of gene expression, and comparing said profile with a second profile of gene expression of second cells known to respond to CD40 stimulation by undergoing apoptosis. If the profile of gene expression of the first cells shows expression levels of genes characteristic of the second profile, said first cells respond to CD40 stimulation by undergoing apoptosis. The method can be performed by analyzing RNA from the first cells and the second cells on one or more arrays prepared for detection and/or quantitation of RNA purified or partially purified from cells.

In another embodiment, the invention is a method of determining whether first cells respond to CD40 stimulation by undergoing apoptosis, said method comprising testing said first cells for levels of expression of one or more genes, and comparing a first set of levels of expression of said one or more genes to a second set of levels of expression of said one or more genes in second cells known to respond to CD40 stimulation by undergoing apoptosis; wherein, if the first set of levels of expression of said one or more genes in said first cells is characteristic of said second set of levels of the second cells, said first cells respond to CD40 stimulation by undergoing apoptosis.

Methods for quantitating levels of gene expression and/or providing means to compare levels of expression of selected genes are known in the art, and any such method previously described can be used where a step of a method requires such quantitation and/or comparison. Methods that rely on nucleic acid hybridization or hybridizable analogs thereof are particularly useful. They can include, for example, analysis by oligonucleotide (or hybridizable analogs thereof) arrays or microarrays, commercially available or custom-made. Other methods that can be used for quantitating and comparing gene expression are RT-PCR, western blotting or immunostaining methods.

In a further embodiment, the invention is a method for determining whether a population of cells is CD40-sensitive, said method comprising quantitating expression of one or more genes in a sample of cells from the population, wherein said one or more genes in diffuse large-cell B-lymphoma (DLCBL) cell lines are differentially regulated between CD40-sensitive DLCBL cell lines and CD40-resistant DLCBL cell lines, and comparing quantities of expression of said one or more genes in said sample to quantities of expression of said one or more genes in CD40-resistant DLCBL cell lines, wherein if said one or more genes are differentially regulated between the cells in the sample and the CD40-resistant DLCBL cell lines, then the population of cells is CD40-sensitive.

The genes to be examined for gene expression can be one or more of any of a number of genes described herein that were found to be expressed constitutively at significantly different levels in CD40-resistant cells as compared to expression of those genes in CD40-sensitive cells. The genes can be one or any number of genes selected from B-cell maturation specific genes and members of the CD40/BCR signaling pathway (see Tables 7A, 7B, 10A and 10B). Preferred genes to examine are RAG1, RAG2, IGLL1, CD9, VPREB1, CD22, CD38, Bruton's tyrosine kinase, VAV1, LYN, LCK and MEK1/MAP2K1. In other methods, the gene to be examined can be RAG1 or VAV1 or both.

Methods to quantitate gene expression are known by persons of skill in the art. One such suitable method is reverse transcription polymerase chain reaction (RT-PCR). Another method that allows quantitating gene expression and comparing levels of expression among or between genes is analysis on arrays. A further method to quantitate expression of a gene is immunohistochemistry, which employs antibodies that can be added to a sample of cells prepared for immunohistochemical testing. The antibodies bind specifically to the protein product of the gene.

Yet another embodiment of the invention is a method for determining whether a population of cells is CD40-sensitive or CD40-resistant, said method comprising testing a sample of cells from said population for the presence or absence of phosphorylated ERK, whereby, if phosphorylated ERK is present, the population of cells is CD40-sensitive, and if phosphorylated ERK is absent, population of cells is CD40-resistant. The method can be carried out by testing the sample of cells by immunohistochemical methods, such as immunoblot of non-denatured lysates from the sample of cells using anti-phospho-ERK antibodies, or immunostaining with immunofluorescent labeled anti-phospho-ERK antibodies or with anti-phospho-ERK antibodies conjugated to a reactive label that can be readily visualized, for example.

Any of the above methods can be carried out on a sample of cells wherein the sample is a tissue biopsy or a fluid sample from a human or animal. Any of the methods can be carried out on a population of cells or a portion of a population of cells wherein the population of cells is a primary culture of cells from a human or animal, or the population of cells is a cell line. The population of cells can comprise B-cell lymphoma cells, diffuse large-cell B-lymphoma cells, cancer cells, for example cancer cells derived from endothelial cells, epithelial cells, fibroblasts, or breast cancer cells, prostate cancer cells, lung cancer cells, or colon cancer cells, for instance.

A further method can be used to identify CD40-sensitive cells, performed by treating said cells to activate CD40, and testing said cells for an increase in the quantity of phosphorylated ERK, whereby if an increase in the quantity of phosphorylated ERK is observed, said cells are CD40-sensitive cells.

Cells in a population of cells, following one or more generations of cell divisions, may change in phenotype and/or genotype relative to the original population of cells. Cells may have been grown in culture for one or more generations or they may have undergone mutation(s) at their normal site in a human or animal. Cancer cells are widely recognized as cells that have undergone genotypic and phenotypic changes so that they differ from their cell of origin. Cells that are derived from a specific cell type means that the original population of cells some generations ago were of that cell type.

Arrays or microarrays for detection and quantitation of RNA are not limited to the use of oligonucleotides as probes. Arrays can include as probes oligonucleotides, peptide nucleic acids, locked nucleic acids, phosphorothioate analogs of oligonucleotides and other analogs or mimics of oligonucleotides that can hybridize to RNA. Such probes can also be used in other methods based on hybridization.

### BRIEF DESCRIPTION OF THE DRAWINGS

FIGS. 1A-1D are graphs showing that antiproliferative and apoptotic effects of CD40 signaling differ among DLCBL lines. OCI-Ly1, OCI-Ly7, OCI-Ly8 or Su-DHL4 cells were seeded at 100,000 cells per mL in supplemented RPMI containing 10 μg/mL anti-CD40 antibody and 10 μg/mL crosslinking secondary antibody (anti-CD40; filled squares), or secondary antibody alone (control; open circles). For assessment of cell viability, aliquots were removed at 24-hour intervals, stained with propidium iodide without prior permeabilization, and analyzed by flow cytometry using a fixed-time setting of 30 seconds to quantitate the number of viable cells per unit volume. Results of two experiments of three replicates each were combined. Error bars represent standard deviations; where these are not visible they are covered by the overlying symbol. Growth of OCI-Ly7 and Su-DHL4 cells was inhibited by crosslinked anti-CD40. For quantitation of apoptosis, cells were stimulated for 48 hours with anti-CD40 or control antibody as described above, and then permeabilized with ethanol prior to staining with propidium iodide. Intracellular DNA content was analyzed by flow cytometry.

FIG. 2 is a bar graph showing the percentage of cells with sub-G1 DNA. Cells containing less DNA than the G1 fraction, representing apoptotic cells, were quantitated. Results from two experiments of three replicates each are shown. Error bars indicate standard deviations; asterisks indicate statistically significant differences between control and anti-CD40 treated cells. The fraction of cells with sub-G1 DNA content increased in OCI-Ly7 and Su-DHL4 cells.

FIGS. 3A-3D are profiles of fluorescence from cells sorted by flow cytometry. CD40 could be detected on all four DLCBL cell lines by flow cytometry. Open profiles indicate fluorescence from phycoerythrin-conjugated anti-CD40 antibody and shaded profiles represent similarly conjugated isotype control antibody. Cells were not permeabilized prior to staining, thereby restricting staining to antigens exposed on the cell surface.

FIGS. 4A and 4B are gene expression profiles of CD40-sensitive and CD40-resistant DLCBL: B-cell markers and CD40 signaling. mRNA from unstimulated DLCBL lines was analyzed on Affymetrix HG-U133A v 2.0 Gene Chips as described in the Materials and Methods section of the Examples. B-cell differentiation markers and genes in the CD40/BCR signaling pathway were compared in triplicate samples of each of the four cell lines. Brackets indicate hierarchical clustering performed with Genesis software (23). The vertical bar indicates a group of co-segregating pre-B cell markers (pre-B). See also Tables 2A, 2B, 3A, 3B, 4A, 4B, 5A, 5B, 6A, 6B, 7A, 7B, 8A, 8B, 9A, 9B, 10A and 10B.

FIGS. 5A-5E are bar graphs showing relative expression levels of genes in the CD40 signaling pathway. Expression of several genes which had shown differential expression by oligonucleotide array analysis was verified by RT-PCR. PCR products were analyzed by gel electrophoresis and photographed with a Syngene gel documentation system. Relative levels of expression were quantitated with GeneTools software by comparison with a standard curve generated by amplification of PCR product from serially diluted cDNA. Results from triplicate samples are shown. Error bars indicate standard deviations. Where these are not visible variations between samples were very small. N.D. indicates not detectable.

FIG. 6 is a diagram illustrating the sites of action of two kinase inhibitors. Two genes involved in CD40-mediated ERK signaling are differentially expressed among CD40-sensitive and CD40-resistant cell lines. Differences in expression levels based on oligonucleotide array analysis are indicated. Fold difference in expression could not be calculated for VAV, as this mRNA was not detectable in CD40-resistant cells.

FIGS. 7A and 7B are images of immunoblots. Activity of LCK and ERK was analyzed with phosphorylation-specific antibodies. LCK was immunoprecipitated from nondenatured cell lysates and detected by Western blotting with phospho-src-family antibody (phospho-src). The blot was stripped and reprobed with an antibody against LCK (LCK). A commercially available Jurkat cell lysate was used as a positive control. This sample was used without prior immunoprecipitation, allowing distinction of LCK from the slightly lower mobility band representing the antibody used in immunoprecipitation. ERK was detected by immunoblotting with an antibody recognizing phosphorylated ERK1/2 (phospho) and the blot was reprobed with a pan-specific anti-ERK1/2 antibody (total).

FIGS. 8A and 8B are graphs of absorbance of cells, as a measure of viability, following the addition of PP1 or U0126. Sensitivity of the cell lines to inhibition of LCK and ERK activity was tested by addition of the src family kinase inhibitor PP1 or the MEK1/2 inhibitor U0126. Cells were incubated for 96 hours in inhibitor concentrations ranging from 10⁻⁴ to 10⁻⁸ M, and viability was assessed by MTT [3-(4,5-dimethylthiazol-2-yl)-2,5-diphenyltetrazolium bromide] assay. Viability is expressed as a percentage of the absorbance at 570 nm as compared to untreated samples. Results of two experiments with quadruplicate samples each were combined. Error bars indicate standard deviations.

FIG. 9 consists of images of stained Western blots. Cytoplasmic cell extracts were prepared from cells stimulated with crosslinked anti-CD40 antibody for 0 to 180 minutes. Total and phosphorylated ERK was detected by Western blot. Blots were first probed with the phospho-specific antibody, then the same membrane was re-probed for total protein with the pan-specific antibody.

FIG. 10 is a bar graph showing the effect of ERK inhibition on CD40-mediated apoptosis. Cells were subjected to CD40 stimulation for 96 hours in the presence of the MEK1/2 inhibitors U0126 (10 μM) or PD98059 (50 μM), or the p38 inhibitor SB203580 (10 μM). Cell viability was quantitated by staining nonpermeabilized cells with propidium iodide, and counting the number of unstained (live) cells per unit volume using a Becton-Dickinson FACScan flow cytometer set to count for 30 seconds. The number of viable cells in anti-CD40-treated samples was expressed as a percentage of the number of viable cells incubated in secondary antibody only. Results from three experiments of four replicates each are shown. Error bars represent standard deviations. In the presence of DMSO (control) or the p38 inhibitor SB203580, the number of viable OCI-Ly7 and Su-DHL4 cells was reduced by CD40 ligation, p<0.01. The MEK1/2 inhibitors U0126 and PD98059 prevented CD40-mediated reduction in viability. Constitutive ERK phosphorylation is correlated with CD40-mediated cell death.

### DETAILED DESCRIPTION OF THE INVENTION

Differences in the effects elicited by CD40 stimulation in B-cell lines suggest that different signal transduction pathways may be expressed in CD40-sensitive and CD40-resistant cells. To assess this, expression microarray studies were performed on CD40-sensitive and CD40-resistant DLCBL cell lines. The data showing that CD40-sensitive and resistant cells display different gene expression profiles suggests that these lines may be derived from two distinct B-cell populations. CD40-sensitive cells overexpressed CD22 and CD38, which are found on mature, activated B-cells (35). CD40-resistant cells expressed high levels of CD9, the recombination activating genes RAG1 and RAG2, and the pre-B cell receptor genes IGLL1 and VPREB1, which are characteristic of pre-B cells at the stage of immunoglobulin rearrangement (36-39), and have also been detected in B-lymphocytes in germinal centers (40). This suggests that the CD40-sensitive OCI-Ly7 and Su-DHL4 cell lines may be derived from mature, activated B-cells whereas the CD40-resistant OCI-Ly1 and OCI-Ly8 lines resemble immature B-cells. Expression of members of the CD40 signaling pathway was investigated to determine the underlying mechanism of CD40-mediated cell death. Failure of OCI-Ly1 and OCI-Ly8 cells to undergo apoptosis upon CD40 stimulation may be the result of defects in the CD40 signal transduction pathway. These two cell lines showed reduced expression of several genes involved in CD40 signaling, including LCK, VAV, and MEK1. Analysis of LCK by RT-PCR and Western blot revealed that although RNA levels differed among CD40-sensitive and CD40-resistant lines, all four cell lines expressed significant amounts of active LCK. This suggests that differences in LCK mRNA levels may not have any functional consequences. However, differences in VAV, a phosphorylation target of LCK, were striking, with strong mRNA expression in CD40-sensitive lines and undetectable levels in CD40-resistant lines. LCK and VAV have been previously shown to maintain constitutive activation of ERK via stimulation of the RAS pathway (31) which is consistent with our observation that ERK was constitutively phosphorylated in CD40-sensitive DLCBL cell lines but permanently inactive in VAV-deficient CD40-resistant lines. Although all four cell lines expressed active LCK, the SRC family inhibitor PP1 was more effective at reducing proliferation of OCI-Ly7 and Su-DHL4 cells. Differential sensitivity to PP1 could result from inhibition of other SRC family kinases or from the differential function of downstream effectors such as VAV, RAS, and ERK. Lack of VAV and inactive ERK in OCI-Ly1 and OCI-Ly8 cells is consistent with a dead-end in the SRC signal transduction pathway. This could render the cells resistant to SRC family kinase inhibitors even if the targeted kinase itself is active.

ERK activation has often been reported to be anti-apoptotic in both lymphoid and non-lymphoid malignancies (34, 41); however, the effect of ERK phosphorylation varies, even among different stimuli in the same cell line (12). Two different ERK inhibitors did not affect the growth of DLCBL cell lines containing activated ERK, which suggests that these lines are not dependent on ERK signaling for survival or proliferation. Addition of ERK inhibitors prior to CD40 stimulation blocked activation-induced cell death, which indicates that overexpression or aberrant activation of a protein in the ERK signaling cascade may sensitize DLCBL cell lines to CD40. The observation reported herein that ERK is constitutively active in these cell lines suggests that the actual death signal must be initiated by a second pathway when CD40 signaling occurs. ERK has been implicated in apoptosis in other systems of activation-induced cell death, both directly and as a predisposing factor. TCR-mediated activation-induced cell death in a TCR hybridoma cell line was shown to be mediated by activation of VAV and ERK (42). Transfection of RAT-1 fibroblasts or MCF-7 human breast cancer cells with the ERK-regulated transcription factor elk-1 did not induce apoptosis directly, but rendered the cells susceptible to killing by a calcium ionophore (38). ERK may function in a similar way in DLCBL cell lines, rendering cells susceptible to a second signal caused by CD40 stimulation. However, the signal is unlikely to be calcium-mediated, as treatment of the cells with the calcium ionophore ionomycin induced cell death in Su-DHL4 but not OCI-Ly7 cells.

The observations herein on two pairs of cell lines are insufficient to determine whether differential expression of maturation-specific genes outside of the LCK-ERK pathway plays a role in determining susceptibility to CD40-mediated cell death; however, they are consistent with a previous report that B-cells at different stages of development differ in the protein phosphorylation patterns elicited by CD40 stimulation (43). As CD40 signaling can promote proliferation and resistance to apoptosis in normal B-cells (44), it is possible that constitutive activation of this pathway in a tumor may lead to a more aggressive phenotype. DLCBL has been shown to segregate into two subtypes with distinct gene expression patterns, one resembling germinal center cells (germinal center type) and the other similar to mature B-cells that have been subjected to CD40 and B-cell receptor stimulation (activated B-cell type) (45). The prognosis was shown to differ significantly, with five-year survival being 76% for germinal center and 34% for activated type DLCBL (46). Neither the prevalence of constitutive ERK activation in either subtype of DLCBL nor correlation with CD40 sensitivity has yet been investigated in tumor tissue. This area may be fruitful for future investigation based not only on our observations, but also on recent development of inhibitors which modulate relevant signal transduction pathways. The SRC-VAV-ERK signal transduction pathway is activated by both CD40 and B-cell receptor signaling (12), and been implicated in proliferation of B-cell malignancies (41). Several proteins in this pathway, including CD40, SRC family kinases, RAS, and MEK are being investigated as targets for anti-tumor therapies (47-50).

The studies herein show that increased expression of genes involved CD40/BCR signal transduction coincided with constitutive ERK activation and susceptibility to CD40-mediated cell death in DLCBL cell lines. A constitutively active phenotype has previously been associated with reduced survival in DLCBL (45). This raises the question whether the poor prognosis of activated type DLCBL may be the result of one or more overactive proteins in the CD40 signal transduction pathway. An interesting but as yet untested hypothesis is that the mechanism underlying the aggressive nature of activated DLCBL may also be its Achilles heel, leaving it vulnerable to therapies targeting the CD40 signal transduction pathway.

### REFERENCES

1. van Kooten C, Banchereau J. CD40-CD40 ligand. J Leukoc Biol 2000; 67:2-17.
2. Uckun F M, Gajl-Peczalska K, Myers D E, Jaszcz W, Haissig S, Ledbetter J A. Temporal association of CD40 antigen expression with discrete stages of human B-cell ontogeny and the efficacy of anti-CD40 immunotoxins against clonogenic B-lineage acute lymphoblastic leukemia as well as B-lineage non-Hodgkin's lymphoma cells. Blood 1990; 76:2449-2456.
3. Tong A W, Stone M J. Prospects for CD40-directed experimental therapy of human cancer. Cancer Gene Ther 2003; 10:1-13.
4. Funakoshi S, Longo D L, Beckwith M, et al. Inhibition of human B-cell lymphoma growth by CD40 stimulation. Blood 1994; 83:2787-2794.
5. Hirano A, Longo D L, Taub D D, et al. Inhibition of human breast carcinoma growth by a soluble recombinant human CD40 ligand. Blood 1999; 93:2999-3007.
6. Vonderheide R H, Dutcher J P, Anderson J E, et al. Phase I study of recombinant human CD40 ligand in cancer patients. J Clin Oncol 2001; 19:3280-3287.
7. Voorzanger-Rousselot N, Favrot M, Blay J Y. Resistance to cytotoxic chemotherapy induced by CD40 ligand in lymphoma cells. Blood 1998; 92:3381-3387.
8. Buske C, Twiling A, Gogowski G, et al. In vitro activation of low-grade non-Hodgkin's lymphoma by murine fibroblasts, IL-4, anti-CD40 antibodies and the soluble CD40 ligand. Leukemia 1997; 11:1862-1867.
9. Gerondakis S, Strasser A. The role of Re1/NF-kappaB transcription factors in B lymphocyte survival. Semin Immunol 2003; 15:159-166.
10. Franklin R A, McCubrey J A. Kinases: positive and negative regulators of apoptosis. Leukemia 2000; 14:2019-2034.
11. Craxton A, Shu G, Graves J D, Saklatvala J, Krebs E G, Clark E A. p38 MAPK is required for CD40-induced gene expression and proliferation in B lymphocytes. J Immunol 1998; 161:3225-3236.
12. Gauld S B, Blair D, Moss C A, Reid S D, Harnett M M. Differential roles for extracellularly regulated kinase-mitogen-activated protein kinase in B cell antigen receptor-induced apoptosis and CD40-mediated rescue of WEHI-231 immature B cells. J Immunol 2002; 168:3855-3864.
13. Chang H, Blondal J A, Benchimol S, Minden M D, Messner H A. p53 mutations, c-myc and bcl-2 rearrangements in human non-Hodgkin's lymphoma cell lines. Leuk Lymphoma 1995; 19:165-171.
14. Epstein A L, Kaplan H S. Feeder layer and nutritional requirements for the establishment and cloning of human malignant lymphoma cell lines. Cancer Res 1979; 39:1748-1759.
15. Clark E A, Yip T C, Ledbetter J A, et al. CDw40 and BLCa-specific monoclonal antibodies detect two distinct molecules which transmit progression signals to human B lymphocytes. Eur J Immunol 1988; 18:451-457.
16. Hollmann A C, Gong Q, Owens T. CD40-mediated apoptosis in murine B-lymphoma lines containing mutated p53. Exp Cell Res 2002; 280:201-211.
17. Hollmann C A, Kittrell F S, Medina D, Butel J S. Wnt-1 and int-2 mammary oncogene effects on the β-catenin pathway in immortalized mouse mammary epithelial cell lines are not sufficient for tumorigenesis. Oncogene 2001; 20:7645-7657.
18. Kurland J F, Voehringer D W, Meyn R E. The MEK/ERK pathway acts upstream of NF kappa B1 (p50) homodimer activity and Bcl-2 expression in a murine B-cell lymphoma cell line. MEK inhibition restores radiation-induced apoptosis. J Biol Chem 2003; 278:32465-32470.
19. Novak J P, Sladek R, Hudson T J. Characterization of variability in large-scale gene expression data: implications for study design. Genomics 2002; 79:104-113.
20. Bolstad B M, Irizarry R A, Astrand M, Speed T P. A comparison of normalization methods for high density oligonucleotide array data based on variance and bias. Bioinformatics 2003; 19:185-193.
21. Benjamini Y, Drai D, Elmer G, Kafkafi N, Golani I. Controlling the false discovery rate in behavior genetics research. Behav Brain Res 2001; 125:279-284.
22. Donze O, Deng J, Curran J, Sladek R, Picard D, Sonenberg N. The protein kinase PKR: a molecular clock that sequentially activates survival and death programs. Embo J 2004; 23:564-571.
23. Sturn A, Quackenbush J, Trajanoski Z. Genesis: cluster analysis of microarray data. Bioinformatics 2002; 18:207-208.
24. Karolchik D, Baertsch R, Diekhans M, et al. The UCSC Genome Browser Database. Nucleic Acids Res 2003; 31:51-54.
25. Rozen S, Skaletsky H. Primer 3 on the WWW for general users and for biologist programmers. Methods Mol Biol 2000; 132:365-386.
26. Altschul S F, Gish W, Miller W, Myers E W, Lipman D J. Basic local alignment search tool. J Mol Biol 1990; 215:403-410.
27. Von Knethen A, Abts H, Kube D, Diehl V, Tesch H. Expression of p56lck in B-cell neoplasias. Leuk Lymphoma 1997; 26:551-562.
28. Grammer A C, Swantek J L, McFarland R D, Miura Y, Geppert T, Lipsky P E. TNF receptor-associated factor-3 signaling mediates activation of p38 and Jun N-terminal kinase, cytokine secretion, and Ig production following ligation of CD40 on human B cells. J Immunol 1998; 161:1183-1193.
29. Salojin K V, Zhang J, Delovitch T L. TCR and CD28 are coupled via ZAP-70 to the activation of the Vav/Rac-1-/PAK-1/p38 MAPK signaling pathway. J Immunol 1999; 163:844-853.
30. Song J S, Haleem-Smith H, Arudchandran R, et al. Tyrosine phosphorylation of Vav stimulates IL-6 production in mast cells by a Rac/c-Jun N-terminal kinase-dependent pathway. J Immunol 1999; 163:802-810.
31. Lin K, Abraham K M. Targets of p56(lck) activity in immature thymoblasts: stimulation of the Ras/Raf/MAPK pathway. Int Immunol 1997; 9:291-306.
32. Liu Y, Bishop A, Witucki L, et al. Structural basis for selective inhibition of Src family kinases by PP1. Chem Biol 1999; 6:671-678.
33. Favata M F, Horiuchi K Y, Manos E J, et al. Identification of a novel inhibitor of mitogen-activated protein kinase kinase. J Biol Chem 1998; 273:18623-18632.
34. Davies C C, Mason J, Wakelam M J, Young L S, Eliopoulos A G. Inhibition of phosphatidylinositol 3-kinase- and ERK MAPK-regulated protein synthesis reveals the pro-apoptotic properties of CD40 ligation in carcinoma cells. J Biol Chem 2004; 279:1010-1019.
35. Perfetti V, Vignarelli M C, Bellotti V, et al. Membrane CD22 defines circulating myeloma-related cells as mature or later B cells. Lab Invest 1997; 77:333-344.
36. Burrows P D, Cooper M D. B cell development and differentiation. Curr Opin Immunol 1997; 9:239-244.
37. Williamson J M, Grigor I, Smith M E, et al. Cluster differentiation antigen expression, proliferative activity and clinical stage in centroblastic centrocytic lymphomas. J Pathol 1986; 150:51-59.
38. Shao N, Chai Y, Cui J Q, et al. Induction of apoptosis by Elk-1 and deltaElk-1 proteins. Oncogene 1998; 17:527-532.
39. Wang Y H, Nomura J, Faye-Petersen O M, Cooper M D. Surrogate light chain production during B cell differentiation: differential intracellular versus cell surface expression. J Immunol 1998; 161:1132-1139.
40. Tarlinton D. Germinal centers: a second childhood for lymphocytes. Curr Biol 1997; 7:R155-159.
41. Zheng B, Fiumara P, Li Y V, et al. MEK/ERK pathway is aberrantly active in Hodgkin disease: a signaling pathway shared by CD30, CD40, and RANK that regulates cell proliferation and survival. Blood 2003; 102:1019-1027.
42. Tanimoto Y, Kizaki H. Proteasome inhibitors block Ras/ERK signaling pathway resulting in the downregulation of Fas ligand expression during activation-induced cell death in T cells. J Biochem (Tokyo) 2002; 131:319-326.
43. Uckun F M, Schieven G L, Dibirdik I, Chandan-Langlie M, Tuel-Ahlgren L, Ledbetter J A. Stimulation of protein tyrosine phosphorylation, phosphoinositide turnover, and multiple previously unidentified serine/threonine-specific protein kinases by the Pan-B-cell receptor CD40/Bp50 at discrete developmental stages of human B-cell ontogeny. J Biol Chem 1991; 266:17478-17485.
44. Gruss H J, Herrmann F, Gattei V, Gloghini A, Pinto A, Carbone A. CD40/CD40 ligand interactions in normal, reactive and malignant lympho-hematopoietic tissues. Leuk Lymphoma 1997; 24:393-422.
45. Alizadeh A A, Eisen M B, Davis R E, et al. Distinct types of diffuse large B-cell lymphoma identified by gene expression profiling. Nature 2000; 403:503-511.
46. Hans C P, Weisenburger D D, Greiner T C, et al. Confirmation of the molecular classification of diffuse large B-cell lymphoma by immunohistochemistry using a tissue microarray. Blood 2004; 103:275-282.
47. Law C L, Gordon K A, Collier J, et al. Preclinical antilymphoma activity of a humanized anti-CD40 monoclonal antibody, SGN-40. Cancer Res 2005; 65:8331-8338.
48. Karp J E, Lancet J E, Kaufmann S H, et al. Clinical and biologic activity of the farnesyltransferase inhibitor R115777 in adults with refractory and relapsed acute leukemias: a phase 1 clinical-laboratory correlative trial. Blood 2001; 97:3361-3369.
49. He H, Hirokawa Y, Levitzki A, Maruta H. An anti-Ras cancer potential of PP1, an inhibitor specific for Src family kinases: in vitro and in vivo studies. Cancer J 2000; 6:243-248.
50. James J A, Smith M A, Court E L, et al. An investigation of the effects of the MEK inhibitor U0126 on apoptosis in acute leukemia. Hematol J 2003; 4:427-432.

### EXAMPLES

### Materials & Methods

### Lymphoma and Hybridoma Cell Lines

Diffuse large-cell lymphoma lines OCI-Ly1, OCI-Ly7, OCI-Ly8, and Su-DHL4 (13, 14) were provided by Dr. Neil Berinstein, Ontario Cancer Institute, Toronto, ON, Canada. A hybridoma line producing anti-human CD40 (clone G28.5) (15) was provided by Dr. Bruce Mazer, McGill University, Montreal, PQ, Canada. The cell lines were maintained in RPMI1640 medium (Sigma, Oakville, ON, Canada) supplemented with 10% bovine growth serum (VWR Canlab, Montreal, PQ, Canada), 0.2 mM glutamine, 0.05 mM β-mercaptoethanol, 100 U/mL penicillin and 100 μg/mL streptomycin in a 5% CO₂ atmosphere at 37° C. Antibodies against human CD40 (clone G28.5) and murine IgG (clone HB58) were purified from hybridoma supernatants with a Protein G sepharose column as described by the manufacturer (Amersham, Baie d'Urfe, PQ, Canada).

### CD40 Stimulation and Assessment of Viability

Cells were resuspended at 1×10⁵ cells/mL in RPMI 1640 medium supplemented as described above. Anti-CD40 antibody G28.5 and secondary crosslinking antibody HB58 were added to a final concentration of 10 μg/mL each, and the cells were incubated at 37° C. At 24 hour intervals aliquots of cells were harvested by centrifugation, washed once in phosphate buffered saline, and resuspended in FACS buffer. Non-permeabilized cells were stained by addition of 1 μg/mL propidium iodide, and the density of viable cells was determined using a FACScan flow cytometer and CellQuest software (Becton Dickinson, Mississauga, ON, Canada) set to count for a fixed 30-second time interval. To determine the fraction of cells that had undergone apoptosis, total intracellular DNA content was measured by propidium iodide staining of ethanol-permeabilized cells as previously described (16). Expression of CD40 on the cell surface was confirmed by staining non-permeabilized cells with a phycoerythrin-linked anti-CD40 antibody (clone 5C3, Becton-Dickinson) as per the manufacturer's directions, followed by flow cytometry.

### Growth Inhibition by Kinase Inhibitors

The src family kinase inhibitor PP1 and the MEK inhibitor U0126 were purchased from Biomol (Plymouth Meeting, Pa., USA), and from Cell Signaling Technology (Beverly, Mass., USA) respectively. Kinase inhibition assays were performed using cells seeded at a density of 5×10⁴ per mL in 96-well plates, to which kinase inhibitors were added to final concentrations of 10⁻⁴ to 10⁻⁸ M. The treated cells were incubated at 37° C. for 96 hours and cell viability was quantitated by MTT assay as previously described (17). For inhibition of CD40-mediated apoptosis, the MEK inhibitors U0126 (10 μM) and PD98059 (50 μM) as well as the p38 inhibitor SB203580 (10 μM) (Cell Signaling Technologies) were added 30 minutes prior to CD40 stimulation. The dose of kinase inhibitors used in this study have been previously shown to mediate target-specific effects in lymphocytes (18).

### Detection of Intracellular Phosphorylated ERK by Flow Cytometry

Cells were permeabilized in Cytofix/Cytoperm (Becton-Dickinson) for 20 minutes, and stained with anti-phospho-ERK antibody (Cell Signaling Technology #9101) and FITC-conjugated anti-rabbit secondary antibody (Cedarlane Laboratories, Hornby, ON, CA) following the manufacturers' instructions. The stained cells were analyzed by flow cytometry with a FACScan flow cytometer and CellQuest software.

### Preparation of Cell Extracts

Nondenatured whole cell lysates were prepared by sonicating cells in nondenaturing lysis buffer (20 mM Tris pH 7.5, 150 mM NaCl, 1 mM EDTA, 1 mM EGTA, 1% Triton-X 100, 1 mM Na₃VO₄) containing a protease inhibitor cocktail (Roche Diagnostics, Laval, PQ, Canada). Cytoplasmic-enriched extracts were prepared by lysing cells in hypotonic lysis buffer (10 mM Hepes pH 7.9, 1.5 mM MgCl₂, 100 mM KCl, 1 mM DTT, 1% protease inhibitor cocktail (Sigma, P8340)) followed by shearing through a 23 G needle to release the nuclei. The nuclei were pelleted at 450×g and the supernatant (cytoplasmic-enriched cell extract) was removed and stored at −80° C. Protein concentration was determined using the BCA protein assay (Pierce, Rockford, Ill., USA).

### Immunoprecipitation and Western Blot:

LCK was immunoprecipitated from nondenatured cell lysate with a polyclonal rabbit antibody (Cell Signaling Technology #2752) as recommended by the manufacturer. Proteins were separated by SDS-PAGE on a 14% polyacrylamide gel at 180V for 1 h and transferred to a nitrocellulose membrane by electrophoretic transfer at 100V for 1 h. Western blots were performed as previously described (16). Primary antibodies against total and phosphorylated ERK, JNK, and p38 (Cell Signaling Technology #9101, #9102, #9151, #9152, #9211 and #9212) were used at a dilution of 1:100, primary antibodies against LCK and activated src family kinases (Cell Signaling Technology #2752 and #2101) were diluted 1:500, and peroxidase-conjugated goat anti-mouse or donkey anti-rabbit secondary antibodies (BioCan Scientific, Mississauga, ON, Canada) were used at a dilution of 1:10000. The compositions of blocking and antibody dilution solutions were as specified by the manufacturer. Blots were immersed in Femto West ECL staining solution (Pierce) for 30 seconds and photographed with a Syngene chemiluminescence imaging system and GeneSnap software. Image size and contrast was adjusted using Canvas software.

### Expression Microarrays

Total RNA was prepared from cultured cells using Trizol reagent according to the manufacturer's instructions (Invitrogen, Carlsbad, Calif.). The integrity of the purified RNA was verified using an Agilent 2100 Bioanalyzer (Agilent Technologies, Palo Alto, Calif.). Probes for microarray analysis were prepared using 10 micrograms of total RNA and hybridized to Affymetrix HG-U113A Gene Chips (Affymetrix, Santa Clara, Calif.) as previously described (19). In order to minimize technical variability, RNA processing steps (RNA extraction, probe labeling and chip hybridization) were performed in parallel for each set of four RNA samples.

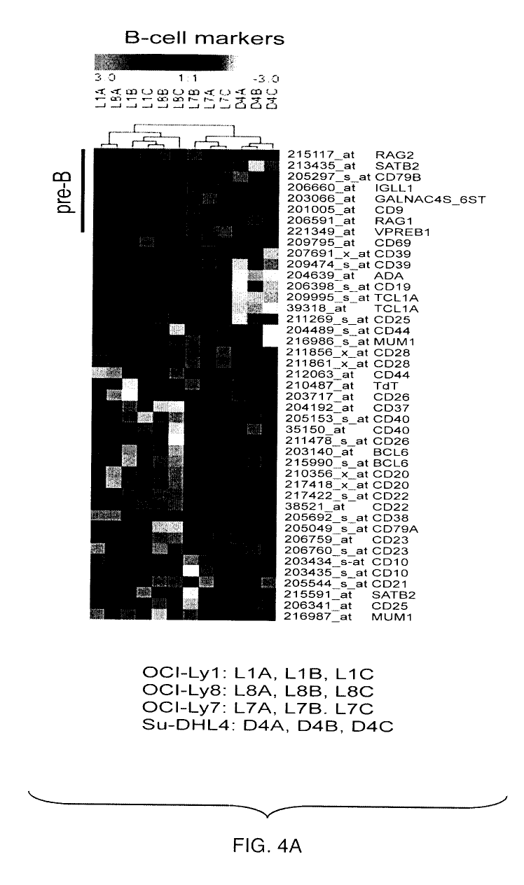

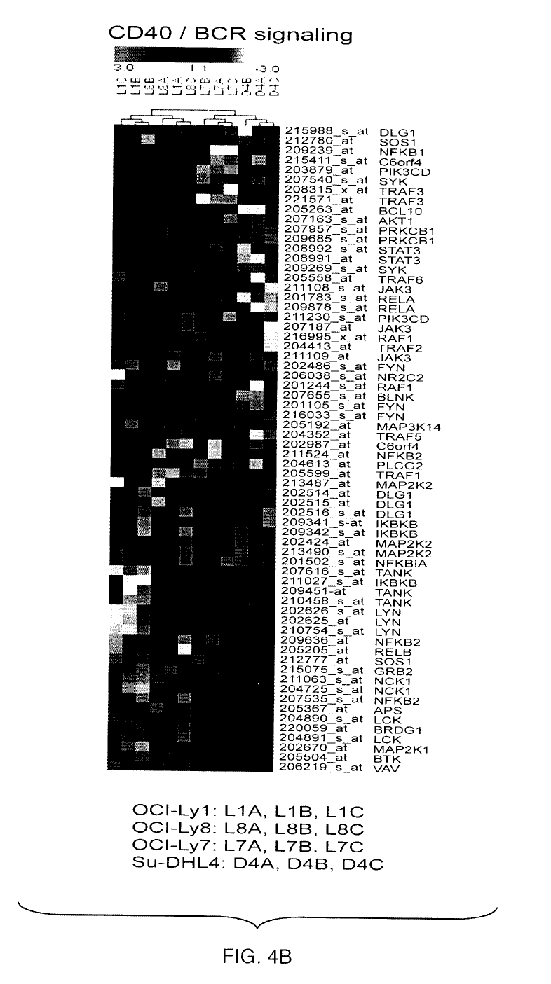

The hybridized arrays were scanned and raw data extracted using the Microarray Analysis Suite 5.0 (MAS5, Affymetrix, Santa Clara, Calif.). The raw data were normalized using RMAExpress (20) (http://stat-www.berkeley.edu/users/bolstad/RMAExpress/RMAExpress.html) and filtered to exclude genes that MAS5 did not identify as “Present” in any expression profile. Differentially expressed genes were identified by performing a t-test between each pair of CD40-sensitive and CD40-resistant lines. False positive error correction was performed to maintain a 10% false discovery rate (FDR) (21). Genes whose expression differed significantly and showed the same direction of change in all four comparisons were considered to be differentially regulated between sensitive and resistant lines. Functional assessment of these expression differences was performed using contingency table analysis, based on hand-curated lists of NFκB targets ((22) and references obtained from http://people.bu.edu/gilmore/nf-kb)) genes involved in CD40 and BCR signal transduction, B-cell maturation markers and the Gene Ontology classification of proteins implicated in apoptosis (Affymetrix, Santa Clara, Calif.). See Tables 4A, 4B, 5A, 5B, 6A, 6B, 7A, 7B, 8A, 8B, 9A, 9B, 10A and 10B. FIGS. 4A and 4B illustrating relevant expression profiles were prepared using Genesis (http://genome.tugraz.at/Software/GenesisCenter.html) (23).

### Semi-Quantitative RT-PCR

RNA isolation and cDNA synthesis was performed as for the microarray analyses. Primers were designed to span introns to ensure specificity for cDNA as opposed to genomic DNA sequences. Intron/exon junctions were identified by use of the UCSC genome browser (http://www.genome.ucsc.edu) (24). Primers were designed using Primer3 software (http://frodo.wi.mit.edu/cgi-bin/primer3/primer3_www.cgi) (25) and their specificity was verified by performing a BLAST search (http://www.ncbi.nlm.nih.gov/BLAST/) of the NCBI “nr” nucleotide sequence database (26). The following primer pairs were used:

(SEQ ID NO:1)

BTK 5′TGATGAAGGGCCTCTCTACG;

(SEQ ID NO:2)

3′CGGTGAGAACTCCCAGGTTT;

(SEQ ID NO:3)

CD9 5′GACTATGGCTCCGATTCGAC;

(SEQ ID NO:4)

3′AGGAAGCCGAAGAACAGTCC;

(SEQ ID NO:5)

GAPDH 5′GAAGGTGAAGGTCGGAGTCA;

(SEQ ID NO:6)

3′GACAAGCTTCCCGTTCTCAG;

(SEQ ID NO:7)

LCK 5′ATGAACTGGTCCGCCATTAC;

(SEQ ID NO:8)

3′CCCGTTGTAGTACCCCATCC;

(SEQ ID NO:9)

RAG1 5′AGAGAGCAGAGAACACACTTTGC;

(SEQ ID NO:10)

3′GATCTCACCCGGAACAGCTT;

(SEQ ID NO:11)

VAV1 5′CACCTGCTGTGAGAAGTTCG

(SEQ ID NO:12)

3′GTCGGACAGGCCACTGTAGAT.

PCR amplification was performed with 0.01 U/μL Taq DNA polymerase (Sigma) in PCR buffer (Sigma) containing 2 mM MgCl₂, 0.25 μg/μL bovine serum albumin (New England Biolabs), and 0.1 mM each dATP, DCTP, dGTP, and dTTP (Amersham). Amplification was performed with 25 cycles (GAPDH, BTK) or 35 cycles (all other primers) of 30 seconds each at 95° C., 58° C., and 72° C. PCR products were detected by gel electrophoresis and ethidium bromide staining. Images were acquired with a Syngene gel documentation system and GeneSnap software. Gene expression was quantitated with GeneTools software, using PCR products amplified from a cDNA dilution series as a standard curve. Images were transferred to Canvas software for adjustment of contrast and image size.

### Example 1

### Susceptibility of DLCBL Cell Lines to CD40-Mediated Cell Death

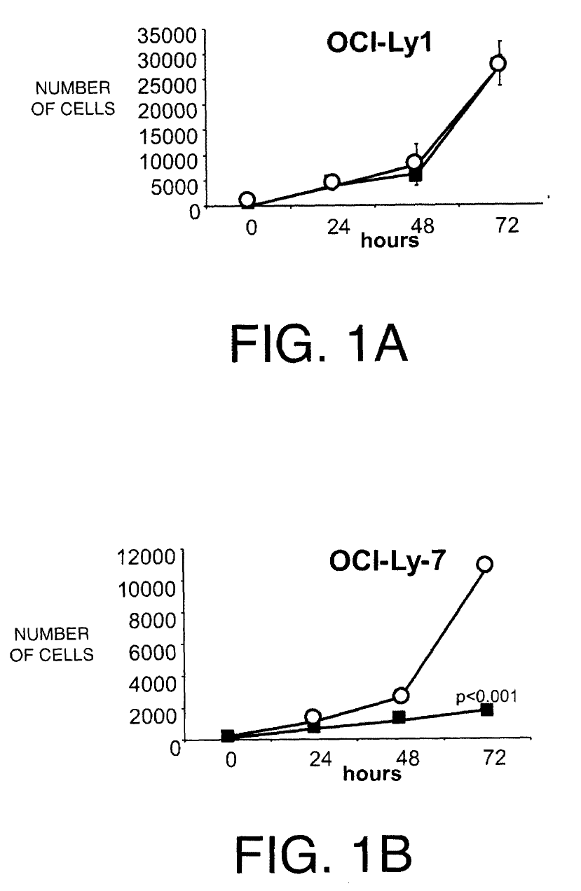

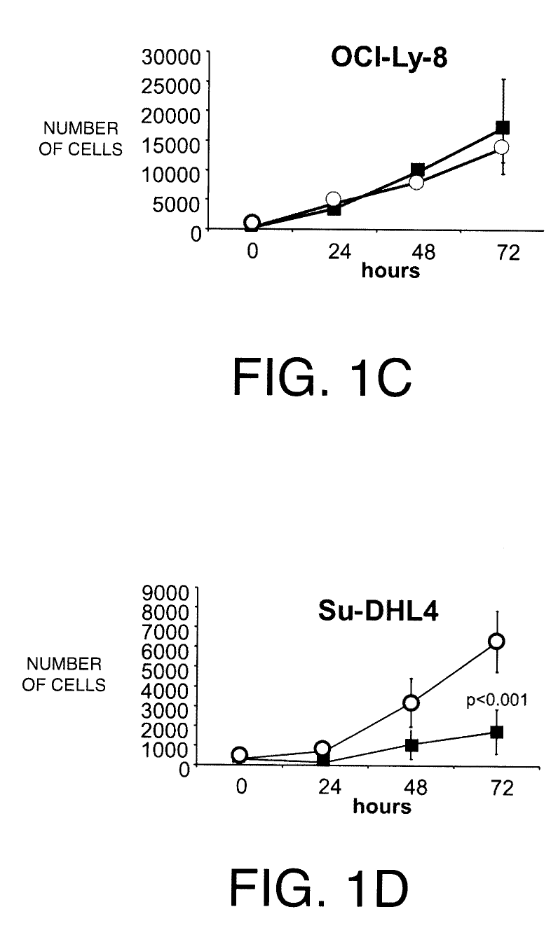

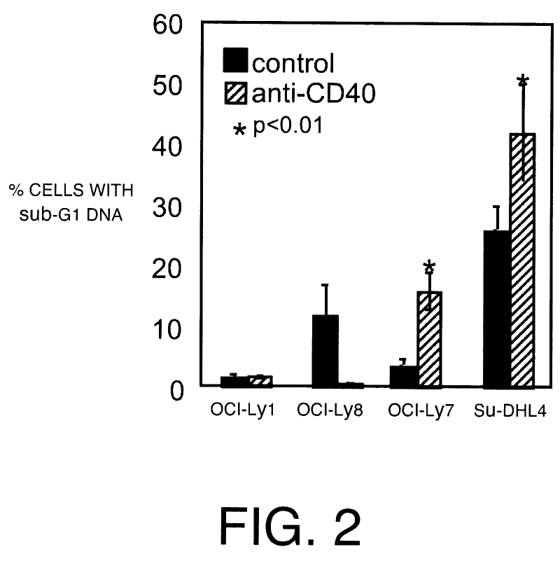

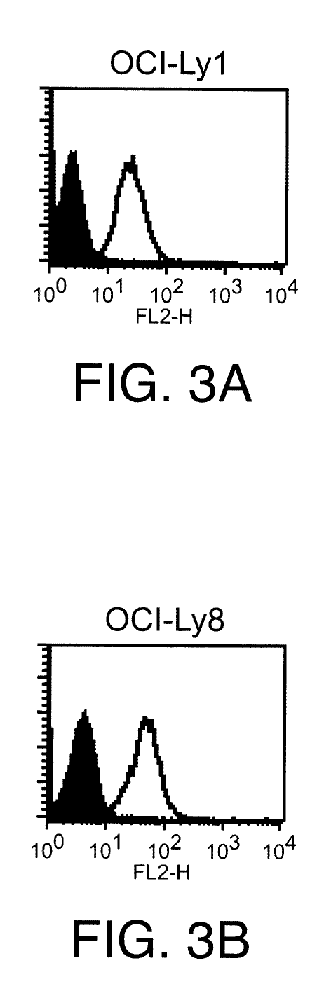

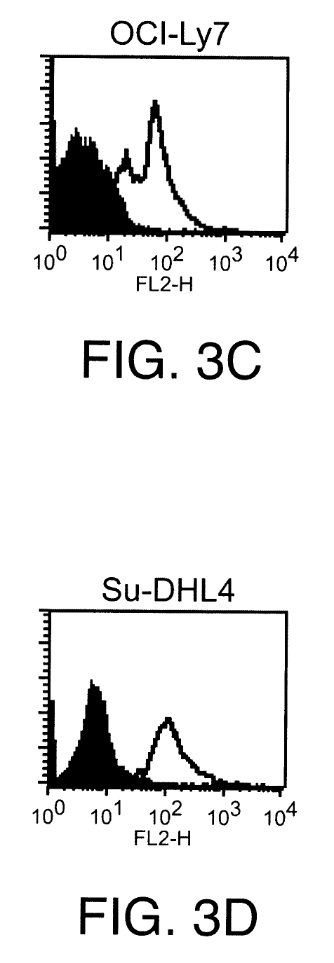

Diffuse large-cell B-lymphoma cell lines were screened for susceptibility to CD40-mediated cell death. The number of viable OCI-Ly7 and Su-DHL4 cells was reduced after 72 hours of exposure to crosslinked anti-CD40 antibody, whereas the viability of OCI-Ly1 and OCI-Ly8 cells was unaffected (FIGS. 1A-1D). CD40 stimulation also increased the proportion of cells with sub-G1 DNA content at 48 hours in OCI-Ly7 and Su-DHL4, but not OCI-Ly1 or OCI-Ly8 (FIG. 2). Expression of CD40 on the cell surface was confirmed by flow cytometry for all four cell lines (FIGS. 3A-3D).

### Example 2

### Gene Expression Profiling of CD40-Sensitive and CD40-Resistant DLCBL Lines

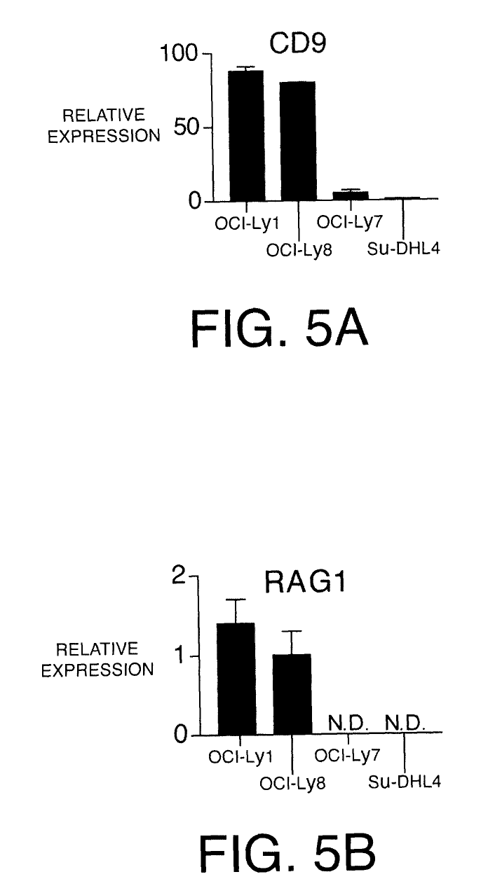

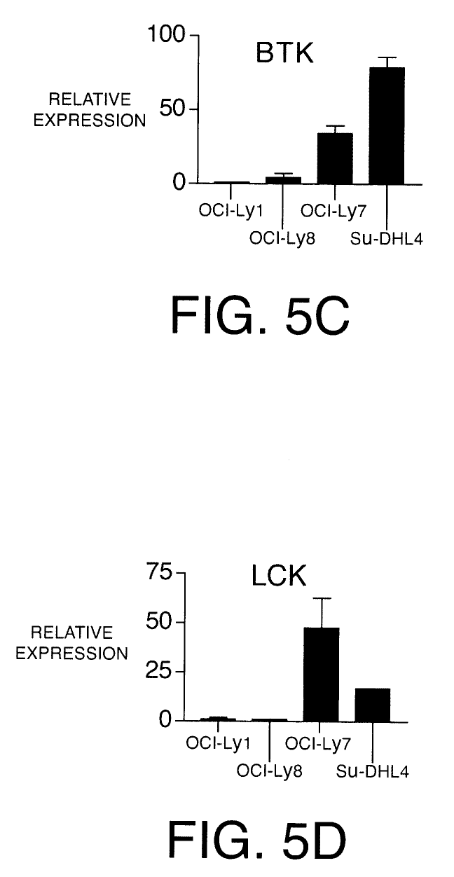

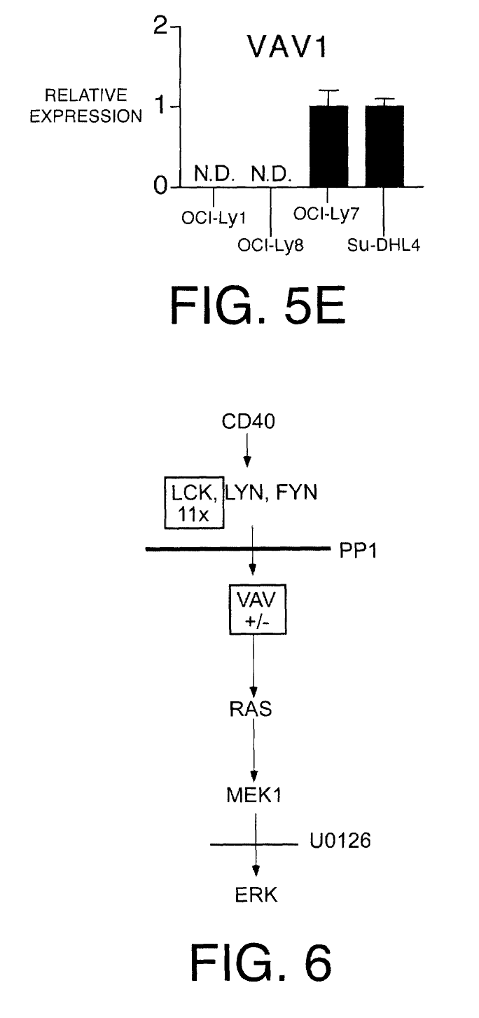

To identify pre-existing gene expression patterns that may predict susceptibility to CD40-mediated cell death, RNA from unstimulated OCI-Ly1, OCI-Ly8, OCI-Ly7, and Su-DHL4 cells was analyzed on Affymetrix U133A oligonucleotide arrays. In total, 304 genes were differentially expressed among CD40-sensitive and CD40-resistant cells (FIGS. 4A and 4B; Tables 2A, 2B, 3A and 3B. Further analysis was restricted to subsets of genes likely to be involved in CD40 signaling, including regulators of apoptosis, B-cell specific genes, and NFκB-regulated genes. Susceptibility to CD40-mediated apoptosis was not associated with differences in expression of pro-apoptotic or anti-apoptotic transcripts, and there was no statistically significant difference in expression of NFκB-regulated genes (Table 1). Differences in expression of transcripts encoding B-cell maturation-specific genes and members of the CD40 signaling pathway were statistically significant. CD40 resistant OCI-Ly1 and OCI-Ly8 cells expressed significantly higher levels of pre-B cell genes including RAG1, RAG2, IGLL1, CD9, and VPREB1, whereas CD40-sensitive OCI-Ly7 and Su-DHL4 cells expressed higher levels of CD22 and CD38. CD40-sensitive cells also expressed higher levels of several genes in the CD40 signaling pathway, including Bruton's tyrosine kinase, VAV, LYN, LCK, and MEK1/MAP2K1. Differential expression of several genes was confirmed by RT-PCT (FIGS. 5A-5E). Transcripts for two of these genes could only be detected in one group of cell lines: RAG1 was easily detectable in CD40-resistant OCI-Ly1 and OCI-Ly8 cells but absent in CD40-sensitive OCI-Ly7 and Su-DHL4 cells, and VAV1 was present in CD40-sensitive but undetectable in CD40-resistant cells.

TABLE 1
Differences in gene expression among CD40-sensitive
and CD40-resistant DLCBL lines

| | number | differences | | | |
|---|---|---|---|---|---|
| Group | of genes | expected | observed | Chi Square | p-value |
| Total unique markers | 8003 | N.A. | 298 | N.A. | N.A. |
| Pre-B markers* | 14 | 0.5 | 5.0 | 38.5 | <0.001 |
| Mature, activated B-cell* | 18 | 0.7 | 3.0 | 8.1 | <0.005 |
| Pan-B cell* | 10 | 0.4 | 1 | 1.1 | n.s. |
| CD40/BCR signaling* | 35 | 1.3 | 5.0 | 10.5 | <0.005 |
| NFκB regulated * | 117 | 4.4 | 6.0 | 0.6 | n.s. |
| Apoptosis* | 90 | 3.4 | 3 | 0.0 | n.s. |
| Anti-apoptosis* | 56 | 1.1 | 0 | 1.1 | n.s. |

N.A. = not applicable, n.s. = not significant.
*Genes in these groups are listed in Tables 4A, 4B, 5A, 5B, 6A, 6B, 7A, 7B, 8A, 8B, 9A, 9B, 10A and 10B.

TABLE 2A
Genes With Increased Expression in CD40-Sensitive Cell Lines

| | Log2 fluorescence intensity | | | | | | | | | | | |
|---|---|---|---|---|---|---|---|---|---|---|---|---|
| | | | | | | | | | | | | D4 |
| Probesets | Ly1 (A1) | Ly1 (A2) | Ly1 (A3) | Ly7 (B1) | Ly7 (B2) | Ly7 (B3) | Ly8 (C1) | Ly8 (C2) | Ly8 (C3) | D4 (D1) | D4 (D2) | (D3) |
| 200011_s_at | 9.37 | 9.43 | 9.33 | 9.70 | 9.59 | 9.63 | 9.31 | 9.23 | 9.40 | 10.31 | 10.30 | 10.42 |
| 200090_at | 8.94 | 8.60 | 8.88 | 9.95 | 9.99 | 9.90 | 8.77 | 8.54 | 8.83 | 10.35 | 10.22 | 10.44 |
| 200625_s_at | 9.62 | 9.46 | 9.54 | 10.25 | 10.12 | 10.13 | 9.77 | 9.77 | 9.66 | 10.06 | 10.22 | 10.15 |
| 200679_x_at | 10.87 | 11.27 | 11.16 | 12.13 | 12.00 | 12.04 | 10.97 | 11.15 | 11.22 | 12.13 | 12.12 | 12.32 |
| 200762_at | 6.64 | 6.56 | 6.91 | 9.22 | 9.03 | 8.98 | 6.48 | 6.50 | 7.17 | 8.51 | 8.16 | 8.78 |
| 200833_s_at | 9.81 | 9.59 | 9.83 | 10.35 | 10.24 | 10.47 | 9.82 | 9.74 | 9.92 | 10.57 | 10.57 | 10.79 |
| 200998_s_at | 5.24 | 5.07 | 5.26 | 6.84 | 7.21 | 6.83 | 6.05 | 5.70 | 5.77 | 6.75 | 6.89 | 7.11 |
| 201029_s_at | 7.24 | 7.18 | 7.18 | 9.63 | 9.66 | 9.37 | 8.31 | 7.88 | 8.11 | 10.81 | 11.04 | 10.86 |
| 201077_s_at | 10.70 | 10.63 | 10.55 | 11.13 | 10.99 | 10.98 | 10.66 | 10.59 | 10.54 | 10.96 | 11.02 | 10.89 |
| 201089_at | 8.18 | 7.80 | 7.79 | 8.87 | 8.64 | 8.84 | 7.96 | 7.78 | 7.97 | 9.21 | 9.21 | 9.51 |
| 201090_x_at | 13.07 | 13.16 | 13.11 | 13.60 | 13.50 | 13.51 | 13.19 | 13.19 | 13.15 | 13.59 | 13.52 | 13.61 |
| 201209_at | 9.60 | 9.38 | 9.66 | 10.43 | 10.38 | 10.40 | 9.67 | 9.39 | 9.63 | 10.63 | 10.37 | 10.62 |
| 201260_s_at | 7.65 | 7.59 | 8.15 | 9.44 | 9.34 | 9.55 | 7.62 | 7.59 | 7.99 | 9.62 | 9.64 | 9.71 |
| 201375_s_at | 8.03 | 7.78 | 8.17 | 9.52 | 9.38 | 9.56 | 8.15 | 7.75 | 8.23 | 9.53 | 9.37 | 9.73 |
| 201405_s_at | 9.04 | 9.20 | 9.02 | 9.39 | 9.43 | 9.52 | 9.25 | 9.14 | 9.23 | 9.68 | 9.77 | 9.80 |
| 201422_at | 9.85 | 9.47 | 9.78 | 10.90 | 10.97 | 10.90 | 9.76 | 9.64 | 9.53 | 11.91 | 12.37 | 12.03 |
| 201453_x_at | 9.97 | 9.88 | 10.00 | 10.55 | 10.55 | 10.60 | 9.92 | 9.73 | 9.87 | 10.44 | 10.53 | 10.49 |
| 201503_at | 9.80 | 9.85 | 9.99 | 10.66 | 10.48 | 10.49 | 10.05 | 9.92 | 9.80 | 10.73 | 10.51 | 10.72 |
| 201561_s_at | 7.98 | 7.71 | 7.60 | 8.68 | 8.46 | 8.34 | 7.96 | 7.83 | 7.68 | 8.67 | 8.48 | 8.73 |
| 201584_s_at | 9.90 | 10.09 | 9.89 | 10.78 | 10.61 | 10.54 | 9.69 | 9.83 | 9.84 | 10.86 | 10.78 | 10.91 |
| 201663_s_at | 8.95 | 9.05 | 8.91 | 10.27 | 10.06 | 9.98 | 8.91 | 8.97 | 9.13 | 10.37 | 10.52 | 10.36 |
| 201664_at | 8.90 | 8.94 | 9.04 | 10.08 | 9.78 | 10.05 | 8.77 | 8.96 | 9.14 | 10.35 | 10.23 | 10.50 |
| 201764_at | 8.92 | 9.00 | 8.74 | 9.51 | 9.54 | 9.37 | 8.84 | 8.84 | 8.92 | 10.18 | 10.15 | 10.12 |
| 201954_at | 9.41 | 9.55 | 9.38 | 10.02 | 10.12 | 10.07 | 9.31 | 9.46 | 9.35 | 10.32 | 10.70 | 10.61 |
| 202016_at | 4.54 | 4.76 | 4.78 | 7.52 | 7.49 | 7.74 | 4.73 | 4.52 | 4.58 | 10.41 | 9.58 | 10.29 |
| 202078_at | 8.41 | 8.45 | 8.33 | 9.57 | 9.70 | 9.64 | 8.25 | 8.35 | 8.51 | 9.68 | 9.59 | 9.83 |
| 202154_x_at | 9.80 | 9.90 | 9.50 | 10.89 | 10.65 | 10.62 | 10.00 | 9.93 | 9.66 | 10.68 | 10.82 | 10.71 |
| 202174_s_at | 6.86 | 6.79 | 6.70 | 8.39 | 7.99 | 8.01 | 6.85 | 6.70 | 6.84 | 8.40 | 7.99 | 8.44 |
| 202263_at | 5.45 | 5.37 | 5.14 | 6.30 | 6.29 | 6.18 | 5.51 | 5.50 | 5.50 | 6.18 | 6.12 | 6.00 |
| 202313_at | 8.38 | 8.26 | 8.32 | 9.18 | 9.01 | 8.98 | 8.39 | 8.32 | 8.20 | 9.26 | 9.55 | 9.30 |
| 202355_s_at | 7.17 | 7.16 | 7.06 | 7.80 | 7.59 | 7.67 | 7.21 | 7.08 | 7.28 | 7.81 | 8.14 | 8.09 |
| 202356_s_at | 7.53 | 7.44 | 7.55 | 7.83 | 7.81 | 7.75 | 7.52 | 7.51 | 7.58 | 8.13 | 8.33 | 8.31 |
| 202415_s_at | 8.09 | 8.36 | 7.98 | 9.19 | 9.13 | 8.92 | 8.28 | 8.33 | 8.23 | 9.03 | 9.32 | 9.14 |
| 202467_s_at | 8.94 | 8.88 | 9.18 | 10.05 | 9.89 | 10.07 | 8.87 | 8.86 | 9.12 | 9.82 | 9.58 | 9.89 |
| 202475_at | 9.26 | 9.37 | 9.22 | 10.27 | 10.21 | 10.23 | 9.26 | 9.25 | 9.31 | 9.86 | 9.97 | 10.00 |
| 202484_s_at | 8.88 | 9.10 | 9.28 | 10.11 | 10.08 | 10.27 | 8.88 | 9.06 | 9.35 | 10.94 | 10.80 | 11.22 |
| 202675_at | 7.76 | 8.03 | 7.95 | 9.19 | 8.88 | 9.09 | 8.01 | 7.77 | 8.19 | 9.27 | 8.98 | 9.26 |
| 202691_at | 8.45 | 8.55 | 8.62 | 9.26 | 9.00 | 9.22 | 8.38 | 8.37 | 8.61 | 9.08 | 9.07 | 9.33 |
| 202736_s_at | 9.89 | 9.89 | 9.78 | 10.04 | 10.12 | 10.14 | 9.78 | 9.61 | 9.80 | 10.53 | 10.78 | 10.79 |
| 2028_s_at | 6.95 | 7.16 | 6.88 | 8.31 | 8.08 | 8.14 | 6.86 | 7.02 | 7.08 | 7.64 | 7.90 | 7.85 |
| 202816_s_at | 6.40 | 6.15 | 6.53 | 8.64 | 8.44 | 8.69 | 6.25 | 5.98 | 6.49 | 7.12 | 7.27 | 7.61 |
| 203046_s_at | 8.39 | 8.49 | 8.45 | 9.02 | 9.01 | 8.87 | 8.38 | 8.42 | 8.39 | 8.99 | 9.11 | 9.17 |
| 203314_at | 7.15 | 7.23 | 7.05 | 7.77 | 7.96 | 7.88 | 6.98 | 7.21 | 7.13 | 7.91 | 8.06 | 7.83 |
| 203534_at | 8.65 | 8.60 | 8.47 | 9.58 | 9.43 | 9.48 | 8.61 | 8.35 | 8.61 | 10.11 | 9.79 | 10.20 |
| 203537_at | 7.89 | 7.62 | 7.65 | 8.79 | 8.78 | 8.65 | 7.63 | 7.38 | 7.67 | 8.91 | 8.69 | 9.07 |
| 203605_at | 7.59 | 7.47 | 7.72 | 8.82 | 8.64 | 8.77 | 7.79 | 7.67 | 7.95 | 8.39 | 8.50 | 8.74 |
| 203729_at | 5.49 | 5.29 | 5.32 | 8.16 | 8.41 | 8.13 | 5.33 | 5.24 | 5.18 | 6.98 | 7.15 | 7.29 |
| 203905_at | 8.59 | 8.68 | 8.76 | 9.41 | 9.51 | 9.46 | 8.72 | 8.67 | 8.74 | 9.94 | 10.28 | 10.13 |
| 203941_at | 6.50 | 6.61 | 6.60 | 7.23 | 7.40 | 7.20 | 6.71 | 6.72 | 6.67 | 7.42 | 7.64 | 7.66 |
| 203972_s_at | 6.80 | 6.80 | 6.94 | 7.85 | 7.80 | 7.95 | 6.85 | 6.85 | 7.12 | 7.70 | 7.81 | 8.06 |
| 204057_at | 7.68 | 7.44 | 7.69 | 9.00 | 8.95 | 8.81 | 7.14 | 7.39 | 7.21 | 9.43 | 9.64 | 9.29 |
| 204581_at | 7.31 | 6.65 | 6.97 | 9.29 | 9.12 | 9.07 | 7.64 | 6.88 | 7.08 | 9.45 | 9.76 | 9.57 |
| 204604_at | 5.47 | 5.43 | 5.71 | 8.58 | 8.55 | 8.14 | 5.64 | 5.56 | 5.23 | 9.11 | 9.42 | 9.48 |
| 204674_at | 8.75 | 8.97 | 9.06 | 10.26 | 10.28 | 10.09 | 8.84 | 9.22 | 9.24 | 10.04 | 10.32 | 10.00 |
| 204867_at | 7.38 | 6.59 | 6.96 | 8.86 | 8.85 | 8.45 | 7.55 | 6.94 | 7.12 | 9.85 | 10.01 | 9.82 |
| 204891_s_at | 6.82 | 6.23 | 6.68 | 10.30 | 10.26 | 10.18 | 6.71 | 6.30 | 6.10 | 9.51 | 9.88 | 9.69 |
| 205022_s_at | 6.08 | 5.78 | 6.02 | 7.04 | 7.25 | 6.80 | 6.03 | 5.95 | 6.09 | 6.62 | 6.79 | 6.94 |
| 205245_at | 6.00 | 5.94 | 6.00 | 6.64 | 6.73 | 6.60 | 5.92 | 6.06 | 5.85 | 6.59 | 6.59 | 6.61 |
| 205356_at | 7.94 | 8.07 | 7.96 | 9.08 | 8.84 | 8.99 | 7.79 | 8.02 | 8.15 | 8.64 | 8.60 | 8.84 |
| 205367_at | 7.63 | 7.61 | 7.50 | 10.59 | 10.54 | 10.27 | 7.14 | 7.60 | 7.42 | 10.20 | 10.20 | 10.16 |
| 205412_at | 9.28 | 9.28 | 9.38 | 9.95 | 10.02 | 10.09 | 9.47 | 9.38 | 9.63 | 10.44 | 10.45 | 10.57 |
| 205457_at | 5.41 | 5.47 | 5.53 | 6.14 | 6.37 | 6.23 | 5.49 | 5.36 | 5.55 | 5.97 | 6.06 | 5.84 |
| 205504_at | 5.84 | 5.90 | 6.11 | 8.32 | 8.16 | 7.96 | 6.01 | 5.67 | 5.89 | 8.53 | 8.41 | 8.56 |
| 205685_at | 6.06 | 5.77 | 5.89 | 6.40 | 6.58 | 6.49 | 5.93 | 5.95 | 6.03 | 7.06 | 7.36 | 7.26 |
| 205692_s_at | 6.94 | 7.34 | 7.45 | 9.32 | 9.34 | 9.36 | 6.89 | 7.41 | 7.26 | 9.01 | 9.14 | 8.98 |
| 205748_s_at | 8.18 | 8.10 | 8.03 | 8.73 | 8.62 | 8.60 | 8.28 | 8.08 | 8.26 | 8.79 | 8.98 | 8.82 |
| 205770_at | 7.11 | 7.24 | 7.26 | 8.30 | 8.62 | 8.32 | 7.23 | 7.29 | 7.42 | 8.76 | 8.91 | 8.76 |
| 206060_s_at | 5.22 | 5.09 | 5.27 | 6.79 | 7.21 | 7.33 | 5.18 | 4.90 | 5.17 | 6.47 | 6.67 | 6.87 |
| 206255_at | 6.46 | 6.73 | 6.48 | 8.89 | 8.95 | 8.84 | 5.88 | 6.01 | 6.12 | 9.20 | 8.91 | 9.24 |
| 206296_x_at | 8.63 | 8.05 | 8.15 | 9.80 | 9.53 | 9.24 | 8.32 | 7.87 | 7.85 | 9.99 | 10.33 | 10.05 |
| 206571_s_at | 7.32 | 7.57 | 7.37 | 8.78 | 8.59 | 8.56 | 7.43 | 7.76 | 7.86 | 9.03 | 9.38 | 9.55 |
| 206976_s_at | 9.34 | 9.59 | 9.84 | 10.63 | 10.25 | 10.52 | 9.73 | 9.92 | 9.73 | 11.06 | 10.58 | 10.96 |
| 207121_s_at | 7.60 | 7.83 | 8.06 | 10.52 | 10.54 | 10.59 | 7.73 | 7.74 | 8.11 | 8.90 | 9.12 | 9.49 |
| 207419_s_at | 7.58 | 7.22 | 7.16 | 8.50 | 8.31 | 8.29 | 7.39 | 7.09 | 7.00 | 8.73 | 9.01 | 8.60 |
| 207480_s_at | 4.61 | 4.83 | 5.00 | 6.17 | 6.16 | 6.12 | 4.81 | 5.12 | 4.91 | 6.54 | 6.50 | 7.20 |
| 207630_s_at | 6.50 | 6.47 | 6.59 | 7.81 | 8.12 | 7.98 | 6.39 | 6.72 | 6.60 | 9.38 | 9.53 | 9.50 |
| 207760_s_at | 7.04 | 7.14 | 6.99 | 8.65 | 8.32 | 8.16 | 7.36 | 6.99 | 7.35 | 8.66 | 8.93 | 9.06 |
| 208459_s_at | 6.86 | 6.98 | 6.86 | 7.96 | 7.94 | 7.75 | 7.11 | 6.98 | 7.01 | 7.96 | 8.06 | 8.08 |
| 208642_s_at | 9.89 | 10.07 | 9.92 | 10.91 | 10.69 | 10.79 | 9.92 | 10.00 | 10.11 | 10.51 | 10.59 | 10.61 |
| 208643_s_at | 9.27 | 9.53 | 9.57 | 10.43 | 10.45 | 10.51 | 9.36 | 9.44 | 9.56 | 10.05 | 10.17 | 10.24 |
| 208644_at | 10.85 | 10.87 | 10.77 | 11.26 | 11.17 | 11.24 | 10.85 | 11.00 | 10.88 | 11.25 | 11.32 | 11.14 |
| 208679_s_at | 10.44 | 10.29 | 10.31 | 11.05 | 10.99 | 10.89 | 10.50 | 10.22 | 10.36 | 10.88 | 10.98 | 11.08 |
| 208857_s_at | 7.38 | 7.19 | 7.50 | 9.18 | 9.03 | 9.09 | 7.22 | 7.12 | 7.44 | 8.46 | 8.27 | 8.69 |
| 208875_s_at | 7.73 | 7.71 | 7.90 | 8.79 | 8.83 | 8.68 | 7.74 | 7.63 | 7.89 | 8.92 | 9.07 | 9.21 |
| 209075_s_at | 8.63 | 8.72 | 8.87 | 9.71 | 9.61 | 9.65 | 8.53 | 8.74 | 8.79 | 9.52 | 9.42 | 9.77 |
| 209229_s_at | 7.66 | 7.88 | 7.54 | 8.59 | 8.36 | 8.56 | 7.70 | 7.83 | 7.86 | 8.68 | 8.84 | 8.91 |
| 209251_x_at | 13.04 | 13.11 | 13.10 | 13.55 | 13.52 | 13.52 | 13.16 | 13.20 | 13.17 | 13.61 | 13.54 | 13.51 |
| 209316_s_at | 7.47 | 7.35 | 7.53 | 8.28 | 8.26 | 8.53 | 7.51 | 7.58 | 7.88 | 8.62 | 8.55 | 8.81 |
| 209471_s_at | 7.88 | 7.43 | 7.71 | 9.07 | 9.08 | 8.94 | 7.58 | 7.28 | 7.54 | 9.23 | 9.23 | 9.31 |
| 209517_s_at | 8.71 | 8.56 | 8.47 | 9.66 | 9.38 | 9.59 | 8.64 | 8.48 | 8.85 | 9.79 | 9.67 | 9.85 |
| 209569_x_at | 5.47 | 5.77 | 5.47 | 7.81 | 8.14 | 7.62 | 5.66 | 5.70 | 5.86 | 9.56 | 10.22 | 9.75 |
| 209714_s_at | 8.76 | 8.51 | 8.31 | 9.67 | 9.38 | 9.49 | 8.67 | 8.49 | 8.80 | 9.65 | 9.40 | 9.63 |
| 209765_at | 5.48 | 5.62 | 6.00 | 7.00 | 7.32 | 7.23 | 5.61 | 5.86 | 5.78 | 6.80 | 7.14 | 7.04 |
| 209932_s_at | 9.62 | 9.77 | 9.74 | 11.06 | 10.83 | 10.98 | 9.47 | 9.58 | 9.83 | 10.74 | 10.57 | 10.85 |
| 210981_s_at | 6.38 | 6.52 | 6.57 | 7.53 | 7.63 | 7.35 | 6.48 | 6.63 | 6.60 | 7.62 | 7.73 | 7.57 |
| 211043_s_at | 8.46 | 8.58 | 8.59 | 9.24 | 9.40 | 9.37 | 8.63 | 8.56 | 8.66 | 9.36 | 9.49 | 9.64 |
| 211066_x_at | 6.88 | 6.96 | 7.12 | 8.78 | 8.96 | 8.77 | 6.69 | 7.25 | 6.47 | 8.43 | 8.77 | 8.94 |
| 211072_x_at | 12.84 | 12.91 | 12.80 | 13.32 | 13.22 | 13.21 | 12.88 | 12.87 | 12.92 | 13.28 | 13.20 | 13.29 |
| 211593_s_at | 6.72 | 6.83 | 6.49 | 8.01 | 7.85 | 7.79 | 6.90 | 6.89 | 6.79 | 7.50 | 7.65 | 7.43 |
| 211686_s_at | 7.63 | 7.68 | 7.87 | 8.52 | 8.36 | 8.43 | 7.76 | 7.81 | 7.77 | 8.34 | 8.52 | 8.52 |
| 211945_s_at | 8.60 | 8.38 | 8.65 | 9.72 | 9.71 | 9.63 | 8.75 | 8.59 | 8.67 | 9.81 | 9.76 | 9.74 |
| 212066_s_at | 8.65 | 8.17 | 8.35 | 9.83 | 9.80 | 9.86 | 8.36 | 8.25 | 8.54 | 9.61 | 9.47 | 9.73 |
| 212085_at | 10.37 | 9.99 | 9.82 | 11.37 | 11.59 | 11.21 | 10.35 | 9.88 | 9.84 | 11.87 | 12.10 | 11.95 |
| 212094_at | 4.40 | 4.54 | 4.51 | 8.25 | 8.01 | 7.83 | 4.76 | 4.50 | 4.55 | 7.21 | 6.53 | 6.96 |
| 212313_at | 6.83 | 6.82 | 6.57 | 7.68 | 7.37 | 7.44 | 6.70 | 6.50 | 6.60 | 8.05 | 8.18 | 8.08 |
| 212350_at | 8.52 | 8.36 | 8.23 | 9.69 | 9.67 | 9.42 | 8.53 | 8.40 | 8.36 | 9.39 | 9.41 | 9.56 |
| 212372_at | 5.27 | 5.50 | 5.12 | 7.31 | 7.35 | 7.31 | 5.35 | 5.53 | 5.36 | 8.37 | 7.97 | 8.41 |
| 212443_at | 6.25 | 6.20 | 6.08 | 6.97 | 6.77 | 6.72 | 6.33 | 6.18 | 6.24 | 6.76 | 6.92 | 7.01 |
| 212573_at | 5.32 | 5.48 | 5.39 | 7.84 | 8.00 | 7.64 | 5.05 | 5.50 | 5.35 | 6.88 | 7.47 | 7.26 |
| 212587_s_at | 6.54 | 6.75 | 7.23 | 8.95 | 9.22 | 9.32 | 6.96 | 6.91 | 7.23 | 9.75 | 9.82 | 10.39 |
| 212588_at | 6.03 | 5.99 | 6.67 | 8.57 | 8.83 | 8.77 | 5.95 | 6.22 | 6.52 | 9.67 | 9.40 | 9.56 |
| 212619_at | 5.28 | 5.12 | 5.21 | 5.77 | 5.66 | 5.78 | 5.25 | 5.15 | 5.30 | 5.99 | 6.10 | 5.85 |
| 212646_at | 8.94 | 9.08 | 9.33 | 10.88 | 10.91 | 10.80 | 9.10 | 9.05 | 8.89 | 11.46 | 11.81 | 11.52 |
| 212656_at | 8.53 | 8.54 | 8.67 | 9.06 | 8.87 | 8.99 | 8.48 | 8.65 | 8.68 | 9.05 | 9.13 | 9.23 |
| 212735_at | 6.78 | 6.62 | 6.58 | 7.35 | 7.23 | 7.22 | 6.48 | 6.60 | 6.56 | 7.48 | 7.67 | 7.69 |
| 212812_at | 6.58 | 6.30 | 6.41 | 8.92 | 9.37 | 8.74 | 6.45 | 6.04 | 6.64 | 7.79 | 8.27 | 8.32 |
| 212826_s_at | 10.03 | 9.73 | 9.68 | 11.13 | 11.40 | 10.98 | 9.93 | 9.75 | 9.70 | 11.72 | 11.84 | 11.78 |
| 213093_at | 6.63 | 6.66 | 6.70 | 8.38 | 8.08 | 8.16 | 6.71 | 6.40 | 6.59 | 8.59 | 8.36 | 8.80 |
| 213106_at | 5.60 | 5.35 | 5.79 | 7.75 | 8.01 | 7.35 | 5.62 | 5.29 | 5.56 | 6.72 | 6.79 | 7.09 |
| 213476_x_at | 10.82 | 10.82 | 10.43 | 11.94 | 11.75 | 11.80 | 10.94 | 10.71 | 10.59 | 11.56 | 11.80 | 11.69 |
| 213504_at | 7.78 | 7.95 | 7.93 | 8.43 | 8.45 | 8.55 | 7.69 | 7.83 | 7.91 | 8.75 | 8.69 | 8.89 |
| 213603_s_at | 9.64 | 9.37 | 9.35 | 10.60 | 10.55 | 10.47 | 9.32 | 9.14 | 9.22 | 10.71 | 11.00 | 10.86 |
| 213646_x_at | 13.20 | 13.25 | 13.09 | 13.72 | 13.59 | 13.60 | 13.16 | 13.20 | 13.24 | 13.64 | 13.60 | 13.65 |
| 213906_at | 4.88 | 4.60 | 4.66 | 8.17 | 7.48 | 7.46 | 4.53 | 4.49 | 4.72 | 8.37 | 8.21 | 8.60 |
| 214157_at | 5.17 | 5.02 | 5.35 | 7.31 | 7.34 | 7.34 | 4.99 | 5.03 | 4.98 | 7.49 | 7.03 | 7.36 |
| 214219_x_at | 8.30 | 7.85 | 7.89 | 9.41 | 9.21 | 8.89 | 8.23 | 7.62 | 7.57 | 9.52 | 10.01 | 9.75 |
| 214339_s_at | 7.79 | 7.44 | 7.78 | 9.09 | 8.83 | 8.74 | 7.92 | 7.33 | 7.21 | 9.17 | 9.58 | 9.55 |
| 214553_s_at | 5.80 | 5.76 | 5.89 | 6.16 | 6.30 | 6.37 | 5.62 | 5.75 | 5.81 | 6.62 | 6.55 | 6.82 |
| 215023_s_at | 5.08 | 5.03 | 5.18 | 5.82 | 5.94 | 6.09 | 5.16 | 4.90 | 5.04 | 5.93 | 6.27 | 6.27 |
| 215158_s_at | 7.59 | 7.63 | 7.64 | 8.25 | 8.40 | 8.16 | 7.64 | 7.46 | 7.50 | 7.96 | 8.06 | 7.98 |
| 215836_s_at | 6.90 | 6.50 | 6.93 | 7.93 | 8.44 | 7.90 | 6.64 | 6.87 | 6.93 | 7.90 | 8.12 | 7.99 |
| 216241_s_at | 9.97 | 9.78 | 9.91 | 10.50 | 10.63 | 10.50 | 9.75 | 9.66 | 9.75 | 10.99 | 10.98 | 10.91 |
| 216321_s_at | 5.82 | 5.76 | 6.16 | 7.65 | 7.57 | 7.58 | 5.29 | 5.44 | 5.78 | 6.98 | 7.38 | 7.57 |
| 217118_s_at | 7.74 | 7.90 | 8.15 | 9.29 | 9.52 | 9.44 | 7.82 | 8.17 | 8.30 | 9.06 | 9.23 | 9.43 |
| 217898_at | 7.72 | 7.69 | 7.34 | 8.99 | 9.00 | 9.10 | 7.62 | 7.61 | 7.81 | 8.85 | 8.94 | 9.32 |
| 217933_s_at | 9.27 | 9.25 | 9.39 | 10.04 | 9.91 | 9.92 | 9.53 | 9.53 | 9.64 | 9.90 | 9.82 | 9.92 |
| 218039_at | 8.92 | 8.90 | 8.77 | 10.40 | 10.04 | 10.21 | 8.79 | 8.92 | 9.06 | 10.48 | 10.12 | 10.44 |
| 218096_at | 7.41 | 7.75 | 8.00 | 9.23 | 8.99 | 9.05 | 7.80 | 7.73 | 8.00 | 9.13 | 9.00 | 9.25 |
| 218250_s_at | 8.74 | 8.60 | 8.70 | 10.06 | 9.99 | 9.98 | 8.83 | 8.59 | 8.73 | 9.95 | 10.02 | 10.14 |
| 218287_s_at | 6.57 | 6.61 | 6.65 | 7.11 | 7.12 | 7.07 | 6.64 | 6.69 | 6.67 | 7.23 | 7.32 | 7.20 |
| 218336_at | 8.46 | 8.77 | 8.67 | 9.43 | 9.19 | 9.40 | 8.61 | 8.52 | 8.77 | 9.79 | 9.63 | 9.97 |
| 218404_at | 5.89 | 6.20 | 5.99 | 7.67 | 7.60 | 7.59 | 6.14 | 6.38 | 6.63 | 8.23 | 8.20 | 8.14 |
| 218473_s_at | 7.48 | 7.41 | 7.37 | 7.94 | 7.90 | 7.91 | 7.44 | 7.45 | 7.32 | 8.20 | 8.13 | 8.11 |
| 218640_s_at | 6.54 | 6.29 | 6.79 | 7.80 | 8.13 | 8.02 | 6.35 | 6.25 | 6.68 | 8.41 | 8.87 | 8.40 |
| 218654_s_at | 9.06 | 9.11 | 9.09 | 9.85 | 9.90 | 9.86 | 8.87 | 8.77 | 8.80 | 9.97 | 10.09 | 10.11 |
| 218680_x_at | 6.26 | 6.60 | 6.81 | 8.56 | 8.60 | 8.48 | 6.50 | 6.47 | 6.77 | 8.28 | 8.03 | 8.30 |
| 218802_at | 8.33 | 8.37 | 8.38 | 9.46 | 9.41 | 9.41 | 8.25 | 8.43 | 8.44 | 9.17 | 9.06 | 9.03 |
| 219220_x_at | 7.86 | 7.92 | 7.75 | 8.76 | 8.75 | 8.76 | 7.88 | 7.84 | 7.90 | 8.84 | 8.86 | 8.95 |
| 219304_s_at | 4.91 | 5.22 | 5.29 | 5.86 | 6.16 | 5.78 | 4.89 | 5.08 | 5.13 | 6.18 | 6.44 | 6.26 |
| 219428_s_at | 6.72 | 6.90 | 6.74 | 7.50 | 7.44 | 7.41 | 6.54 | 6.61 | 6.76 | 7.83 | 7.66 | 7.71 |
| 219551_at | 7.09 | 7.43 | 7.37 | 8.78 | 8.53 | 8.49 | 7.11 | 7.16 | 7.39 | 8.04 | 7.92 | 8.15 |
| 219911_s_at | 6.55 | 7.05 | 6.73 | 8.15 | 8.04 | 7.93 | 6.82 | 7.12 | 6.79 | 8.27 | 8.45 | 8.30 |
| 220059_at | 8.32 | 8.08 | 8.17 | 10.14 | 10.19 | 10.19 | 8.09 | 7.95 | 7.95 | 9.89 | 9.72 | 9.93 |
| 221059_s_at | 8.14 | 7.89 | 8.04 | 9.25 | 9.06 | 9.26 | 8.04 | 7.87 | 8.21 | 9.84 | 9.80 | 9.93 |
| 221080_s_at | 6.78 | 6.82 | 6.67 | 7.70 | 7.47 | 7.39 | 6.97 | 6.76 | 6.84 | 7.83 | 8.06 | 7.76 |
| 221483_s_at | 8.93 | 8.86 | 8.93 | 9.94 | 9.79 | 9.79 | 8.81 | 8.90 | 8.87 | 9.71 | 9.63 | 9.89 |
| 221558_s_at | 7.50 | 7.50 | 7.64 | 10.19 | 10.30 | 10.31 | 7.12 | 7.29 | 7.79 | 9.60 | 9.28 | 9.71 |
| 221692_s_at | 7.80 | 7.95 | 8.07 | 9.03 | 9.02 | 8.82 | 7.85 | 7.89 | 7.85 | 8.77 | 8.73 | 8.72 |
| 221893_s_at | 6.81 | 6.49 | 6.76 | 8.88 | 8.63 | 8.40 | 6.61 | 6.70 | 6.54 | 7.91 | 7.93 | 8.35 |
| 222065_s_at | 6.15 | 6.17 | 6.02 | 7.43 | 7.23 | 7.15 | 6.28 | 6.21 | 6.01 | 6.90 | 7.11 | 6.91 |
| 222199_s_at | 6.94 | 6.94 | 6.84 | 7.33 | 7.30 | 7.38 | 7.04 | 7.04 | 6.97 | 7.50 | 7.66 | 7.53 |
| 32541_at | 4.63 | 4.49 | 4.60 | 5.61 | 5.46 | 5.68 | 4.53 | 4.34 | 4.47 | 5.52 | 5.82 | 5.89 |
| 35820_at | 8.26 | 8.13 | 8.01 | 8.95 | 8.83 | 8.58 | 8.13 | 7.89 | 8.04 | 9.75 | 9.73 | 9.72 |
| 35974_at | 7.77 | 8.03 | 8.34 | 9.68 | 9.75 | 9.56 | 7.79 | 8.25 | 8.40 | 9.31 | 9.72 | 9.42 |
| 36564_at | 8.10 | 7.92 | 7.74 | 9.01 | 9.14 | 8.94 | 8.14 | 7.91 | 7.81 | 9.09 | 9.16 | 9.46 |
| 38521_at | 8.75 | 8.60 | 8.51 | 9.72 | 9.86 | 9.76 | 8.71 | 8.54 | 8.42 | 9.89 | 10.19 | 10.05 |
| 39835_at | 8.36 | 8.30 | 8.26 | 9.32 | 9.16 | 9.11 | 8.20 | 7.98 | 8.11 | 9.33 | 9.48 | 9.40 |
| 44120_at | 7.28 | 7.12 | 7.18 | 8.52 | 8.55 | 8.58 | 7.01 | 6.97 | 7.10 | 8.32 | 8.28 | 8.41 |
| 200911_s_at | 7.34 | 7.20 | 7.19 | 9.69 | 9.63 | 9.63 | 7.65 | 7.42 | 7.82 | 9.32 | 9.41 | 9.69 |
| 201028_s_at | 5.78 | 5.51 | 5.49 | 8.02 | 8.00 | 7.83 | 6.45 | 6.08 | 6.35 | 9.35 | 9.74 | 9.46 |
| 201328_at | 5.16 | 4.90 | 4.97 | 6.25 | 6.14 | 6.22 | 5.12 | 4.92 | 5.04 | 7.37 | 7.66 | 8.08 |
| 201425_at | 5.63 | 5.67 | 5.49 | 6.12 | 6.12 | 6.04 | 5.58 | 5.78 | 5.60 | 7.46 | 7.19 | 7.39 |
| 201718_s_at | 6.50 | 6.34 | 6.46 | 7.41 | 7.28 | 7.22 | 6.72 | 6.36 | 6.66 | 7.52 | 7.42 | 7.68 |
| 201909_at | 6.61 | 6.56 | 6.49 | 11.15 | 11.15 | 11.32 | 6.52 | 6.35 | 6.65 | 11.36 | 11.53 | 11.41 |
| 202118_s_at | 3.87 | 3.93 | 4.15 | 8.64 | 8.64 | 8.67 | 3.95 | 3.98 | 4.01 | 7.79 | 7.85 | 8.25 |
| 202119_s_at | 4.66 | 4.50 | 4.69 | 10.02 | 9.91 | 9.70 | 4.45 | 4.51 | 4.54 | 9.37 | 9.70 | 9.53 |
| 202359_s_at | 4.16 | 4.26 | 4.21 | 4.82 | 4.63 | 4.70 | 4.14 | 4.25 | 4.10 | 4.59 | 4.62 | 4.70 |
| 202367_at | 6.64 | 6.35 | 6.60 | 8.00 | 7.93 | 7.87 | 6.53 | 6.65 | 6.25 | 8.22 | 8.31 | 8.00 |
| 202391_at | 5.87 | 5.92 | 5.16 | 8.31 | 8.14 | 7.81 | 5.90 | 5.61 | 5.69 | 9.88 | 10.45 | 9.94 |
| 202729_s_at | 5.92 | 6.14 | 6.19 | 8.39 | 8.59 | 8.34 | 5.92 | 5.96 | 6.07 | 7.78 | 7.83 | 7.97 |
| 202848_s_at | 6.61 | 6.74 | 6.65 | 7.53 | 7.43 | 7.64 | 6.61 | 6.61 | 6.45 | 7.74 | 7.77 | 7.75 |
| 203072_at | 5.05 | 4.89 | 5.18 | 5.89 | 5.76 | 5.87 | 5.09 | 4.90 | 4.96 | 6.15 | 6.52 | 6.35 |
| 203744_at | 5.59 | 5.75 | 5.66 | 8.77 | 8.47 | 8.85 | 5.61 | 5.82 | 5.75 | 8.08 | 7.83 | 8.34 |
| 203805_s_at | 6.39 | 6.53 | 6.44 | 8.09 | 7.96 | 7.88 | 6.57 | 6.69 | 6.59 | 6.94 | 7.02 | 7.08 |
| 203814_s_at | 4.87 | 4.99 | 4.68 | 6.87 | 6.60 | 6.60 | 4.88 | 4.85 | 4.80 | 7.71 | 7.75 | 8.19 |
| 204081_at | 5.47 | 5.58 | 5.48 | 6.19 | 6.48 | 6.31 | 5.37 | 5.41 | 5.44 | 7.00 | 7.37 | 7.21 |
| 204141_at | 4.16 | 4.15 | 4.03 | 9.09 | 8.74 | 8.53 | 4.13 | 3.98 | 4.18 | 8.82 | 8.34 | 8.83 |
| 204197_s_at | 5.43 | 5.37 | 5.62 | 8.43 | 8.38 | 8.25 | 5.43 | 5.64 | 5.42 | 7.73 | 8.37 | 8.47 |
| 204198_s_at | 4.66 | 4.37 | 4.58 | 8.71 | 8.68 | 8.47 | 4.54 | 4.62 | 4.57 | 8.25 | 8.59 | 8.57 |
| 204529_s_at | 4.00 | 4.07 | 3.94 | 5.26 | 4.93 | 5.13 | 4.04 | 3.93 | 3.68 | 7.61 | 7.39 | 7.85 |
| 204890_s_at | 6.02 | 5.83 | 5.85 | 8.82 | 8.88 | 8.99 | 6.16 | 6.07 | 5.98 | 8.20 | 8.66 | 8.47 |
| 205000_at | 4.26 | 4.34 | 4.26 | 8.37 | 7.29 | 8.03 | 3.79 | 4.09 | 4.39 | 7.90 | 7.62 | 7.88 |
| 205120_s_at | 4.86 | 4.73 | 4.63 | 6.62 | 6.28 | 6.69 | 4.70 | 4.56 | 4.83 | 6.30 | 5.97 | 6.53 |
| 205718_at | 5.75 | 5.51 | 5.13 | 8.62 | 8.80 | 8.15 | 5.74 | 5.83 | 5.67 | 7.80 | 7.83 | 7.87 |
| 205903_s_at | 5.44 | 5.02 | 5.01 | 6.93 | 6.73 | 6.59 | 5.56 | 5.07 | 5.16 | 6.14 | 6.26 | 6.42 |
| 206219_s_at | 6.09 | 6.19 | 5.90 | 8.23 | 8.18 | 8.01 | 6.40 | 6.15 | 6.04 | 8.44 | 8.86 | 9.03 |
| 206641_at | 4.61 | 4.74 | 4.44 | 7.47 | 8.13 | 7.40 | 4.55 | 4.51 | 4.66 | 6.82 | 7.58 | 7.39 |
| 206700_s_at | 5.30 | 5.46 | 5.33 | 6.52 | 6.67 | 6.56 | 5.11 | 5.19 | 5.22 | 6.24 | 6.42 | 6.58 |
| 207000_s_at | 5.19 | 5.18 | 5.20 | 6.73 | 6.64 | 6.39 | 5.31 | 5.40 | 5.13 | 6.56 | 6.73 | 6.81 |
| 207238_s_at | 6.36 | 6.31 | 6.45 | 8.13 | 8.55 | 8.18 | 6.65 | 6.58 | 6.59 | 9.05 | 9.22 | 9.11 |
| 207621_s_at | 6.50 | 6.47 | 6.29 | 7.50 | 7.44 | 7.26 | 6.59 | 6.19 | 6.45 | 7.32 | 7.06 | 7.24 |
| 208119_s_at | 5.40 | 5.42 | 5.49 | 5.86 | 5.82 | 5.90 | 5.33 | 5.38 | 5.21 | 6.87 | 6.70 | 6.77 |
| 208190_s_at | 4.54 | 4.43 | 4.57 | 6.28 | 6.57 | 6.46 | 4.69 | 4.43 | 4.72 | 6.37 | 6.63 | 6.69 |
| 208502_s_at | 6.78 | 7.26 | 7.31 | 8.28 | 8.98 | 8.45 | 6.53 | 7.02 | 6.34 | 8.68 | 9.52 | 9.22 |
| 209200_at | 4.86 | 4.86 | 4.89 | 5.19 | 5.12 | 5.21 | 4.88 | 4.88 | 4.83 | 7.88 | 9.00 | 8.55 |
| 209372_x_at | 7.72 | 7.76 | 7.47 | 8.44 | 8.32 | 8.46 | 7.86 | 7.69 | 7.69 | 8.36 | 8.43 | 8.50 |
| 209447_at | 5.74 | 5.37 | 5.54 | 6.44 | 6.55 | 6.42 | 5.44 | 5.47 | 5.34 | 7.00 | 7.51 | 7.42 |
| 209570_s_at | 5.24 | 5.00 | 4.92 | 6.55 | 6.83 | 6.52 | 5.15 | 5.09 | 5.10 | 8.40 | 8.96 | 8.77 |
| 209967_s_at | 5.79 | 5.49 | 5.41 | 7.34 | 7.27 | 7.06 | 5.62 | 5.55 | 5.45 | 8.83 | 9.13 | 9.01 |
| 209980_s_at | 6.44 | 6.29 | 6.19 | 7.31 | 7.48 | 7.28 | 6.38 | 6.15 | 6.31 | 7.09 | 7.21 | 7.17 |
| 210258_at | 4.01 | 3.82 | 4.02 | 10.27 | 10.50 | 10.68 | 3.84 | 3.74 | 3.86 | 10.40 | 10.22 | 10.28 |
| 211543_s_at | 5.48 | 5.22 | 5.20 | 6.66 | 6.61 | 6.51 | 5.44 | 5.13 | 5.27 | 6.80 | 7.04 | 6.79 |
| 212254_s_at | 4.56 | 4.53 | 4.64 | 6.67 | 7.48 | 7.11 | 4.34 | 4.90 | 4.45 | 6.04 | 6.45 | 6.19 |
| 212592_at | 4.44 | 5.43 | 4.51 | 10.54 | 10.70 | 10.57 | 4.57 | 4.69 | 4.37 | 10.66 | 9.93 | 10.44 |
| 213029_at | 4.69 | 4.83 | 4.85 | 5.35 | 5.54 | 5.66 | 4.83 | 4.76 | 4.90 | 5.48 | 5.60 | 5.83 |
| 213457_at | 4.66 | 5.02 | 4.84 | 7.61 | 7.66 | 7.34 | 4.93 | 4.96 | 4.73 | 8.91 | 9.37 | 9.51 |
| 213533_at | 5.47 | 5.69 | 5.95 | 6.71 | 7.00 | 6.83 | 5.45 | 6.05 | 5.61 | 8.15 | 8.26 | 8.46 |
| 214508_x_at | 6.31 | 6.11 | 6.64 | 7.52 | 7.83 | 7.63 | 6.22 | 6.41 | 6.36 | 9.00 | 9.46 | 9.50 |
| 214669_x_at | 5.77 | 5.73 | 5.75 | 9.90 | 9.95 | 9.97 | 5.78 | 5.35 | 6.15 | 9.42 | 9.51 | 9.16 |
| 214836_x_at | 7.95 | 8.27 | 7.93 | 10.54 | 10.90 | 10.46 | 7.48 | 8.03 | 7.98 | 11.55 | 11.68 | 11.37 |
| 215016_x_at | 4.19 | 4.20 | 4.33 | 6.45 | 7.17 | 6.73 | 4.24 | 4.53 | 4.41 | 5.78 | 6.04 | 6.28 |
| 215903_s_at | 6.38 | 6.17 | 6.05 | 7.93 | 7.52 | 7.73 | 6.34 | 6.46 | 6.46 | 7.29 | 7.05 | 7.29 |
| 215967_s_at | 5.79 | 5.65 | 5.83 | 6.41 | 6.24 | 6.21 | 5.61 | 5.60 | 5.76 | 6.93 | 6.85 | 6.76 |
| 217422_s_at | 6.48 | 6.12 | 6.21 | 8.47 | 8.56 | 8.33 | 6.65 | 5.98 | 5.96 | 8.61 | 9.38 | 8.76 |
| 218017_s_at | 6.07 | 6.19 | 6.24 | 7.08 | 7.26 | 7.07 | 6.04 | 6.15 | 5.96 | 6.99 | 7.08 | 7.10 |
| 218412_s_at | 5.37 | 5.65 | 5.48 | 7.49 | 7.55 | 7.21 | 5.63 | 5.93 | 5.49 | 6.98 | 6.91 | 6.76 |
| 218573_at | 4.60 | 4.62 | 4.48 | 5.09 | 5.25 | 5.35 | 4.59 | 4.70 | 4.61 | 5.47 | 5.40 | 5.68 |
| 218723_s_at | 4.98 | 4.98 | 4.60 | 8.42 | 9.04 | 8.41 | 4.67 | 4.74 | 4.82 | 7.79 | 8.08 | 7.44 |
| 218858_at | 4.92 | 4.96 | 4.97 | 9.15 | 9.68 | 9.24 | 4.92 | 5.00 | 5.04 | 9.10 | 9.07 | 9.16 |
| 219165_at | 4.97 | 4.68 | 4.69 | 6.34 | 6.30 | 6.31 | 5.17 | 5.10 | 4.82 | 7.28 | 7.80 | 7.92 |
| 219299_at | 4.97 | 5.26 | 5.26 | 6.16 | 6.17 | 6.44 | 5.11 | 5.21 | 5.12 | 5.71 | 5.95 | 5.98 |
| 219855_at | 6.44 | 6.55 | 6.21 | 9.17 | 9.23 | 8.97 | 6.18 | 6.29 | 6.33 | 7.48 | 7.59 | 7.68 |
| 220432_s_at | 3.89 | 3.83 | 3.73 | 4.63 | 4.90 | 4.64 | 3.74 | 3.86 | 3.89 | 4.32 | 4.55 | 4.54 |
| 221003_s_at | 5.52 | 5.57 | 5.72 | 6.36 | 6.38 | 6.20 | 5.76 | 5.64 | 5.68 | 6.95 | 7.33 | 6.97 |
| 221651_x_at | 7.68 | 7.66 | 7.69 | 12.88 | 12.95 | 12.92 | 7.92 | 7.45 | 8.17 | 12.63 | 12.62 | 12.64 |
| 221671_x_at | 7.73 | 7.73 | 7.73 | 12.92 | 12.99 | 12.96 | 7.67 | 7.11 | 8.16 | 12.59 | 12.71 | 12.61 |
| 222307_at | 5.48 | 5.49 | 5.36 | 6.19 | 5.99 | 6.19 | 5.46 | 5.31 | 5.34 | 6.11 | 6.10 | 6.09 |
| 203722_at | 5.88 | 5.76 | 5.76 | 6.15 | 6.06 | 6.18 | 5.71 | 5.70 | 5.71 | 6.67 | 6.67 | 6.78 |
| 219998_at | 4.86 | 4.87 | 4.81 | 5.10 | 5.20 | 5.20 | 4.81 | 4.85 | 4.84 | 5.06 | 5.14 | 5.14 |
| 221487_s_at | 6.06 | 6.07 | 6.21 | 6.62 | 6.80 | 6.76 | 6.17 | 5.90 | 6.12 | 6.67 | 6.53 | 6.67 |

TABLE 2B
Genes With Increased Expression in CD40-Sensitive Cell Lines

| | t-test(two-tailed) | | | | | Gene | | |
|---|---|---|---|---|---|---|---|---|
| Probesets | pAB | pAD | pBC | pCD | Measured | Symbol | UniGene ID | Gene Title |
| FDR Cutoff | 1.0E−02 | 8.2E−03 | 1.2E−02 | 9.4E−03 | | | | |
| 200011_s_at | 4.2E−03 | 8.2E−05 | 7.4E−03 | 1.1E−04 | Yes | ARF3 | Hs.119177 | ADP-ribosyiation factor 3 |
| 200090_at | 6.4E−03 | 7.4E−04 | 3.0E−03 | 2.0E−04 | Yes | FNTA | Hs.356463 | farnesyltransferase CAAX box, alpha |
| 200625_s_at | 5.8E−04 | 7.3E−04 | 1.5E−03 | 2.4E−03 | Yes | CAP1 | Hs.104125 | CAP, adenylate cyclase-associated protein 1 |
| | | | | | | | | (yeast) |
| 200679_x_at | 9.6E−03 | 3.7E−03 | 1.5E−03 | 4.5E−04 | Yes | HMGB1 | Hs.434102 | high-mobility group box 1 |
| 200762_at | 1.3E−04 | 2.4E−03 | 5.4E−03 | 4.4E−03 | Yes | DPYSL2 | Hs.173381 | dihydropyrimidinase-like 2 |
| 200833_s_at | 3.8E−03 | 1.0E−03 | 3.8E−03 | 1.3E−03 | Yes | RAP1B | Hs.374418 | RAP1B, member of RAS oncogene family |
| 200998_s_at | 1.2E−03 | 4.9E−04 | 2.6E−03 | 2.0E−03 | Yes | CKAP4 | Hs.74368 | cytoskeleton-associated protein 4 |
| 201029_s_at | 9.4E−04 | 1.3E−04 | 1.1E−03 | 2.2E−04 | Yes | CD99 | Hs.283477 | CD99 antigen |
| 201077_s_at | 3.8E−03 | 4.8E−03 | 2.9E−03 | 2.2E−03 | Yes | NHP2L1 | Hs.182255 | NHP2 non-histone chromosome protein |
| | | | | | | | | 2-like 1(S. cerevisiae) |
| 201089_at | 9.0E−03 | 1.4E−03 | 7.8E−04 | 6.4E−04 | Yes | ATP6V1B2 | Hs.295917 | ATPase, H+ transporting, lysosomal 56/58 |
| | | | | | | | | kDa, V1 subunit B, isoform 2 |
| 201090_x_at | 4.8E−04 | 2.4E−04 | 2.2E−03 | 1.0E−03 | Yes | K-ALPHA-1 | Hs.446608 | tubulin, alpha, ubiquitous |
| 201209_at | 8.5E−03 | 1.2E−03 | 9.2E−03 | 1.3E−03 | Yes | HDAC1 | Hs.88556 | histone deacetylase 1 |
| 201260_s_at | 6.8E−03 | 7.9E−03 | 1.7E−03 | 3.2E−03 | Yes | SYPL | Hs.80919 | synaptophysin-like protein |
| 201375_s_at | 1.6E−03 | 5.6E−04 | 5.6E−03 | 2.0E−03 | Yes | PPP2CB | Hs.80350 | protein phosphatase 2 (formerly 2A), |
| | | | | | | | | catalytic subunit, beta isoform |
| 201405_s_at | 9.2E−03 | 1.3E−03 | 8.7E−03 | 4.2E−04 | Yes | COPS6 | Hs.15591 | COP9 constitutive photomorphogenic |
| | | | | | | | | homolog subunit 6 (Arabidopsis) |
| 201422_at | 7.0E−03 | 2.0E−04 | 1.0E−03 | 5.5E−04 | Yes | IFI30 | Hs.14623 | interferon, gamma-inducible protein 30 |
| 201453_x_at | 8.4E−04 | 4.8E−04 | 3.7E−03 | 2.8E−03 | Yes | RHEB | Hs.279903 | Ras homolog enriched in brain |
| 201503_at | 1.3E−03 | 1.4E−03 | 2.8E−03 | 2.0E−03 | Yes | G3BP | Hs.48549 | Ras-GTPase-activating protein |
| | | | | | | | | SH3-domain-binding protein |
| 201561_s_at | 8.8E−03 | 5.0E−03 | 6.8E−03 | 1.9E−03 | Yes | CLSTN1 | Hs.29665 | calsyntenin 1 |
| 201584_s_at | 2.3E−03 | 9.9E−04 | 1.1E−03 | 1.2E−04 | Yes | DDX39 | Hs.311609 | DEAD (Asp-Glu-Ala-Asp) box polypeptide |
| | | | | | | | | 39 |
| 201663_s_at | 1.9E−03 | 3.9E−05 | 7.7E−04 | 1.1E−04 | Yes | SMC4L1 | Hs.50758 | SMC4 structural maintenance of |
| | | | | | | | | chromosomes 4-like 1 (yeast) |
| 201664_at | 3.1E−03 | 5.2E−04 | 2.2E−03 | 6.9E−04 | Yes | SMC4L1 | Hs.50758 | SMC4 structural maintenance of |
| | | | | | | | | chromosomes 4-like 1 (yeast) |
| 201764_at | 4.2E−03 | 2.2E−03 | 2.1E−03 | 9.8E−06 | Yes | MGC5576 | Hs.103834 | hypothetical protein MGC5576 |
| 201954_at | 1.4E−03 | 4.1E−03 | 5.1E−04 | 4.2E−03 | Yes | ARPC1B | Hs.433506 | actin related protein 2/3 complex, |
| | | | | | | | | subunit 16, 41 kDa |
| 202016_at | 1.4E−05 | 1.1E−03 | 1.3E−05 | 1.4E−03 | Yes | MEST | Hs.440459 | mesoderm specific transcript homolog |
| | | | | | | | | (mouse) |
| 202078_at | 2.1E−05 | 4.7E−04 | 6.4E−04 | 2.1E−04 | Yes | COPS3 | Hs.6076 | COP9 constitutive photomorphogenic |
| | | | | | | | | homolog subunit 3 (Arabidopsis) |
| 202154_x_at | 3.4E−03 | 7.3E−03 | 3.6E−03 | 6.7E−03 | Yes | TUBB4 | Hs.511743 | tubulin, beta, 4 |
| 202174_s_at | 4.6E−03 | 4.7E−03 | 4.6E−03 | 4.7E−03 | Yes | PCM1 | Hs.348501 | pericentriolar material 1 |
| 202263_at | 4.0E−03 | 4.0E−03 | 2.6E−03 | 8.4E−03 | Yes | CYB5R1 | Hs.334832 | cytochrome b5 reductase 1 (B5R.1) |
| 202313_at | 1.4E−03 | 3.1E−03 | 8.5E−04 | 1.3E−03 | Yes | PPP2R2A | Hs.512628 | protein phosphatase 2 (formerly 2A), |
| | | | | | | | | regulatory subunit B (PR 52), alpha isoform |
| 202355_s_at | 3.2E−03 | 8.1E−03 | 4.1E−03 | 5.2E−03 | Yes | GTF2F1 | Hs.68257 | general transcription factor IIF, |
| | | | | | | | | polypeptide 1, 74 kDa |
| 202356_s_at | 2.9E−03 | 1.8E−03 | 1.0E−03 | 4.2E−03 | Yes | GTF2F1 | Hs.68257 | general transcription factor IIF, |
| | | | | | | | | polypeptide 1, 74 kDa |
| 202415_s_at | 3.6E−03 | 2.7E−03 | 5.3E−03 | 4.6E−03 | Yes | HSPBP1 | Hs.53066 | hsp70-interacting protein |
| 202467_s_at | 1.7E−03 | 4.2E−03 | 8.9E−04 | 2.9E−03 | Yes | TRIP15 | Hs.30212 | thyroid receptor interacting protein 15 |
| 202475_at | 6.1E−04 | 4.3E−04 | 3.2E−06 | 1.1E−03 | Yes | NIFIE14 | Hs.9234 | seven transmembrane domain protein |
| 202484_s_at | 3.7E−03 | 3.4E−04 | 7.8E−03 | 5.3E−04 | Yes | MBD2 | Hs.25674 | methyl-CpG binding domain protein 2 |
| 202675_at | 7.9E−04 | 6.6E−04 | 2.9E−03 | 2.0E−03 | Yes | SDHB | Hs.64 | succinate dehydrogenase complex, subunit |
| | | | | | | | | B, iron sulfur (Ip) |
| 202691_at | 5.3E−03 | 7.0E−03 | 3.3E−03 | 3.8E−03 | Yes | SNRPD1 | Hs.86948 | small nuclear ribonucleoprotein D1 |
| | | | | | | | | polypeptide 16 kDa |
| 202736_s_at | 8.4E−03 | 3.5E−03 | 12E−02 | 1.1E−03 | Yes | LSM4 | Hs.76719 | LSM4 homolog, U6 small nuclear RNA |
| | | | | | | | | associated (S. cerevisiae) |
| 2028_s_at | 5.3E−04 | 2.4E−03 | 2.5E−04 | 1.6E−03 | Yes | E2F1 | Hs.96055 | E2F transcription factor 1 |
| 202816_s_at | 1.9E−04 | 6.9E−03 | 7.0E−04 | 5.9E−03 | Yes | SS18 | Hs.404263 | synovial sarcoma translocation, |
| | | | | | | | | chromosome 18 |
| 203046_s_at | 1.8E−03 | 1.4E−03 | 5.3E−03 | 4.4E−03 | Yes | TIMELESS | Hs.118631 | timeless homolog (Drosophila) |
| 203314_at | 7.2E−04 | 9.7E−04 | 1.1E−03 | 9.4E−04 | Yes | PGPL | Hs.522840 | pseudoautosomal GTP-binding protein-like |
| 203534_at | 2.4E−04 | 2.6E−03 | 2.2E−03 | 1.0E−03 | Yes | LSM1 | Hs.425311 | LSM1 homolog, U6 small nuclear RNA |
| | | | | | | | | associated (S. cerevisiae) |
| 203537_at | 1.7E−03 | 1.5E−03 | 1.5E−03 | 8.9E−04 | Yes | PRPSAP2 | Hs.13339 | phosphoribosyl pyrophosphate synthetase- |
| | | | | | | | | associated protein 2 |
| 203605_at | 3.3E−04 | 2.6E−03 | 1.2E−03 | 5.7E−03 | Yes | SRP54 | Hs.49346 | signal recognition particle 54 kDa |
| 203729_at | 2.9E−05 | 2.1E−04 | 9.4E−05 | 4.5E−04 | Yes | EMP3 | Hs.9999 | epithelial membrane protein 3 |
| 203905_at | 4.8E−04 | 1.1E−03 | 9.2E−05 | 3.7E−03 | Yes | PARN | Hs.43445 | poly(A)-specific ribonuclease (deadenylation |
| | | | | | | | | nuclease) |
| 203941_at | 2.1E−03 | 1.8E−03 | 8.7E−03 | 5.8E−03 | Yes | FLJ10871 | Hs.15562 | hypothetical protein FLJ10871 |
| 203972_s_at | 9.2E−05 | 4.9E−03 | 3.0E−03 | 3.2E−03 | Yes | PEX3 | Hs.7277 | peroxisomal biogenesis factor 3 |
| 204057_at | 3.8E−04 | 1.9E−04 | 9.3E−05 | 1.2E−04 | Yes | ICSBP1 | Hs.14453 | interferon consensus sequence binding |
| | | | | | | | | protein 1 |
| 204581_at | 3.8E−03 | 1.4E−03 | 8.7E−03 | 3.9E−03 | Yes | CD22 | Hs.262150 | CD22 antigen |
| 204604_at | 2.4E−04 | 2.4E−05 | 1.1E−04 | 2.4E−05 | Yes | PFTK1 | Hs.57856 | PFTAIRE protein kinase 1 |
| 204674_at | 7.7E−04 | 1.0E−03 | 5.5E−03 | 4.1E−03 | Yes | LRMP | Hs.124922 | lymphoid-restricted membrane protein |
| 204867_at | 5.7E−03 | 4.1E−03 | 3.3E−03 | 2.2E−03 | Yes | GCHFR | Hs.245644 | GTP cyclohydrolase I feedback regulatory |
| | | | | | | | | protein |
| 204891_s_at | 1.6E−03 | 3.6E−04 | 1.5E−03 | 3.3E−04 | Yes | LCK | Hs.1765 | lymphocyte-specific protein tyrosine kinase |
| 205022_s_at | 3.7E−03 | 3.1E−03 | 1.0E−02 | 6.7E−03 | Yes | CHES1 | Hs.211773 | checkpoint suppressor 1 |
| 205245_at | 6.6E−04 | 2.2E−04 | 1.3E−03 | 7.8E−03 | Yes | PARD6A | Hs.112933 | par-6 partitioning defective 6 homolog alpha |
| | | | | | | | | (C.elegans) |
| 205356_at | 6.9E−04 | 3.2E−03 | 2.5E−03 | 7.0E−03 | Yes | USP13 | Hs.85482 | ubiquitin specific protease 13 (isopeptidase |
| | | | | | | | | T-3) |
| 205367_at | 2.9E−04 | 7.2E−05 | 8.6E−05 | 2.1E−03 | Yes | APS | Hs.371366 | adaptor protein with pleckstrin homology |
| | | | | | | | | and src homology 2 domains |
| 205412_at | 2.4E−04 | 4.7E−05 | 7.0E−03 | 9.2E−04 | Yes | ACAT1 | Hs.37 | acetyl-Coenzyme A acetyltransferase 1 |
| | | | | | | | | (acetoacetyl Coenzyme A thiolase) |
| 205457_at | 1.9E−03 | 6.4E−03 | 9.9E−04 | 4.8E−03 | Yes | MGC4614 | Hs.300691 | hypothetical protein MGC4614 |
| 205504_at | 1.2E−04 | 7.0E−05 | 9.4E−05 | 2.2E−04 | Yes | BTK | Hs.159494 | Bruton agammaglobulinemia tyrosine kinase |
| 205685_at | 7.1E−03 | 4.1E−04 | 2.8E−03 | 2.2E−03 | Yes | CD86 | Hs.27954 | CD86 antigen (CD28 antigen ligand 2, B7-2 |
| | | | | | | | | antigen) |
| 205692_s_at | 5.2E−03 | 4.2E−03 | 4.9E−03 | 3.9E−03 | Yes | CD38 | Hs.174944 | CD38 antigen (p45) |
| 205748_s_at | 8.2E−04 | 8.3E−04 | 7.3E−03 | 1.7E−03 | Yes | RNF126 | Hs.69554 | ring finger protein 126 |
| 205770_at | 2.5E−03 | 1.9E−05 | 2.4E−03 | 3.7E−05 | Yes | GSR | Hs.414334 | glutathione reductase |
| 206060_s_at | 4.2E−03 | 2.0E−03 | 1.5E−03 | 5.7E−04 | Yes | PTPN22 | Hs.87860 | protein tyrosine phosphatase, non-receptor |
| | | | | | | | | type 22 (lymphoid) |
| 206255_at | 3.6E−04 | 5.8E−05 | 5.6E−05 | 4.6E−05 | Yes | BLK | Hs.389900 | B lymphoid tyrosine kinase |
| 206296_x_at | 6.8E−03 | 2.2E−03 | 2.5E−03 | 6.8E−04 | Yes | MAP4K1 | Hs.95424 | mitogen-activated protein kinase kinase |
| | | | | | | | | kinase kinase 1 |
| 206571_s_at | 3.1E−04 | 1.6E−03 | 6.8E−03 | 1.3E−03 | Yes | MAP4K4 | Hs.3628 | mitogen-activated protein kinase kinase |
| | | | | | | | | kinase kinase 4 |
| 206976_s_at | 9.8E−03 | 3.4E−03 | 1.2E−02 | 9.1E−03 | Yes | HSPH1 | Hs.36927 | heat shock 105 kDa/110 kDa protein 1 |
| 207121_s_at | 2.0E−03 | 4.2E−03 | 1.8E−03 | 4.6E−03 | Yes | MAPK6 | Hs.271980 | mitogen-activated protein kinase 6 |
| 207419_s_at | 6.1E−03 | 1.3E−03 | 2.4E−03 | 6.7E−04 | Yes | RAC2 | Hs.301175 | ras-related C3 botulinum toxin substrate |
| | | | | | | | | 2 (rho family, small GTP binding protein |
| | | | | | | | | Rac2) |
| 207480_s_at | 6.1E−03 | 5.0E−03 | 4.6E−03 | 7.8E−03 | Yes | MEIS2 | Hs.362805 | Meis1, myeloid ecotropic viral integration |
| | | | | | | | | site 1 homolog 2 (mouse) |
| 207630_s_at | 1.1E−03 | 1.6E−06 | 4.4E−04 | 1.4E−04 | Yes | CREM | Hs.231975 | cAMP responsive element modulator |
| 207760_s_at | 7.3E−03 | 1.7E−03 | 4.1E−03 | 6.5E−04 | Yes | NCOR2 | Hs.287994 | nuclear receptor co-repressor 2 |
| 208459_s_at | 7.4E−04 | 3.2E−05 | 1.2E−03 | 4.9E−05 | Yes | XPO7 | Hs.172685 | exportin 7 |
| 208642_s_at | 6.2E−04 | 2.0E−03 | 7.9E−04 | 2.8E−03 | Yes | XRCC5 | Hs.257082 | X-ray repair complementing defective |
| | | | | | | | | repair in Chinese hamster cells 5 |
| | | | | | | | | (double-strand-break rejoining; Ku |
| | | | | | | | | autoantigen, 80 kDa) |
| 208643_s_at | 5.6E−03 | 5.9E−03 | 9.0E−04 | 9.7E−04 | Yes | XRCC5 | Hs.257082 | X-ray repair complementing defective |
| | | | | | | | | repair in Chinese hamster cells 5 |
| | | | | | | | | (double-strand-break rejoining; Ku |
| | | | | | | | | autoantigen, 80 kDa) |
| 208644_at | 5.8E−04 | 5.5E−03 | 8.3E−03 | 9.4E−03 | Yes | ADPRT | Hs.177766 | ADP-ribosyltransferase (NAD+; poly |
| | | | | | | | | (ADP-ribose) polymerase) |
| 208679_s_at | 7.8E−04 | 1.3E−03 | 5.7E−03 | 4.7E−03 | Yes | ARPC2 | Hs.83583 | actin related protein 2/3 complex, |
| | | | | | | | | subunit 2, 34 kDa |
| 208857_s_at | 4.6E−04 | 2.6E−03 | 5.3E−04 | 1.9E−03 | Yes | PCMT1 | Hs.79137 | protein-L-isoaspartate (D-aspartate) O- |
| | | | | | | | | methyltransferase |
| 208875_s_at | 3.2E−04 | 3.8E−04 | 1.2E−03 | 3.2E−04 | Yes | PAK2 | Hs.284275 | p21 (CDKN1A)-activated kinase 2 |
| 209075_s_at | 2.3E−03 | 4.4E−03 | 2.9E−03 | 3.3E−03 | Yes | NIFU | Hs.350702 | nitrogen fixation cluster-like |
| 209229_s_at | 3.8E−03 | 1.3E−03 | 2.4E−03 | 5.0E−04 | Yes | KIAA1115 | Hs.411875 | KIAA1115 protein |
| 209251_x_at | 4.8E−04 | 3.0E−04 | 2.3E−05 | 2.6E−03 | Yes | TUBA6 | Hs.406578 | tubulin alpha 6 |
| 209316_s_at | 2.1E−03 | 4.6E−04 | 9.0E−03 | 2.8E−03 | Yes | HBS1L | Hs.221040 | HBS1-like (S. cerevisiae) |
| 209471_s_at | 5.0E−03 | 5.2E−03 | 8.2E−04 | 1.4E−03 | Yes | FNTA | Hs.356463 | farnesyltransferase, CAAX box, alpha |
| 209517_s_at | 1.0E−03 | 2.8E−04 | 3.7E−03 | 3.2E−03 | Yes | ASH2L | Hs.6856 | ash2 (absent, small, or homeotic)-like |
| | | | | | | | | (Drosophila) |
| 209569_x_at | 5.4E−04 | 3.0E−04 | 2.0E−03 | 1.0E−03 | Yes | D4S234E | Hs.79404 | DNA segment on chromosome 4 (unique) |
| | | | | | | | | 234 expressed sequence |
| 209714_s_at | 5.2E−03 | 4.8E−03 | 2.4E−03 | 1.9E−03 | Yes | CDKN3 | Hs.84113 | cyclin-dependent kinase inhibitor 3 (CDK2- |
| | | | | | | | | associated dual specificity phosphatase) |
| 209765_at | 2.8E−03 | 4.0E−03 | 3.6E−04 | 8.3E−04 | Yes | ADAM19 | Hs.289368 | a disintegrin and metalloproteinase domain |
| | | | | | | | | 19 (meltrin beta) |
| 209932_s_at | 2.6E−04 | 1.5E−03 | 9.0E−04 | 1.5E−03 | Yes | DUT | Hs.367676 | dUTP pyrophosphatase |
| 210981_s_at | 1.1E−03 | 1.1E−04 | 1.9E−03 | 8.0E−05 | Yes | GPRK6 | Hs.235116 | G protein-coupled receptor kinase 6 |
| 211043_s_at | 3.5E−04 | 2.3E−03 | 8.3E−04 | 4.5E−03 | Yes | CLTB | Hs.380749 | clathrin, light polypeptide (Lcb) |
| 211066_x_at | 4.7E−05 | 2.3E−03 | 9.1E−03 | 4.1E−03 | Yes | PCDHGC3 | Hs.283794 | protocadherin gamma subfamily C, 3 |
| 211072_x_at | 1.0E−03 | 5.9E−04 | 2.9E−03 | 7.0E−04 | Yes | K-ALPHA-1 | Hs.446608 | tubulin, alpha, ubiquitous |
| 211593_s_at | 1.2E−03 | 3.8E−03 | 8.0E−04 | 2.5E−03 | Yes | MAST205 | Hs.101474 | microtubule associated testis specific |
| | | | | | | | | serine/threonine protein kinase |
| 211686_s_at | 2.3E−03 | 1.7E−03 | 2.5E−03 | 5.2E−03 | Yes | LOC84549 | Hs.77135 | RNA binding protein |
| 211945_s_at | 2.8E−03 | 2.8E−03 | 1.9E−04 | 2.4E−04 | Yes | ITGB1 | Hs.287797 | integrin, beta 1 (fibronectin receptor, beta |
| | | | | | | | | polypeptide, antigen CD29 includes MDF2, |
| | | | | | | | | MSK12) |
| 212066_s_at | 8.6E−03 | 4.6E−03 | 2.4E−03 | 4.4E−04 | Yes | USP34 | Hs.507665 | ubiquitin specific protease 34 |
| 212085_at | 4.0E−03 | 2.9E−03 | 3.7E−03 | 2.8E−03 | Yes | SLC25A6 | Hs.350927 | solute carrier family 25 (mitochondrial |
| | | | | | | | | carrier; adenine nucleotide translocator), |
| | | | | | | | | member 6 |
| 212094_at | 3.9E−04 | 5.0E−03 | 6.1E−05 | 3.0E−03 | Yes | PEG10 | Hs.137476 | paternally expressed 10 |
| 212313_at | 3.9E−03 | 9.8E−04 | 2.4E−03 | 6.7E−05 | Yes | MGC29816 | Hs.5019 | hypothetical protein MGC29816 |
| 212350_at | 5.9E−04 | 9.5E−04 | 1.1E−03 | 1.7E−04 | Yes | TBC1D1 | Hs.372659 | TBC1 (tre-2/USP6, BUB2, cdc16) domain |
| | | | | | | | | family, member 1 |
| 212372_at | 2.6E−03 | 1.1E−04 | 5.4E−04 | 6.8E−04 | Yes | MYH10 | Hs.280311 | myosin, heavy polypeptide 10, non-muscle |
| 212443_at | 4.0E−03 | 2.1E−03 | 6.5E−03 | 3.3E−03 | Yes | KIAA0540 | Hs.437043 | KIAA0540 protein |
| 212573_at | 3.6E−04 | 6.2E−03 | 1.6E−04 | 1.3E−03 | Yes | KIAA0830 | Hs.167115 | KIAA0830 protein |
| 212587_s_at | 1.8E−03 | 3.9E−04 | 1.5E−04 | 1.1E−03 | Yes | PTPRC | Hs.444324 | protein tyrosine phosphatase, receptor |
| | | | | | | | | type, C |
| 212588_at | 3.9E−03 | 1.9E−03 | 1.1E−03 | 4.5E−04 | Yes | PTPRC | Hs.444324 | protein tyrosine phosphatase, receptor |
| | | | | | | | | type, C |
| 212619_at | 9.6E−04 | 1.6E−03 | 8.7E−04 | 2.0E−03 | Yes | KIAA0286 | Hs.14912 | KIAA0286 protein |
| 212646_at | 2.6E−03 | 9.7E−05 | 1.5E−04 | 1.6E−04 | Yes | RAFTLIN | Hs.436432 | raft-linking protein |
| 212656_at | 6.0E−03 | 1.4E−03 | 1.1E−02 | 2.8E−03 | Yes | DKFZP586 | Hs.435643 | hepatocellularcarcinoma-associated antigen |
| | | | | | | D0919 | | HCA557a |
| 212735_at | 2.1E−03 | 4.7E−04 | 2.1E−04 | 7.0E−04 | Yes | KIAA0226 | Hs.499355 | KIAA0226 gene product |
| 212812_at | 1.6E−03 | 3.3E−03 | 5.1E−04 | 2.0E−03 | Yes | — | Hs.288232 | Homo sapiens cDNA: FLJ22642 fis, clone |
| | | | | | | | | HSI06970 |
| 212826_s_at | 1.3E−03 | 1.4E−03 | 1.9E−03 | 1.6E−04 | Yes | SLC25A6 | Hs.350927 | solute carrier family 25 (mitochondrial |
| | | | | | | | | carrier; adenine nucleotide translocator), |
| | | | | | | | | member 6 |
| 213093_at | 2.3E−03 | 3.7E−03 | 2.0E−04 | 3.8E−04 | Yes | PRKCA | Hs.349611 | protein kinase C, alpha |
| 213106_at | 1.4E−03 | 1.8E−03 | 1.8E−03 | 8.5E−04 | Yes | ATP8A1 | Hs.291385 | ATPase, aminophospholipid transporter |
| | | | | | | | | (APLT), Class I, type 8A, member 1 |
| 213476_x_at | 5.9E−03 | 6.7E−03 | 2.1E−03 | 2.5E−03 | Yes | TUBB4 | Hs.511743 | tubulin, beta, 4 |
| 213504_at | 1.3E−03 | 4.7E−04 | 2.0E−03 | 4.0E−04 | Yes | COPS6 | Hs.15591 | COP9 constitutive photomorphogenic |
| | | | | | | | | homolog subunit 6 (Arabidopsis) |
| 213603_s_at | 3.1E−03 | 3.8E−04 | 9.1E−05 | 2.7E−04 | Yes | RAC2 | Hs.301175 | ras-related C3 botulinum toxin substrate |
| | | | | | | | | 2 (rho family, small GTP binding protein |
| | | | | | | | | Rac2) |
| 213646_x_at | 2.1E−03 | 6.8E−03 | 2.6E−03 | 2.8E−04 | Yes | K-ALPHA-1 | Hs.446608 | tubulin, alpha, ubiquitous |
| 213906_at | 2.8E−03 | 2.7E−05 | 3.2E−03 | 4.6E−05 | Yes | MYBL1 | Hs.300592 | v-myb myeloblastosis viral oncogene |
| | | | | | | | | homolog (avian)-like 1 |
| 214157_at | 1.8E−03 | 4.7E−04 | 2.6E−08 | 3.4E−03 | Yes | GNAS | Hs.157307 | GNAS complex locus |
| 214219_x_at | 5.2E−03 | 1.0E−03 | 8.4E−03 | 2.7E−03 | Yes | MAP4K1 | Hs.95424 | mitogen-activated protein kinase kinase |
| | | | | | | | | kinase kinase 1 |
| 214339_s_at | 1.5E−03 | 6.0E−04 | 1.2E−02 | 3.4E−03 | Yes | MAP4K1 | Hs.95424 | mitogen-activated protein kinase kinase |
| | | | | | | | | kinase kinase 1 |
| 214553_s_at | 5.7E−03 | 3.3E−03 | 2.8E−03 | 1.2E−03 | Yes | ARPP-19 | Hs.7351 | cyclic AMP phosphoprotein, 19 kD |
| 215023_s_at | 2.2E−03 | 5.9E−03 | 1.1E−03 | 2.2E−03 | Yes | PEX1 | Hs.164682 | peroxisome biogenesis factor 1 |
| 215158_s_at | 8.7E−03 | 1.8E−03 | 1.3E−03 | 4.2E−03 | Yes | DEDD | Hs.169681 | death effector domain containing |
| 215836_s_at | 4.8E−03 | 5.1E−03 | 7.5E−03 | 6.9E−04 | Yes | PCDHGC3 | Hs.283794 | protocadherin gamma subfamily C, 3 |
| 216241_s_at | 1.1E−03 | 7.6E−04 | 1.8E−04 | 7.2E−06 | Yes | TCEA1 | Hs.78869 | transcription elongation factor A (SII), 1 |
| 216321_s_at | 4.2E−03 | 3.9E−03 | 3.8E−03 | 1.5E−03 | Yes | NR3C1 | Hs.512414 | nuclear receptor subfamily 3, group C, |
| | | | | | | | | member 1 (glucocorticoid receptor) |
| 217118_s_at | 1.3E−03 | 1.3E−03 | 4.2E−03 | 3.9E−03 | Yes | KIAA0930 | Hs.454533 | KIAA0930 protein |
| 217898_at | 4.0E−03 | 1.7E−03 | 2.4E−04 | 4.4E−03 | Yes | LOC56851 | Hs.4245 | chromosome 15 hypothetical ATG/GTP |
| | | | | | | | | binding protein |
| 217933_s_at | 3.9E−04 | 5.5E−04 | 2.4E−03 | 2.8E−03 | Yes | LAP3 | Hs.182579 | leucine aminopeptidase 3 |
| 218039_at | 1.6E−03 | 2.1E−03 | 7.7E−04 | 9.4E−04 | Yes | NUSAP1 | Hs.279905 | nucleolar and spindle associated protein 1 |
| 218096_at | 7.1E−03 | 6.4E−03 | 3.8E−04 | 3.4E−04 | Yes | LPAAT-e | Hs.281895 | acid acyltransferase-epsilon |
| 218250_s_at | 4.5E−05 | 6.4E−05 | 1.1E−03 | 1.7E−04 | Yes | CNOT7 | Hs.170553 | CCR4-NOT transcription complex, subunit 7 |
| 218287_s_at | 1.3E−04 | 3.9E−04 | 5.3E−05 | 1.3E−03 | Yes | EIF2C1 | Hs.309452 | eukaryotic translation initiation factor |
| | | | | | | | | 2C, 1 |
| 218336_at | 4.6E−03 | 9.8E−04 | 2.5E−03 | 9.4E−04 | Yes | PFDN2 | Hs.298229 | prefoldin 2 |
| 218404_at | 1.8E−03 | 8.5E−04 | 1.1E−02 | 5.0E−03 | Yes | SNX10 | Hs.418132 | sorting nexin 10 |
| 218473_s_at | 2.1E−03 | 7.9E−05 | 4.5E−03 | 2.7E−04 | Yes | FLJ22329 | Hs.418795 | hypothetical protein FLJ22329 |
| 218640_s_at | 2.0E−03 | 6.9E−04 | 8.9E−04 | 5.2E−04 | Yes | PLEKHF2 | Hs.29724 | pleckstrin homology domain containing, |
| | | | | | | | | family F (with FYVE domain) member 2 |
| 218654_s_at | 2.2E−06 | 8.7E−04 | 1.0E−04 | 4.7E−05 | Yes | MRPS33 | Hs.83006 | mitochondrial ribosomal protein S33 |
| 218680_x_at | 4.6E−03 | 2.5E−03 | 7.9E−04 | 2.5E−04 | Yes | HYPK | Hs.511978 | Huntingtin interacting protein K |
| 218802_at | 9.1E−07 | 1.7E−03 | 2.1E−03 | 1.1E−03 | Yes | FLJ20647 | Hs.234149 | hypothetical protein FLJ20647 |
| 219220_x_at | 3.2E−03 | 2.2E−04 | 3.5E−04 | 1.0E−04 | Yes | MRPS22 | Hs.512649 | mitochondrial ribosomal protein S22 |
| 219304_s_at | 8.4E−03 | 2.2E−03 | 4.8E−03 | 2.8E−04 | Yes | SCDGF-B | Hs.112885 | spinal cord-derived growth factor-B |
| 219428_s_at | 2.9E−03 | 3.0E−04 | 2.4E−03 | 2.3E−04 | Yes | PXMP4 | Hs.436924 | peroxisomal membrane protein 4, 24 kDa |
| 219551_at | 8.1E−04 | 6.8E−03 | 4.0E−04 | 2.1E−03 | Yes | EAF2 | Hs.383018 | ELL associated factor 2 |
| 219911_s_at | 5.7E−03 | 3.8E−03 | 1.9E−03 | 1.1E−03 | Yes | SLCO4A1 | Hs.235782 | solute carrier organic anion transporter |
| | | | | | | | | family, member 4A1 |
| 220059_at | 7.0E−04 | 6.3E−05 | 1.2E−04 | 3.9E−05 | Yes | BRDG1 | Hs.121128 | BCR downstream signaling 1 |
| 221059_s_at | 2.8E−04 | 1.6E−04 | 1.2E−03 | 9.6E−04 | Yes | CHST6 | Hs.157439 | carbohydrate (N-acetylglucosamine 6-O) |
| | | | | | | | | sulfotransferase 6 |
| 221080_s_at | 5.3E−03 | 1.8E−03 | 5.6E−03 | 1.3E−03 | Yes | FLJ22757 | Hs.236449 | hypothetical protein FLJ22757 |
| 221483_s_at | 6.8E−04 | 5.8E−03 | 3.3E−04 | 4.1E−03 | Yes | ARPP-19 | Hs.7351 | cyclic AMP phosphoprotein, 19 kD |
| 221558_s_at | 2.0E−06 | 1.8E−03 | 3.8E−03 | 1.8E−03 | Yes | LEF1 | Hs.44865 | lymphoid enhancer-binding factor 1 |
| 221692_s_at | 6.4E−04 | 7.5E−03 | 2.7E−03 | 2.0E−06 | Yes | MRPL34 | Hs.238808 | mitochondrial ribosomal protein L34 |
| 221893_s_at | 5.7E−04 | 2.2E−03 | 2.3E−03 | 5.8E−03 | Yes | ADCK2 | Hs.210397 | aarF domain containing kinase 2 |
| 222065_s_at | 8.6E−04 | 9.6E−04 | 6.5E−04 | 1.7E−03 | Yes | FLII | Hs.445182 | flightless I homolog (Drosophila) |
| 222199_s_at | 6.0E−04 | 8.4E−04 | 6.8E−04 | 2.8E−03 | Yes | BIN3 | Hs.68090 | bridging integrator 3 |
| 32541_at | 4.8E−04 | 4.2E−03 | 2.2E−04 | 2.3E−03 | Yes | PPP3CC | Hs.75206 | protein phosphatase 3 (formerly 2B), |
| | | | | | | | | catalytic subunit, gamma isoform |
| | | | | | | | | (calcineurin A gamma) |
| 35820_at | 1.0E−02 | 1.8E−03 | 6.0E−03 | 1.5E−03 | Yes | GM2A | Hs.387156 | GM2 ganglioside activator protein |
| 35974_at | 5.6E−03 | 2.8E−03 | 9.1E−03 | 5.5E−03 | Yes | LRMP | Hs.124922 | lymphoid-restricted membrane protein |
| 36564_at | 2.0E−03 | 1.1E−03 | 1.9E−03 | 1.1E−03 | Yes | FLJ90005 | Hs.511807 | hypothetical protein FLJ90005 |
| 38521_at | 4.5E−04 | 3.0E−04 | 1.3E−03 | 2.6E−04 | Yes | CD22 | Hs.262150 | CD22 antigen |
| 39835_at | 1.5E−03 | 1.1E−04 | 2.6E−04 | 1.7E−04 | Yes | SBF1 | Hs.112049 | SET binding factor 1 |
| 44120_at | 3.2E−04 | 5.3E−05 | 9.0E−05 | 1.9E−05 | Yes | ADCK2 | Hs.210397 | aarF domain containing kinase 2 |
| 200911_s_at | 4.6E−05 | 6.9E−04 | 2.6E−03 | 3.4E−04 | Yes | TACC1 | Hs.279245 | transforming, acidic coiled-coil |
| | | | | | | | | containing protein 1 |
| 201028_s_at | 8.6E−05 | 1.8E−05 | 7.4E−04 | 3.6E−05 | Yes | CD99 | Hs.283477 | CD99 antigen |
| 201328_at | 1.5E−03 | 2.6E−03 | 3.3E−04 | 3.7E−03 | Yes | ETS2 | Hs.292477 | v-ets erythroblastosis virus E26 oncogene |
| | | | | | | | | homolog 2 (avian) |
| 201425_at | 3.9E−03 | 1.3E−04 | 1.0E−02 | 1.1E−04 | Yes | ALDH2 | Hs.331141 | aldehyde dehydrogenase 2 family |
| | | | | | | | | (mitochondrial) |
| 201718_s_at | 3.2E−04 | 5.5E−04 | 1.1E−02 | 3.5E−03 | Yes | EPB41L2 | Hs.440387 | erythrocyte membrane protein band |
| | | | | | | | | 4.1-like 2 |
| 201909_at | 2.5E−06 | 7.2E−07 | 8.1E−06 | 1.2E−05 | Yes | RPS4Y | Hs.180911 | ribosomal protein S4, Y-linked |
| 202118_s_at | 3.0E−04 | 1.0E−04 | 2.8E−07 | 1.2E−03 | Yes | CPNE3 | Hs.14158 | copine III |
| 202119_s_at | 8.1E−06 | 1.2E−05 | 1.4E−04 | 1.7E−04 | Yes | CPNE3 | Hs.14158 | copine III |
| 202359_s_at | 3.7E−03 | 8.2E−04 | 1.7E−03 | 1.5E−03 | Yes | SNX19 | Hs.409862 | sorting nexin 19 |
| 202367_at | 1.5E−03 | 2.3E−04 | 3.8E−03 | 5.0E−04 | Yes | CUTL1 | Hs.438974 | cut-like 1, CCAAT displacement protein |
| | | | | | | | | (Drosophila) |
| 202391_at | 2.4E−03 | 2.3E−04 | 6.0E−04 | 2.8E−04 | Yes | BASP1 | Hs.511745 | brain abundant, membrane attached signal |
| | | | | | | | | protein 1 |
| 202729_s_at | 3.2E−05 | 1.4E−04 | 5.8E−05 | 2.1E−05 | Yes | LTBP1 | Hs.241257 | latent transforming growth factor beta |
| | | | | | | | | binding protein 1 |
| 202848_s_at | 6.9E−04 | 6.7E−04 | 2.7E−04 | 1.4E−03 | Yes | GPRK6 | Hs.235116 | G protein-coupled receptor kinase 6 |
| 203072_at | 3.8E−03 | 9.2E−04 | 4.0E−04 | 1.6E−03 | Yes | MYO1E | Hs.437459 | myosin IE |
| 203744_at | 3.3E−04 | 1.7E−03 | 1 6E−04 | 1.2E−03 | Yes | HMGB3 | Hs.19114 | high-mobility group box 3 |
| 203805_s_at | 8.2E−05 | 7.3E−04 | 1.8E−04 | 2.2E−03 | Yes | FANCA | Hs.284153 | Fanconi anemia, complementation group A |
| 203814_s_at | 1.3E−04 | 2.9E−04 | 1.3E−03 | 2.1E−03 | Yes | NQO2 | Hs.441039 | NAD(P)H dehydrogenase, quinone 2 |
| 204081_at | 4.3E−03 | 1.9E−03 | 5.6E−03 | 2.7E−03 | Yes | NRGN | Hs.232004 | neurogranin (protein kinase C substrate, |
| | | | | | | | | RC3) |
| 204141_at | 6.7E−04 | 6.5E−04 | 3.7E−04 | 3.5E−04 | Yes | TUBB | Hs.512712 | tubulin, beta polypeptide |
| 204197_s_at | 1.3E−05 | 4.0E−03 | 9.9E−06 | 4.3E−03 | Yes | RUNX3 | Hs.170019 | runt-related transcription factor 3 |
| 204198_s_at | 4.8E−06 | 1.5E−05 | 1.2E−04 | 5.1E−04 | Yes | RUNX3 | Hs.170019 | runt-related transcription factor 3 |
| 204529_s_at | 3.5E−03 | 6.6E−04 | 1.1E−03 | 3.9E−05 | Yes | TOX | Hs.439767 | thymus high mobility group box protein |
| | | | | | | | | TOX |
| 204890_s_at | 4.0E−06 | 6.5E−04 | 2.6E−06 | 1.0E−03 | Yes | LCK | Hs.1765 | lymphocyte-specific protein tyrosine |
| | | | | | | | | kinase |
| 205000_at | 7.4E−03 | 2.6E−04 | 1.6E−03 | 3.3E−04 | Yes | DDX3Y | Hs.99120 | DEAD (Asp-Glu-Ala-Asp) box polypeptide |
| | | | | | | | | 3, Y-linked |
| 205120_s_at | 1.1E−03 | 5.0E−03 | 7.1E−04 | 3.7E−03 | Yes | SGCB | Hs.438953 | sarcoglycan, beta (43 kDa dystrophin- |
| | | | | | | | | associated glycoprotein) |
| 205718_at | 3.3E−04 | 5.4E−03 | 3.2E−03 | 7.5E−05 | Yes | ITGB7 | Hs.1741 | integrin, beta 7 |
| 205903_s_at | 1.3E−03 | 5.2E−03 | 2.1E−03 | 8.6E−03 | Yes | KCNN3 | Hs.89230 | potassium intermediate/small conductance |
| | | | | | | | | calcium-activated channel, subfamily N, |
| | | | | | | | | member 3 |
| 206219_s_at | 6.2E−05 | 9.8E−04 | 2.9E−04 | 6.7E−04 | Yes | VAV1 | Hs.116237 | vav 1 oncogene |
| 206641_at | 2.4E−03 | 3.1E−03 | 4.3E−03 | 5.5E−03 | Yes | TNFRSF17 | Hs.2556 | tumor necrosis factor receptor superfamily, |
| | | | | | | | | member 17 |
| 206700_s_at | 6.0E−05 | 2.5E−03 | 2.6E−05 | 3.1E−03 | Yes | SMCY | Hs.80358 | Smcy homolog, Y-linked (mouse) |
| 207000_s_at | 5.2E−03 | 2.2E−03 | 7.2E−04 | 2.0E−04 | Yes | PPP3CC | Hs.75206 | protein phosphatase 3 (formerly 2B), |
| | | | | | | | | catalytic subunit, gamma isoform |
| | | | | | | | | (calcineurin A gamma) |
| 207238_s_at | 2.7E−03 | 2.5E−06 | 5.4E−03 | 4.6E−05 | Yes | PTPRC | Hs.444324 | protein tyrosine phosphatase, receptor |
| | | | | | | | | type, C |
| 207621_s_at | 5.4E−04 | 1.6E−03 | 4.3E−03 | 7.7E−03 | Yes | PEMT | Hs.15192 | phosphatidylethanolamine N-methyl- |
| | | | | | | | | transferase |
| 208119_s_at | 4.4E−04 | 8.0E−05 | 2.8E−03 | 3.2E−05 | Yes | ZNF505 | Hs.515284 | zinc finger protein 505 |
| 208190_s_at | 3.4E−04 | 6.2E−04 | 1.4E−04 | 1.4E−04 | Yes | LISCH7 | Hs.312129 | liver-specific bHLH-Zip transcription |
| | | | | | | | | factor |
| 208502_s_at | 6.6E−03 | 3.7E−03 | 2.7E−03 | 1.6E−03 | Yes | PITX1 | Hs.84136 | paired-like homeodomain transcription |
| | | | | | | | | factor 1 |
| 209200_at | 3.1E−03 | 8.1E−03 | 1.6E−03 | 8.0E−03 | Yes | MEF2C | Hs.368950 | MADS box transcription enhancer factor 2, |
| | | | | | | | | polypeptide C (myocyte enhancer factor 2C) |
| 209372_x_at | 5.6E−03 | 5.2E−03 | 1.0E−03 | 9.2E−04 | Yes | TUBB | Hs.512712 | tubulin, beta polypeptide |
| 209447_at | 6.9E−03 | 1.4E−03 | 5.4E−05 | 4.6E−03 | Yes | SYNE1 | Hs.282117 | spectrin repeat containing, nuclear |
| | | | | | | | | envelope 1 |
| 209570_s_at | 3.3E−04 | 2.1E−04 | 3.2E−03 | 1.9E−03 | Yes | D4S234E | Hs.79404 | DNA segment on chromosome 4 (unique) |
| | | | | | | | | 234 expressed sequence |
| 209967_s_at | 5.1E−04 | 3.3E−05 | 2.9E−04 | 3.9E−05 | Yes | CREM | Hs.231975 | cAMP responsive element modulator |
| 209980_s_at | 4.1E−04 | 1.9E−03 | 3.1E−04 | 1.3E−03 | Yes | SHMT1 | Hs.293636 | serine hydroxymethyltransferase 1 |
| | | | | | | | | (soluble) |
| 210258_at | 1.0E−05 | 4.1E−07 | 8.6E−05 | 2.6E−07 | Yes | RGS13 | Hs.17165 | regulator of G-protein signalling 13 |
| 211543_s_at | 1.2E−03 | 2.2E−04 | 1.0E−03 | 2.0E−04 | Yes | GPRK6 | Hs.235116 | G protein-coupled receptor kinase 6 |
| 212254_s_at | 7.8E−03 | 3.3E−03 | 1.4E−03 | 2.2E−03 | Yes | BPAG1 | Hs.443518 | bullous pemphigoid antigen 1, 230/240 kDa |
| 212592_at | 2.5E−03 | 2.9E−04 | 1.0E−05 | 2.9E−04 | Yes | IGJ | Hs.381568 | immunoglobulin J polypeptide, linker |
| | | | | | | | | protein for immunoglobulin alpha and mu |
| | | | | | | | | polypeptides |
| 213029_at | 5.2E−03 | 6.0E−03 | 7.5E−03 | 8.3E−03 | Yes | NFIB | Hs.302690 | nuclear factor I/B |
| 213457_at | 4.9E−05 | 1.6E−04 | 5.5E−05 | 5.0E−04 | Yes | MFHAS1 | Hs.379414 | malignant fibrous histiocytoma amplified |
| | | | | | | | | sequence 1 |
| 213533_at | 4.2E−03 | 2.6E−04 | 1.2E−02 | 1.1E−03 | Yes | D4S234E | Hs.79404 | DNA segment on chromosome 4 (unique) |
| | | | | | | | | 234 expressed sequence |
| 214508_x_at | 4.2E−03 | 1.9E−04 | 5.7E−04 | 1.1E−03 | Yes | CREM | Hs.231975 | cAMP responsive element modulator |
| 214669_x_at | 6.1E−08 | 7.4E−04 | 2.8E−03 | 1.1E−03 | Yes | — | Hs.525893 | Homo sapiens cDNA FLJ26296 fis, clone |
| | | | | | | | | DMC07192, highly similar to Ig kappa chain |
| | | | | | | | | V-III region HAH precursor |
| 214836_x_at | 1.7E−04 | 2.2E−05 | 3.3E−04 | 3.5E−04 | Yes | — | Hs.525895 | Homo sapiens cDNA FLJ39619 fis, clone |
| | | | | | | | | SMINT2000984, highly similar to IG |
| | | | | | | | | KAPPA CHAIN V-II REGION GM607 |
| | | | | | | | | PRECURSOR. |
| 215016_x_at | 4.8E−03 | 3.6E−03 | 3.1E−03 | 1.8E−03 | Yes | BPAG1 | Hs.443518 | bullous pemphigoid antigen 1, 230/240 kDa |
| 215903_s_at | 6.9E−04 | 1.5E−03 | 4.5E−03 | 3.4E−03 | Yes | MAST205 | Hs.101474 | microtubule associated testis specific |
| | | | | | | | | serine/threonine protein kinase |
| 215967_s_at | 2.9E−03 | 1.3E−04 | 1.5E−03 | 7.0E−05 | Yes | LY9 | Hs.403857 | lymphocyte antigen 9 |
| 217422_s_at | 2.4E−04 | 2.7E−03 | 6.2E−03 | 1.2E−03 | Yes | CD22 | Hs.262150 | CD22 antigen |
| 218017_s_at | 3.5E−04 | 2.7E−04 | 2.2E−04 | 3.0E−04 | Yes | FLJ32731 | Hs.191320 | hypothetical protein FLJ32731 |
| 218412_s_at | 1.8E−04 | 2.3E−04 | 6.3E−04 | 3.9E−03 | Yes | GTF2IRD1 | Hs.430854 | GTF2I repeat domain containing 1 |
| 218573_at | 4.4E−03 | 2.5E−03 | 7.6E−03 | 4.1E−03 | Yes | MAGEH1 | Hs.279819 | APR-1 protein |
| 218723_s_at | 3.5E−04 | 4.2E−04 | 2.0E−03 | 2.5E−03 | Yes | RGC32 | Hs.76640 | RGC32 protein |
| 218858_at | 1.3E−03 | 4.4E−07 | 9.6E−04 | 1.6E−07 | Yes | FLJ12428 | Hs.87729 | hypothetical protein FLJ12428 |
| 219165_at | 3.1E−03 | 1.2E−03 | 6.1E−03 | 1.2E−03 | Yes | PDLIM2 | Hs.375560 | PDZ and LIM domain 2 (mystique) |
| 219299_at | 1.1E−03 | 4.9E−03 | 3.1E−03 | 6.6E−03 | Yes | FLJ20772 | Hs.9925 | hypothetical protein FLJ20772 |
| 219855_at | 4.0E−05 | 1.3E−03 | 4.0E−05 | 9.4E−05 | Yes | NUDT11 | Hs.200016 | nudix (nucleoside diphosphate linked moiety |
| | | | | | | | | X)-type motif 11 |
| 220432_s_at | 2.8E−03 | 3.8E−03 | 3.0E−03 | 4.1E−03 | Yes | CYP39A1 | Hs.387367 | cytochrome P450, family 39, subfamily A, |
| | | | | | | | | polypeptide 1 |
| 221003_s_at | 1.1E−03 | 1.8E−03 | 1.7E−03 | 4.5E−03 | Yes | FLJ12577 | Hs.87159 | hypothetical protein FLJ12577 |
| 221651_x_at | 1.4E−06 | 1.2E−10 | 1.6E−03 | 1.9E−03 | Yes | — | — | — |
| 221671_x_at | 1.7E−05 | 5.7E−05 | 3.1E−03 | 3.3E−03 | Yes | — | Hs.377975 | Homo sapiens immunoglobulin kappa light |
| | | | | | | | | chain mRNA, partial cds |
| 222307_at | 2.1E−03 | 3.6E−03 | 1.3E−03 | 3.6E−03 | Yes | — | Hs.293219 | Homo sapiens cDNA FLJ40384 fis, clone |
| | | | | | | | | TESTI2035796. |
| 203722_at | 3.8E−03 | 8.4E−05 | 6.9E−03 | 1.4E−03 | No | ALDH4A1 | Hs.77448 | aldehyde dehydrogenase 4 family, member |
| | | | | | | | | A1 |
| 219998_at | 3.0E−03 | 1.7E−03 | 4.3E−03 | 2.2E−03 | No | HSPC159 | Hs.372208 | HSPC159 protein |
| 221487_s_at | 1.1E−03 | 1.6E−03 | 4.2E−03 | 8.1E−03 | No | ENSA | Hs.511916 | endosulfine alpha |

TABLE 3A
Genes With Reduced Expression in CD40-Sensitive Cell Lines

| | Log2 fluorescence intensity | | | | | | | | | | | |
|---|---|---|---|---|---|---|---|---|---|---|---|---|
| | | | | | | | | | | | | D4 |
| Probesets | Ly1 (A1) | Ly1 (A2) | Ly1 (A3) | Ly7 (B1) | Ly7 (B2) | Ly7 (B3) | Ly8 (C1) | Ly8 (C2) | Ly8 (C3) | D4 (D1) | D4 (D2) | (D3) |
| 200068_s_at | 11.55 | 11.59 | 11.58 | 11.36 | 11.29 | 11.33 | 11.54 | 11.50 | 11.46 | 11.10 | 11.03 | 11.12 |
| 200670_at | 9.90 | 9.51 | 10.05 | 7.15 | 7.18 | 6.83 | 10.00 | 9.39 | 9.51 | 7.84 | 8.10 | 7.97 |
| 200701_at | 9.15 | 8.73 | 9.02 | 7.92 | 8.05 | 7.83 | 9.19 | 8.73 | 8.79 | 7.60 | 7.84 | 7.82 |
| 200736_s_at | 10.16 | 9.88 | 9.93 | 9.46 | 9.47 | 9.25 | 10.48 | 10.24 | 10.32 | 9.01 | 9.19 | 9.02 |
| 200765_x_at | 7.24 | 7.35 | 7.27 | 6.39 | 6.26 | 6.25 | 7.89 | 7.69 | 7.88 | 4.96 | 5.26 | 4.97 |
| 200814_at | 11.23 | 11.07 | 11.11 | 10.08 | 10.11 | 9.72 | 11.39 | 11.31 | 11.15 | 9.27 | 9.52 | 9.31 |
| 200846_s_at | 10.32 | 10.20 | 10.21 | 10.01 | 9.94 | 9.99 | 10.33 | 10.27 | 10.36 | 9.88 | 9.98 | 9.98 |
| 200887_s_at | 8.85 | 8.58 | 8.62 | 7.98 | 7.58 | 7.65 | 8.68 | 8.76 | 8.89 | 7.28 | 7.06 | 7.32 |
| 200905_x_at | 10.71 | 10.51 | 10.44 | 8.84 | 8.76 | 8.82 | 10.60 | 10.37 | 10.45 | 7.96 | 8.09 | 8.08 |
| 200936_at | 13.09 | 13.10 | 13.08 | 12.67 | 12.70 | 12.72 | 13.12 | 13.07 | 13.17 | 12.87 | 12.92 | 12.89 |
| 200941_at | 8.93 | 8.75 | 8.68 | 8.02 | 7.60 | 7.79 | 8.94 | 8.91 | 8.98 | 7.68 | 7.63 | 7.98 |
| 201011_at | 8.78 | 8.70 | 8.80 | 8.43 | 8.50 | 8.39 | 8.85 | 8.88 | 9.00 | 8.55 | 8.44 | 8.52 |
| 201160_s_at | 10.78 | 10.64 | 10.85 | 10.21 | 10.01 | 10.16 | 10.72 | 10.68 | 10.91 | 5.15 | 5.16 | 5.18 |
| 201161_s_at | 9.93 | 9.63 | 9.57 | 8.77 | 8.90 | 8.58 | 10.01 | 9.76 | 9.50 | 5.58 | 5.61 | 5.17 |
| 201200_at | 9.71 | 9.46 | 9.66 | 8.50 | 8.91 | 8.89 | 9.99 | 9.89 | 9.75 | 8.43 | 8.18 | 8.64 |
| 201204_s_at | 8.32 | 8.44 | 8.93 | 7.02 | 6.75 | 7.19 | 8.67 | 8.70 | 8.46 | 7.20 | 6.92 | 7.29 |
| 201206_s_at | 8.01 | 8.19 | 8.16 | 6.60 | 6.77 | 6.20 | 8.28 | 8.39 | 8.02 | 6.49 | 6.91 | 6.67 |
| 201226_at | 10.47 | 10.43 | 10.39 | 9.23 | 9.27 | 9.21 | 10.48 | 10.34 | 10.46 | 10.07 | 10.15 | 10.12 |
| 201263_at | 9.79 | 9.91 | 9.59 | 8.72 | 8.90 | 8.55 | 10.22 | 9.92 | 9.87 | 7.74 | 8.00 | 8.28 |
| 201307_at | 9.03 | 9.14 | 9.22 | 7.75 | 7.71 | 7.77 | 8.91 | 8.93 | 9.13 | 6.67 | 6.96 | 6.87 |
| 201398_s_at | 10.71 | 10.97 | 10.99 | 9.80 | 9.77 | 9.93 | 10.77 | 10.90 | 10.84 | 9.34 | 9.30 | 9.55 |
| 201399_s_at | 10.49 | 10.25 | 10.36 | 9.13 | 9.20 | 9.45 | 10.24 | 10.05 | 10.25 | 8.37 | 8.56 | 8.59 |
| 201470_at | 10.34 | 10.47 | 10.20 | 5.78 | 5.46 | 5.80 | 10.52 | 10.53 | 10.57 | 8.93 | 8.75 | 8.96 |
| 201487_at | 9.57 | 9.89 | 9.84 | 8.74 | 8.66 | 8.76 | 9.45 | 9.69 | 9.68 | 8.63 | 8.60 | 8.76 |
| 201499_s_at | 10.04 | 9.96 | 10.06 | 9.43 | 9.25 | 9.29 | 10.00 | 9.92 | 10.11 | 9.35 | 9.30 | 9.52 |
| 201566_x_at | 8.27 | 8.30 | 8.07 | 7.46 | 7.54 | 7.66 | 8.77 | 9.00 | 8.56 | 6.89 | 6.73 | 6.82 |
| 201590_x_at | 9.41 | 9.46 | 9.20 | 7.12 | 6.89 | 6.92 | 9.49 | 9.28 | 9.26 | 7.00 | 6.97 | 6.73 |
| 201601_x_at | 10.14 | 9.56 | 9.84 | 7.96 | 7.90 | 7.87 | 10.08 | 9.97 | 10.05 | 6.93 | 6.95 | 6.88 |
| 201649_at | 9.26 | 9.15 | 9.06 | 8.16 | 8.54 | 8.32 | 9.34 | 9.24 | 9.18 | 5.72 | 5.44 | 5.23 |
| 201746_at | 9.96 | 9.88 | 10.01 | 9.17 | 9.14 | 9.09 | 9.99 | 9.94 | 9.86 | 9.33 | 9.49 | 9.47 |
| 201874_at | 8.65 | 8.67 | 8.51 | 7.63 | 7.84 | 7.49 | 8.49 | 8.47 | 8.33 | 7.32 | 7.26 | 7.28 |
| 201937_s_at | 8.28 | 8.24 | 7.98 | 7.65 | 7.54 | 7.46 | 8.22 | 8.16 | 8.23 | 6.94 | 7.17 | 6.86 |
| 201968_s_at | 9.90 | 9.45 | 9.75 | 8.53 | 8.30 | 8.34 | 10.01 | 9.60 | 9.89 | 7.28 | 7.59 | 7.56 |
| 202096_s_at | 9.04 | 8.67 | 8.55 | 7.26 | 7.16 | 7.16 | 9.08 | 8.86 | 8.89 | 6.28 | 6.06 | 6.06 |
| 202123_s_at | 8.35 | 8.27 | 8.44 | 7.06 | 6.75 | 7.09 | 8.21 | 8.19 | 8.37 | 7.72 | 7.78 | 7.90 |
| 202261_at | 8.45 | 8.44 | 8.35 | 8.03 | 8.13 | 8.00 | 8.55 | 8.39 | 8.47 | 8.18 | 8.14 | 8.06 |
| 202279_at | 10.46 | 10.39 | 10.33 | 9.96 | 9.87 | 9.78 | 10.83 | 10.78 | 10.84 | 9.96 | 9.96 | 10.09 |
| 202315_s_at | 8.91 | 9.16 | 9.06 | 7.33 | 7.36 | 7.13 | 9.12 | 9.05 | 8.71 | 7.02 | 6.91 | 6.89 |
| 202382_s_at | 7.66 | 7.40 | 7.34 | 6.90 | 6.62 | 6.74 | 7.77 | 7.58 | 7.46 | 4.95 | 5.16 | 4.96 |
| 202429_s_at | 8.69 | 8.33 | 8.33 | 6.48 | 6.11 | 6.45 | 8.27 | 8.42 | 8.43 | 6.21 | 6.23 | 6.81 |
| 202443_x_at | 7.54 | 7.27 | 7.22 | 6.68 | 6.36 | 6.41 | 7.68 | 7.49 | 7.39 | 4.66 | 4.84 | 4.93 |
| 202447_at | 8.94 | 8.90 | 8.98 | 8.55 | 8.46 | 8.40 | 8.90 | 8.88 | 8.92 | 7.41 | 7.41 | 7.73 |
| 202457_s_at | 8.98 | 8.52 | 8.87 | 7.08 | 6.82 | 7.34 | 8.70 | 8.42 | 8.94 | 7.48 | 7.32 | 7.74 |
| 202536_at | 6.73 | 6.78 | 7.17 | 5.90 | 5.69 | 6.05 | 7.11 | 6.96 | 7.50 | 4.24 | 4.27 | 4.34 |
| 202696_at | 8.63 | 8.47 | 8.57 | 8.33 | 8.21 | 8.18 | 8.64 | 8.55 | 8.62 | 8.10 | 8.25 | 8.24 |
| 202732_at | 9.53 | 9.63 | 9.36 | 7.48 | 7.88 | 7.41 | 9.14 | 9.48 | 9.33 | 6.59 | 7.03 | 6.85 |
| 202811_at | 7.98 | 7.82 | 8.04 | 6.68 | 6.70 | 6.49 | 7.80 | 7.93 | 7.98 | 6.91 | 6.98 | 7.18 |
| 202982_s_at | 7.96 | 7.90 | 7.81 | 6.53 | 6.57 | 6.55 | 7.83 | 7.69 | 7.85 | 5.02 | 4.88 | 4.71 |
| 203031_s_at | 9.17 | 8.91 | 8.84 | 7.06 | 7.05 | 6.71 | 8.96 | 8.80 | 8.66 | 7.53 | 7.72 | 7.73 |
| 203113_s_at | 11.40 | 11.29 | 11.21 | 10.75 | 10.77 | 10.75 | 11.45 | 11.23 | 11.34 | 10.48 | 10.71 | 10.76 |
| 203142_s_at | 9.52 | 9.43 | 9.39 | 8.05 | 8.12 | 7.98 | 9.56 | 9.21 | 9.24 | 8.18 | 8.17 | 8.23 |
| 203217_s_at | 8.27 | 7.88 | 8.31 | 6.11 | 6.53 | 6.65 | 8.29 | 8.06 | 8.15 | 6.39 | 6.59 | 6.34 |
| 203335_at | 8.36 | 8.34 | 8.58 | 4.78 | 4.62 | 4.75 | 8.75 | 8.33 | 8.32 | 4.27 | 4.32 | 4.34 |
| 203343_at | 7.22 | 7.64 | 7.64 | 6.30 | 6.60 | 6.64 | 7.23 | 7.44 | 7.64 | 5.30 | 5.93 | 5.96 |
| 203459_s_at | 7.77 | 7.61 | 7.54 | 7.18 | 7.07 | 6.91 | 7.60 | 7.49 | 7.63 | 6.75 | 6.55 | 6.41 |
| 203659_s_at | 7.56 | 7.54 | 7.97 | 6.43 | 6.42 | 6.64 | 7.45 | 7.38 | 7.71 | 5.96 | 6.07 | 6.35 |
| 203825_at | 8.10 | 8.25 | 7.95 | 6.94 | 6.97 | 6.96 | 7.96 | 8.06 | 7.95 | 6.94 | 6.99 | 6.99 |
| 203957_at | 7.85 | 7.83 | 7.84 | 7.14 | 7.31 | 7.17 | 7.78 | 8.04 | 7.98 | 5.89 | 6.09 | 6.18 |
| 204003_s_at | 8.03 | 8.12 | 8.04 | 7.55 | 7.34 | 7.41 | 7.76 | 7.89 | 7.93 | 7.07 | 7.02 | 7.19 |
| 204044_at | 7.91 | 7.68 | 7.81 | 6.17 | 6.54 | 6.44 | 8.35 | 8.23 | 7.87 | 6.71 | 6.97 | 6.82 |
| 204168_at | 7.91 | 7.98 | 7.95 | 6.83 | 6.90 | 6.87 | 8.16 | 8.21 | 8.29 | 7.16 | 7.21 | 7.20 |
| 204172_at | 6.16 | 6.10 | 6.51 | 4.77 | 4.76 | 4.83 | 6.05 | 6.05 | 6.38 | 3.87 | 4.57 | 4.49 |
| 204220_at | 9.58 | 9.35 | 9.28 | 8.69 | 8.23 | 8.47 | 9.44 | 9.14 | 9.41 | 7.38 | 7.13 | 7.52 |
| 204234_s_at | 7.75 | 7.48 | 7.28 | 6.52 | 6.42 | 6.36 | 7.61 | 7.16 | 7.42 | 6.00 | 6.33 | 6.29 |
| 204256_at | 7.12 | 7.57 | 8.02 | 5.14 | 5.45 | 5.33 | 7.07 | 7.58 | 7.74 | 4.74 | 4.75 | 5.30 |
| 204279_at | 10.03 | 10.03 | 10.06 | 8.83 | 9.06 | 8.96 | 10.09 | 9.94 | 10.06 | 5.49 | 5.88 | 5.75 |
| 204326_x_at | 10.66 | 10.55 | 10.74 | 7.84 | 8.05 | 7.93 | 10.79 | 10.65 | 10.45 | 9.44 | 9.28 | 9.42 |
| 204386_s_at | 10.92 | 11.04 | 11.05 | 10.20 | 10.07 | 10.03 | 11.00 | 11.07 | 11.02 | 10.41 | 10.21 | 10.42 |
| 204418_x_at | 7.57 | 7.57 | 7.42 | 6.44 | 6.66 | 6.67 | 7.37 | 7.35 | 7.12 | 6.77 | 6.50 | 6.47 |
| 204565_at | 9.31 | 9.15 | 9.07 | 7.55 | 7.59 | 7.51 | 9.18 | 8.84 | 9.12 | 6.36 | 6.31 | 6.69 |
| 204766_s_at | 11.23 | 10.84 | 11.06 | 8.35 | 7.85 | 7.99 | 11.02 | 10.41 | 10.57 | 8.52 | 8.62 | 8.73 |
| 204769_s_at | 7.91 | 8.05 | 7.79 | 6.83 | 7.05 | 7.05 | 7.76 | 7.84 | 7.87 | 6.10 | 6.19 | 6.19 |
| 204779_s_at | 8.11 | 8.13 | 8.06 | 7.44 | 7.30 | 7.27 | 8.24 | 8.23 | 8.58 | 6.94 | 6.95 | 7.03 |
| 204806_x_at | 10.73 | 10.67 | 10.38 | 8.83 | 8.96 | 8.54 | 10.45 | 10.30 | 10.31 | 7.67 | 7.82 | 7.49 |
| 204937_s_at | 8.57 | 8.55 | 8.62 | 7.61 | 7.47 | 7.55 | 8.75 | 8.61 | 8.79 | 5.72 | 5.86 | 6.24 |
| 205048_s_at | 8.88 | 8.65 | 8.92 | 6.13 | 6.37 | 6.19 | 9.07 | 9.15 | 8.77 | 6.33 | 5.82 | 6.60 |
| 205297_s_at | 10.93 | 10.97 | 10.91 | 9.82 | 9.84 | 9.68 | 10.91 | 10.80 | 10.64 | 9.51 | 9.79 | 9.71 |
| 205659_at | 6.94 | 7.23 | 7.38 | 6.06 | 6.40 | 6.44 | 7.24 | 7.56 | 7.73 | 6.08 | 6.31 | 6.27 |
| 206928_at | 6.49 | 6.45 | 6.68 | 5.92 | 5.81 | 6.05 | 6.67 | 6.37 | 6.69 | 5.72 | 5.55 | 5.88 |
| 207181_s_at | 6.65 | 6.64 | 6.83 | 6.24 | 6.01 | 6.12 | 6.78 | 6.80 | 6.81 | 5.49 | 5.27 | 5.07 |
| 207304_at | 4.80 | 4.81 | 4.96 | 4.47 | 4.44 | 4.35 | 4.95 | 4.86 | 5.03 | 4.30 | 4.35 | 4.36 |
| 207585_s_at | 11.22 | 11.31 | 11.37 | 10.89 | 10.90 | 10.84 | 11.28 | 11.29 | 11.46 | 10.67 | 10.73 | 10.80 |
| 207777_s_at | 6.27 | 6.09 | 6.18 | 4.81 | 4.55 | 4.62 | 6.47 | 6.23 | 6.26 | 5.06 | 4.91 | 4.78 |
| 207809_s_at | 8.69 | 8.51 | 8.58 | 8.01 | 8.05 | 8.04 | 8.73 | 8.63 | 8.75 | 8.13 | 8.02 | 8.25 |
| 208490_x_at | 7.95 | 8.04 | 7.69 | 6.88 | 6.87 | 7.11 | 7.91 | 7.82 | 7.95 | 7.21 | 7.01 | 7.24 |
| 208527_x_at | 6.57 | 6.46 | 6.47 | 5.60 | 5.62 | 5.52 | 6.69 | 6.57 | 6.44 | 5.51 | 5.81 | 5.64 |
| 208579_x_at | 9.06 | 8.78 | 8.87 | 7.67 | 7.47 | 7.59 | 9.32 | 9.02 | 9.20 | 7.38 | 7.66 | 7.70 |
| 208581_x_at | 10.94 | 10.48 | 10.68 | 8.95 | 9.18 | 8.79 | 10.84 | 10.43 | 10.47 | 9.54 | 9.85 | 9.65 |
| 208690_s_at | 10.01 | 9.98 | 9.74 | 7.90 | 7.94 | 7.68 | 10.10 | 10.26 | 10.33 | 6.86 | 6.99 | 6.98 |
| 208729_x_at | 12.62 | 12.49 | 12.18 | 9.61 | 9.63 | 9.55 | 12.28 | 12.26 | 12.09 | 8.01 | 8.43 | 8.04 |
| 208812_x_at | 12.57 | 12.47 | 12.47 | 11.36 | 11.55 | 11.37 | 12.49 | 12.38 | 12.41 | 7.97 | 8.09 | 8.03 |
| 208918_s_at | 9.26 | 9.13 | 9.32 | 8.57 | 8.44 | 8.48 | 9.08 | 8.90 | 9.12 | 8.02 | 8.15 | 8.35 |
| 209040_s_at | 10.14 | 10.23 | 10.31 | 9.50 | 9.61 | 9.63 | 10.20 | 10.16 | 10.31 | 7.41 | 7.62 | 7.86 |
| 209112_at | 7.60 | 7.22 | 7.56 | 4.71 | 4.75 | 5.04 | 7.34 | 7.32 | 7.74 | 5.16 | 5.22 | 5.80 |
| 209124_at | 9.60 | 9.57 | 9.51 | 9.16 | 8.93 | 9.01 | 9.72 | 9.82 | 9.83 | 9.34 | 9.22 | 9.27 |
| 209138_x_at | 12.64 | 12.79 | 12.71 | 6.60 | 6.83 | 6.90 | 12.81 | 12.92 | 12.67 | 9.57 | 10.33 | 9.63 |
| 209140_x_at | 13.05 | 12.93 | 13.01 | 10.68 | 10.95 | 10.64 | 13.05 | 12.96 | 12.89 | 8.24 | 8.59 | 8.46 |
| 209175_at | 7.32 | 7.45 | 7.66 | 6.95 | 6.67 | 6.67 | 7.53 | 7.63 | 7.64 | 6.60 | 6.64 | 6.92 |
| 209201_x_at | 11.47 | 11.31 | 11.07 | 9.68 | 9.77 | 9.27 | 11.34 | 11.22 | 10.55 | 9.32 | 9.43 | 8.91 |
| 209472_at | 8.01 | 7.93 | 8.25 | 5.69 | 5.52 | 5.44 | 8.28 | 8.05 | 8.03 | 6.65 | 6.75 | 7.01 |
| 209476_at | 10.41 | 10.47 | 10.59 | 10.12 | 9.99 | 10.09 | 10.37 | 10.56 | 10.45 | 10.00 | 9.76 | 10.02 |
| 209591_s_at | 8.09 | 8.13 | 7.69 | 6.43 | 6.95 | 6.78 | 9.09 | 8.80 | 8.50 | 5.69 | 6.10 | 5.75 |
| 209970_x_at | 7.75 | 8.08 | 7.53 | 5.51 | 5.56 | 5.28 | 7.44 | 7.98 | 7.85 | 5.44 | 5.42 | 5.20 |
| 209995_s_at | 11.95 | 12.19 | 12.19 | 10.79 | 11.03 | 10.72 | 12.13 | 12.27 | 12.15 | 8.59 | 8.80 | 8.85 |
| 210136_at | 7.98 | 7.94 | 8.02 | 7.00 | 6.68 | 6.88 | 8.62 | 8.26 | 8.27 | 6.12 | 6.22 | 6.31 |
| 210844_x_at | 7.74 | 8.03 | 7.68 | 6.98 | 6.95 | 7.11 | 8.35 | 8.16 | 8.46 | 5.77 | 5.60 | 5.57 |
| 211061_s_at | 9.41 | 9.28 | 9.69 | 8.10 | 8.51 | 8.23 | 9.55 | 9.38 | 9.15 | 7.45 | 8.20 | 7.71 |
| 211089_s_at | 6.09 | 6.08 | 6.26 | 5.46 | 5.35 | 5.56 | 5.96 | 6.16 | 6.19 | 5.37 | 5.54 | 5.58 |
| 211366_x_at | 8.01 | 8.27 | 8.09 | 5.91 | 6.04 | 5.89 | 8.06 | 8.05 | 8.33 | 5.83 | 6.13 | 5.67 |
| 211528_x_at | 11.97 | 11.71 | 11.85 | 10.27 | 10.51 | 10.06 | 11.95 | 11.71 | 11.50 | 8.78 | 9.28 | 8.87 |
| 211529_x_at | 12.05 | 11.67 | 11.65 | 10.28 | 10.45 | 10.02 | 11.73 | 11.63 | 11.48 | 8.67 | 9.12 | 8.55 |
| 211530_x_at | 10.43 | 10.30 | 10.28 | 9.19 | 9.49 | 9.38 | 10.13 | 10.25 | 9.97 | 8.63 | 8.77 | 8.66 |
| 211799_x_at | 10.60 | 10.58 | 10.41 | 8.50 | 9.02 | 8.61 | 10.56 | 10.34 | 10.21 | 6.38 | 7.01 | 6.59 |
| 211911_x_at | 12.27 | 12.11 | 12.01 | 9.49 | 9.50 | 9.28 | 12.20 | 11.97 | 11.83 | 7.81 | 7.96 | 7.69 |
| 211919_s_at | 11.52 | 11.20 | 11.09 | 9.77 | 9.75 | 9.40 | 11.31 | 11.17 | 10.58 | 9.44 | 9.42 | 9.03 |
| 211989_at | 9.42 | 9.47 | 9.28 | 8.74 | 8.75 | 8.58 | 9.39 | 9.41 | 9.61 | 8.55 | 8.68 | 8.83 |
| 212057_at | 9.16 | 9.14 | 9.32 | 7.09 | 6.77 | 7.02 | 9.35 | 9.32 | 8.96 | 6.97 | 7.41 | 7.48 |
| 212071_s_at | 10.87 | 11.02 | 10.95 | 8.73 | 8.62 | 8.74 | 10.80 | 10.94 | 10.89 | 4.78 | 5.45 | 5.25 |
| 212149_at | 9.42 | 9.01 | 9.46 | 8.28 | 8.03 | 8.29 | 9.29 | 8.99 | 9.46 | 5.18 | 5.09 | 5.85 |
| 212382_at | 7.45 | 7.94 | 8.47 | 5.20 | 5.31 | 5.62 | 7.72 | 8.13 | 8.50 | 4.76 | 4.97 | 5.23 |
| 212385_at | 7.45 | 7.66 | 7.79 | 6.43 | 6.27 | 6.42 | 7.57 | 7.66 | 8.04 | 6.03 | 6.36 | 6.17 |
| 212386_at | 10.10 | 10.18 | 10.39 | 8.61 | 8.59 | 8.78 | 10.29 | 10.46 | 10.45 | 6.64 | 7.37 | 7.24 |
| 212387_at | 8.77 | 8.61 | 8.89 | 7.31 | 7.19 | 7.22 | 8.93 | 9.01 | 9.06 | 6.07 | 6.29 | 6.34 |
| 212408_at | 8.51 | 8.17 | 8.47 | 7.09 | 7.12 | 7.36 | 8.44 | 8.16 | 8.50 | 6.92 | 6.92 | 7.44 |
| 212446_s_at | 5.54 | 5.36 | 5.36 | 3.63 | 3.80 | 3.86 | 5.39 | 5.46 | 5.89 | 3.80 | 3.82 | 3.78 |
| 212507_at | 7.71 | 7.47 | 7.74 | 6.39 | 6.37 | 6.51 | 7.47 | 7.73 | 7.89 | 6.14 | 6.00 | 6.54 |
| 212547_at | 7.80 | 7.94 | 7.89 | 6.72 | 6.52 | 6.58 | 7.83 | 7.92 | 8.09 | 6.52 | 6.74 | 6.91 |
| 212739_s_at | 10.60 | 10.47 | 10.47 | 8.69 | 8.86 | 8.60 | 10.57 | 10.38 | 10.27 | 6.39 | 5.60 | 6.02 |
| 212774_at | 8.74 | 8.65 | 9.00 | 7.10 | 6.99 | 7.50 | 8.67 | 8.84 | 9.10 | 5.74 | 5.84 | 6.27 |
| 212792_at | 6.10 | 6.37 | 6.29 | 5.46 | 5.49 | 5.56 | 6.53 | 6.54 | 6.45 | 5.68 | 5.62 | 5.35 |
| 212798_s_at | 8.29 | 8.33 | 8.51 | 6.44 | 6.20 | 6.62 | 8.29 | 8.42 | 8.57 | 6.12 | 6.13 | 6.40 |
| 212946_at | 8.76 | 8.42 | 8.49 | 7.22 | 7.56 | 7.47 | 8.67 | 8.22 | 8.59 | 6.28 | 6.15 | 6.40 |
| 213020_at | 5.61 | 5.70 | 5.66 | 5.39 | 5.42 | 5.28 | 5.64 | 5.72 | 5.76 | 5.39 | 5.47 | 5.38 |
| 213116_at | 7.40 | 7.52 | 7.74 | 5.87 | 6.02 | 5.88 | 7.47 | 7.53 | 7.72 | 5.68 | 5.54 | 5.62 |
| 213233_s_at | 8.79 | 8.57 | 8.74 | 7.14 | 7.45 | 7.12 | 8.78 | 8.90 | 8.56 | 7.11 | 7.30 | 7.16 |
| 213293_s_at | 8.61 | 8.63 | 9.05 | 7.83 | 7.73 | 7.59 | 8.84 | 9.12 | 8.93 | 7.24 | 7.36 | 7.49 |
| 213357_at | 8.43 | 8.33 | 8.64 | 8.03 | 7.79 | 7.90 | 8.72 | 8.40 | 8.60 | 7.00 | 6.87 | 7.26 |
| 213361_at | 6.49 | 6.45 | 6.35 | 6.08 | 5.97 | 5.85 | 6.60 | 6.41 | 6.72 | 5.51 | 5.65 | 5.55 |
| 213435_at | 6.68 | 6.84 | 7.27 | 5.62 | 5.52 | 5.94 | 7.01 | 7.31 | 7.28 | 5.24 | 4.63 | 5.11 |
| 213793_s_at | 6.83 | 6.31 | 6.61 | 5.04 | 5.23 | 5.20 | 6.60 | 6.09 | 6.60 | 4.26 | 4.45 | 4.31 |
| 213891_s_at | 9.12 | 9.15 | 9.28 | 7.28 | 7.27 | 7.51 | 9.30 | 9.35 | 9.47 | 5.41 | 5.67 | 5.90 |
| 214022_s_at | 10.24 | 9.80 | 9.85 | 7.86 | 8.05 | 7.67 | 10.10 | 10.14 | 10.02 | 6.52 | 6.52 | 6.51 |
| 214032_at | 7.58 | 7.49 | 7.36 | 5.75 | 5.83 | 5.79 | 7.78 | 7.52 | 7.54 | 5.55 | 5.87 | 5.53 |
| 214172_x_at | 6.23 | 6.11 | 6.26 | 5.23 | 5.42 | 5.37 | 6.26 | 6.39 | 6.23 | 5.10 | 5.07 | 4.87 |
| 214369_s_at | 8.61 | 8.51 | 8.49 | 7.88 | 7.64 | 7.67 | 8.17 | 8.15 | 8.36 | 6.66 | 6.46 | 6.81 |
| 214394_x_at | 11.62 | 11.69 | 11.68 | 11.30 | 11.30 | 11.37 | 11.78 | 11.70 | 11.82 | 11.37 | 11.31 | 11.37 |
| 214459_x_at | 12.47 | 12.49 | 12.46 | 11.42 | 11.67 | 11.41 | 12.54 | 12.37 | 12.26 | 8.26 | 8.86 | 8.86 |
| 214512_s_at | 10.28 | 10.44 | 10.44 | 7.82 | 8.00 | 7.92 | 10.09 | 10.32 | 10.42 | 8.48 | 8.52 | 8.73 |
| 214677_x_at | 13.42 | 13.48 | 13.31 | 7.13 | 6.91 | 7.06 | 13.31 | 13.41 | 13.41 | 9.71 | 10.24 | 9.85 |
| 214749_s_at | 9.55 | 9.46 | 9.57 | 8.76 | 8.74 | 8.72 | 9.38 | 9.30 | 9.31 | 6.99 | 6.52 | 6.89 |
| 214835_s_at | 7.93 | 7.92 | 8.33 | 6.59 | 6.67 | 6.29 | 7.77 | 8.21 | 8.07 | 6.84 | 6.94 | 7.07 |
| 214916_x_at | 8.71 | 8.55 | 8.48 | 7.93 | 8.06 | 7.82 | 8.51 | 8.37 | 8.28 | 4.96 | 5.53 | 5.27 |
| 215121_x_at | 12.69 | 12.81 | 12.63 | 7.09 | 7.04 | 7.67 | 12.61 | 12.72 | 12.67 | 9.64 | 10.21 | 9.61 |
| 215516_at | 4.64 | 4.43 | 4.42 | 4.05 | 3.91 | 4.02 | 4.32 | 4.35 | 4.46 | 4.09 | 3.94 | 4.00 |
| 215772_x_at | 8.32 | 8.05 | 8.63 | 6.43 | 6.59 | 6.62 | 8.64 | 8.37 | 8.27 | 7.08 | 7.01 | 7.40 |
| 215946_x_at | 9.45 | 10.25 | 10.15 | 6.14 | 6.52 | 6.35 | 9.37 | 9.73 | 9.78 | 7.00 | 7.41 | 6.77 |
| 216526_x_at | 12.46 | 12.36 | 12.44 | 11.35 | 11.52 | 11.28 | 12.40 | 12.36 | 12.30 | 7.97 | 7.98 | 7.86 |
| 216733_s_at | 8.74 | 8.51 | 8.97 | 5.02 | 5.17 | 5.35 | 8.26 | 8.11 | 8.29 | 5.34 | 5.38 | 5.41 |
| 216973_s_at | 8.07 | 8.20 | 8.08 | 7.57 | 7.51 | 7.42 | 8.31 | 8.29 | 8.61 | 6.85 | 7.21 | 7.05 |
| 216976_s_at | 7.27 | 7.20 | 7.20 | 6.32 | 6.39 | 6.39 | 7.67 | 7.24 | 7.55 | 6.14 | 6.05 | 5.85 |
| 217028_at | 10.94 | 11.03 | 11.08 | 9.61 | 9.69 | 9.31 | 10.99 | 10.98 | 10.65 | 9.27 | 9.15 | 9.06 |
| 217436_x_at | 9.84 | 9.56 | 9.53 | 7.91 | 8.38 | 8.22 | 9.78 | 9.39 | 9.37 | 7.30 | 7.54 | 7.52 |
| 217456_x_at | 9.30 | 9.09 | 9.12 | 7.62 | 7.65 | 7.54 | 9.06 | 8.89 | 9.02 | 6.80 | 7.11 | 6.96 |
| 217809_at | 11.42 | 11.35 | 11.46 | 10.34 | 10.24 | 10.27 | 11.37 | 11.33 | 11.45 | 10.99 | 10.96 | 11.10 |
| 217839_at | 9.87 | 9.61 | 9.56 | 9.02 | 8.94 | 8.84 | 9.68 | 9.54 | 9.58 | 8.09 | 8.12 | 7.85 |
| 217911_s_at | 7.04 | 7.17 | 7.41 | 5.54 | 4.84 | 5.01 | 8.02 | 7.90 | 7.53 | 4.87 | 4.90 | 4.80 |
| 217940_s_at | 8.53 | 8.41 | 8.38 | 7.03 | 7.24 | 7.17 | 8.65 | 8.30 | 8.60 | 7.24 | 7.50 | 7.63 |
| 217950_at | 9.21 | 8.99 | 9.06 | 8.31 | 8.06 | 8.19 | 9.21 | 8.87 | 9.11 | 8.01 | 8.00 | 8.21 |
| 217988_at | 9.98 | 9.46 | 9.52 | 7.84 | 7.67 | 7.52 | 9.70 | 9.38 | 9.37 | 7.88 | 8.29 | 8.17 |
| 218026_at | 9.30 | 9.33 | 9.29 | 8.59 | 8.50 | 8.61 | 9.44 | 9.39 | 9.61 | 8.57 | 8.49 | 8.71 |
| 218093_s_at | 8.39 | 8.29 | 8.68 | 6.34 | 6.67 | 6.70 | 7.80 | 7.76 | 8.19 | 6.85 | 6.74 | 7.08 |
| 218100_s_at | 8.05 | 7.94 | 8.36 | 6.25 | 5.99 | 6.11 | 8.58 | 8.56 | 8.53 | 4.84 | 5.28 | 5.82 |
| 218187_s_at | 8.45 | 8.39 | 8.39 | 7.56 | 7.31 | 7.34 | 8.31 | 8.31 | 8.40 | 7.48 | 7.17 | 7.34 |
| 218224_at | 7.21 | 7.30 | 7.85 | 4.08 | 4.31 | 4.29 | 7.70 | 7.39 | 7.49 | 4.22 | 4.14 | 4.37 |
| 218276_s_at | 6.55 | 6.49 | 6.39 | 5.84 | 5.61 | 5.66 | 7.26 | 7.03 | 7.00 | 5.24 | 5.20 | 5.39 |
| 218285_s_at | 7.37 | 7.59 | 7.68 | 5.86 | 5.86 | 5.95 | 7.44 | 7.46 | 7.77 | 6.01 | 6.02 | 6.32 |
| 218316_at | 8.86 | 8.73 | 8.91 | 8.47 | 8.33 | 8.42 | 8.80 | 8.85 | 8.66 | 7.95 | 8.00 | 7.87 |
| 218343_s_at | 7.51 | 7.20 | 7.47 | 6.40 | 6.18 | 6.20 | 7.54 | 7.28 | 7.52 | 5.91 | 5.76 | 6.22 |
| 218358_at | 9.73 | 9.86 | 10.01 | 8.58 | 8.32 | 8.67 | 9.64 | 9.66 | 9.51 | 8.64 | 8.35 | 8.65 |
| 218377_s_at | 8.68 | 8.77 | 8.70 | 7.98 | 8.16 | 8.03 | 8.67 | 8.63 | 8.59 | 8.29 | 8.32 | 8.27 |
| 218490_s_at | 4.57 | 4.60 | 4.72 | 4.29 | 4.31 | 4.25 | 4.73 | 4.61 | 4.60 | 3.87 | 3.87 | 3.88 |
| 218491_s_at | 9.04 | 9.02 | 8.74 | 8.32 | 8.14 | 8.08 | 8.90 | 8.69 | 8.80 | 7.96 | 7.66 | 8.08 |
| 218543_s_at | 7.65 | 7.40 | 7.72 | 6.47 | 6.27 | 6.45 | 7.87 | 7.65 | 7.73 | 5.96 | 5.64 | 5.98 |
| 218557_at | 9.39 | 9.27 | 9.28 | 7.87 | 7.84 | 7.77 | 9.44 | 9.11 | 9.15 | 7.28 | 7.20 | 7.40 |
| 218633_x_at | 8.66 | 8.70 | 8.75 | 7.93 | 7.81 | 7.93 | 8.59 | 8.64 | 8.67 | 7.01 | 7.09 | 7.37 |
| 218648_at | 7.91 | 7.89 | 7.77 | 5.87 | 6.13 | 6.37 | 7.75 | 7.88 | 7.87 | 6.37 | 6.08 | 6.29 |
| 218735_s_at | 7.74 | 8.04 | 8.29 | 6.75 | 7.00 | 6.67 | 8.00 | 8.13 | 8.28 | 6.16 | 6.40 | 6.59 |
| 218738_s_at | 9.42 | 9.35 | 9.63 | 8.74 | 8.59 | 8.59 | 9.37 | 9.53 | 9.59 | 8.71 | 8.65 | 8.82 |
| 218773_s_at | 8.82 | 8.40 | 8.76 | 7.03 | 7.25 | 7.13 | 8.78 | 8.35 | 8.53 | 7.44 | 7.59 | 7.92 |
| 219008_at | 6.90 | 6.61 | 7.27 | 5.37 | 4.92 | 4.79 | 7.03 | 6.61 | 7.10 | 5.60 | 5.25 | 5.32 |
| 219184_x_at | 7.30 | 7.30 | 7.29 | 6.35 | 6.31 | 6.20 | 7.15 | 7.54 | 7.15 | 5.95 | 5.99 | 5.91 |
| 219209_at | 5.38 | 5.30 | 5.58 | 4.82 | 4.53 | 4.76 | 5.59 | 5.76 | 5.92 | 4.30 | 4.49 | 4.27 |
| 219215_s_at | 8.52 | 8.48 | 8.63 | 6.70 | 6.92 | 7.14 | 8.64 | 8.64 | 8.52 | 6.88 | 7.05 | 7.06 |
| 219248_at | 6.96 | 7.16 | 7.31 | 5.92 | 6.06 | 6.24 | 6.98 | 7.17 | 7.02 | 6.27 | 6.51 | 6.38 |
| 219471_at | 11.50 | 11.61 | 11.68 | 10.17 | 10.60 | 10.29 | 11.55 | 11.75 | 11.53 | 8.28 | 8.93 | 8.22 |
| 219489_s_at | 9.71 | 9.92 | 10.01 | 5.69 | 5.25 | 5.82 | 9.87 | 9.65 | 9.87 | 6.35 | 5.53 | 6.07 |
| 219563_at | 8.96 | 9.27 | 9.42 | 4.98 | 5.11 | 4.79 | 8.37 | 8.48 | 8.29 | 4.74 | 4.36 | 4.28 |
| 219664_s_at | 10.37 | 9.75 | 9.82 | 8.06 | 8.06 | 7.70 | 10.36 | 9.93 | 9.82 | 8.33 | 8.14 | 7.88 |
| 219767_s_at | 6.00 | 5.85 | 6.11 | 5.32 | 5.48 | 5.34 | 6.01 | 5.82 | 5.94 | 5.19 | 5.30 | 5.32 |
| 219822_at | 6.85 | 6.89 | 7.03 | 5.44 | 5.43 | 5.48 | 6.92 | 6.83 | 7.04 | 5.36 | 5.43 | 5.87 |
| 220741_s_at | 9.37 | 9.31 | 9.37 | 7.72 | 7.54 | 7.45 | 9.31 | 9.21 | 9.45 | 8.13 | 8.06 | 8.18 |
| 221234_s_at | 9.73 | 9.80 | 9.57 | 8.75 | 8.81 | 8.65 | 9.76 | 9.86 | 9.53 | 8.08 | 8.27 | 7.91 |
| 221614_s_at | 7.82 | 7.18 | 7.29 | 5.07 | 5.11 | 5.05 | 7.64 | 7.30 | 7.29 | 5.35 | 5.14 | 5.38 |
| 221847_at | 7.49 | 7.26 | 7.35 | 5.94 | 5.92 | 5.94 | 7.47 | 7.15 | 7.71 | 6.14 | 6.05 | 6.40 |
| 221874_at | 7.45 | 6.91 | 7.51 | 5.03 | 5.13 | 5.02 | 7.48 | 7.10 | 7.18 | 5.09 | 5.46 | 5.18 |
| 221875_x_at | 11.07 | 10.99 | 10.93 | 9.17 | 9.49 | 9.05 | 11.03 | 10.87 | 10.69 | 8.19 | 8.56 | 8.26 |
| 222146_s_at | 7.71 | 7.86 | 7.40 | 5.84 | 6.21 | 5.70 | 7.60 | 7.66 | 7.77 | 4.92 | 5.50 | 5.26 |
| 35671_at | 8.76 | 8.71 | 8.67 | 8.15 | 8.12 | 8.19 | 8.85 | 8.72 | 8.83 | 8.16 | 8.22 | 8.29 |
| 36830_at | 7.17 | 7.42 | 7.37 | 5.98 | 6.04 | 6.24 | 6.93 | 7.22 | 7.48 | 5.91 | 6.07 | 6.30 |
| 36936_at | 8.33 | 8.35 | 8.35 | 7.73 | 7.52 | 7.55 | 8.16 | 8.14 | 8.08 | 7.56 | 7.59 | 7.56 |
| 37384_at | 9.02 | 8.90 | 9.04 | 7.57 | 7.81 | 7.69 | 8.98 | 8.89 | 8.75 | 7.35 | 7.77 | 7.72 |
| 39318_at | 12.14 | 12.32 | 12.26 | 11.06 | 11.18 | 10.93 | 12.27 | 12.49 | 12.25 | 8.75 | 8.94 | 8.48 |
| 40148_at | 5.44 | 5.46 | 5.58 | 4.86 | 5.04 | 4.99 | 5.40 | 5.68 | 5.75 | 4.54 | 4.46 | 4.46 |
| 200660_at | 8.56 | 8.49 | 8.21 | 5.40 | 5.06 | 5.36 | 8.56 | 8.51 | 8.45 | 5.81 | 5.38 | 5.40 |
| 200671_s_at | 7.16 | 7.40 | 7.20 | 5.65 | 5.58 | 5.87 | 7.30 | 7.10 | 7.61 | 5.20 | 5.14 | 4.88 |
| 200672_x_at | 8.93 | 9.32 | 8.97 | 7.43 | 7.73 | 7.81 | 8.85 | 9.09 | 9.24 | 6.32 | 7.01 | 6.75 |
| 200704_at | 8.62 | 8.23 | 8.24 | 5.83 | 6.09 | 5.91 | 8.96 | 8.80 | 8.50 | 6.19 | 6.29 | 6.00 |
| 200706_s_at | 7.13 | 6.99 | 7.11 | 5.71 | 5.61 | 5.62 | 7.31 | 7.01 | 7.14 | 5.89 | 5.91 | 5.84 |
| 200782_at | 7.51 | 7.44 | 7.83 | 4.72 | 5.12 | 5.13 | 8.78 | 8.85 | 9.29 | 5.09 | 5.33 | 5.65 |
| 200923_at | 9.47 | 9.06 | 9.04 | 5.66 | 5.77 | 5.93 | 9.34 | 9.30 | 9.07 | 5.73 | 5.78 | 5.56 |
| 201005_at | 10.54 | 10.55 | 10.64 | 4.88 | 4.53 | 4.83 | 10.91 | 10.82 | 10.60 | 5.25 | 4.95 | 4.80 |
| 201426_s_at | 10.07 | 9.77 | 9.84 | 5.98 | 5.82 | 5.88 | 10.04 | 9.78 | 9.63 | 5.88 | 6.01 | 5.26 |
| 201485_s_at | 8.03 | 7.98 | 7.98 | 7.22 | 7.12 | 7.26 | 8.16 | 7.84 | 8.21 | 5.27 | 5.35 | 5.34 |
| 201792_at | 8.18 | 8.49 | 8.69 | 5.99 | 5.42 | 5.82 | 8.50 | 8.77 | 8.58 | 6.36 | 5.74 | 5.91 |
| 202218_s_at | 7.55 | 7.32 | 7.86 | 5.92 | 5.77 | 5.88 | 7.89 | 7.50 | 7.42 | 6.24 | 6.24 | 5.88 |
| 202283_at | 10.43 | 9.33 | 10.17 | 5.54 | 5.28 | 5.59 | 10.37 | 9.42 | 9.62 | 6.16 | 5.84 | 6.11 |
| 202719_s_at | 6.49 | 6.41 | 6.60 | 5.41 | 5.13 | 5.38 | 6.53 | 6.63 | 6.58 | 5.99 | 5.89 | 6.01 |
| 202855_s_at | 6.48 | 6.13 | 6.22 | 4.99 | 4.91 | 4.92 | 6.93 | 6.55 | 7.01 | 4.82 | 5.27 | 5.00 |
| 202856_s_at | 6.18 | 6.07 | 6.32 | 4.75 | 4.26 | 4.55 | 6.63 | 6.50 | 6.34 | 4.61 | 4.59 | 4.65 |
| 203045_at | 8.37 | 8.65 | 8.33 | 6.69 | 7.14 | 6.94 | 8.41 | 8.87 | 8.25 | 6.76 | 6.91 | 6.52 |
| 203063_at | 8.44 | 8.27 | 8.60 | 7.11 | 7.32 | 6.95 | 8.45 | 8.23 | 8.17 | 7.02 | 7.34 | 7.16 |
| 203068_at | 6.88 | 6.58 | 6.72 | 4.89 | 5.15 | 5.17 | 6.88 | 6.70 | 7.05 | 5.25 | 5.48 | 5.42 |
| 203211_s_at | 7.33 | 7.38 | 7.27 | 5.32 | 5.17 | 5.16 | 7.50 | 7.54 | 7.53 | 5.76 | 5.67 | 5.63 |
| 203212_s_at | 8.24 | 7.99 | 7.83 | 6.21 | 5.74 | 6.36 | 8.16 | 8.03 | 8.27 | 7.00 | 6.69 | 7.03 |
| 203222_s_at | 4.89 | 4.92 | 4.78 | 4.44 | 4.27 | 4.33 | 5.05 | 5.10 | 5.09 | 4.50 | 4.35 | 4.39 |
| 203305_at | 8.41 | 8.31 | 8.36 | 5.42 | 5.67 | 5.90 | 9.17 | 9.07 | 9.21 | 5.76 | 6.22 | 5.83 |
| 203408_s_at | 8.28 | 7.85 | 7.91 | 6.70 | 6.31 | 6.54 | 8.22 | 7.73 | 7.95 | 6.76 | 6.46 | 6.62 |
| 203557_s_at | 7.38 | 7.33 | 7.19 | 5.60 | 5.83 | 5.66 | 7.56 | 7.50 | 7.37 | 6.52 | 6.71 | 6.57 |
| 203708_at | 7.61 | 8.10 | 8.86 | 5.13 | 5.50 | 5.11 | 8.10 | 8.38 | 8.07 | 5.15 | 5.45 | 5.00 |
| 203853_s_at | 6.37 | 6.51 | 6.52 | 5.07 | 5.21 | 5.09 | 5.43 | 5.56 | 5.46 | 5.27 | 5.13 | 5.20 |
| 204305_at | 7.97 | 8.06 | 7.69 | 6.50 | 6.67 | 6.50 | 7.74 | 7.90 | 7.91 | 6.65 | 6.73 | 6.81 |
| 204821_at | 5.79 | 5.80 | 6.03 | 4.72 | 4.47 | 4.63 | 5.59 | 5.58 | 5.75 | 4.70 | 4.41 | 4.27 |
| 204994_at | 8.12 | 8.07 | 8.25 | 5.34 | 5.86 | 5.54 | 8.38 | 8.15 | 8.19 | 5.51 | 6.15 | 5.79 |
| 205173_x_at | 5.53 | 5.47 | 5.79 | 4.41 | 4.57 | 4.51 | 5.59 | 5.60 | 5.70 | 4.47 | 4.64 | 4.38 |
| 205181_at | 6.17 | 6.31 | 6.21 | 5.35 | 5.61 | 5.57 | 6.21 | 6.10 | 6.41 | 5.45 | 5.67 | 5.38 |
| 205366_s_at | 5.65 | 5.53 | 5.57 | 4.73 | 4.99 | 4.96 | 5.46 | 5.69 | 5.79 | 5.15 | 4.99 | 4.90 |
| 205401_at | 7.13 | 7.29 | 7.47 | 4.57 | 4.71 | 4.85 | 7.49 | 7.43 | 7.89 | 5.63 | 5.56 | 5.97 |
| 205668_at | 6.46 | 6.60 | 7.07 | 4.14 | 4.66 | 4.69 | 6.29 | 6.60 | 6.66 | 4.75 | 4.62 | 4.51 |
| 205691_at | 8.97 | 9.21 | 9.50 | 6.40 | 7.01 | 7.11 | 8.74 | 8.81 | 8.64 | 5.98 | 6.41 | 6.24 |
| 205790_at | 8.04 | 7.91 | 7.79 | 5.72 | 5.68 | 5.79 | 7.88 | 7.53 | 7.92 | 6.37 | 6.02 | 6.05 |
| 205932_s_at | 6.08 | 5.86 | 6.03 | 5.10 | 5.28 | 5.02 | 6.22 | 6.15 | 5.84 | 4.87 | 5.19 | 4.85 |
| 206082_at | 7.66 | 7.32 | 7.79 | 5.36 | 5.84 | 5.45 | 7.55 | 7.68 | 7.32 | 5.38 | 5.20 | 5.17 |
| 206261_at | 6.56 | 6.92 | 7.25 | 4.87 | 5.19 | 5.29 | 6.53 | 6.74 | 6.42 | 5.52 | 5.27 | 5.15 |
| 206437_at | 8.10 | 8.40 | 814 | 6.54 | 6.39 | 6.05 | 7.92 | 7.85 | 8.00 | 6.34 | 6.49 | 6.07 |
| 206554_x_at | 5.68 | 5.61 | 5.56 | 4.83 | 4.83 | 4.89 | 5.80 | 5.75 | 5.70 | 4.64 | 4.75 | 4.52 |
| 206591_at | 7.55 | 8.15 | 8.76 | 4.39 | 4.15 | 4.25 | 8.34 | 8.84 | 8.68 | 4.23 | 4.00 | 4.32 |
| 206660_at | 10.68 | 11.35 | 10.55 | 5.41 | 4.82 | 4.94 | 9.07 | 10.28 | 10.32 | 5.10 | 4.93 | 5.30 |
| 206698_at | 7.52 | 7.70 | 8.28 | 4.28 | 4.31 | 4.26 | 7.40 | 7.79 | 7.74 | 4.31 | 4.57 | 4.31 |
| 206866_at | 6.71 | 6.83 | 6.67 | 5.57 | 5.33 | 5.27 | 6.60 | 6.43 | 6.25 | 5.62 | 5.55 | 5.33 |
| 208146_s_at | 7.20 | 6.74 | 6.94 | 5.35 | 5.55 | 5.11 | 6.42 | 6.58 | 6.70 | 5.31 | 5.44 | 5.37 |
| 208964_s_at | 7.75 | 7.57 | 7.71 | 5.82 | 6.30 | 5.96 | 7.98 | 7.88 | 7.41 | 6.08 | 6.48 | 5.93 |
| 209129_at | 7.17 | 7.66 | 7.38 | 5.34 | 4.82 | 4.98 | 7.33 | 7.36 | 7.25 | 5.60 | 4.88 | 5.00 |
| 209310_s_at | 7.06 | 6.66 | 7.29 | 5.21 | 4.80 | 4.95 | 6.61 | 6.44 | 6.84 | 5.08 | 4.93 | 4.97 |
| 209398_at | 6.73 | 6.61 | 6.92 | 6.11 | 5.86 | 6.05 | 7.01 | 6.65 | 6.99 | 6.04 | 6.03 | 5.90 |
| 209590_at | 7.34 | 7.81 | 7.51 | 6.30 | 6.17 | 6.36 | 8.10 | 8.52 | 7.92 | 6.14 | 6.21 | 5.94 |
| 209728_at | 9.38 | 9.04 | 9.49 | 6.91 | 6.87 | 7.18 | 9.38 | 9.02 | 9.20 | 7.25 | 6.86 | 7.07 |
| 209790_s_at | 6.67 | 6.85 | 6.71 | 5.30 | 5.23 | 5.19 | 6.99 | 7.04 | 7.33 | 6.29 | 6.19 | 6.40 |
| 210427_x_at | 9.55 | 9.37 | 8.94 | 6.88 | 6.91 | 6.73 | 9.15 | 9.03 | 9.17 | 6.90 | 7.23 | 6.60 |
| 210715_s_at | 9.21 | 8.81 | 9.23 | 6.01 | 5.82 | 5.74 | 8.88 | 8.72 | 9.19 | 5.87 | 6.52 | 5.70 |
| 210830_s_at | 5.03 | 4.96 | 4.88 | 4.15 | 4.30 | 4.31 | 5.02 | 5.00 | 5.24 | 4.40 | 4.25 | 4.43 |
| 211367_s_at | 5.99 | 6.23 | 6.16 | 4.32 | 4.02 | 4.13 | 6.12 | 6.25 | 6.63 | 4.38 | 4.41 | 4.14 |
| 211368_s_at | 7.23 | 7.48 | 7.16 | 4.88 | 4.89 | 4.78 | 7.07 | 7.48 | 7.78 | 4.84 | 4.75 | 4.56 |
| 211812_s_at | 5.54 | 5.76 | 6.09 | 4.23 | 4.26 | 4.43 | 5.31 | 5.66 | 5.58 | 4.46 | 4.37 | 4.23 |
| 212268_at | 8.22 | 8.12 | 8.07 | 5.91 | 5.97 | 5.78 | 8.50 | 8.10 | 8.52 | 6.92 | 7.12 | 7.22 |
| 212442_s_at | 8.75 | 8.85 | 8.52 | 5.88 | 5.73 | 6.27 | 8.76 | 8.72 | 8.97 | 6.11 | 5.74 | 5.82 |
| 212561_at | 7.80 | 7.39 | 7.20 | 6.10 | 6.31 | 6.50 | 7.81 | 7.39 | 7.62 | 6.56 | 6.20 | 5.96 |
| 213147_at | 7.84 | 8.00 | 7.80 | 6.32 | 6.58 | 6.51 | 7.34 | 7.65 | 7.54 | 6.14 | 6.27 | 6.34 |
| 213150_at | 6.85 | 7.41 | 7.76 | 4.40 | 4.74 | 4.66 | 6.47 | 6.59 | 7.01 | 4.35 | 4.60 | 4.65 |
| 213371_at | 6.96 | 6.96 | 6.89 | 4.58 | 4.93 | 4.61 | 7.43 | 7.11 | 7.02 | 4.95 | 4.78 | 4.59 |
| 213419_at | 6.52 | 6.47 | 6.71 | 5.72 | 5.99 | 5.81 | 6.60 | 6.44 | 6.84 | 5.02 | 5.46 | 5.04 |
| 213502_x_at | 9.93 | 10.40 | 9.72 | 6.52 | 6.78 | 6.55 | 9.60 | 9.86 | 9.68 | 7.01 | 7.06 | 7.30 |
| 213503_x_at | 9.33 | 9.29 | 9.11 | 6.93 | 6.99 | 6.80 | 9.21 | 9.20 | 9.24 | 7.11 | 7.21 | 6.83 |
| 213566_at | 7.24 | 6.91 | 7.15 | 4.80 | 5.20 | 4.83 | 7.09 | 6.63 | 7.05 | 4.95 | 4.85 | 4.89 |
| 213568_at | 4.82 | 4.76 | 4.79 | 4.11 | 4.16 | 4.22 | 4.81 | 4.88 | 4.92 | 4.10 | 4.02 | 4.05 |
| 213572_s_at | 7.30 | 6.68 | 7.22 | 4.48 | 4.52 | 4.44 | 7.15 | 7.00 | 7.42 | 5.55 | 5.32 | 5.72 |
| 213625_at | 5.83 | 5.88 | 5.85 | 5.33 | 5.28 | 5.42 | 5.66 | 5.59 | 5.61 | 5.12 | 5.13 | 5.18 |
| 213788_s_at | 5.89 | 6.20 | 6.17 | 5.22 | 5.13 | 5.46 | 5.93 | 6.09 | 5.97 | 5.44 | 5.22 | 5.49 |
| 215071_s_at | 9.91 | 8.89 | 8.75 | 5.01 | 5.37 | 5.26 | 10.11 | 8.63 | 9.06 | 5.62 | 5.24 | 5.58 |
| 215379_x_at | 11.62 | 11.63 | 11.58 | 5.86 | 5.97 | 5.95 | 11.58 | 11.63 | 11.46 | 8.05 | 8.92 | 8.29 |
| 216565_x_at | 7.47 | 7.34 | 7.50 | 6.52 | 6.71 | 6.39 | 7.57 | 7.50 | 7.37 | 6.85 | 6.88 | 6.74 |
| 217022_s_at | 8.41 | 8.43 | 8.70 | 5.16 | 4.79 | 4.95 | 8.98 | 9.42 | 8.50 | 5.39 | 5.20 | 5.07 |
| 217360_x_at | 7.17 | 7.42 | 7.18 | 5.76 | 5.81 | 5.51 | 7.39 | 7.34 | 6.76 | 5.93 | 5.61 | 5.61 |
| 217759_at | 9.36 | 9.34 | 9.61 | 4.88 | 5.28 | 5.17 | 9.17 | 9.18 | 9.34 | 5.68 | 5.82 | 6.07 |
| 217760_at | 8.00 | 7.80 | 7.56 | 4.84 | 4.75 | 4.80 | 7.87 | 7.65 | 7.59 | 5.26 | 5.12 | 5.27 |
| 217977_at | 8.24 | 8.28 | 8.29 | 7.97 | 7.98 | 7.91 | 8.26 | 8.26 | 8.33 | 8.08 | 8.13 | 8.07 |
| 218005_at | 10.08 | 10.07 | 10.21 | 5.41 | 5.61 | 5.37 | 10.28 | 10.09 | 9.97 | 8.54 | 8.72 | 8.67 |
| 218006_s_at | 7.90 | 8.08 | 8.61 | 4.67 | 4.71 | 4.65 | 8.13 | 8.23 | 8.03 | 5.82 | 6.15 | 6.21 |
| 218154_at | 8.00 | 7.97 | 7.94 | 6.95 | 6.85 | 6.86 | 7.96 | 7.88 | 7.83 | 6.82 | 6.67 | 6.47 |
| 218457_s_at | 5.49 | 5.36 | 5.56 | 4.85 | 4.93 | 5.03 | 5.53 | 5.41 | 5.53 | 5.15 | 5.03 | 4.92 |
| 218503_at | 6.52 | 6.59 | 6.71 | 5.28 | 5.45 | 5.60 | 6.66 | 6.70 | 7.21 | 5.30 | 5.26 | 5.09 |
| 218618_s_at | 6.53 | 6.15 | 6.50 | 5.16 | 5.45 | 5.49 | 6.83 | 6.83 | 6.60 | 4.91 | 4.90 | 5.08 |
| 218676_s_at | 7.89 | 7.44 | 7.50 | 5.73 | 5.52 | 5.71 | 7.79 | 7.35 | 7.87 | 5.10 | 5.28 | 4.85 |
| 218689_at | 7.06 | 7.09 | 7.50 | 6.04 | 6.39 | 6.15 | 7.24 | 7.00 | 6.96 | 5.82 | 6.33 | 6.10 |
| 218729_at | 7.33 | 7.31 | 6.98 | 4.88 | 4.70 | 5.13 | 7.17 | 6.85 | 7.03 | 5.29 | 5.49 | 5.35 |
| 219143_s_at | 8.66 | 9.03 | 8.70 | 5.42 | 5.03 | 5.30 | 9.04 | 8.82 | 8.48 | 5.40 | 5.37 | 5.06 |
| 219362_at | 5.71 | 5.62 | 5.66 | 5.22 | 5.02 | 5.19 | 5.66 | 5.81 | 5.94 | 4.83 | 4.60 | 4.92 |
| 219684_at | 7.69 | 7.89 | 7.74 | 5.58 | 5.45 | 5.32 | 7.60 | 7.71 | 7.91 | 5.76 | 5.67 | 5.10 |
| 219734_at | 4.89 | 4.56 | 4.89 | 3.82 | 4.01 | 4.00 | 4.66 | 4.66 | 4.87 | 3.87 | 3.81 | 4.01 |
| 219953_s_at | 6.78 | 7.01 | 6.88 | 5.69 | 5.93 | 5.81 | 6.66 | 6.46 | 6.77 | 5.68 | 5.92 | 5.57 |
| 220104_at | 5.56 | 5.64 | 5.78 | 4.91 | 5.12 | 4.87 | 5.74 | 6.13 | 5.92 | 4.84 | 4.90 | 4.76 |
| 220230_s_at | 6.25 | 6.03 | 5.90 | 5.23 | 5.32 | 5.40 | 5.96 | 6.20 | 6.15 | 5.32 | 5.28 | 5.16 |
| 220235_s_at | 9.03 | 8.80 | 9.04 | 4.08 | 4.13 | 4.15 | 8.85 | 8.71 | 8.92 | 5.71 | 5.91 | 6.56 |
| 220784_s_at | 6.05 | 5.80 | 6.04 | 4.49 | 4.31 | 4.65 | 5.86 | 5.58 | 5.90 | 4.74 | 4.35 | 4.55 |
| 221081_s_at | 7.68 | 7.82 | 8.21 | 5.48 | 5.55 | 5.38 | 7.35 | 7.55 | 7.25 | 6.27 | 6.33 | 6.14 |
| 221349_at | 7.42 | 7.98 | 8.39 | 3.97 | 4.10 | 3.83 | 7.19 | 7.15 | 7.10 | 4.75 | 4.85 | 4.35 |
| 221641_s_at | 7.16 | 7.32 | 7.50 | 5.60 | 6.19 | 5.85 | 7.84 | 7.82 | 7.88 | 5.82 | 5.78 | 5.71 |
| 221666_s_at | 8.11 | 8.25 | 8.15 | 5.95 | 5.54 | 5.61 | 8.25 | 7.98 | 8.13 | 5.49 | 5.41 | 5.20 |
| 221690_s_at | 7.11 | 7.15 | 7.54 | 5.42 | 5.46 | 5.57 | 7.43 | 7.35 | 7.34 | 5.47 | 5.52 | 5.40 |
| 36030_at | 7.98 | 8.10 | 8.39 | 6.83 | 7.22 | 7.17 | 8.18 | 8.13 | 8.20 | 7.40 | 7.19 | 7.49 |
| 51158_at | 5.40 | 5.54 | 5.45 | 4.86 | 4.90 | 4.91 | 5.53 | 5.70 | 5.52 | 5.14 | 4.94 | 4.94 |
| 207714_s_at | 7.38 | 7.55 | 7.30 | 6.27 | 6.32 | 6.51 | 7.61 | 7.56 | 7.33 | 6.76 | 6.88 | 6.67 |
| 208816_x_at | 7.91 | 7.98 | 8.01 | 6.91 | 6.96 | 6.75 | 8.01 | 7.87 | 7.89 | 7.15 | 7.11 | 7.26 |
| 211566_x_at | 7.57 | 7.44 | 7.39 | 7.01 | 6.99 | 6.95 | 7.42 | 7.33 | 7.32 | 7.15 | 7.11 | 7.03 |
| 212235_at | 5.90 | 5.93 | 5.80 | 4.96 | 5.24 | 5.01 | 6.19 | 6.33 | 6.14 | 4.83 | 5.00 | 4.98 |

TABLE 3B
Genes With Reduced Expression in CD40-Sensitive Cell Lines

| | t-test (two-tailed) | | | | | |
|---|---|---|---|---|---|---|
| Probesets | pAB | pAD | pBC | pCD | Measured | Gene Symbol |
| FDR Cutoff | 1.0E−02 | 8.2E−03 | 1.2E−02 | 9.4E−03 | | |
| 200068_s_at | 1.7E−03 | 1.0E−03 | 5.6E−03 | 5.2E−04 | Yes | CANX |
| 200670_at | 3.2E−04 | 2.5E−03 | 9.0E−04 | 6.1E−03 | Yes | XBP1 |
| 200701_at | 4.7E−03 | 2.2E−03 | 1.1E−02 | 5.6E−03 | Yes | NPC2 |
| 200736_s_at | 6.2E−03 | 1.4E−03 | 7.7E−04 | 2.1E−04 | Yes | GPX1 |
| 200765_x_at | 1.2E−04 | 7.0E−04 | 8.0E−05 | 6.1E−05 | Yes | CTNNA1 |
| 200814_at | 5.5E−03 | 1.6E−04 | 2.2E−03 | 5.9E−05 | Yes | PSME1 |
| 200846_s_at | 9.4E−03 | 4.9E−03 | 6.9E−04 | 1.2E−03 | Yes | PPP1CA |
| 200887_s_at | 4.8E−03 | 2.4E−04 | 5.1E−03 | 1.7E−04 | Yes | STAT1 |
| 200905_x_at | 1.1E−03 | 9.5E−05 | 5.7E−04 | 2.8E−05 | Yes | HLA-E |
| 200936_at | 1.1E−04 | 1.3E−03 | 1.2E−03 | 6.6E−03 | Yes | RPL8 |
| 200941_at | 4.2E−03 | 2.5E−03 | 9.5E−03 | 7.2E−03 | Yes | HSBP1 |
| 201011_at | 1.9E−03 | 4.3E−03 | 1.6E−03 | 2.6E−03 | Yes | RPN1 |
| 201160_s_at | 1.9E−03 | 9.0E−05 | 2.7E−03 | 1.4E−04 | Yes | CSDA |
| 201161_s_at | 2.8E−03 | 3.1E−05 | 7.3E−03 | 3.0E−05 | Yes | CSDA |
| 201200_at | 9.9E−03 | 3.5E−03 | 4.9E−03 | 2.2E−03 | Yes | CREG |
| 201204_s_at | 3.5E−03 | 5.5E−03 | 1.1E−03 | 7.6E−04 | Yes | RRBP1 |
| 201206_s_at | 6.1E−03 | 2.2E−03 | 1.9E−03 | 7.3E−04 | Yes | RRBP1 |
| 201226_at | 2.9E−06 | 5.8E−04 | 2.8E−04 | 7.7E−03 | Yes | NDUFB8 |
| 201263_at | 1.8E−03 | 1.6E−03 | 1.0E−03 | 8.5E−04 | Yes | TARS |
| 201307_at | 6.6E−04 | 7.6E−05 | 2.0E−03 | 5.4E−05 | Yes | FLJ10849 |
| 201398_s_at | 1.7E−03 | 2.7E−04 | 1.4E−04 | 6.0E−04 | Yes | TRAM1 |
| 201399_s_at | 1.2E−03 | 4.3E−05 | 2.3E−03 | 5.9E−05 | Yes | TRAM1 |
| 201470_at | 1.3E−05 | 1.6E−04 | 4.5E−04 | 1.0E−03 | Yes | GSTO1 |
| 201487_at | 5.9E−03 | 2.7E−03 | 3.6E−03 | 1.2E−03 | Yes | CTSC |
| 201499_s_at | 1.1E−03 | 4.3E−03 | 9.0E−04 | 2.3E−03 | Yes | USP7 |
| 201566_x_at | 2.6E−03 | 2.8E−04 | 3.9E−03 | 1.8E−03 | Yes | ID2 |
| 201590_x_at | 2.6E−05 | 3.3E−05 | 2.1E−05 | 3.4E−05 | Yes | ANXA2 |
| 201601_x_at | 6.5E−03 | 2.9E−03 | 2.0E−06 | 1.1E−06 | Yes | IFITM1 |
| 201649_at | 7.0E−03 | 3.6E−04 | 6.7E−03 | 5.3E−04 | Yes | UBE2L6 |
| 201746_at | 2.2E−04 | 1.6E−03 | 2.5E−04 | 1.8E−03 | Yes | TP53 |
| 201874_at | 3.8E−03 | 4.6E−04 | 7.0E−03 | 6.0E−04 | Yes | FLJ21047 |
| 201937_s_at | 9.3E−03 | 8.5E−04 | 2.8E−03 | 4.1E−03 | Yes | DNPEP |
| 201968_s_at | 3.3E−03 | 2.9E−04 | 1.4E−03 | 1.4E−04 | Yes | PGM1 |
| 202096_s_at | 6.4E−03 | 6.2E−04 | 2.2E−04 | 1.0E−05 | Yes | BZRP |
| 202123_s_at | 2.0E−03 | 1.5E−03 | 1.9E−03 | 4.0E−03 | Yes | ABL1 |
| 202261_at | 2.6E−03 | 4.1E−03 | 2.7E−03 | 5.2E−03 | Yes | TCFL1 |
| 202279_at | 2.0E−03 | 3.0E−03 | 1.4E−03 | 1.0E−03 | Yes | C14orf2 |
| 202315_s_at | 6.9E−05 | 9.0E−05 | 9.9E−04 | 1.8E−03 | Yes | BCR |
| 202382_s_at | 5.2E−03 | 6.7E−05 | 2.3E−03 | 3.8E−05 | Yes | GNPDA1 |
| 202429_s_at | 2.4E−04 | 2.1E−03 | 1.0E−03 | 6.9E−03 | Yes | PPP3CA |
| 202443_x_at | 3.5E−03 | 5.1E−05 | 1.4E−03 | 2.2E−05 | Yes | NOTCH2 |
| 202447_at | 1.8E−03 | 3.8E−03 | 6.8E−03 | 5.4E−03 | Yes | DECR1 |
| 202457_s_at | 1.1E−03 | 2.4E−03 | 1.6E−03 | 4.2E−03 | Yes | PPP3CA |
| 202536_at | 5.4E−03 | 1.9E−03 | 4.0E−03 | 2.3E−03 | Yes | DKFZP564O123 |
| 202696_at | 9.2E−03 | 5.8E−03 | 5.5E−03 | 4.2E−03 | Yes | OSR1 |
| 202732_at | 1.3E−03 | 2.2E−04 | 1.2E−03 | 1.7E−04 | Yes | PKIG |
| 202811_at | 1.5E−04 | 1.1E−03 | 1.6E−04 | 1.5E−03 | Yes | AMSH |
| 202982_s_at | 4.2E−04 | 1.0E−04 | 1.0E−03 | 5.1E−05 | Yes | ZAP128 |
| 203031_s_at | 2.1E−04 | 8.1E−04 | 3.3E−04 | 6.8E−04 | Yes | UROS |
| 203113_s_at | 9.2E−03 | 5.6E−03 | 1.0E−02 | 4.1E−03 | Yes | EEF1D |
| 203142_s_at | 1.7E−05 | 1.5E−04 | 3.5E−03 | 8.0E−03 | Yes | AP3B1 |
| 203217_s_at | 1.5E−03 | 1.5E−03 | 4.0E−03 | 7.7E−05 | Yes | SIAT9 |
| 203335_at | 1.2E−05 | 1.5E−04 | 4.4E−04 | 8.9E−04 | Yes | PHYH |
| 203343_at | 6.3E−03 | 3.9E−03 | 4.9E−03 | 5.3E−03 | Yes | UGDH |
| 203459_s_at | 5.0E−03 | 1.5E−03 | 9.0E−03 | 4.1E−03 | Yes | — |
| 203659_s_at | 4.7E−03 | 1.1E−03 | 1.8E−03 | 8.9E−04 | Yes | RFP2 |
| 203825_at | 5.1E−03 | 4.2E−03 | 5.7E−04 | 1.3E−04 | Yes | BRD3 |
| 203957_at | 6.1E−03 | 2.2E−03 | 2.4E−03 | 8.6E−05 | Yes | E2F6 |
| 204003_s_at | 3.5E−03 | 3.9E−04 | 6.5E−03 | 4.7E−04 | Yes | NUPL2 |
| 204044_at | 9.2E−04 | 7.1E−04 | 9.1E−04 | 4.0E−03 | Yes | QPRT |
| 204168_at | 3.0E−06 | 1.3E−05 | 5.0E−05 | 2.2E−04 | Yes | MGST2 |
| 204172_at | 6.1E−03 | 3.9E−03 | 4.9E−03 | 5.5E−03 | Yes | CPO |
| 204220_at | 6.7E−03 | 2.1E−04 | 8.6E−03 | 2.4E−04 | Yes | GMFG |
| 204234_s_at | 8.9E−03 | 2.0E−03 | 1.1E−02 | 2.5E−03 | Yes | ZNF195 |
| 204256_at | 7.4E−03 | 1.8E−03 | 3.3E−03 | 8.0E−04 | Yes | ELOVL6 |
| 204279_at | 3.2E−03 | 6.5E−04 | 3.8E−04 | 1.5E−04 | Yes | PSMB9 |
| 204326_x_at | 5.4E−06 | 7.7E−05 | 9.0E−05 | 1.5E−03 | Yes | MT1X |
| 204386_s_at | 2.1E−04 | 3.0E−03 | 7.9E−04 | 6.0E−03 | Yes | MRP63 |
| 204418_x_at | 8.7E−04 | 2.8E−03 | 3.4E−03 | 5.4E−03 | Yes | GSTM2 |
| 204565_at | 6.4E−04 | 2.1E−04 | 3.6E−03 | 9.8E−05 | Yes | THEM2 |
| 204766_s_at | 1.6E−04 | 3.2E−04 | 4.6E−04 | 4.4E−03 | Yes | NUDT1 |
| 204769_s_at | 8.7E−04 | 4.7E−04 | 2.3E−03 | 3.9E−06 | Yes | TAP2 |
| 204779_s_at | 2.1E−03 | 1.6E−05 | 5.2E−03 | 4.8E−03 | Yes | HOXB7 |
| 204806_x_at | 4.4E−04 | 3.5E−05 | 2.6E−03 | 1.5E−04 | Yes | HLA-F |
| 204937_s_at | 2.0E−04 | 2.9E−03 | 1.2E−04 | 1.2E−03 | Yes | ZNF274 |
| 205048_s_at | 2.1E−05 | 3.7E−03 | 1.2E−04 | 1.7E−03 | Yes | — |
| 205297_s_at | 7.0E−04 | 3.1E−03 | 1.1E−03 | 6.3E−04 | Yes | CD79B |
| 205659_at | 7.2E−03 | 6.1E−03 | 3.4E−03 | 4.6E−03 | Yes | HDAC9 |
| 206928_at | 3.2E−03 | 3.0E−03 | 9.9E−03 | 3.8E−03 | Yes | ZNF124 |
| 207181_s_at | 3.2E−03 | 1.9E−03 | 9.0E−03 | 5.8E−03 | Yes | CASP7 |
| 207304_at | 3.4E−03 | 4.5E−03 | 1.4E−03 | 2.4E−03 | Yes | ZNF45 |
| 207585_s_at | 4.9E−03 | 8.0E−04 | 9.4E−03 | 1.7E−03 | Yes | RPL36AL |
| 207777_s_at | 2.4E−04 | 5.8E−04 | 1.1E−04 | 2.4E−04 | Yes | SP140 |
| 207809_s_at | 6.1E−03 | 7.1E−03 | 1.0E−03 | 4.8E−03 | Yes | ATP6AP1 |
| 208490_x_at | 2.7E−03 | 6.5E−03 | 2.3E−03 | 3.2E−03 | Yes | HIST1H2BG |
| 208527_x_at | 4.7E−05 | 4.8E−03 | 1.9E−03 | 1.5E−03 | Yes | HIST1H2BE |
| 208579_x_at | 3.6E−04 | 6.3E−04 | 2.5E−04 | 3.1E−04 | Yes | H2BFS |
| 208581_x_at | 6.9E−04 | 4.9E−03 | 7.8E−04 | 6.3E−03 | Yes | MT1X |
| 208690_s_at | 6.0E−05 | 8.4E−05 | 3.0E−05 | 1.5E−05 | Yes | PDLIM1 |
| 208729_x_at | 1.7E−03 | 2.2E−05 | 1.2E−04 | 1.8E−04 | Yes | HLA-B |
| 208812_x_at | 5.3E−04 | 8.6E−08 | 7.1E−04 | 9.3E−08 | Yes | HLA-C |
| 208918_s_at | 7.1E−04 | 1.7E−03 | 5.5E−03 | 2.6E−03 | Yes | FLJ13052 |
| 209040_s_at | 6.1E−04 | 8.3E−04 | 5.0E−04 | 9.3E−04 | Yes | PSMB8 |
| 209112_at | 8.7E−05 | 2.3E−03 | 1.6E−04 | 1.9E−03 | Yes | CDKN1B |
| 209124_at | 8.2E−03 | 3.5E−03 | 1.9E−03 | 5.5E−04 | Yes | MYD88 |
| 209138_x_at | 1.4E−05 | 5.8E−03 | 1.3E−06 | 4.0E−03 | Yes | IGL@ |
| 209140_x_at | 6.0E−04 | 1.4E−04 | 3.6E−04 | 6.9E−05 | Yes | HLA-B |
| 209175_at | 6.3E−03 | 5.9E−03 | 5.5E−03 | 7.1E−03 | Yes | SEC23IP |
| 209201_x_at | 1.2E−03 | 7.3E−04 | 1.1E−02 | 5.6E−03 | Yes | CXCR4 |
| 209472_at | 5.4E−05 | 9.6E−04 | 1.9E−05 | 8.5E−04 | Yes | LOC56267 |
| 209476_at | 3.7E−03 | 7.7E−03 | 5.3E−03 | 8.8E−03 | Yes | TXNDC |
| 209591_s_at | 4.1E−03 | 3.9E−04 | 8.9E−04 | 2.7E−04 | Yes | BMP7 |
| 209970_x_at | 8.9E−04 | 1.0E−03 | 1.0E−03 | 1.2E−03 | Yes | CASP1 |
| 209995_s_at | 6.0E−04 | 7.8E−06 | 1.4E−03 | 2.9E−05 | Yes | TCL1A |
| 210136_at | 4.5E−03 | 2.0E−04 | 6.9E−04 | 6.6E−04 | Yes | MBP |
| 210844_x_at | 8.1E−03 | 2.8E−04 | 8.0E−04 | 3.8E−05 | Yes | CTNNA1 |
| 211061_s_at | 2.3E−03 | 6.0E−03 | 3.0E−03 | 7.5E−03 | Yes | MGAT2 |
| 211089_s_at | 1.3E−03 | 1.8E−03 | 2.6E−03 | 3.4E−03 | Yes | NEK3 |
| 211366_x_at | 8.1E−05 | 5.4E−04 | 2.4E−04 | 3.5E−04 | Yes | CASP1 |
| 211528_x_at | 1.5E−03 | 5.1E−04 | 1.4E−03 | 1.9E−04 | Yes | HLA-G |
| 211529_x_at | 1.1E−03 | 2.4E−04 | 1.7E−03 | 1.1E−03 | Yes | HLA-G |
| 211530_x_at | 1.9E−03 | 1.5E−05 | 3.2E−03 | 7.0E−04 | Yes | HLA-G |
| 211799_x_at | 3.5E−03 | 9.9E−04 | 1.9E−03 | 3.3E−04 | Yes | HLA-C |
| 211911_x_at | 1.3E−05 | 2.4E−06 | 9.6E−05 | 1.3E−05 | Yes | HLA-B |
| 211919_s_at | 7.8E−04 | 4.6E−04 | 1.1E−02 | 5.1E−03 | Yes | CXCR4 |
| 211989_at | 8.8E−04 | 3.5E−03 | 1.3E−03 | 2.2E−03 | Yes | SMARCE1 |
| 212057_at | 1.9E−04 | 3.4E−03 | 2.1E−04 | 9.0E−04 | Yes | KIAA0182 |
| 212071_s_at | 3.9E−06 | 7.0E−04 | 2.7E−06 | 7.8E−04 | Yes | SPTBN1 |
| 212149_at | 5.6E−03 | 4.8E−04 | 5.2E−03 | 5.7E−04 | Yes | KIAA0143 |
| 212382_at | 5.7E−03 | 3.5E−03 | 1.4E−03 | 8.1E−04 | Yes | TCF4 |
| 212385_at | 1.5E−03 | 4.7E−04 | 5.5E−03 | 1.6E−03 | Yes | TCF4 |
| 212386_at | 2.1E−04 | 2.0E−03 | 3.4E−05 | 2.9E−03 | Yes | TCF4 |
| 212387_at | 5.9E−04 | 2.5E−05 | 5.0E−06 | 1.2E−04 | Yes | TCF4 |
| 212408_at | 1.2E−03 | 5.9E−03 | 1.1E−03 | 6.4E−03 | Yes | LAP1B |
| 212446_s_at | 6.4E−05 | 9.6E−04 | 2.6E−03 | 7.5E−03 | Yes | LOC253782 |
| 212507_at | 1.2E−03 | 4.3E−03 | 4.7E−03 | 2.6E−03 | Yes | RW1 |
| 212547_at | 1.4E−04 | 4.7E−03 | 2.5E−04 | 1.4E−03 | Yes | — |
| 212739_s_at | 1.7E−04 | 1.9E−03 | 1.4E−04 | 8.7E−04 | Yes | NME4 |
| 212774_at | 1.7E−03 | 3.0E−04 | 1.3E−03 | 2.0E−04 | Yes | ZNF238 |
| 212792_at | 6.2E−03 | 6.5E−03 | 1.5E−05 | 7.7E−03 | Yes | KIAA0877 |
| 212798_s_at | 6.6E−04 | 8.2E−05 | 3.8E−04 | 5.9E−05 | Yes | DKFZP564O043 |
| 212946_at | 1.4E−03 | 1.1E−04 | 4.4E−03 | 7.1E−04 | Yes | KIAA0564 |
| 213020_at | 8.5E−03 | 2.9E−03 | 4.1E−03 | 2.6E−03 | Yes | GOSR1 |
| 213116_at | 6.7E−04 | 7.9E−04 | 1.5E−04 | 2.1E−04 | Yes | NEK3 |
| 213233_s_at | 7.7E−04 | 7.3E−05 | 4.9E−04 | 6.5E−04 | Yes | KIAA1354 |
| 213293_s_at | 8.1E−03 | 3.2E−03 | 3.9E−04 | 1.5E−04 | Yes | TRIM22 |
| 213357_at | 8.8E−03 | 7.4E−04 | 5.7E−03 | 5.6E−04 | Yes | — |
| 213361_at | 6.3E−03 | 1.5E−04 | 7.4E−03 | 2.7E−03 | Yes | PCTAIRE2BP |
| 213435_at | 6.2E−03 | 1.6E−03 | 1.0E−03 | 1.8E−03 | Yes | SATB2 |
| 213793_s_at | 5.3E−03 | 1.8E−03 | 1.0E−02 | 3.2E−03 | Yes | HOMER1 |
| 213891_s_at | 1.6E−04 | 6.7E−04 | 9.5E−05 | 5.3E−04 | Yes | TCF4 |
| 214022_s_at | 3.8E−04 | 1.6E−03 | 1.1E−03 | 9.0E−05 | Yes | IFITM1 |
| 214032_at | 3.5E−04 | 5.5E−04 | 1.0E−03 | 2.2E−04 | Yes | ZAP70 |
| 214172_x_at | 4.0E−04 | 4.7E−04 | 2.6E−04 | 2.9E−04 | Yes | RYK |
| 214369_s_at | 2.4E−03 | 1.0E−03 | 7.9E−03 | 4.4E−04 | Yes | RASGRP2 |
| 214394_x_at | 4.2E−04 | 4.9E−04 | 1.2E−03 | 2.0E−03 | Yes | EEF1D |
| 214459_x_at | 7.3E−03 | 2.8E−03 | 1.7E−03 | 9.0E−04 | Yes | HLA-C |
| 214512_s_at | 5.3E−06 | 9.6E−05 | 2.2E−04 | 2.3E−04 | Yes | PC4 |
| 214677_x_at | 3.9E−07 | 9.9E−04 | 3.0E−06 | 1.5E−03 | Yes | IGLJ3 |
| 214749_s_at | 6.2E−04 | 1.8E−03 | 2.5E−04 | 2.6E−03 | Yes | FLJ20811 |
| 214835_s_at | 1.1E−03 | 5.6E−03 | 1.0E−03 | 5.3E−03 | Yes | SUCLG2 |
| 214916_x_at | 2.6E−03 | 6.5E−04 | 9.5E−03 | 7.9E−04 | Yes | — |
| 215121_x_at | 7.2E−04 | 2.7E−03 | 1.1E−03 | 3.9E−03 | Yes | — |
| 215516_at | 7.3E−03 | 7.7E−03 | 3.0E−03 | 4.0E−03 | Yes | LAMB4 |
| 215772_x_at | 4.3E−03 | 6.3E−03 | 5.4E−04 | 1.6E−03 | Yes | SUCLG2 |
| 215946_x_at | 1.6E−03 | 1.1E−03 | 4.8E−05 | 6.5E−04 | Yes | — |
| 216526_x_at | 1.7E−03 | 2.1E−07 | 2.2E−03 | 2.6E−07 | Yes | HLA-C |
| 216733_s_at | 5.9E−05 | 1.2E−03 | 6.5E−05 | 5.6E−05 | Yes | GATM |
| 216973_s_at | 6.3E−04 | 3.9E−03 | 5.5E−03 | 7.4E−04 | Yes | HOXB7 |
| 216976_s_at | 1.2E−05 | 3.2E−03 | 1.1E−02 | 1.2E−03 | Yes | RYK |
| 217028_at | 2.8E−03 | 4.0E−05 | 1.1E−03 | 8.6E−04 | Yes | — |
| 217436_x_at | 1.6E−03 | 1.0E−04 | 2.2E−03 | 5.9E−04 | Yes | — |
| 217456_x_at | 2.5E−04 | 6.4E−05 | 6.9E−05 | 1.8E−04 | Yes | HLA-E |
| 217809_at | 1.7E−05 | 2.2E−03 | 2.1E−05 | 3.0E−03 | Yes | BZW2 |
| 217839_at | 5.6E−03 | 2.3E−04 | 8.8E−04 | 6.7E−04 | Yes | TFG |
| 217911_s_at | 3.2E−03 | 1.1E−03 | 8.4E−04 | 1.7E−03 | Yes | BAG3 |
| 217940_s_at | 1.4E−04 | 7.1E−03 | 1.4E−03 | 2.7E−03 | Yes | FLJ10769 |
| 217950_at | 8.2E−04 | 4.5E−04 | 3.2E−03 | 2.3E−03 | Yes | NOSIP |
| 217988_at | 1.4E−03 | 2.2E−03 | 2.4E−04 | 1.1E−03 | Yes | C14orf18 |
| 218026_at | 1.1E−03 | 6.6E−03 | 1.1E−03 | 6.3E−04 | Yes | HSPC009 |
| 218093_s_at | 3.3E−04 | 6.1E−04 | 2.0E−03 | 5.2E−03 | Yes | ANKRD10 |
| 218100_s_at | 5.3E−04 | 3.9E−03 | 6.5E−04 | 7.3E−03 | Yes | ESRRBL1 |
| 218187_s_at | 4.2E−03 | 4.8E−03 | 3.3E−03 | 4.0E−03 | Yes | FLJ20989 |
| 218224_at | 1.5E−03 | 1.7E−03 | 1.4E−05 | 1.8E−05 | Yes | PNMA1 |
| 218276_s_at | 1.3E−03 | 1.1E−04 | 2.3E−04 | 1.1E−04 | Yes | SAV1 |
| 218285_s_at | 1.4E−03 | 5.0E−04 | 2.4E−03 | 6.3E−04 | Yes | DHRS6 |
| 218316_at | 4.2E−03 | 3.0E−04 | 9.7E−03 | 6.2E−04 | Yes | TIMM9 |
| 218343_s_at | 1.0E−03 | 1.5E−03 | 4.3E−04 | 1.7E−03 | Yes | GTF3C3 |
| 218358_at | 7.9E−04 | 5.7E−04 | 3.7E−03 | 2.6E−03 | Yes | MGC11256 |
| 218377_s_at | 1.5E−03 | 6.1E−04 | 2.7E−03 | 8.0E−04 | Yes | C21orf6 |
| 218490_s_at | 1.0E−02 | 3.7E−03 | 6.2E−03 | 2.9E−03 | Yes | ZNF302 |
| 218491_s_at | 4.4E−03 | 3.5E−03 | 3.1E−03 | 8.4E−03 | Yes | THY28 |
| 218543_s_at | 1.1E−03 | 3.4E−04 | 1.2E−04 | 4.5E−04 | Yes | ZC3HDC1 |
| 218557_at | 1.1E−05 | 3.0E−05 | 3.0E−03 | 3.9E−04 | Yes | NIT2 |
| 218633_x_at | 2.1E−04 | 3.3E−03 | 4.1E−04 | 4.0E−03 | Yes | FLJ11342 |
| 218648_at | 3.9E−03 | 5.3E−04 | 4.0E−03 | 5.4E−04 | Yes | FLJ21868 |
| 218735_s_at | 5.3E−03 | 1.6E−03 | 5.7E−04 | 6.6E−04 | Yes | AF020591 |
| 218738_s_at | 2.7E−03 | 3.8E−03 | 6.4E−04 | 9.5E−04 | Yes | RNF138 |
| 218773_s_at | 2.1E−03 | 6.4E−03 | 2.3E−03 | 9.2E−03 | Yes | PILB |
| 219008_at | 2.0E−03 | 5.2E−03 | 1.4E−03 | 1.9E−03 | Yes | FLJ21820 |
| 219184_x_at | 1.9E−03 | 2.3E−04 | 1.0E−02 | 7.9E−03 | Yes | TIMM22 |
| 219209_at | 4.3E−03 | 7.4E−04 | 1.3E−03 | 4.4E−04 | Yes | MDA5 |
| 219215_s_at | 2.9E−03 | 5.0E−05 | 3.2E−03 | 6.7E−05 | Yes | SLC39A4 |
| 219248_at | 1.5E−03 | 5.1E−03 | 1.7E−03 | 1.9E−03 | Yes | C2orf8 |
| 219471_at | 4.7E−03 | 3.7E−03 | 3.0E−03 | 2.9E−03 | Yes | C13orf18 |
| 219489_s_at | 2.2E−04 | 1.5E−03 | 3.7E−04 | 2.0E−03 | Yes | RHBDL2 |
| 219563_at | 3.9E−05 | 1.8E−05 | 3.4E−05 | 3.4E−04 | Yes | C14orf139 |
| 219664_s_at | 1.9E−03 | 2.4E−03 | 7.9E−04 | 1.0E−03 | Yes | DECR2 |
| 219767_s_at | 3.9E−03 | 3.3E−03 | 1.9E−03 | 1.0E−03 | Yes | CRYZL1 |
| 219822_at | 6.3E−04 | 7.5E−03 | 7.3E−04 | 7.1E−03 | Yes | MTRF1 |
| 220741_s_at | 1.0E−03 | 4.5E−05 | 7.8E−05 | 6.4E−04 | Yes | PPA2 |
| 221234_s_at | 5.9E−04 | 4.7E−04 | 3.2E−03 | 3.4E−04 | Yes | BACH2 |
| 221614_s_at | 6.4E−03 | 3.8E−03 | 1.9E−03 | 2.6E−04 | Yes | RPH3AL |
| 221847_at | 2.0E−03 | 1.6E−03 | 1.1E−02 | 5.0E−03 | Yes | TAS2R14 |
| 221874_at | 6.0E−03 | 1.9E−03 | 1.4E−03 | 2.4E−04 | Yes | KIAA1324 |
| 221875_x_at | 3.1E−03 | 6.3E−04 | 8.5E−04 | 8.3E−05 | Yes | HLA-F |
| 222146_s_at | 1.1E−03 | 4.3E−04 | 3.8E−03 | 2.4E−03 | Yes | TCF4 |
| 35671_at | 1.3E−04 | 6.1E−04 | 6.0E−04 | 4.1E−04 | Yes | GTF3C1 |
| 36830_at | 3.5E−04 | 1.5E−03 | 9.0E−03 | 6.4E−03 | Yes | MIPEP |
| 36936_at | 7.6E−03 | 6.4E−06 | 1.0E−02 | 2.0E−04 | Yes | TSTA3 |
| 37384_at | 3.1E−04 | 4.9E−03 | 2.7E−04 | 3.5E−03 | Yes | PPM1F |
| 39318_at | 3.2E−04 | 3.8E−04 | 2.7E−04 | 1.2E−04 | Yes | TCL1A |
| 40148_at | 1.9E−03 | 1.6E−04 | 1.2E−02 | 5.8E−03 | Yes | APBB2 |
| 200660_at | 3.1E−05 | 1.3E−04 | 4.7E−04 | 1.4E−03 | Yes | S100A11 |
| 200671_s_at | 2.2E−04 | 1.1E−04 | 1.8E−03 | 5.0E−04 | Yes | SPTBN1 |
| 200672_x_at | 1.2E−03 | 1.3E−03 | 9.7E−04 | 1.5E−03 | Yes | SPTBN1 |
| 200704_at | 3.3E−04 | 3.3E−04 | 2.6E−04 | 2.4E−04 | Yes | LITAF |
| 200706_s_at | 2.2E−05 | 1.8E−04 | 1.2E−03 | 3.1E−03 | Yes | LITAF |
| 200782_at | 1.5E−04 | 5.9E−04 | 5.6E−05 | 9.4E−05 | Yes | ANXA5 |
| 200923_at | 1.8E−04 | 2.7E−04 | 7.5E−06 | 7.5E−06 | Yes | LGALS3BP |
| 201005_at | 1.1E−04 | 2.7E−04 | 2.4E−06 | 1.0E−05 | Yes | CD9 |
| 201426_s_at | 3.3E−05 | 1.0E−03 | 2.2E−04 | 5.5E−04 | Yes | VIM |
| 201485_s_at | 9.7E−04 | 9.1E−07 | 1.0E−02 | 1.1E−03 | Yes | RCN2 |
| 201792_at | 3.1E−04 | 6.0E−04 | 8.2E−04 | 1.5E−03 | Yes | AEBP1 |
| 202218_s_at | 5.1E−03 | 2.2E−03 | 4.1E−03 | 1.6E−03 | Yes | FADS2 |
| 202283_at | 3.1E−03 | 4.1E−03 | 2.1E−03 | 2.9E−03 | Yes | SERPINF1 |
| 202719_s_at | 8.4E−04 | 2.1E−03 | 2.4E−03 | 3.0E−04 | Yes | TES |
| 202855_s_at | 4.0E−03 | 2.1E−03 | 4.4E−03 | 7.7E−04 | Yes | SLC16A3 |
| 202856_s_at | 2.0E−03 | 1.2E−03 | 8.7E−04 | 1.4E−03 | Yes | SLC16A3 |
| 203045_at | 1.0E−03 | 3.8E−04 | 3.3E−03 | 2.7E−03 | Yes | NINJ1 |
| 203063_at | 8.0E−04 | 6.8E−04 | 1.2E−03 | 8.9E−04 | Yes | PPM1F |
| 203068_at | 2.0E−04 | 3.6E−04 | 1.9E−04 | 4.8E−04 | Yes | KIAA0469 |
| 203211_s_at | 2.1E−05 | 6.2E−06 | 2.8E−04 | 1.4E−04 | Yes | MTMR2 |
| 203212_s_at | 1.9E−03 | 2.5E−03 | 3.7E−03 | 1.4E−03 | Yes | MTMR2 |
| 203222_s_at | 1.5E−03 | 2.0E−03 | 2.5E−03 | 2.4E−03 | Yes | TLE1 |
| 203305_at | 1.8E−03 | 2.5E−03 | 7.7E−04 | 1.0E−03 | Yes | F13A1 |
| 203408_s_at | 1.1E−03 | 1.7E−03 | 1.6E−03 | 2.7E−03 | Yes | SATB1 |
| 203557_s_at | 6.9E−05 | 1.1E−03 | 4.7E−05 | 4.2E−04 | Yes | PCBD |
| 203708_at | 8.7E−03 | 8.0E−03 | 8.6E−05 | 1.0E−04 | Yes | PDE4B |
| 203853_s_at | 3.3E−05 | 4.2E−05 | 3.5E−03 | 7.4E−03 | Yes | GAB2 |
| 204305_at | 1.9E−03 | 3.9E−03 | 8.3E−05 | 1.3E−04 | Yes | MIPEP |
| 204821_at | 3.0E−04 | 1.5E−03 | 4.7E−04 | 4.6E−03 | Yes | BTN3A3 |
| 204994_at | 1.4E−03 | 3.7E−03 | 8.1E−04 | 2.5E−03 | Yes | MX2 |
| 205173_x_at | 2.5E−03 | 1.2E−03 | 6.5E−05 | 1.1E−03 | Yes | CD58 |
| 205181_at | 4.0E−03 | 5.5E−03 | 4.0E−03 | 4.2E−03 | Yes | ZNF193 |
| 205366_s_at | 6.4E−03 | 7.4E−03 | 4.6E−03 | 8.3E−03 | Yes | HOXB6 |
| 205401_at | 4.6E−05 | 8.5E−04 | 2.8E−04 | 6.4E−04 | Yes | AGPS |
| 205668_at | 1.0E−03 | 3.6E−03 | 1.4E−03 | 4.6E−04 | Yes | LY75 |
| 205691_at | 1.5E−03 | 1.4E−04 | 1.1E−02 | 7.7E−04 | Yes | SYNGR3 |
| 205790_at | 2.0E−04 | 4.4E−04 | 2.1E−03 | 6.3E−04 | Yes | SCAP1 |
| 205932_s_at | 1.1E−03 | 2.8E−03 | 4.1E−03 | 2.3E−03 | Yes | MSX1 |
| 206082_at | 5.5E−04 | 7.8E−04 | 6.9E−04 | 1.7E−04 | Yes | HCP5 |
| 206261_at | 3.2E−03 | 5.4E−03 | 1.1E−03 | 1.1E−03 | Yes | ZNF239 |
| 206437_at | 8.6E−04 | 3.3E−04 | 5.0E−03 | 2.5E−03 | Yes | EDG6 |
| 206554_x_at | 2.0E−04 | 8.7E−04 | 2.6E−05 | 1.0E−03 | Yes | SETMAR |
| 206591_at | 6.2E−03 | 4.9E−03 | 1.5E−04 | 5.3E−05 | Yes | RAG1 |
| 206660_at | 8.7E−05 | 4.3E−04 | 2.5E−03 | 4.9E−03 | Yes | IGLL1 |
| 206698_at | 4.0E−03 | 1.8E−03 | 1.1E−03 | 6.1E−05 | Yes | XK |
| 206866_at | 9.8E−04 | 1.0E−03 | 1.6E−03 | 2.4E−03 | Yes | CDH4 |
| 208146_s_at | 9.3E−04 | 4.3E−03 | 2.5E−03 | 1.2E−03 | Yes | CPVL |
| 208964_s_at | 3.0E−03 | 6.6E−03 | 1.9E−03 | 2.8E−03 | Yes | FADS1 |
| 209129_at | 3.5E−04 | 2.1E−03 | 3.2E−03 | 9.3E−03 | Yes | TRIP6 |
| 209310_s_at | 1.5E−03 | 6.0E−03 | 6.1E−04 | 2.0E−03 | Yes | CASP4 |
| 209398_at | 3.6E−03 | 5.6E−03 | 5.2E−03 | 9.2E−03 | Yes | HIST1H1C |
| 209590_at | 5.1E−03 | 2.0E−03 | 5.1E−03 | 2.5E−03 | Yes | BMP7 |
| 209728_at | 2.7E−04 | 2.6E−04 | 1.0E−04 | 1.6E−04 | Yes | HLA-DRB3 |
| 209790_s_at | 1.1E−04 | 5.7E−03 | 1.7E−03 | 5.6E−03 | Yes | CASP6 |
| 210427_x_at | 2.9E−03 | 7.4E−04 | 8.7E−06 | 4.6E−03 | Yes | ANXA2 |
| 210715_s_at | 1.7E−04 | 1.4E−03 | 1.9E−04 | 1.7E−03 | Yes | SPINT2 |
| 210830_s_at | 6.8E−04 | 1.5E−03 | 1.5E−03 | 2.2E−03 | Yes | PON2 |
| 211367_s_at | 8.7E−05 | 1.1E−04 | 8.7E−04 | 1.1E−03 | Yes | CASP1 |
| 211368_s_at | 4.8E−04 | 3.8E−05 | 5.3E−03 | 2.2E−03 | Yes | CASP1 |
| 211812_s_at | 5.5E−03 | 5.7E−03 | 1.4E−03 | 1.5E−03 | Yes | B3GALT3 |
| 212268_at | 8.5E−06 | 1.9E−03 | 9.3E−04 | 2.5E−03 | Yes | SERPINB1 |
| 212442_s_at | 4.0E−04 | 4.9E−05 | 6.5E−04 | 6.2E−05 | Yes | LOC253782 |
| 212561_at | 8.4E−03 | 8.1E−03 | 1.5E−03 | 4.4E−03 | Yes | RAB6IP1 |
| 213147_at | 2.0E−04 | 4.4E−05 | 1.0E−03 | 6.5E−04 | Yes | HOXA10 |
| 213150_at | 4.2E−03 | 4.7E−03 | 1.0E−03 | 1.2E−03 | Yes | HOXA10 |
| 213371_at | 1.8E−03 | 1.5E−03 | 1.3E−04 | 1.4E−04 | Yes | LDB3 |
| 213419_at | 2.5E−03 | 3.4E−03 | 7.3E−03 | 1.7E−03 | Yes | — |
| 213502_x_at | 1.1E−03 | 1.4E−03 | 1.1E−05 | 2.9E−05 | Yes | — |
| 213503_x_at | 1.7E−05 | 2.7E−04 | 3.5E−04 | 2.4E−03 | Yes | ANXA2 |
| 213566_at | 2.6E−04 | 8.3E−04 | 6.0E−04 | 4.0E−03 | Yes | RNASE6 |
| 213568_at | 2.8E−04 | 3.0E−05 | 1.2E−04 | 9.1E−05 | Yes | OSR2 |
| 213572_s_at | 5.2E−03 | 5.0E−03 | 1.6E−03 | 6.2E−04 | Yes | SERPINB1 |
| 213625_at | 3.8E−03 | 4.1E−05 | 1.1E−02 | 8.7E−05 | Yes | P1P373C6 |
| 213788_s_at | 4.1E−03 | 6.0E−03 | 7.5E−03 | 6.7E−03 | Yes | — |
| 215071_s_at | 5.1E−03 | 5.4E−03 | 8.6E−03 | 9.3E−03 | Yes | — |
| 215379_x_at | 1.1E−06 | 6.3E−03 | 6.8E−07 | 5.2E−03 | Yes | IGLJ3 |
| 216565_x_at | 3.5E−03 | 7.4E−04 | 2.2E−03 | 1.3E−03 | Yes | — |
| 217022_s_at | 1.9E−05 | 1.4E−05 | 1.6E−03 | 2.4E−03 | Yes | MGC27165 |
| 217360_x_at | 2.4E−04 | 4.5E−04 | 8.9E−03 | 7.9E−03 | Yes | — |
| 217759_at | 1.8E−05 | 2.5E−05 | 1.2E−04 | 1.6E−04 | Yes | TRIM44 |
| 217760_at | 1.2E−03 | 8.1E−04 | 3.4E−04 | 9.5E−05 | Yes | TRIM44 |
| 217977_at | 7.5E−04 | 2.7E−03 | 5.0E−04 | 3.7E−03 | Yes | SEPX1 |
| 218005_at | 4.2E−06 | 4.1E−05 | 3.5E−06 | 4.7E−04 | Yes | ZNF22 |
| 218006_s_at | 3.5E−03 | 2.6E−03 | 1.0E−04 | 7.6E−04 | Yes | ZNF22 |
| 218154_at | 9.8E−05 | 4.8E−03 | 4.1E−05 | 2.8E−03 | Yes | FLJ12150 |
| 218457_s_at | 2.6E−03 | 7.5E−03 | 1.3E−03 | 6.5E−03 | Yes | DNMT3A |
| 218503_at | 1.2E−03 | 8.9E−05 | 5.6E−03 | 6.2E−03 | Yes | KIAA1797 |
| 218618_s_at | 3.5E−03 | 2.4E−03 | 6.4E−04 | 8.0E−05 | Yes | FAD104 |
| 218676_s_at | 1.5E−03 | 1.9E−04 | 2.5E−03 | 3.1E−04 | Yes | PCTP |
| 218689_at | 5.6E−03 | 5.3E−03 | 3.1E−03 | 8.7E−03 | Yes | FANCF |
| 218729_at | 1.7E−04 | 6.6E−04 | 2.7E−04 | 3.4E−04 | Yes | LXN |
| 219143_s_at | 2.8E−05 | 2.6E−05 | 1.2E−04 | 1.5E−04 | Yes | Rpp25 |
| 219362_at | 6.1E−03 | 7.8E−03 | 3.5E−03 | 1.3E−03 | Yes | — |
| 219684_at | 2.7E−05 | 4.9E−03 | 5.1E−05 | 3.1E−03 | Yes | IFRG28 |
| 219734_at | 6.1E−03 | 5.6E−03 | 1.3E−03 | 1.0E−03 | Yes | FLJ20174 |
| 219953_s_at | 3.7E−04 | 1.5E−03 | 2.6E−03 | 3.0E−03 | Yes | C11orf17 |
| 220104_at | 2.6E−03 | 9.0E−04 | 3.7E−03 | 5.7E−03 | Yes | ZC3HAV1 |
| 220230_s_at | 8.5E−03 | 7.4E−03 | 1.5E−03 | 1.3E−03 | Yes | CYB5R2 |
| 220235_s_at | 1.3E−04 | 4.4E−03 | 5.0E−05 | 6.0E−03 | Yes | RIF1 |
| 220784_s_at | 4.0E−04 | 8.6E−04 | 7.9E−04 | 1.3E−03 | Yes | UTS2 |
| 221081_s_at | 2.2E−03 | 4.4E−03 | 2.7E−04 | 9.1E−04 | Yes | FLJ22457 |
| 221349_at | 3.1E−03 | 1.7E−03 | 1.5E−04 | 3.0E−03 | Yes | VPREB1 |
| 221641_s_at | 4.1E−03 | 2.1E−03 | 6.9E−03 | 7.6E−06 | Yes | ACATE2 |
| 221666_s_at | 9.7E−04 | 1.0E−04 | 2.5E−04 | 2.1E−05 | Yes | ASC |
| 221690_s_at | 3.2E−03 | 3.9E−03 | 1.9E−05 | 2.8E−06 | Yes | NALP2 |
| 36030_at | 3.3E−03 | 8.1E−03 | 1.0E−02 | 8.4E−03 | Yes | HOM-TES-103 |
| 51158_at | 2.0E−03 | 7.1E−03 | 5.0E−03 | 2.8E−03 | Yes | — |
| 207714_s_at | 5.4E−04 | 2.8E−03 | 6.2E−04 | 3.2E−03 | No | SERPINH1 |
| 208816_x_at | 7.8E−04 | 2.9E−04 | 3.8E−04 | 2.7E−04 | No | — |
| 211566_x_at | 6.9E−03 | 6.7E−03 | 1.6E−03 | 5.5E−03 | No | — |
| 212235_at | 4.1E−03 | 2.2E−04 | 7.0E−04 | 7.9E−05 | No | PLXND1 |

| Probesets | UniGene ID | Gene Title |
|---|---|---|
| FDR Cutoff | | |
| 200068_s_at | Hs.155560 | calnexin |
| 200670_at | Hs.437638 | X-box binding protein 1 |
| 200701_at | Hs.433222 | Niemann-Pick disease, type C2 |
| 200736_s_at | Hs.76686 | glutathione peroxidase 1 |
| 200765_x_at | Hs.254321 | catenin (cadherin-associated protein), alpha |
| | | 1, 102 kDa |
| 200814_at | Hs.75348 | proteasome (prosome, macropain) activator |
| | | subunit 1 (PA28 alpha) |
| 200846_s_at | Hs.183994 | protein phosphatase 1, catalytic subunit, |
| | | alpha isoform |
| 200887_s_at | Hs.21486 | signal transducer and activator of |
| | | transcription 1, 91 kDa |
| 200905_x_at | Hs.381008 | major histocompatibility complex, class I, E |
| 200936_at | Hs.178551 | ribosomal protein L8 |
| 200941_at | Hs.250899 | heat shock factor binding protein 1 |
| 201011_at | Hs.2280 | ribophorin I |
| 201160_s_at | Hs.221889 | cold shock domain protein A |
| 201161_s_at | Hs.221889 | cold shock domain protein A |
| 201200_at | Hs.5710 | cellular repressor of E1A-stimulated genes |
| 201204_s_at | Hs.98614 | ribosome binding protein 1 homolog 180 kDa |
| | | (dog) |
| 201206_s_at | Hs.98614 | ribosome binding protein 1 homolog 180 kDa |
| | | (dog) |
| 201226_at | Hs.198273 | NADH dehydrogenase (ubiquinone) 1 beta |
| | | subcomplex, 8, 19 kDa |
| 201263_at | Hs.84131 | threonyl-tRNA synthetase |
| 201307_at | Hs.386784 | hypothetical protein FLJ10849 |
| 201398_s_at | Hs.4147 | translocation associated membrane protein 1 |
| 201399_s_at | Hs.4147 | translocation associated membrane protein 1 |
| 201470_at | Hs.11465 | glutathione S-transferase omega 1 |
| 201487_at | Hs.128065 | cathepsin C |
| 201499_s_at | Hs.386939 | ubiquitin specific protease 7 (herpes virus- |
| | | associated) |
| 201566_x_at | Hs.180919 | inhibitor of DNA binding 2, dominant |
| | | negative helix-loop-helix protein |
| 201590_x_at | Hs.462864 | annexin A2 |
| 201601_x_at | Hs.458414 | interferon induced transmembrane protein 1 |
| | | (9-27) |
| 201649_at | Hs.425777 | ubiquitin-conjugating enzyme E2L 6 |
| 201746_at | Hs.408312 | tumor protein p53 (Li-Fraumeni syndrome) |
| 201874_at | Hs.512729 | hypothetical protein FLJ21047 |
| 201937_s_at | Hs.258551 | aspartyl aminopeptidase |
| 201968_s_at | Hs.1869 | phosphoglucomutase 1 |
| 202096_s_at | Hs.202 | benzodiazapine receptor (peripheral) |
| 202123_s_at | Hs.446504 | v-abl Abelson murine leukemia viral |
| | | oncogene homolog 1 |
| 202261_at | Hs.2430 | transcription factor-like 1 |
| 202279_at | Hs.109052 | chromosome 14 open reading frame 2 |
| 202315_s_at | Hs.446394 | breakpoint cluster region |
| 202382_s_at | Hs.278500 | glucosamine-6-phosphate deaminase 1 |
| 202429_s_at | Hs.272458 | protein phosphatase 3 (formerly 2B), |
| | | catalytic subunit, alpha isoform (calcineurin A |
| | | alpha) |
| 202443_x_at | Hs.8121 | Notch homolog 2 (Drosophila) |
| 202447_at | Hs.414754 | 2,4-dienoyl CoA reductase 1, mitochondrial |
| 202457_s_at | Hs.272458 | protein phosphatase 3 (formerly 2B), |
| | | catalytic subunit, alpha isoform (calcineurin A |
| | | alpha) |
| 202536_at | Hs.11449 | DKFZP564O123 protein |
| 202696_at | Hs.95220 | oxidative-stress responsive 1 |
| 202732_at | Hs.3407 | protein kinase (cAMP-dependent, catalytic) |
| | | inhibitor gamma |
| 202811_at | Hs.12479 | associated molecule with the SH3 domain of |
| | | STAM |
| 202982_s_at | Hs.446685 | peroxisomal long-chain acyl-coA |
| | | thioesterase |
| 203031_s_at | Hs.75593 | uroporphyrinogen III synthase (congenital |
| | | erythropoietic porphyria) |
| 203113_s_at | Hs.334798 | eukaryotic translation elongation factor 1 |
| | | delta (guanine nucleotide exchange protein) |
| 203142_s_at | Hs.446648 | adaptor-related protein complex 3, beta 1 |
| | | subunit |
| 203217_s_at | Hs.415117 | sialyltransferase 9 (CMP- |
| | | NeuAc:lactosylceramide alpha-2,3- |
| | | sialyltransferase; GM3 synthase) |
| 203335_at | Hs.172887 | phytanoyl-CoA hydroxylase (Refsum |
| | | disease) |
| 203343_at | Hs.28309 | UDP-glucose dehydrogenase |
| 203459_s_at | Hs.188838 | vacuolar protein sorting protein 16 |
| 203659_s_at | Hs.436922 | ret finger protein 2 |
| 203825_at | Hs.86896 | bromodomain containing 3 |
| 203957_at | Hs.135465 | E2F transcription factor 6 |
| 204003_s_at | Hs.408241 | nucleoporin like 2 |
| 204044_at | Hs.335116 | quinolinate phosphoribosyltransferase |
| | | (nicotinate-nucleotide pyrophosphorylase |
| | | (carboxylating)) |
| 204168_at | Hs.81874 | microsomal glutathione S-transferase 2 |
| 204172_at | Hs.89866 | coproporphyrinogen oxidase |
| | | (coproporphyria, harderoporphyria) |
| 204220_at | Hs.5210 | glia maturation factor, gamma |
| 204234_s_at | Hs.104382 | zinc finger protein 195 |
| 204256_at | Hs.211556 | ELOVL family member 6, elongation of long |
| | | chain fatty acids (FEN1/Elo2, SUR4/Elo3- |
| | | like, yeast) |
| 204279_at | Hs.381081 | proteasome (prosome, macropain) subunit, |
| | | beta type, 9 (large multifunctional protease |
| | | 2) |
| 204326_x_at | Hs.374950 | metallothionein 1X |
| 204386_s_at | Hs.458367 | mitochondrial ribosomal protein 63 |
| 204418_x_at | Hs.279837 | glutathione S-transferase M2 (muscle) |
| 204565_at | Hs.9676 | thioesterase superfamily member 2 |
| 204766_s_at | Hs.413078 | nudix (nucleoside diphosphate linked moiety |
| | | X)-type motif 1 |
| 204769_s_at | Hs.502 | transporter 2, ATP-binding cassette, sub- |
| | | family B (MDR/TAP) |
| 204779_s_at | Hs.436181 | homeo box B7 |
| 204806_x_at | Hs.411958 | major histocompatibility complex, class I, F |
| 204937_s_at | Hs.83761 | zinc finger protein 274 |
| 205048_s_at | — | — |
| 205297_s_at | Hs.89575 | CD79B antigen(immunoglobulin-associated |
| | | beta) |
| 205659_at | Hs.487662 | histone deacetylase 9 |
| 206928_at | Hs.458362 | zinc finger protein 124 (HZF-16) |
| 207181_s_at | Hs.9216 | caspase 7, apoptosis-related cysteine |
| | | protease |
| 207304_at | Hs.381285 | zinc finger protein 45 (a Kruppel-associated |
| | | box (KRAB) domain polypeptide) |
| 207585_s_at | Hs.444749 | ribosomal protein L36a-like |
| 207777_s_at | Hs.399826 | SP140 nuclear body protein |
| 207809_s_at | Hs.6551 | ATPase, H+ transporting, lysosomal |
| | | accessory protein 1 |
| 208490_x_at | Hs.182137 | histone 1, H2bg |
| 208527_x_at | Hs.182138 | histone 1, H2be |
| 208579_x_at | Hs.473961 | H2B histone family, member S |
| 208581_x_at | Hs.374950 | metallothionein 1X |
| 208690_s_at | Hs.75807 | PDZ and LIM domain 1 (elfin) |
| 208729_x_at | Hs.77961 | major histocompatibility complex, class I, B |
| 208812_x_at | Hs.274485 | major histocompatibility complex, class I, C |
| 208918_s_at | Hs.220324 | NAD kinase |
| 209040_s_at | Hs.180062 | proteasome (prosome, macropain) subunit, |
| | | beta type, 8 (large multifunctional protease |
| | | 7) |
| 209112_at | Hs.238990 | cyclin-dependent kinase inhibitor 1B (p27, |
| | | Kip1) |
| 209124_at | Hs.82116 | myeloid differentiation primary response |
| | | gene (88) |
| 209138_x_at | Hs.458262 | immunoglobulin lambda locus |
| 209140_x_at | Hs.77961 | major histocompatibility complex, class I, B |
| 209175_at | Hs.300208 | SEC23 interacting protein |
| 209201_x_at | Hs.421986 | chemokine (C—X—C motif) receptor 4 |
| 209472_at | Hs.134460 | hypothetical protein 669 |
| 209476_at | Hs.125221 | thioredoxin domain containing |
| 209591_s_at | Hs.170195 | bone morphogenetic protein 7 (osteogenic |
| | | protein 1) |
| 209970_x_at | Hs.2490 | caspase 1, apoptosis-related cysteine |
| | | protease (interleukin 1, beta, convertase) |
| 209995_s_at | Hs.2484 | T-cell leukemia/lymphoma 1A |
| 210136_at | Hs.408543 | myelin basic protein |
| 210844_x_at | Hs.254321 | catenin (cadherin-associated protein), alpha |
| | | 1, 102 kDa |
| 211061_s_at | Hs.93338 | mannosyl (alpha-1,6-)-glycoprotein beta-1,2- |
| | | N-acetylglucosaminyltransferase |
| 211089_s_at | Hs.2236 | NIMA (never in mitosis gene a)-related |
| | | kinase 3 |
| 211366_x_at | Hs.2490 | caspase 1, apoptosis-related cysteine |
| | | protease (interleukin 1, beta, convertase) |
| 211528_x_at | Hs.512152 | HLA-G histocompatibility antigen, class I, G |
| 211529_x_at | Hs.512152 | HLA-G histocompatibility antigen, class I, G |
| 211530_x_at | Hs.512152 | HLA-G histocompatibility antigen, class I, G |
| 211799_x_at | Hs.274485 | major histocompatibility complex, class I, C |
| 211911_x_at | Hs.77961 | major histocompatibility complex, class I, B |
| 211919_s_at | Hs.421986 | chemokine (C—X—C motif) receptor 4 |
| 211989_at | Hs.437546 | SWI/SNF related, matrix associated, actin |
| | | dependent regulator of chromatin, subfamily |
| | | e, member 1 |
| 212057_at | Hs.222171 | KIAA0182 protein |
| 212071_s_at | Hs.205401 | spectrin, beta, non-erythrocytic 1 |
| 212149_at | Hs.84087 | KIAA0143 protein |
| 212382_at | Hs.359289 | transcription factor 4 |
| 212385_at | Hs.359289 | transcription factor 4 |
| 212386_at | Hs.359289 | transcription factor 4 |
| 212387_at | Hs.359289 | transcription factor 4 |
| 212408_at | Hs.234265 | lamina-associated polypeptide 1B |
| 212446_s_at | Hs.503941 | hypothetical protein LOC253782 |
| 212507_at | Hs.318783 | RW1 protein |
| 212547_at | Hs.6580 | Homo sapiens clone 23718 mRNA sequence |
| 212739_s_at | Hs.9235 | non-metastatic cells 4, protein expressed in |
| 212774_at | Hs.446677 | zinc finger protein 238 |
| 212792_at | Hs.408623 | KIAA0877 protein |
| 212798_s_at | Hs.112605 | hypothetical protein DKFZp564O043 |
| 212946_at | Hs.405457 | KIAA0564 protein |
| 213020_at | Hs.124436 | golgi SNAP receptor complex member 1 |
| 213116_at | Hs.2236 | NIMA (never in mitosis gene a)-related |
| | | kinase 3 |
| 213233_s_at | Hs.147717 | KIAA1354 protein |
| 213293_s_at | Hs.318501 | tripartite motif-containing 22 |
| 213357_at | Hs.356224 | Homo sapiens cDNA clone IMAGE: 4446165, |
| | | partial cds |
| 213361_at | Hs.416543 | tudor repeat associator with PCTAIRE 2 |
| 213435_at | Hs.412327 | SATB family member 2 |
| 213793_s_at | Hs.129051 | homer homolog 1 (Drosophila) |
| 213891_s_at | Hs.359289 | transcription factor 4 |
| 214022_s_at | Hs.458414 | interferon induced transmembrane protein 1 |
| | | (9-27) |
| 214032_at | Hs.234569 | zeta-chain (TCR) associated protein kinase |
| | | 70 kDa |
| 214172_x_at | Hs.285346 | RYK receptor-like tyrosine kinase |
| 214369_s_at | Hs.99491 | RAS guanyl releasing protein 2 (calcium and |
| | | DAG-regulated) |
| 214394_x_at | Hs.334798 | eukaryotic translation elongation factor 1 |
| | | delta (guanine nucleotide exchange protein) |
| 214459_x_at | Hs.274485 | major histocompatibility complex, class I, C |
| 214512_s_at | Hs.229641 | activated RNA polymerase II transcription |
| | | cofactor 4 |
| 214677_x_at | Hs.449601 | immunoglobulin lambda joining 3 |
| 214749_s_at | Hs.83530 | hypothetical protein FLJ20811 |
| 214835_s_at | Hs.446476 | succinate-CoA ligase, GDP-forming, beta |
| | | subunit |
| 214916_x_at | Hs.448957 | Homo sapiens partial mRNA for IgM |
| | | immunoglobulin heavy chain variable region |
| | | (IGHV gene), clone LIBPM376 |
| 215121_x_at | Hs.356861 | Homo sapiens cDNA FLJ26905 fis, clone |
| | | RCT01427, highly similar to Ig lambda chain |
| | | C regions |
| 215516_at | Hs.62022 | laminin, beta 4 |
| 215772_x_at | Hs.446476 | succinate-CoA ligase, GDP-forming, beta |
| | | subunit |
| 215946_x_at | Hs.272302 | Homo sapiens, clone IMAGE: 5728597, |
| | | mRNA |
| 216526_x_at | Hs.274485 | major histocompatibility complex, class I, C |
| 216733_s_at | Hs.75335 | glycine amidinotransferase (L- |
| | | arginine:glycine amidinotransferase) |
| 216973_s_at | Hs.436181 | homeo box B7 |
| 216976_s_at | Hs.285346 | RYK receptor-like tyrosine kinase |
| 217028_at | — | — |
| 217436_x_at | — | — |
| 217456_x_at | Hs.381008 | major histocompatibility complex, class I, E |
| 217809_at | Hs.5216 | basic leucine zipper and W2 domains 2 |
| 217839_at | Hs.446568 | TRK-fused gene |
| 217911_s_at | Hs.15259 | BCL2-associated athanogene 3 |
| 217940_s_at | Hs.8083 | hypothetical protein FLJ10769 |
| 217950_at | Hs.7236 | nitric oxide synthase interacting protein |
| 217988_at | Hs.107003 | chromosome 14 open reading frame 18 |
| 218026_at | Hs.16059 | HSPC009 protein |
| 218093_s_at | Hs.164969 | ankyrin repeat domain 10 |
| 218100_s_at | Hs.170318 | estrogen-related receptor beta like 1 |
| 218187_s_at | Hs.169615 | hypothetical protein FLJ20989 |
| 218224_at | Hs.194709 | paraneoplastic antigen MA1 |
| 218276_s_at | Hs.257341 | salvador homolog 1 (Drosophila) |
| 218285_s_at | Hs.124696 | dehydrogenase/reductase (SDR family) |
| | | member 6 |
| 218316_at | Hs.440525 | translocase of inner mitochondrial membrane |
| | | 9 homolog (yeast) |
| 218343_s_at | Hs.512838 | general transcription factor IIIC, polypeptide |
| | | 3, 102 kDa |
| 218358_at | Hs.28029 | hypothetical protein MGC11256 |
| 218377_s_at | Hs.34136 | chromosome 21 open reading frame 6 |
| 218490_s_at | Hs.436350 | zinc finger protein 302 |
| 218491_s_at | Hs.13645 | likely ortholog of the mouse thymocyte |
| | | protein Thy28 |
| 218543_s_at | Hs.12646 | zinc finger CCCH type domain containing 1 |
| 218557_at | Hs.439152 | Nit protein 2 |
| 218633_x_at | Hs.266514 | hypothetical protein FLJ11342 |
| 218648_at | Hs.434956 | hypothetical protein FLJ21868 |
| 218735_s_at | Hs.438994 | zinc finger protein |
| 218738_s_at | Hs.180403 | ring finger protein 138 |
| 218773_s_at | Hs.461420 | pilin-like transcription factor |
| 219008_at | Hs.63300 | hypothetical protein FLJ21820 |
| 219184_x_at | Hs.87595 | translocase of inner mitochondrial membrane |
| | | 22 homolog (yeast) |
| 219209_at | Hs.389539 | melanoma differentiation associated protein-5 |
| 219215_s_at | Hs.411274 | solute carrier family 39 (zinc transporter), |
| | | member 4 |
| 219248_at | Hs.368545 | chromosome 2 open reading frame 8 |
| 219471_at | Hs.413071 | chromosome 13 open reading frame 18 |
| 219489_s_at | Hs.133999 | rhomboid, veinlet-like 2 (Drosophila) |
| 219563_at | Hs.41502 | chromosome 14 open reading frame 139 |
| 219664_s_at | Hs.15898 | 2,4-dienoyl CoA reductase 2, peroxisomal |
| 219767_s_at | Hs.352671 | crystallin, zeta (quinone reductase)-like 1 |
| 219822_at | Hs.348472 | mitochondrial translational release factor 1 |
| 220741_s_at | Hs.421825 | inorganic pyrophosphatase 2 |
| 221234_s_at | Hs.88414 | BTB and GNC homology 1, basic leucine |
| | | zipper transcription factor 2 |
| 221614_s_at | Hs.437436 | rabphilin 3A-like (without C2 domains) |
| 221847_at | Hs.278469 | taste receptor, type 2, member 14 |
| 221874_at | Hs.104696 | maba1 |
| 221875_x_at | Hs.411958 | major histocompatibility complex, class I, F |
| 222146_s_at | Hs.359289 | transcription factor 4 |
| 35671_at | Hs.331 | general transcription factor IIIC, polypeptide |
| | | 1, alpha 220 kDa |
| 36830_at | Hs.68583 | mitochondrial intermediate peptidase |
| 36936_at | Hs.404119 | tissue specific transplantation antigen P35B |
| 37384_at | Hs.278441 | protein phosphatase 1F (PP2C domain |
| | | containing) |
| 39318_at | Hs.2484 | T-cell leukemia/lymphoma 1A |
| 40148_at | Hs.324125 | amyloid beta (A4) precursor protein-binding, |
| | | family B, member 2 (Fe65-like) |
| 200660_at | Hs.417004 | S100 calcium binding protein A11 |
| | | (calgizzarin) |
| 200671_s_at | Hs.205401 | spectrin, beta, non-erythrocytic 1 |
| 200672_x_at | Hs.205401 | spectrin, beta, non-erythrocytic 1 |
| 200704_at | Hs.76507 | lipopolysaccharide-induced TNF factor |
| 200706_s_at | Hs.76507 | lipopolysaccharide-induced TNF factor |
| 200782_at | Hs.145741 | annexin A5 |
| 200923_at | Hs.79339 | lectin, galactoside-binding, soluble, 3 binding |
| | | protein |
| 201005_at | Hs.387579 | CD9 antigen (p24) |
| 201426_s_at | Hs.435800 | vimentin |
| 201485_s_at | Hs.79088 | reticulocalbin 2, EF-hand calcium binding |
| | | domain |
| 201792_at | Hs.439463 | AE binding protein 1 |
| 202218_s_at | Hs.388164 | fatty acid desaturase 2 |
| 202283_at | Hs.173594 | serine (or cysteine) proteinase inhibitor, |
| | | clade F (alpha-2 antiplasmin, pigment |
| | | epithelium derived factor), member 1 |
| 202719_s_at | Hs.129129 | testis derived transcript (3 LIM domains) |
| 202855_s_at | Hs.386678 | solute carrier family 16 (monocarboxylic acid |
| | | transporters), member 3 |
| 202856_s_at | Hs.386678 | solute carrier family 16 (monocarboxylic acid |
| | | transporters), member 3 |
| 203045_at | Hs.11342 | ninjurin 1 |
| 203063_at | Hs.278441 | protein phosphatase 1F (PP2C domain |
| | | containing) |
| 203068_at | Hs.7764 | KIAA0469 gene product |
| 203211_s_at | Hs.181326 | myotubularin related protein 2 |
| 203212_s_at | Hs.181326 | myotubularin related protein 2 |
| 203222_s_at | Hs.406491 | transducin-like enhancer of split 1 (E(sp1) |
| | | homolog, Drosophila) |
| 203305_at | Hs.80424 | coagulation factor XIII, A1 polypeptide |
| 203408_s_at | Hs.416026 | special AT-rich sequence binding protein 1 |
| | | (binds to nuclear matrix/scaffold-associating |
| | | DNA's) |
| 203557_s_at | Hs.3192 | 6-pyruvoyl-tetrahydropterin |
| | | synthase/dimerization cofactor of hepatocyte |
| | | nuclear factor 1 alpha (TCF1) |
| 203708_at | Hs.188 | phosphodiesterase 4B, cAMP-specific |
| | | (phosphodiesterase E4 dunce homolog, |
| | | Drosophila) |
| 203853_s_at | Hs.30687 | GRB2-associated binding protein 2 |
| 204305_at | Hs.68583 | mitochondrial intermediate peptidase |
| 204821_at | Hs.167741 | butyrophilin, subfamily 3, member A3 |
| 204994_at | Hs.926 | myxovirus (influenza virus) resistance 2 |
| | | (mouse) |
| 205173_x_at | Hs.75626 | CD58 antigen, (lymphocyte function- |
| | | associated antigen 3) |
| 205181_at | Hs.100921 | zinc finger protein 193 |
| 205366_s_at | Hs.98428 | homeo box B6 |
| 205401_at | Hs.407933 | alkylglycerone phosphate synthase |
| 205668_at | Hs.153563 | lymphocyte antigen 75 |
| 205691_at | Hs.435277 | synaptogyrin 3 |
| 205790_at | Hs.411942 | src family associated phosphoprotein 1 |
| 205932_s_at | Hs.424414 | msh homeo box homolog 1 (Drosophila) |
| 206082_at | Hs.511759 | HLA complex P5 |
| 206261_at | Hs.25040 | zinc finger protein 239 |
| 206437_at | Hs.159543 | endothelial differentiation, G-protein-coupled |
| | | receptor 6 |
| 206554_x_at | Hs.265855 | SET domain and mariner transposase fusion |
| | | gene |
| 206591_at | Hs.73958 | recombination activating gene 1 |
| 206660_at | Hs.348935 | immunoglobulin lambda-like polypeptide 1 |
| 206698_at | Hs.78919 | Kell blood group precursor (McLeod |
| | | phenotype) |
| 206866_at | Hs.376792 | cadherin 4, type 1, R-cadherin (retinal) |
| 208146_s_at | Hs.95594 | carboxypeptidase, vitellogenic-like |
| 208964_s_at | Hs.503546 | fatty acid desaturase 1 |
| 209129_at | Hs.380230 | thyroid hormone receptor interactor 6 |
| 209310_s_at | Hs.74122 | caspase 4, apoptosis-related cysteine |
| | | protease |
| 209398_at | Hs.7644 | histone 1, H1c |
| 209590_at | Hs.170195 | bone morphogenetic protein 7 (osteogenic |
| | | protein 1) |
| 209728_at | Hs.308026 | major histocompatibility complex, class II, |
| | | DR beta 3 |
| 209790_s_at | Hs.3280 | caspase 6, apoptosis-related cysteine |
| | | protease |
| 210427_x_at | Hs.462864 | annexin A2 |
| 210715_s_at | Hs.31439 | serine protease inhibitor, Kunitz type, 2 |
| 210830_s_at | Hs.165598 | paraoxonase 2 |
| 211367_s_at | Hs.2490 | caspase 1, apoptosis-related cysteine |
| | | protease (interleukin 1, beta, convertase) |
| 211368_s_at | Hs.2490 | caspase 1, apoptosis-related cysteine |
| | | protease (interleukin 1, beta, convertase) |
| 211812_s_at | Hs.418062 | UDP-Gal:betaGlcNAc beta 1,3- |
| | | galactosyltransferase, polypeptide 3 |
| 212268_at | Hs.381167 | serine (or cysteine) proteinase inhibitor |
| | | clade B (ovalbumin), member 1 |
| 212442_s_at | Hs.503941 | hypothetical protein LOC253782 |
| 212561_at | Hs.26797 | RAB6 interacting protein 1 |
| 213147_at | Hs.110637 | homeo box A10 |
| 213150_at | Hs.110637 | homeo box A10 |
| 213371_at | Hs.49998 | LIM domain binding 3 |
| 213419_at | — | — |
| 213502_x_at | Hs.272302 | Homo sapiens, clone IMAGE: 5728597, |
| | | mRNA |
| 213503_x_at | Hs.462864 | annexin A2 |
| 213566_at | Hs.23262 | ribonuclease, RNase A family, k6 |
| 213568_at | Hs.152823 | odd-skipped-related 2A protein |
| 213572_s_at | Hs.381167 | serine (or cysteine) proteinase inhibitor, |
| | | clade B (ovalbumin), member 1 |
| 213625_at | Hs.44720 | hypothetical protein P1 p373c6 |
| 213788_s_at | Hs.6580 | Homo sapiens clone 23718 mRNA sequence |
| 215071_s_at | — | — |
| 215379_x_at | Hs.449601 | immunoglobulin lambda joining 3 |
| 216565_x_at | — | — |
| 217022_s_at | Hs.366 | hypothetical protein MGC27165 |
| 217360_x_at | — | — |
| 217759_at | Hs.14512 | tripartite motif-containing 44 |
| 217760_at | Hs.14512 | tripartite motif-containing 44 |
| 217977_at | Hs.279623 | selenoprotein X, 1 |
| 218005_at | Hs.108642 | zinc finger protein 22 (KOX 15) |
| 218006_s_at | Hs.108642 | zinc finger protein 22 (KOX 15) |
| 218154_at | Hs.118983 | hypothetical protein FLJ12150 |
| 218457_s_at | Hs.241565 | DNA (cytosine-5-)-methyltransferase 3 alpha |
| 218503_at | Hs.257696 | KIAA1797 |
| 218618_s_at | Hs.299883 | FAD104 |
| 218676_s_at | Hs.285218 | phosphatidylcholine transfer protein |
| 218689_at | Hs.65328 | Fanconi anemia, complementation group F |
| 218729_at | Hs.124491 | latexin protein |
| 219143_s_at | Hs.8562 | RNase P protein subunit p25 |
| 219362_at | Hs.325923 | Homo sapiens transcribed sequence with |
| | | strong similarity to protein ref: NP_078911.1 |
| | | (H. sapiens) hypothetical protein FLJ22643 |
| | | [Homo sapiens] |
| 219684_at | Hs.43388 | 28 kD interferon responsive protein |
| 219734_at | Hs.272416 | hypothetical protein FLJ20174 |
| 219953_s_at | Hs.131180 | chromosome 11 open reading frame 17 |
| 220104_at | Hs.35254 | zinc finger CCCH type, antiviral 1 |
| 220230_s_at | Hs.414362 | cytochrome b5 reductase b5R.2 |
| 220235_s_at | Hs.25245 | receptor-interacting factor 1 |
| 220784_s_at | Hs.162200 | urotensin 2 |
| 221081_s_at | Hs.447624 | hypothetical protein FLJ22457 |
| 221349_at | Hs.247979 | pre-B lymphocyte gene 1 |
| 221641_s_at | Hs.298885 | likely ortholog of mouse acyl-Coenzyme A |
| | | thioesterase 2, mitochondrial |
| 221666_s_at | Hs.197875 | apoptosis-associated speck-like protein |
| | | containing a CARD |
| 221690_s_at | Hs.369279 | NACHT, leucine rich repeat and PYD |
| | | containing 2 |
| 36030_at | Hs.46659 | HOM-TES-103 tumor antigen-like |
| 51158_at | Hs.27373 | Homo sapiens, clone IMAGE: 4816940, |
| | | mRNA |
| 207714_s_at | Hs.241579 | serine (or cysteine) proteinase inhibitor, |
| | | clade H (heat shock protein 47), member 1, |
| | | (collagen binding protein 1) |
| 208816_x_at | Hs.437110 | Human lipocortin (LIP) 2 pseudogene |
| | | mRNA, complete cds-like region. |
| 211566_x_at | — | — |
| 212235_at | Hs.301685 | plexin D1 |

TABLE 4A
NFκB-Regulated Genes (See tables reference 1)

| | Log2 fluorescence intensity | | | | | | | | | | | |
|---|---|---|---|---|---|---|---|---|---|---|---|---|
| | | | | | | | | | | | | D4 |
| Probesets | Ly1 (A1) | Ly1 (A2) | Ly1 (A3) | Ly7 (B1) | Ly7 (B2) | Ly7 (B3) | Ly8 (C1) | Ly8 (C2) | Ly8 (C3) | D4 (D1) | D4 (D2) | (D3) |
| 205481_at | 5.59 | 6.35 | 5.96 | 5.42 | 5.21 | 5.36 | 5.63 | 6.59 | 6.09 | 5.66 | 5.40 | 5.42 |
| 216220_s_at | 5.39 | 5.61 | 5.43 | 5.33 | 5.38 | 5.22 | 5.31 | 5.82 | 5.51 | 5.34 | 5.33 | 5.21 |
| 202834_at | 5.58 | 5.84 | 5.84 | 5.79 | 6.06 | 5.92 | 5.79 | 5.94 | 5.74 | 6.04 | 6.02 | 6.03 |
| 204151_x_at | 6.84 | 6.88 | 7.03 | 6.63 | 6.91 | 6.98 | 6.88 | 6.99 | 6.92 | 6.90 | 7.04 | 6.74 |
| 216594_x_at | 6.35 | 6.32 | 6.33 | 6.17 | 6.32 | 6.45 | 6.28 | 6.42 | 6.16 | 6.49 | 6.65 | 6.24 |
| 204445_s_at | 7.06 | 6.70 | 6.75 | 6.37 | 6.41 | 6.19 | 7.05 | 6.65 | 6.46 | 6.54 | 6.72 | 6.55 |
| 204446_s_at | 8.83 | 8.54 | 9.09 | 7.84 | 7.92 | 8.09 | 8.89 | 8.88 | 9.00 | 8.17 | 8.15 | 8.85 |
| 214366_s_at | 6.87 | 6.47 | 6.50 | 6.14 | 5.92 | 6.06 | 7.27 | 6.33 | 6.41 | 6.17 | 6.31 | 6.39 |
| 214953_s_at | 4.78 | 5.06 | 5.02 | 4.53 | 5.03 | 5.06 | 4.63 | 5.05 | 4.79 | 4.81 | 5.35 | 5.10 |
| 211621_at | 4.47 | 4.64 | 4.79 | 4.51 | 4.65 | 4.62 | 4.52 | 4.55 | 4.53 | 4.58 | 4.61 | 4.57 |
| 203174_s_at | 7.70 | 7.86 | 7.69 | 7.88 | 7.93 | 7.79 | 7.74 | 7.78 | 7.77 | 8.15 | 7.84 | 7.80 |
| 215984_s_at | 6.01 | 6.48 | 6.24 | 6.53 | 6.66 | 6.51 | 6.32 | 6.29 | 6.45 | 6.43 | 6.20 | 6.25 |
| 201891_s_at | 12.00 | 12.00 | 12.13 | 12.01 | 12.13 | 11.98 | 11.99 | 12.32 | 12.04 | 12.14 | 11.85 | 11.88 |
| 216231_s_at | 12.17 | 12.10 | 12.09 | 12.06 | 12.15 | 12.20 | 12.12 | 12.16 | 12.13 | 12.08 | 12.00 | 11.92 |
| 203685_at | 4.75 | 4.61 | 4.79 | 4.33 | 4.46 | 4.42 | 4.62 | 4.65 | 4.68 | 4.48 | 4.58 | 4.41 |
| 207005_s_at | 6.62 | 6.60 | 6.62 | 6.35 | 6.12 | 5.92 | 7.06 | 6.73 | 6.62 | 6.53 | 6.85 | 6.23 |
| 205681_at | 4.31 | 4.32 | 4.29 | 4.59 | 4.61 | 4.49 | 4.45 | 4.48 | 4.30 | 6.84 | 8.14 | 7.47 |
| 202076_at | 8.63 | 8.09 | 8.59 | 8.79 | 8.32 | 8.73 | 8.67 | 8.13 | 8.76 | 7.56 | 7.54 | 8.28 |
| 210538_s_at | 4.64 | 4.75 | 4.80 | 6.89 | 6.65 | 6.57 | 4.49 | 4.35 | 4.62 | 4.95 | 5.43 | 5.21 |
| 205114_s_at | 4.61 | 4.61 | 4.69 | 4.63 | 5.15 | 4.59 | 4.61 | 4.81 | 4.46 | 4.99 | 6.04 | 4.94 |
| 204103_at | 6.95 | 6.97 | 6.93 | 6.16 | 6.49 | 6.36 | 6.80 | 7.08 | 6.54 | 6.61 | 7.13 | 6.62 |
| 201700_at | 9.37 | 9.74 | 9.72 | 10.56 | 10.72 | 10.78 | 9.35 | 9.64 | 9.48 | 8.39 | 8.57 | 8.77 |
| 204118_at | 9.63 | 8.78 | 9.07 | 9.76 | 10.02 | 9.44 | 9.69 | 8.64 | 8.84 | 9.94 | 10.36 | 9.91 |
| 209795_at | 4.56 | 4.42 | 4.80 | 4.00 | 4.14 | 4.03 | 4.36 | 4.48 | 4.27 | 4.07 | 4.06 | 4.06 |
| 209619_at | 12.37 | 11.96 | 12.10 | 12.07 | 12.17 | 11.94 | 12.11 | 11.63 | 11.56 | 12.67 | 12.83 | 12.63 |
| 207176_s_at | 6.11 | 6.33 | 6.32 | 6.31 | 6.25 | 6.39 | 6.13 | 6.37 | 6.25 | 6.58 | 6.49 | 6.50 |
| 204440_at | 7.44 | 7.55 | 7.48 | 7.19 | 7.41 | 7.33 | 7.53 | 7.60 | 7.15 | 8.55 | 9.42 | 8.62 |
| 202246_s_at | 10.32 | 10.61 | 10.25 | 10.63 | 10.70 | 10.45 | 10.37 | 10.53 | 10.35 | 10.50 | 10.55 | 10.32 |
| 203198_at | 7.63 | 7.79 | 7.90 | 8.06 | 7.91 | 7.96 | 7.68 | 7.75 | 7.82 | 7.36 | 7.11 | 7.11 |
| 202284_s_at | 8.10 | 7.23 | 7.18 | 6.96 | 8.45 | 6.88 | 8.74 | 7.87 | 7.04 | 6.85 | 6.54 | 6.42 |
| 208485_x_at | 5.64 | 5.22 | 5.33 | 5.39 | 5.51 | 5.40 | 5.30 | 5.36 | 5.30 | 5.31 | 5.25 | 4.96 |
| 209508_x_at | 6.13 | 6.12 | 6.08 | 6.12 | 6.31 | 5.96 | 6.16 | 6.13 | 6.03 | 5.98 | 5.95 | 5.74 |
| 209939_x_at | 6.86 | 6.88 | 7.01 | 7.80 | 7.75 | 7.78 | 6.85 | 7.01 | 7.04 | 6.40 | 6.16 | 6.11 |
| 210563_x_at | 6.61 | 6.84 | 6.71 | 7.62 | 7.36 | 7.39 | 6.74 | 6.73 | 6.85 | 6.59 | 6.34 | 6.51 |
| 211316_x_at | 6.52 | 6.42 | 6.35 | 6.52 | 6.68 | 6.33 | 6.48 | 6.64 | 6.28 | 6.06 | 5.97 | 5.95 |
| 211317_s_at | 4.50 | 4.60 | 4.70 | 4.66 | 4.65 | 4.71 | 4.51 | 4.60 | 4.59 | 4.42 | 4.51 | 4.47 |
| 211862_x_at | 5.34 | 5.18 | 5.07 | 5.44 | 5.23 | 5.21 | 5.02 | 5.04 | 5.05 | 4.80 | 4.82 | 4.56 |
| 214618_at | 4.41 | 4.69 | 4.77 | 4.70 | 4.55 | 4.85 | 4.68 | 4.69 | 4.73 | 4.81 | 4.98 | 4.74 |
| 209716_at | 5.79 | 5.84 | 5.75 | 5.61 | 5.86 | 5.82 | 5.93 | 5.85 | 5.72 | 5.85 | 5.93 | 5.85 |
| 200838_at | 5.36 | 5.36 | 5.27 | 5.18 | 5.00 | 5.04 | 5.41 | 5.50 | 5.37 | 5.35 | 5.15 | 5.18 |
| 200839_s_at | 6.35 | 6.15 | 6.04 | 5.43 | 5.33 | 5.59 | 6.11 | 6.30 | 6.56 | 5.87 | 5.66 | 5.63 |
| 213274_s_at | 4.90 | 4.91 | 5.00 | 4.66 | 5.02 | 5.02 | 4.86 | 5.12 | 4.95 | 4.84 | 5.01 | 4.83 |
| 204470_at | 4.01 | 4.12 | 4.00 | 3.99 | 4.18 | 4.03 | 3.96 | 4.06 | 3.97 | 3.89 | 4.12 | 4.00 |
| 210163_at | 3.84 | 3.75 | 3.75 | 3.79 | 3.86 | 3.78 | 3.73 | 3.78 | 3.81 | 3.76 | 3.81 | 3.82 |
| 214974_x_at | 3.82 | 3.75 | 3.86 | 3.75 | 3.95 | 3.84 | 3.70 | 3.79 | 3.78 | 3.81 | 3.89 | 3.81 |
| 203700_s_at | 5.40 | 5.39 | 5.32 | 5.45 | 5.26 | 5.44 | 5.30 | 5.50 | 5.37 | 5.55 | 5.57 | 5.35 |
| 201041_s_at | 6.21 | 6.25 | 6.14 | 5.90 | 5.81 | 5.72 | 6.52 | 6.52 | 6.34 | 5.50 | 5.12 | 5.20 |
| 203692_s_at | 7.19 | 6.93 | 6.66 | 7.29 | 7.06 | 6.97 | 7.05 | 6.91 | 7.07 | 7.15 | 7.02 | 7.17 |
| 203693_s_at | 7.74 | 7.57 | 8.21 | 8.08 | 7.60 | 8.05 | 7.49 | 7.68 | 7.86 | 7.78 | 7.11 | 8.18 |
| 211551_at | 5.94 | 5.90 | 5.89 | 5.71 | 6.15 | 5.96 | 5.76 | 5.82 | 5.80 | 5.98 | 6.04 | 5.78 |
| 201694_s_at | 5.28 | 5.32 | 5.40 | 5.19 | 5.37 | 5.44 | 5.27 | 5.32 | 5.47 | 6.19 | 5.34 | 5.75 |
| 214766_s_at | 5.28 | 5.51 | 5.55 | 6.06 | 5.57 | 5.87 | 5.38 | 5.38 | 5.59 | 4.76 | 4.60 | 5.05 |
| 201313_at | 4.81 | 4.57 | 4.51 | 7.05 | 7.14 | 6.55 | 4.66 | 4.84 | 4.55 | 4.88 | 4.81 | 4.70 |
| 205767_at | 3.77 | 3.79 | 3.77 | 3.77 | 3.75 | 3.74 | 3.85 | 3.79 | 3.78 | 3.78 | 3.77 | 3.76 |
| 202081_at | 9.51 | 9.31 | 9.03 | 10.39 | 10.23 | 10.27 | 9.52 | 9.16 | 9.15 | 10.35 | 10.75 | 10.50 |
| 205756_s_at | 5.46 | 5.28 | 5.32 | 5.06 | 5.29 | 5.22 | 5.25 | 5.25 | 5.34 | 5.13 | 5.30 | 5.27 |
| 214701_s_at | 5.44 | 5.40 | 5.21 | 5.48 | 5.39 | 5.28 | 5.63 | 5.48 | 5.31 | 5.52 | 5.64 | 5.43 |
| 202275_at | 7.07 | 7.09 | 6.80 | 7.02 | 7.11 | 6.67 | 7.11 | 7.11 | 6.89 | 7.27 | 7.49 | 7.32 |
| 207574_s_at | 7.55 | 6.78 | 6.81 | 6.37 | 6.11 | 6.33 | 7.73 | 6.90 | 7.36 | 7.45 | 7.12 | 7.56 |
| 209304_x_at | 7.93 | 7.32 | 6.99 | 6.93 | 6.71 | 6.64 | 7.80 | 7.38 | 7.66 | 7.92 | 7.70 | 7.83 |
| 209305_s_at | 7.57 | 6.94 | 6.82 | 6.59 | 6.37 | 6.71 | 7.51 | 6.69 | 7.25 | 7.40 | 7.40 | 7.39 |
| 202269_x_at | 4.45 | 5.13 | 5.03 | 3.96 | 3.99 | 3.97 | 4.66 | 5.02 | 5.11 | 3.98 | 3.95 | 3.87 |
| 202270_at | 4.47 | 4.42 | 4.92 | 3.79 | 3.76 | 3.92 | 4.53 | 4.62 | 5.11 | 3.91 | 3.81 | 3.92 |
| 200651_at | 12.85 | 12.93 | 13.02 | 12.61 | 12.79 | 12.73 | 13.12 | 13.03 | 13.01 | 12.85 | 12.91 | 12.85 |
| 222034_at | 5.06 | 5.16 | 5.28 | 5.45 | 5.31 | 5.31 | 5.25 | 5.11 | 5.25 | 5.38 | 5.07 | 5.46 |
| 200824_at | 10.68 | 10.43 | 10.32 | 11.31 | 11.31 | 11.17 | 10.62 | 10.37 | 10.54 | 10.94 | 11.08 | 10.94 |
| 208729_x_at | 12.62 | 12.49 | 12.18 | 9.61 | 9.63 | 9.55 | 12.28 | 12.26 | 12.09 | 8.01 | 8.43 | 8.04 |
| 209140_x_at | 13.05 | 12.93 | 13.01 | 10.68 | 10.95 | 10.64 | 13.05 | 12.96 | 12.89 | 8.24 | 8.59 | 8.46 |
| 211911_x_at | 12.27 | 12.11 | 12.01 | 9.49 | 9.50 | 9.28 | 12.20 | 11.97 | 11.83 | 7.81 | 7.96 | 7.69 |
| 200943_at | 11.40 | 11.49 | 11.48 | 11.74 | 11.63 | 11.66 | 11.26 | 11.62 | 11.44 | 11.65 | 11.60 | 11.63 |
| 200944_s_at | 11.32 | 11.47 | 11.55 | 11.63 | 11.61 | 11.69 | 11.40 | 11.68 | 11.50 | 11.72 | 11.63 | 11.69 |
| 202638_s_at | 8.64 | 7.77 | 7.46 | 7.18 | 7.29 | 6.90 | 7.99 | 7.52 | 7.28 | 7.32 | 7.87 | 7.25 |
| 215485_s_at | 6.87 | 6.62 | 6.39 | 6.69 | 6.41 | 6.63 | 6.82 | 6.57 | 6.43 | 6.77 | 6.99 | 6.49 |
| 210354_at | 4.51 | 4.61 | 4.52 | 4.46 | 4.57 | 4.57 | 4.43 | 4.67 | 4.43 | 4.67 | 4.73 | 4.57 |
| 202718_at | 9.02 | 6.64 | 7.87 | 5.46 | 5.55 | 5.74 | 8.21 | 6.43 | 7.16 | 5.80 | 5.58 | 5.57 |
| 207901_at | 4.00 | 4.00 | 3.99 | 3.97 | 4.10 | 3.96 | 3.90 | 3.97 | 3.92 | 4.01 | 3.94 | 3.99 |
| 208200_at | 4.85 | 4.98 | 4.97 | 4.80 | 4.84 | 4.94 | 4.86 | 4.98 | 4.91 | 5.02 | 4.93 | 4.96 |
| 205207_at | 4.73 | 4.82 | 4.86 | 4.76 | 4.90 | 4.82 | 4.92 | 4.79 | 4.74 | 4.91 | 4.84 | 4.75 |
| 202531_at | 7.23 | 7.83 | 7.69 | 6.08 | 6.64 | 6.37 | 7.36 | 7.91 | 7.11 | 5.89 | 6.37 | 6.03 |
| 203275_at | 6.41 | 6.61 | 6.81 | 6.03 | 6.11 | 6.08 | 6.61 | 6.52 | 6.60 | 4.37 | 5.36 | 4.62 |
| 204562_at | 8.22 | 8.60 | 8.59 | 8.69 | 9.38 | 8.42 | 8.35 | 8.44 | 7.86 | 6.36 | 6.28 | 6.01 |
| 216986_s_at | 6.51 | 6.37 | 6.35 | 6.45 | 6.80 | 6.42 | 6.55 | 6.43 | 6.23 | 6.43 | 6.42 | 6.01 |
| 208436_s_at | 6.62 | 5.66 | 5.62 | 5.28 | 5.20 | 5.19 | 6.65 | 5.82 | 6.12 | 5.31 | 4.93 | 5.00 |
| 213281_at | 4.36 | 4.50 | 4.48 | 4.33 | 4.39 | 4.32 | 4.41 | 4.47 | 4.41 | 4.40 | 4.43 | 4.37 |
| 201473_at | 7.65 | 7.49 | 7.49 | 7.56 | 7.32 | 7.43 | 7.46 | 7.25 | 7.43 | 8.06 | 8.38 | 8.13 |
| 207029_at | 4.03 | 4.08 | 4.03 | 3.88 | 4.14 | 4.00 | 4.05 | 4.06 | 3.93 | 4.00 | 4.06 | 4.02 |
| 216264_s_at | 4.59 | 4.66 | 4.42 | 4.60 | 4.61 | 4.69 | 4.59 | 4.44 | 4.47 | 4.80 | 4.68 | 4.48 |
| 207339_s_at | 6.04 | 5.63 | 5.88 | 6.78 | 6.47 | 6.58 | 5.99 | 5.80 | 5.68 | 6.82 | 7.57 | 6.95 |
| 213975_s_at | 4.87 | 5.48 | 5.69 | 4.30 | 4.17 | 4.18 | 5.57 | 6.22 | 6.92 | 4.27 | 4.39 | 4.33 |
| 208037_s_at | 6.64 | 6.46 | 6.49 | 6.40 | 6.49 | 6.61 | 6.54 | 6.47 | 6.43 | 6.72 | 6.66 | 6.55 |
| 206296_x_at | 8.63 | 8.05 | 8.15 | 9.80 | 9.53 | 9.24 | 8.32 | 7.87 | 7.85 | 9.99 | 10.33 | 10.05 |
| 214219_x_at | 8.30 | 7.85 | 7.89 | 9.41 | 9.21 | 8.89 | 8.23 | 7.62 | 7.57 | 9.52 | 10.01 | 9.75 |
| 214339_s_at | 7.79 | 7.44 | 7.78 | 9.09 | 8.83 | 8.74 | 7.92 | 7.33 | 7.21 | 9.17 | 9.58 | 9.55 |
| 201126_s_at | 7.87 | 7.81 | 7.67 | 7.65 | 7.70 | 7.28 | 7.65 | 7.90 | 7.56 | 7.26 | 7.14 | 7.03 |
| 203936_s_at | 5.85 | 6.14 | 6.00 | 6.13 | 5.94 | 6.04 | 6.08 | 6.12 | 6.07 | 6.23 | 6.22 | 6.12 |
| 202086_at | 5.28 | 5.17 | 5.31 | 5.47 | 5.32 | 5.27 | 5.42 | 5.37 | 5.29 | 5.49 | 5.29 | 5.26 |
| 204798_at | 7.85 | 9.53 | 9.60 | 7.15 | 7.27 | 7.39 | 7.90 | 9.44 | 8.38 | 6.59 | 7.23 | 7.08 |
| 215152_at | 3.85 | 4.08 | 3.91 | 3.91 | 3.91 | 3.86 | 3.98 | 3.94 | 3.90 | 3.90 | 3.90 | 3.86 |
| 202431_s_at | 10.47 | 10.56 | 10.92 | 9.94 | 10.06 | 9.69 | 10.34 | 10.50 | 9.93 | 9.96 | 10.46 | 9.66 |
| 209239_at | 8.35 | 8.59 | 8.52 | 7.22 | 7.63 | 7.16 | 8.37 | 8.51 | 8.19 | 8.15 | 8.49 | 8.00 |
| 207535_s_at | 5.46 | 5.26 | 5.22 | 6.18 | 6.01 | 5.92 | 5.38 | 5.08 | 5.09 | 5.51 | 5.90 | 5.73 |
| 201502_s_at | 9.28 | 8.98 | 8.75 | 9.09 | 9.14 | 8.67 | 9.01 | 8.78 | 8.68 | 9.09 | 9.69 | 9.61 |
| 201468_s_at | 6.10 | 6.24 | 6.14 | 6.15 | 5.99 | 6.05 | 6.00 | 6.26 | 6.21 | 6.83 | 6.57 | 6.61 |
| 210519_s_at | 6.22 | 6.59 | 6.26 | 5.79 | 6.08 | 6.04 | 6.32 | 6.44 | 6.44 | 7.55 | 7.37 | 7.57 |
| 201866_s_at | 5.88 | 5.84 | 6.06 | 6.95 | 7.02 | 6.82 | 5.72 | 5.83 | 5.92 | 6.19 | 6.47 | 6.30 |
| 211671_s_at | 6.23 | 6.16 | 6.46 | 8.03 | 7.78 | 7.72 | 5.79 | 5.73 | 6.03 | 6.48 | 7.23 | 7.14 |
| 216321_s_at | 5.82 | 5.76 | 6.16 | 7.65 | 7.57 | 7.58 | 5.29 | 5.44 | 5.78 | 6.98 | 7.38 | 7.57 |
| 204622_x_at | 4.46 | 4.53 | 4.61 | 4.36 | 4.43 | 4.49 | 4.41 | 4.52 | 4.57 | 4.49 | 4.50 | 4.41 |
| 216248_s_at | 4.30 | 4.22 | 4.23 | 4.10 | 4.05 | 4.08 | 4.24 | 4.18 | 4.46 | 4.13 | 4.12 | 4.05 |
| 210004_at | 3.87 | 3.93 | 3.88 | 3.78 | 3.95 | 3.94 | 3.82 | 3.94 | 3.82 | 3.83 | 3.93 | 3.76 |
| 121_at | 8.57 | 8.75 | 8.62 | 8.48 | 8.89 | 8.82 | 8.53 | 8.56 | 8.50 | 8.77 | 8.93 | 8.62 |
| 203557_s_at | 7.38 | 7.33 | 7.19 | 5.60 | 5.83 | 5.66 | 7.56 | 7.50 | 7.37 | 6.52 | 6.71 | 6.57 |
| 201202_at | 10.96 | 10.86 | 10.78 | 11.44 | 11.45 | 11.49 | 10.87 | 10.89 | 10.98 | 11.17 | 10.60 | 11.06 |
| 213791_at | 4.72 | 4.99 | 5.02 | 4.62 | 4.99 | 4.95 | 4.73 | 4.75 | 4.76 | 4.78 | 4.90 | 4.84 |
| 200737_at | 10.76 | 10.08 | 10.46 | 9.46 | 9.45 | 9.54 | 10.75 | 10.13 | 10.39 | 9.36 | 9.73 | 9.60 |
| 200738_s_at | 11.89 | 11.48 | 11.89 | 11.19 | 11.21 | 11.05 | 12.00 | 11.68 | 11.73 | 11.33 | 11.18 | 11.25 |
| 217356_s_at | 11.83 | 11.28 | 11.78 | 11.05 | 10.93 | 10.95 | 12.00 | 11.41 | 11.61 | 11.07 | 11.00 | 10.95 |
| 217383_at | 6.41 | 5.80 | 6.53 | 5.53 | 5.55 | 5.93 | 5.83 | 5.82 | 6.32 | 5.80 | 5.66 | 6.07 |
| 209193_at | 5.75 | 5.66 | 5.64 | 5.51 | 5.69 | 5.75 | 5.83 | 5.87 | 5.50 | 5.61 | 5.71 | 5.54 |
| 210145_at | 3.86 | 3.92 | 4.04 | 3.79 | 3.88 | 3.74 | 3.75 | 3.69 | 3.74 | 3.77 | 3.75 | 3.80 |
| 201979_s_at | 7.80 | 7.88 | 7.65 | 7.74 | 7.81 | 7.80 | 7.68 | 7.62 | 7.77 | 7.39 | 7.65 | 7.53 |
| 214617_at | 5.12 | 5.12 | 5.17 | 5.23 | 5.27 | 5.32 | 5.13 | 5.16 | 5.16 | 5.29 | 5.20 | 5.15 |
| 202545_at | 6.69 | 6.49 | 6.36 | 7.95 | 7.92 | 7.61 | 7.05 | 6.64 | 6.57 | 7.05 | 7.33 | 7.17 |
| 204279_at | 10.03 | 10.03 | 10.06 | 8.83 | 9.06 | 8.96 | 10.09 | 9.94 | 10.06 | 5.49 | 5.88 | 5.75 |
| 211661_x_at | 6.44 | 6.59 | 6.42 | 7.18 | 7.61 | 7.33 | 6.35 | 6.57 | 6.31 | 6.98 | 7.31 | 6.74 |
| 211892_s_at | 4.87 | 4.75 | 4.78 | 4.69 | 4.77 | 4.82 | 4.84 | 4.82 | 4.83 | 5.04 | 4.84 | 4.82 |
| 206035_at | 5.01 | 5.39 | 5.49 | 5.27 | 5.56 | 5.75 | 5.13 | 5.13 | 5.30 | 5.63 | 5.68 | 5.88 |
| 206036_s_at | 5.83 | 5.45 | 5.71 | 6.54 | 6.47 | 6.01 | 5.88 | 5.48 | 5.39 | 6.28 | 7.65 | 6.67 |
| 205205_at | 7.31 | 7.02 | 6.65 | 7.70 | 7.99 | 7.46 | 7.25 | 6.85 | 6.45 | 7.51 | 7.92 | 7.51 |
| 209544_at | 4.32 | 4.30 | 4.29 | 4.30 | 4.15 | 4.33 | 4.31 | 4.25 | 4.23 | 4.29 | 4.38 | 4.41 |
| 209545_s_at | 8.73 | 8.32 | 8.08 | 7.95 | 7.82 | 7.89 | 8.39 | 8.18 | 8.47 | 8.14 | 8.21 | 8.11 |
| 200872_at | 8.54 | 7.54 | 8.17 | 6.39 | 5.93 | 6.11 | 7.98 | 7.03 | 7.20 | 6.46 | 6.21 | 6.15 |
| 203186_s_at | 6.45 | 5.90 | 6.24 | 5.14 | 4.78 | 4.89 | 6.78 | 6.50 | 6.93 | 7.20 | 7.54 | 7.49 |
| 215223_s_at | 7.53 | 7.57 | 7.64 | 7.26 | 7.09 | 7.61 | 7.81 | 7.41 | 7.80 | 6.54 | 6.54 | 6.98 |
| 216841_s_at | 4.99 | 5.12 | 5.61 | 5.35 | 5.18 | 5.93 | 5.13 | 5.04 | 5.38 | 4.58 | 4.22 | 4.67 |
| 221477_s_at | 7.26 | 7.05 | 7.29 | 7.09 | 6.85 | 7.05 | 7.35 | 7.04 | 7.31 | 6.36 | 6.20 | 6.49 |
| 201471_s_at | 9.09 | 8.46 | 8.37 | 9.21 | 9.04 | 8.89 | 9.25 | 8.54 | 8.75 | 8.95 | 9.47 | 9.37 |
| 208048_at | 4.55 | 4.60 | 4.36 | 4.64 | 4.51 | 4.40 | 4.58 | 4.63 | 4.36 | 4.58 | 4.72 | 4.63 |
| 202307_s_at | 8.38 | 8.04 | 8.16 | 7.43 | 7.47 | 7.53 | 8.20 | 8.00 | 8.32 | 6.69 | 6.91 | 7.01 |
| 208829_at | 9.72 | 9.27 | 9.30 | 9.45 | 9.04 | 9.09 | 9.56 | 9.23 | 9.01 | 9.00 | 9.23 | 9.19 |
| 207199_at | 6.51 | 6.49 | 6.56 | 7.55 | 7.20 | 7.19 | 6.06 | 6.18 | 5.96 | 5.94 | 5.72 | 5.33 |
| 215775_at | 5.09 | 5.43 | 5.35 | 5.34 | 5.30 | 5.68 | 5.21 | 5.30 | 5.28 | 5.34 | 5.34 | 5.37 |
| 216005_at | 4.06 | 4.13 | 4.12 | 4.12 | 4.12 | 4.22 | 4.05 | 4.13 | 4.12 | 4.24 | 4.16 | 4.18 |
| 202643_s_at | 5.55 | 5.58 | 5.49 | 6.45 | 6.41 | 6.37 | 5.45 | 5.43 | 5.12 | 5.70 | 5.71 | 5.85 |
| 202644_s_at | 5.97 | 6.21 | 6.10 | 7.05 | 7.32 | 7.10 | 5.88 | 6.03 | 5.70 | 6.31 | 6.63 | 6.37 |
| 215346_at | 6.64 | 6.15 | 6.16 | 6.82 | 6.86 | 6.41 | 6.44 | 6.49 | 5.92 | 6.97 | 6.90 | 6.83 |
| 35150_at | 8.56 | 8.35 | 8.22 | 8.42 | 8.72 | 8.53 | 8.28 | 8.41 | 8.06 | 8.67 | 8.99 | 8.50 |
| 207536_s_at | 5.11 | 5.46 | 5.06 | 5.16 | 5.17 | 5.31 | 5.18 | 5.22 | 5.17 | 5.17 | 5.31 | 5.20 |
| 202687_s_at | 4.47 | 4.44 | 4.24 | 3.95 | 4.07 | 4.00 | 4.33 | 4.39 | 4.41 | 4.06 | 4.09 | 4.01 |
| 202688_at | 4.95 | 4.86 | 4.95 | 4.28 | 4.36 | 4.31 | 5.09 | 4.80 | 5.22 | 4.24 | 4.25 | 4.37 |
| 214329_x_at | 3.74 | 3.82 | 3.96 | 3.58 | 3.66 | 3.63 | 3.75 | 3.91 | 3.88 | 3.58 | 3.77 | 3.63 |
| 201746_at | 9.96 | 9.88 | 10.01 | 9.17 | 9.14 | 9.09 | 9.99 | 9.94 | 9.86 | 9.33 | 9.49 | 9.47 |
| 211300_s_at | 9.65 | 9.10 | 9.44 | 8.24 | 8.39 | 8.36 | 9.59 | 9.17 | 9.28 | 8.45 | 8.69 | 8.62 |
| 205599_at | 5.00 | 5.55 | 5.30 | 5.47 | 6.03 | 5.60 | 5.07 | 5.44 | 5.16 | 5.51 | 5.90 | 5.51 |
| 213191_at | 5.10 | 5.39 | 5.63 | 6.02 | 5.92 | 5.58 | 5.06 | 5.47 | 5.29 | 5.85 | 5.91 | 5.69 |
| 203109_at | 9.75 | 9.71 | 9.42 | 9.97 | 9.93 | 9.87 | 9.82 | 9.75 | 9.65 | 9.89 | 9.88 | 9.84 |
| 208997_s_at | 10.61 | 10.38 | 10.34 | 9.65 | 9.89 | 9.37 | 10.64 | 10.36 | 10.00 | 10.14 | 10.53 | 10.07 |
| 208998_at | 11.18 | 11.18 | 11.18 | 10.36 | 10.57 | 10.16 | 11.29 | 11.19 | 10.86 | 10.95 | 11.01 | 10.98 |
| 204881_s_at | 7.73 | 7.02 | 7.11 | 8.21 | 7.78 | 8.01 | 7.67 | 7.06 | 7.62 | 7.70 | 8.30 | 8.50 |
| 221765_at | 4.35 | 4.27 | 4.49 | 4.28 | 4.38 | 4.42 | 4.36 | 4.04 | 4.41 | 4.36 | 4.79 | 5.04 |
| 203868_s_at | 4.31 | 4.32 | 4.39 | 4.31 | 4.51 | 4.47 | 4.24 | 4.52 | 4.24 | 4.24 | 4.43 | 4.34 |
| 209946_at | 4.94 | 4.99 | 5.00 | 5.03 | 5.12 | 5.10 | 5.21 | 5.01 | 5.12 | 5.05 | 5.21 | 5.11 |
| 201426_s_at | 10.07 | 9.77 | 9.84 | 5.98 | 5.82 | 5.88 | 10.04 | 9.78 | 9.63 | 5.88 | 6.01 | 5.26 |
| 210301_at | 7.44 | 7.71 | 7.26 | 7.55 | 7.47 | 7.61 | 7.59 | 7.63 | 7.52 | 7.82 | 7.96 | 7.69 |
| 208544_at | 5.90 | 5.78 | 5.57 | 5.80 | 6.02 | 5.82 | 5.58 | 5.85 | 5.66 | 6.00 | 6.02 | 5.77 |
| 210081_at | 4.55 | 4.57 | 4.45 | 4.62 | 4.69 | 4.62 | 4.64 | 4.66 | 4.50 | 4.83 | 4.71 | 4.66 |
| 217046_s_at | 5.11 | 5.04 | 4.75 | 5.16 | 5.03 | 4.95 | 4.98 | 4.83 | 4.94 | 5.14 | 4.90 | 5.12 |
| 207206_s_at | 5.37 | 5.46 | 5.42 | 5.42 | 5.43 | 5.36 | 5.30 | 5.35 | 5.51 | 5.53 | 5.64 | 5.38 |
| 213952_s_at | 5.21 | 5.20 | 5.11 | 5.18 | 5.15 | 5.16 | 5.23 | 5.19 | 5.11 | 5.26 | 5.45 | 5.23 |
| 206516_at | 6.15 | 5.80 | 5.82 | 6.19 | 5.78 | 6.08 | 6.17 | 5.79 | 6.01 | 6.29 | 6.07 | 6.76 |
| 205820_s_at | 4.71 | 4.86 | 4.69 | 4.87 | 4.65 | 4.96 | 4.95 | 4.82 | 4.95 | 4.99 | 5.04 | 4.94 |
| 200602_at | 3.63 | 3.70 | 3.69 | 3.64 | 3.64 | 3.68 | 3.64 | 3.70 | 3.67 | 3.77 | 3.88 | 3.85 |
| 211277_x_at | 5.04 | 5.10 | 5.01 | 5.09 | 5.20 | 5.13 | 5.05 | 5.12 | 5.11 | 4.99 | 5.05 | 5.10 |
| 211110_s_at | 5.07 | 5.08 | 5.01 | 5.02 | 5.15 | 5.26 | 5.06 | 5.07 | 5.07 | 5.21 | 5.14 | 5.03 |
| 207367_at | 5.71 | 5.82 | 5.69 | 5.60 | 5.89 | 5.80 | 5.87 | 5.78 | 5.63 | 6.13 | 5.87 | 5.64 |
| 217904_s_at | 4.65 | 4.78 | 4.52 | 4.65 | 4.97 | 4.73 | 4.79 | 4.60 | 4.72 | 4.67 | 4.88 | 4.58 |
| 203684_s_at | 5.83 | 5.85 | 5.80 | 5.67 | 5.80 | 5.73 | 5.90 | 5.99 | 5.83 | 5.84 | 6.00 | 5.70 |
| 207004_at | 5.45 | 5.49 | 5.32 | 5.60 | 5.63 | 5.42 | 5.40 | 5.56 | 5.41 | 5.52 | 5.47 | 5.50 |
| 207510_at | 5.12 | 5.46 | 5.17 | 5.16 | 5.37 | 5.20 | 5.12 | 5.30 | 5.12 | 5.41 | 5.38 | 5.27 |
| 202357_s_at | 4.55 | 4.39 | 4.47 | 4.65 | 4.59 | 4.57 | 4.65 | 4.64 | 4.58 | 4.74 | 4.61 | 4.61 |
| 211920_at | 5.80 | 5.88 | 5.33 | 5.76 | 5.79 | 5.49 | 5.72 | 5.73 | 5.42 | 5.90 | 5.52 | 5.65 |
| 201261_x_at | 5.15 | 5.21 | 5.28 | 5.32 | 5.52 | 5.04 | 5.13 | 5.39 | 5.10 | 5.28 | 5.31 | 5.03 |
| 201262_s_at | 4.69 | 4.59 | 4.48 | 4.67 | 4.59 | 4.55 | 4.59 | 4.62 | 4.51 | 4.59 | 4.78 | 4.58 |
| 205289_at | 3.98 | 4.08 | 4.01 | 3.96 | 4.12 | 4.03 | 3.99 | 4.08 | 3.92 | 3.90 | 4.11 | 3.99 |
| 205290_s_at | 4.94 | 4.88 | 4.90 | 5.10 | 4.90 | 5.09 | 4.97 | 5.02 | 5.05 | 5.52 | 5.32 | 5.05 |
| 204439_at | 5.00 | 4.85 | 4.95 | 4.58 | 4.66 | 4.86 | 4.84 | 5.14 | 5.22 | 4.78 | 4.73 | 4.72 |
| 217767_at | 6.01 | 5.89 | 5.72 | 6.15 | 5.95 | 6.01 | 6.17 | 6.08 | 5.96 | 6.34 | 6.05 | 5.98 |
| 220066_at | 4.51 | 4.50 | 4.51 | 4.55 | 4.57 | 4.59 | 4.54 | 4.61 | 4.47 | 4.73 | 4.60 | 4.62 |
| 203065_s_at | 5.33 | 5.46 | 5.39 | 5.38 | 5.47 | 5.42 | 5.57 | 5.57 | 5.27 | 5.52 | 5.70 | 5.38 |
| 212097_at | 5.10 | 5.15 | 5.17 | 5.02 | 5.20 | 5.10 | 4.99 | 5.15 | 5.07 | 5.07 | 5.19 | 5.13 |
| 210133_at | 5.43 | 5.43 | 5.32 | 5.44 | 5.41 | 5.37 | 5.35 | 5.47 | 5.46 | 5.55 | 5.79 | 5.59 |
| 216598_s_at | 5.03 | 5.14 | 5.10 | 5.14 | 5.20 | 5.10 | 4.96 | 4.98 | 5.08 | 5.12 | 5.25 | 5.15 |
| 205476_at | 4.09 | 4.17 | 4.09 | 4.08 | 4.23 | 4.06 | 4.04 | 4.11 | 4.04 | 4.08 | 4.17 | 4.07 |
| 207861_at | 7.29 | 7.31 | 7.11 | 7.12 | 7.15 | 7.01 | 7.13 | 7.17 | 6.89 | 7.13 | 7.34 | 7.12 |
| 1405_i_at | 4.35 | 4.39 | 4.42 | 4.48 | 4.36 | 4.48 | 4.44 | 4.37 | 4.56 | 4.52 | 4.61 | 4.69 |
| 204655_at | 4.82 | 4.86 | 4.96 | 4.78 | 4.88 | 5.17 | 4.84 | 5.02 | 4.72 | 4.75 | 4.93 | 4.82 |
| 208711_s_at | 4.90 | 4.80 | 4.89 | 4.93 | 5.00 | 4.97 | 4.95 | 4.98 | 4.94 | 4.93 | 5.06 | 4.88 |
| 208712_at | 4.25 | 4.23 | 4.27 | 4.25 | 4.18 | 4.29 | 4.33 | 4.23 | 4.27 | 4.39 | 4.24 | 4.34 |
| 214019_at | 4.61 | 4.60 | 4.69 | 4.55 | 4.47 | 4.58 | 4.64 | 4.58 | 4.59 | 4.69 | 4.74 | 4.67 |
| 206991_s_at | 6.64 | 6.92 | 6.75 | 6.91 | 6.80 | 6.93 | 6.65 | 6.84 | 6.80 | 6.86 | 7.11 | 6.92 |
| 206337_at | 4.34 | 4.66 | 4.27 | 4.41 | 4.44 | 4.43 | 4.56 | 4.52 | 4.40 | 4.63 | 4.54 | 4.45 |
| 207277_at | 6.18 | 6.33 | 6.46 | 6.24 | 6.53 | 6.59 | 6.50 | 6.58 | 6.61 | 6.71 | 6.77 | 6.65 |
| 207278_s_at | 5.28 | 5.21 | 5.07 | 5.22 | 5.09 | 5.21 | 5.20 | 5.21 | 5.27 | 5.66 | 5.35 | 5.25 |
| 206804_at | 5.58 | 5.83 | 5.76 | 5.63 | 5.72 | 5.70 | 5.57 | 5.73 | 5.73 | 6.05 | 6.01 | 5.71 |
| 210564_x_at | 5.67 | 5.47 | 5.43 | 5.94 | 5.68 | 5.73 | 5.60 | 5.55 | 5.62 | 5.40 | 5.54 | 5.48 |
| 214486_x_at | 6.39 | 6.54 | 6.38 | 6.43 | 6.58 | 6.39 | 6.30 | 6.43 | 6.28 | 6.22 | 6.34 | 6.06 |
| 202403_s_at | 5.95 | 5.90 | 5.84 | 5.97 | 5.95 | 5.95 | 5.81 | 5.88 | 5.95 | 6.37 | 5.99 | 6.05 |
| 202404_s_at | 4.10 | 4.07 | 4.29 | 4.26 | 4.32 | 4.26 | 4.28 | 4.16 | 4.20 | 4.14 | 4.05 | 4.27 |
| 205753_at | 5.13 | 5.22 | 5.15 | 5.03 | 5.24 | 5.26 | 4.98 | 5.15 | 5.11 | 5.06 | 5.22 | 4.87 |
| 37020_at | 4.31 | 4.27 | 4.21 | 4.36 | 4.21 | 4.29 | 4.33 | 4.17 | 4.27 | 4.37 | 4.40 | 4.26 |
| 207082_at | 6.21 | 6.31 | 6.53 | 6.17 | 6.35 | 6.22 | 6.38 | 6.36 | 6.02 | 6.24 | 6.29 | 6.18 |
| 210557_x_at | 5.02 | 5.09 | 4.98 | 5.03 | 5.09 | 4.96 | 5.09 | 5.18 | 4.86 | 5.16 | 5.24 | 5.04 |
| 211839_s_at | 5.49 | 5.51 | 5.31 | 5.53 | 5.38 | 5.58 | 5.48 | 5.44 | 5.46 | 5.99 | 5.51 | 5.52 |
| 210228_at | 5.06 | 5.13 | 5.13 | 5.12 | 5.24 | 5.09 | 5.12 | 5.38 | 5.08 | 5.26 | 5.28 | 5.01 |
| 210229_s_at | 5.88 | 5.98 | 5.68 | 5.85 | 5.97 | 5.88 | 5.86 | 5.95 | 5.68 | 6.06 | 6.11 | 5.93 |
| 207442_at | 6.31 | 6.37 | 6.23 | 6.32 | 6.34 | 6.12 | 6.11 | 6.26 | 6.17 | 6.34 | 6.41 | 6.27 |
| 213275_x_at | 4.87 | 4.83 | 4.86 | 4.51 | 4.63 | 4.51 | 4.84 | 5.06 | 5.00 | 4.67 | 4.70 | 4.66 |
| 202087_s_at | 4.53 | 4.76 | 4.63 | 4.57 | 4.72 | 4.59 | 4.63 | 4.60 | 4.57 | 4.58 | 4.62 | 4.57 |
| 204533_at | 5.06 | 5.35 | 5.29 | 4.91 | 5.20 | 5.09 | 4.95 | 5.31 | 4.98 | 5.07 | 5.20 | 4.89 |
| 211122_s_at | 3.90 | 3.74 | 3.82 | 3.78 | 3.83 | 3.82 | 3.73 | 3.78 | 3.82 | 3.79 | 3.87 | 3.86 |
| 209774_x_at | 4.05 | 4.11 | 4.13 | 4.19 | 4.16 | 4.22 | 4.11 | 4.29 | 4.12 | 4.12 | 4.29 | 4.07 |
| 207852_at | 3.65 | 3.72 | 3.76 | 3.65 | 3.75 | 3.67 | 3.67 | 3.73 | 3.63 | 3.60 | 3.60 | 3.64 |
| 215101_s_at | 4.05 | 4.05 | 4.11 | 4.08 | 4.06 | 4.07 | 4.02 | 4.10 | 4.04 | 4.07 | 4.00 | 4.07 |
| 203699_s_at | 5.06 | 5.10 | 5.09 | 4.92 | 4.93 | 5.12 | 5.03 | 5.05 | 5.04 | 5.09 | 5.00 | 5.02 |
| 210819_x_at | 4.74 | 4.86 | 4.84 | 4.66 | 4.77 | 4.92 | 4.68 | 4.86 | 4.74 | 4.83 | 4.93 | 4.72 |
| 211215_x_at | 7.20 | 7.24 | 6.88 | 7.07 | 7.24 | 7.32 | 7.09 | 7.11 | 7.08 | 7.40 | 7.25 | 7.14 |
| 201044_x_at | 5.14 | 5.08 | 4.86 | 5.27 | 4.91 | 5.01 | 5.02 | 5.12 | 4.98 | 5.30 | 4.93 | 4.98 |
| 201983_s_at | 5.30 | 5.24 | 5.12 | 5.42 | 5.14 | 5.30 | 5.27 | 5.08 | 5.19 | 5.58 | 5.44 | 5.28 |
| 201984_s_at | 5.89 | 5.97 | 5.73 | 5.93 | 5.82 | 5.90 | 5.88 | 6.07 | 5.86 | 5.96 | 5.90 | 6.04 |
| 210984_x_at | 7.14 | 7.01 | 7.06 | 7.07 | 7.28 | 7.22 | 7.02 | 7.02 | 7.05 | 7.51 | 7.31 | 7.07 |
| 211550_at | 5.46 | 5.46 | 5.54 | 5.44 | 5.54 | 5.48 | 5.41 | 5.53 | 5.48 | 5.59 | 5.67 | 5.45 |
| 211607_x_at | 6.46 | 6.35 | 6.27 | 6.21 | 6.42 | 6.37 | 6.31 | 6.34 | 6.26 | 6.81 | 6.68 | 6.31 |
| 201693_s_at | 5.32 | 5.36 | 5.48 | 5.34 | 5.59 | 5.59 | 5.35 | 5.61 | 5.29 | 5.53 | 5.47 | 5.42 |
| 201808_s_at | 5.74 | 5.84 | 5.74 | 5.55 | 5.62 | 5.91 | 5.74 | 5.85 | 5.86 | 5.90 | 5.71 | 5.62 |
| 201809_s_at | 6.23 | 6.07 | 6.17 | 5.82 | 5.61 | 5.60 | 6.53 | 6.48 | 6.28 | 5.90 | 5.79 | 5.67 |
| 207257_at | 4.52 | 4.56 | 4.40 | 4.78 | 4.52 | 4.50 | 4.58 | 4.42 | 4.59 | 4.70 | 4.60 | 4.46 |
| 204363_at | 4.37 | 4.49 | 4.46 | 4.30 | 4.49 | 4.37 | 4.28 | 4.46 | 4.42 | 4.41 | 4.45 | 4.46 |
| 206759_at | 4.75 | 4.58 | 4.59 | 5.06 | 4.88 | 4.78 | 4.63 | 4.51 | 4.49 | 5.09 | 4.88 | 4.68 |
| 206760_s_at | 5.79 | 5.92 | 5.84 | 6.05 | 5.96 | 5.97 | 5.89 | 5.79 | 5.82 | 6.00 | 6.09 | 5.90 |
| 210495_x_at | 5.42 | 5.31 | 5.43 | 5.16 | 5.55 | 5.34 | 5.16 | 5.39 | 5.08 | 5.19 | 5.47 | 5.18 |
| 211719_x_at | 4.63 | 4.78 | 4.75 | 4.47 | 4.77 | 4.69 | 4.59 | 4.68 | 4.60 | 4.57 | 4.72 | 4.62 |
| 212464_s_at | 4.64 | 4.71 | 4.69 | 4.69 | 4.85 | 4.69 | 4.59 | 4.87 | 4.48 | 4.66 | 4.83 | 4.74 |
| 214702_at | 3.84 | 3.91 | 3.88 | 3.94 | 3.93 | 3.96 | 3.89 | 4.05 | 3.89 | 3.91 | 3.95 | 4.04 |
| 216442_x_at | 4.89 | 5.00 | 4.92 | 4.69 | 5.19 | 4.75 | 4.71 | 4.89 | 4.70 | 4.88 | 4.84 | 4.77 |
| 205278_at | 4.16 | 4.23 | 4.22 | 3.99 | 4.12 | 4.12 | 4.09 | 4.10 | 4.02 | 4.04 | 4.04 | 4.00 |
| 206669_at | 5.07 | 4.97 | 4.95 | 5.09 | 4.85 | 5.00 | 4.92 | 4.95 | 5.05 | 5.10 | 4.94 | 4.95 |
| 206670_s_at | 4.69 | 4.95 | 4.62 | 4.58 | 4.54 | 4.55 | 4.56 | 4.62 | 4.60 | 4.67 | 4.64 | 4.65 |
| 213560_at | 5.74 | 5.81 | 5.58 | 5.53 | 5.79 | 5.52 | 5.60 | 5.68 | 5.57 | 5.63 | 5.63 | 5.58 |
| 220821_at | 4.23 | 4.20 | 4.23 | 4.17 | 4.38 | 4.23 | 4.16 | 4.32 | 4.19 | 4.22 | 4.19 | 4.15 |
| 205914_s_at | 5.27 | 5.18 | 4.85 | 5.44 | 5.09 | 5.20 | 5.32 | 5.06 | 5.18 | 5.63 | 5.34 | 5.14 |
| 205915_x_at | 6.00 | 6.07 | 5.78 | 5.97 | 5.99 | 6.05 | 5.67 | 6.00 | 5.97 | 6.17 | 6.13 | 6.01 |
| 210781_x_at | 6.13 | 6.30 | 6.23 | 6.12 | 6.38 | 6.33 | 6.12 | 6.33 | 6.22 | 6.24 | 6.41 | 6.23 |
| 210782_x_at | 5.80 | 5.85 | 5.24 | 5.55 | 5.82 | 5.37 | 5.92 | 5.61 | 5.41 | 5.87 | 5.49 | 5.48 |
| 211125_x_at | 5.36 | 5.54 | 5.31 | 5.22 | 5.33 | 5.43 | 5.30 | 5.50 | 5.26 | 5.39 | 5.57 | 5.36 |
| 206534_at | 5.54 | 5.56 | 5.44 | 5.43 | 5.66 | 5.57 | 5.40 | 5.54 | 5.54 | 5.68 | 5.65 | 5.51 |
| 221942_s_at | 5.81 | 6.10 | 5.85 | 5.91 | 5.87 | 6.09 | 5.77 | 5.98 | 5.95 | 6.21 | 6.27 | 6.03 |
| 207316_at | 4.84 | 4.91 | 4.81 | 4.86 | 4.99 | 4.98 | 4.91 | 4.87 | 4.90 | 5.01 | 4.97 | 4.87 |
| 205919_at | 5.92 | 5.95 | 5.93 | 6.18 | 5.89 | 6.27 | 6.19 | 5.94 | 6.11 | 6.49 | 6.09 | 6.27 |
| 217683_at | 5.72 | 5.76 | 5.56 | 5.67 | 5.73 | 5.78 | 5.68 | 5.59 | 5.69 | 5.84 | 5.73 | 5.63 |
| 203665_at | 6.03 | 6.15 | 6.14 | 5.92 | 5.99 | 6.11 | 6.23 | 6.33 | 6.18 | 6.21 | 6.07 | 5.91 |
| 210439_at | 4.62 | 4.71 | 4.72 | 4.82 | 4.80 | 4.77 | 4.71 | 4.82 | 4.74 | 4.71 | 4.75 | 4.70 |
| 201631_s_at | 5.33 | 5.28 | 5.26 | 5.58 | 5.57 | 5.71 | 5.37 | 5.46 | 5.24 | 5.68 | 5.40 | 5.44 |
| 205302_at | 3.75 | 3.84 | 3.80 | 3.65 | 3.80 | 3.80 | 3.63 | 3.79 | 3.72 | 3.74 | 3.73 | 3.69 |
| 207433_at | 4.25 | 4.44 | 4.35 | 4.24 | 4.49 | 4.29 | 4.20 | 4.42 | 4.15 | 4.19 | 4.36 | 4.23 |
| 206924_at | 4.30 | 4.42 | 4.30 | 4.18 | 4.22 | 4.32 | 4.28 | 4.27 | 4.24 | 4.32 | 4.48 | 4.24 |
| 206926_s_at | 6.07 | 6.41 | 6.09 | 6.41 | 6.35 | 6.19 | 6.04 | 6.32 | 6.02 | 6.44 | 6.44 | 6.09 |
| 207844_at | 5.13 | 5.30 | 4.82 | 5.29 | 5.26 | 5.16 | 5.09 | 5.23 | 5.14 | 5.69 | 5.18 | 4.99 |
| 205992_s_at | 3.86 | 3.88 | 3.88 | 3.79 | 4.13 | 3.99 | 3.83 | 3.81 | 3.86 | 3.82 | 3.88 | 3.86 |
| 210118_s_at | 4.46 | 4.52 | 4.44 | 4.35 | 4.41 | 4.48 | 4.34 | 4.46 | 4.45 | 4.63 | 4.51 | 4.45 |
| 205067_at | 6.78 | 6.98 | 6.81 | 7.02 | 6.92 | 6.95 | 6.84 | 6.89 | 7.01 | 7.16 | 7.03 | 6.96 |
| 39402_at | 5.03 | 5.28 | 5.06 | 5.07 | 5.15 | 5.07 | 5.03 | 5.05 | 4.95 | 5.19 | 5.34 | 5.11 |
| 212657_s_at | 5.77 | 6.07 | 5.76 | 5.61 | 5.81 | 5.83 | 5.66 | 5.85 | 5.77 | 5.81 | 6.02 | 5.71 |
| 212659_s_at | 5.39 | 5.40 | 4.78 | 5.21 | 5.36 | 5.26 | 5.45 | 5.33 | 5.16 | 5.81 | 5.42 | 5.35 |
| 216243_s_at | 6.68 | 6.64 | 6.55 | 6.66 | 6.84 | 6.70 | 6.58 | 6.67 | 6.48 | 6.79 | 6.92 | 6.55 |
| 216244_at | 4.55 | 4.46 | 4.35 | 4.72 | 4.56 | 4.57 | 4.66 | 4.47 | 4.60 | 4.68 | 4.56 | 4.61 |
| 216245_at | 4.96 | 5.03 | 4.96 | 4.85 | 5.12 | 5.01 | 4.93 | 5.08 | 4.88 | 5.06 | 5.12 | 4.97 |
| 207849_at | 3.81 | 3.82 | 3.88 | 3.82 | 3.86 | 3.85 | 3.84 | 3.86 | 3.86 | 3.83 | 3.96 | 3.83 |
| 206341_at | 4.41 | 4.38 | 4.35 | 4.32 | 4.23 | 4.32 | 4.47 | 4.27 | 4.43 | 4.50 | 4.52 | 4.29 |
| 211269_s_at | 5.99 | 5.92 | 6.07 | 5.93 | 6.13 | 5.99 | 6.01 | 6.15 | 5.98 | 5.84 | 5.90 | 5.96 |
| 202859_x_at | 5.08 | 5.14 | 5.19 | 5.01 | 5.06 | 5.10 | 4.98 | 5.10 | 5.10 | 5.09 | 5.12 | 5.15 |
| 208193_at | 4.39 | 4.45 | 4.37 | 4.37 | 4.47 | 4.40 | 4.40 | 4.38 | 4.26 | 4.41 | 4.41 | 4.38 |
| 216987_at | 3.88 | 3.74 | 3.80 | 3.79 | 3.74 | 3.79 | 3.78 | 3.72 | 3.82 | 3.80 | 3.77 | 3.80 |
| 201464_x_at | 5.07 | 4.75 | 5.09 | 4.70 | 4.88 | 4.90 | 5.09 | 4.99 | 4.94 | 5.02 | 4.87 | 4.98 |
| 201465_s_at | 5.07 | 5.02 | 4.93 | 5.15 | 4.96 | 5.02 | 5.34 | 5.08 | 5.11 | 5.09 | 5.16 | 5.14 |
| 201466_s_at | 4.20 | 4.21 | 4.23 | 4.16 | 4.12 | 4.23 | 4.30 | 4.13 | 4.28 | 4.22 | 4.23 | 4.29 |
| 211124_s_at | 3.93 | 3.93 | 3.95 | 3.88 | 3.86 | 3.95 | 3.97 | 3.95 | 3.93 | 3.90 | 4.01 | 3.91 |
| 204582_s_at | 4.38 | 4.24 | 4.20 | 4.26 | 4.27 | 4.23 | 4.39 | 4.37 | 4.29 | 4.51 | 4.41 | 4.31 |
| 204583_x_at | 6.28 | 6.34 | 6.07 | 6.28 | 6.23 | 6.43 | 6.38 | 6.35 | 6.16 | 6.38 | 6.44 | 6.20 |
| 204734_at | 4.53 | 4.48 | 4.50 | 4.69 | 4.51 | 4.55 | 4.52 | 4.64 | 4.49 | 4.84 | 4.43 | 4.56 |
| 208188_at | 6.06 | 5.95 | 5.81 | 6.18 | 6.13 | 6.12 | 5.98 | 5.85 | 5.99 | 6.42 | 6.23 | 6.17 |
| 211652_s_at | 6.68 | 6.70 | 6.80 | 6.73 | 6.63 | 6.88 | 6.64 | 6.58 | 6.62 | 7.06 | 7.17 | 6.73 |
| 214461_at | 4.78 | 4.72 | 4.87 | 4.70 | 4.78 | 4.86 | 4.67 | 4.83 | 4.76 | 4.87 | 4.82 | 4.84 |
| 212531_at | 5.68 | 5.67 | 5.38 | 5.85 | 5.30 | 5.47 | 5.33 | 5.39 | 5.55 | 6.04 | 5.53 | 5.64 |
| 208949_s_at | 5.14 | 5.12 | 5.17 | 5.19 | 5.06 | 5.19 | 5.19 | 5.26 | 5.22 | 5.35 | 5.40 | 5.24 |
| 206975_at | 5.18 | 5.31 | 5.23 | 5.18 | 5.25 | 5.13 | 5.49 | 5.45 | 5.12 | 5.38 | 6.09 | 5.30 |
| 204475_at | 3.80 | 3.78 | 3.82 | 3.75 | 3.81 | 3.75 | 3.78 | 3.82 | 3.88 | 3.80 | 3.96 | 3.85 |
| 205828_at | 4.05 | 4.13 | 4.05 | 3.95 | 4.19 | 4.16 | 4.02 | 4.15 | 4.05 | 4.08 | 4.27 | 4.13 |
| 205970_at | 5.51 | 5.56 | 5.27 | 5.36 | 5.52 | 5.34 | 5.53 | 5.63 | 5.37 | 5.62 | 5.45 | 5.25 |
| 204673_at | 6.27 | 6.12 | 5.94 | 6.26 | 6.26 | 6.28 | 6.32 | 6.30 | 6.16 | 6.55 | 6.22 | 6.25 |
| 209636_at | 5.26 | 5.15 | 4.96 | 6.02 | 5.62 | 5.38 | 5.43 | 5.18 | 5.00 | 5.55 | 5.84 | 5.51 |
| 211524_at | 4.38 | 4.48 | 4.55 | 4.30 | 4.55 | 4.48 | 4.31 | 4.47 | 4.37 | 4.40 | 4.44 | 4.43 |
| 203828_s_at | 5.71 | 5.66 | 5.41 | 5.59 | 5.55 | 5.78 | 5.71 | 5.48 | 5.52 | 5.95 | 5.70 | 5.55 |
| 205440_s_at | 4.00 | 4.05 | 4.20 | 3.88 | 4.06 | 4.10 | 4.02 | 4.08 | 4.10 | 3.92 | 4.06 | 4.02 |
| 201467_s_at | 5.18 | 5.12 | 4.90 | 4.90 | 5.06 | 4.44 | 5.19 | 5.05 | 4.87 | 5.56 | 5.41 | 5.23 |
| 204621_s_at | 4.72 | 4.67 | 4.43 | 4.52 | 4.42 | 4.48 | 4.79 | 4.62 | 4.68 | 4.65 | 4.57 | 4.54 |
| 205040_at | 4.76 | 4.66 | 4.45 | 4.94 | 4.69 | 4.67 | 4.97 | 4.81 | 4.65 | 4.97 | 4.85 | 4.73 |
| 205041_s_at | 4.20 | 4.07 | 4.04 | 4.24 | 4.02 | 4.27 | 4.30 | 4.19 | 4.19 | 4.32 | 4.20 | 4.21 |
| 214465_at | 4.57 | 4.52 | 4.52 | 4.46 | 4.57 | 4.57 | 4.50 | 4.44 | 4.60 | 4.61 | 4.66 | 4.58 |
| 207921_x_at | 4.81 | 4.91 | 5.09 | 5.02 | 4.97 | 4.88 | 4.85 | 4.91 | 5.00 | 5.14 | 5.11 | 4.86 |
| 207923_x_at | 4.94 | 4.93 | 4.82 | 5.04 | 4.91 | 4.92 | 4.85 | 4.89 | 4.73 | 5.09 | 4.98 | 5.03 |
| 207924_x_at | 4.72 | 4.69 | 4.75 | 4.87 | 4.84 | 4.84 | 4.90 | 5.07 | 4.79 | 5.01 | 4.98 | 5.01 |
| 209552_at | 6.20 | 6.24 | 5.87 | 6.38 | 6.27 | 6.19 | 6.21 | 6.17 | 6.02 | 6.55 | 6.38 | 6.20 |
| 214528_s_at | 4.38 | 4.37 | 4.39 | 4.46 | 4.17 | 4.53 | 4.40 | 4.32 | 4.43 | 4.73 | 4.59 | 4.39 |
| 221990_at | 5.68 | 5.86 | 5.72 | 5.87 | 5.89 | 5.75 | 5.90 | 5.76 | 5.70 | 5.87 | 6.14 | 5.78 |
| 204200_s_at | 4.81 | 5.00 | 4.95 | 4.95 | 4.95 | 4.87 | 4.92 | 4.83 | 4.91 | 4.94 | 4.88 | 4.90 |
| 216055_at | 4.71 | 4.90 | 4.80 | 4.85 | 4.89 | 4.89 | 4.77 | 4.86 | 4.97 | 5.14 | 4.95 | 4.84 |
| 216061_x_at | 5.53 | 5.71 | 5.61 | 5.76 | 5.87 | 5.89 | 5.67 | 5.67 | 5.67 | 5.75 | 5.82 | 5.71 |
| 206803_at | 5.00 | 5.01 | 4.95 | 4.79 | 5.01 | 5.19 | 4.86 | 5.13 | 4.98 | 5.11 | 5.21 | 5.10 |
| 204213_at | 6.40 | 6.66 | 6.74 | 6.63 | 6.73 | 6.63 | 6.45 | 6.69 | 6.63 | 6.86 | 6.87 | 6.59 |
| 203649_s_at | 5.22 | 5.22 | 5.18 | 5.30 | 5.33 | 5.23 | 5.14 | 5.38 | 5.21 | 5.30 | 5.36 | 5.23 |
| 205479_s_at | 5.16 | 5.29 | 5.18 | 5.08 | 5.20 | 5.16 | 5.31 | 5.38 | 5.02 | 5.19 | 5.15 | 5.11 |
| 211668_s_at | 5.34 | 5.52 | 5.46 | 5.57 | 5.44 | 5.49 | 5.52 | 5.59 | 5.55 | 5.76 | 5.40 | 5.62 |
| 205125_at | 8.59 | 8.57 | 8.41 | 8.54 | 8.45 | 8.40 | 8.59 | 8.54 | 8.56 | 8.67 | 8.56 | 8.40 |
| 205720_at | 5.19 | 5.32 | 5.11 | 5.28 | 5.42 | 5.14 | 5.49 | 5.40 | 5.21 | 5.39 | 5.44 | 5.24 |
| 215705_at | 6.03 | 6.32 | 6.21 | 6.13 | 6.33 | 6.22 | 6.03 | 6.36 | 6.06 | 6.43 | 6.28 | 6.19 |
| 206278_at | 4.81 | 4.73 | 4.29 | 4.72 | 5.37 | 4.52 | 4.62 | 4.56 | 4.17 | 4.65 | 4.99 | 4.48 |
| 211663_x_at | 5.54 | 5.56 | 5.53 | 5.54 | 5.73 | 5.70 | 5.56 | 5.89 | 5.55 | 5.91 | 5.78 | 5.65 |
| 211748_x_at | 6.87 | 6.90 | 6.74 | 7.00 | 7.08 | 6.91 | 6.87 | 7.10 | 6.73 | 7.11 | 7.05 | 6.83 |
| 212187_x_at | 6.73 | 6.84 | 6.58 | 6.93 | 6.87 | 6.83 | 6.82 | 6.92 | 6.74 | 7.29 | 6.94 | 6.79 |
| 207388_s_at | 6.54 | 6.71 | 6.28 | 6.78 | 6.58 | 6.52 | 6.49 | 6.64 | 6.50 | 6.96 | 6.83 | 6.64 |
| 210367_s_at | 5.71 | 6.07 | 6.06 | 5.92 | 6.16 | 6.09 | 5.82 | 6.15 | 5.92 | 6.06 | 5.96 | 5.91 |
| 208131_s_at | 4.77 | 4.78 | 4.63 | 4.76 | 4.74 | 4.90 | 4.74 | 4.71 | 4.66 | 5.06 | 4.86 | 4.74 |
| 210702_s_at | 5.51 | 5.51 | 5.28 | 5.35 | 5.42 | 5.39 | 5.41 | 5.43 | 5.34 | 5.51 | 5.47 | 5.38 |
| 204748_at | 4.08 | 4.17 | 4.11 | 4.11 | 4.06 | 4.15 | 4.19 | 4.12 | 4.16 | 4.39 | 4.21 | 4.25 |
| 204201_s_at | 3.87 | 3.85 | 3.86 | 3.84 | 3.91 | 3.83 | 3.83 | 3.86 | 3.83 | 3.84 | 3.88 | 3.85 |
| 206157_at | 3.60 | 3.72 | 3.77 | 3.73 | 3.72 | 3.71 | 3.63 | 3.75 | 3.72 | 3.69 | 3.85 | 3.67 |
| 216994_s_at | 4.48 | 4.47 | 4.32 | 4.40 | 4.42 | 4.38 | 4.35 | 4.47 | 4.39 | 4.55 | 4.66 | 4.42 |
| 221282_x_at | 5.65 | 5.74 | 5.76 | 5.52 | 5.81 | 5.66 | 5.47 | 6.01 | 5.49 | 5.69 | 5.72 | 5.72 |
| 221283_at | 5.67 | 5.58 | 5.19 | 5.61 | 5.45 | 5.38 | 5.40 | 5.38 | 5.32 | 5.79 | 5.61 | 5.57 |
| 217728_at | 4.72 | 4.63 | 4.63 | 4.79 | 4.51 | 4.70 | 4.68 | 4.58 | 4.66 | 5.02 | 4.79 | 4.76 |
| 208607_s_at | 4.82 | 4.91 | 4.51 | 4.86 | 4.64 | 4.49 | 4.85 | 4.86 | 4.56 | 4.68 | 4.55 | 4.50 |
| 214456_x_at | 5.88 | 5.99 | 5.56 | 5.66 | 5.93 | 5.66 | 5.65 | 5.83 | 5.69 | 5.82 | 5.83 | 5.54 |
| 202071_at | 5.07 | 5.18 | 5.31 | 4.92 | 5.15 | 5.25 | 5.18 | 5.37 | 5.15 | 5.27 | 5.36 | 5.19 |
| 206211_at | 3.82 | 3.91 | 3.90 | 3.92 | 3.94 | 3.91 | 3.87 | 3.93 | 3.87 | 3.93 | 3.95 | 3.92 |
| 206049_at | 6.64 | 6.62 | 6.57 | 6.45 | 6.70 | 6.87 | 6.42 | 6.86 | 6.40 | 6.54 | 6.72 | 6.57 |
| 202627_s_at | 5.08 | 5.08 | 4.92 | 4.93 | 5.06 | 5.06 | 5.04 | 5.03 | 4.76 | 5.05 | 4.79 | 4.91 |
| 202628_s_at | 4.66 | 4.68 | 4.46 | 4.68 | 4.68 | 4.69 | 4.55 | 4.84 | 4.52 | 4.69 | 4.65 | 4.61 |
| 210073_at | 3.86 | 3.83 | 3.75 | 3.91 | 3.88 | 3.92 | 3.81 | 3.88 | 3.85 | 3.84 | 3.79 | 3.80 |
| 215078_at | 3.99 | 4.00 | 4.03 | 4.04 | 3.96 | 4.01 | 3.98 | 3.94 | 4.04 | 4.04 | 3.93 | 3.99 |
| 202935_s_at | 4.10 | 4.16 | 4.12 | 4.14 | 4.15 | 4.14 | 4.04 | 4.15 | 4.19 | 4.19 | 4.28 | 4.20 |
| 202936_s_at | 4.54 | 4.61 | 4.58 | 4.42 | 4.56 | 4.53 | 4.50 | 4.52 | 4.53 | 4.58 | 4.51 | 4.47 |
| 213112_s_at | 6.08 | 6.09 | 5.81 | 5.99 | 6.00 | 5.98 | 5.87 | 6.13 | 5.80 | 6.02 | 6.14 | 6.17 |
| 203010_at | 7.15 | 7.44 | 7.25 | 6.53 | 6.70 | 6.58 | 7.25 | 7.18 | 7.12 | 6.68 | 6.71 | 6.62 |
| 208049_s_at | 4.69 | 4.55 | 4.68 | 4.84 | 4.83 | 4.77 | 4.80 | 4.73 | 4.76 | 4.75 | 4.81 | 4.76 |
| 210637_at | 5.27 | 5.61 | 5.24 | 5.65 | 5.32 | 5.51 | 5.48 | 5.34 | 5.45 | 5.58 | 5.59 | 5.55 |
| 210294_at | 4.60 | 4.68 | 4.72 | 4.52 | 4.50 | 4.58 | 4.65 | 4.83 | 4.63 | 4.69 | 4.67 | 4.56 |
| 209277_at | 3.75 | 3.57 | 3.55 | 3.61 | 3.71 | 3.64 | 3.66 | 3.65 | 3.56 | 3.60 | 3.81 | 3.62 |
| 209278_s_at | 4.45 | 4.52 | 4.45 | 4.37 | 4.49 | 4.55 | 4.41 | 4.41 | 4.42 | 4.42 | 4.52 | 4.37 |
| 211003_x_at | 5.33 | 5.35 | 5.13 | 5.36 | 5.34 | 5.44 | 5.34 | 5.25 | 5.42 | 5.46 | 5.42 | 5.44 |
| 211573_x_at | 5.59 | 5.49 | 5.24 | 5.55 | 5.37 | 5.70 | 5.57 | 5.57 | 5.63 | 5.90 | 5.78 | 5.61 |
| 216183_at | 6.00 | 6.12 | 5.99 | 5.89 | 6.07 | 5.95 | 5.99 | 6.05 | 5.76 | 6.08 | 6.14 | 5.90 |
| 201107_s_at | 5.12 | 5.07 | 4.95 | 5.13 | 4.90 | 5.11 | 5.07 | 5.15 | 5.04 | 5.40 | 5.25 | 5.30 |
| 201108_s_at | 5.46 | 5.59 | 5.52 | 5.44 | 5.70 | 5.65 | 5.68 | 5.75 | 5.62 | 5.74 | 5.62 | 5.73 |
| 201109_s_at | 4.14 | 4.34 | 4.16 | 4.02 | 4.39 | 4.11 | 4.21 | 4.25 | 4.06 | 4.09 | 4.24 | 4.10 |
| 201110_s_at | 3.73 | 3.78 | 3.73 | 3.76 | 3.80 | 3.82 | 3.73 | 3.72 | 3.77 | 3.77 | 3.81 | 3.75 |
| 203083_at | 5.33 | 5.47 | 5.32 | 5.19 | 5.23 | 5.23 | 5.23 | 5.52 | 5.28 | 5.26 | 5.44 | 5.19 |
| 201645_at | 3.99 | 4.19 | 3.98 | 3.94 | 4.06 | 4.05 | 3.96 | 4.08 | 3.99 | 3.95 | 4.14 | 4.08 |
| 207113_s_at | 5.42 | 5.40 | 5.17 | 5.28 | 5.49 | 5.28 | 5.34 | 5.43 | 5.25 | 6.06 | 6.22 | 5.75 |
| 202509_s_at | 5.42 | 5.58 | 5.37 | 5.52 | 5.57 | 5.54 | 5.57 | 5.44 | 5.49 | 5.74 | 5.79 | 5.55 |
| 202510_s_at | 6.40 | 6.66 | 6.51 | 6.58 | 6.58 | 6.60 | 6.55 | 6.55 | 6.44 | 6.59 | 6.72 | 6.41 |
| 203508_at | 4.42 | 4.41 | 4.30 | 4.34 | 4.34 | 4.36 | 4.45 | 4.58 | 4.40 | 4.48 | 4.29 | 4.35 |
| 205153_s_at | 5.94 | 5.42 | 5.24 | 6.04 | 6.24 | 6.03 | 5.75 | 5.47 | 5.33 | 6.25 | 6.50 | 6.10 |
| 222292_at | 4.59 | 4.65 | 4.73 | 4.57 | 4.65 | 4.52 | 4.51 | 4.47 | 4.60 | 4.66 | 4.67 | 4.85 |
| 211786_at | 4.61 | 4.53 | 4.49 | 4.64 | 4.53 | 4.64 | 4.52 | 4.65 | 4.62 | 4.71 | 4.76 | 4.67 |
| 207892_at | 5.42 | 5.61 | 5.56 | 5.39 | 5.63 | 5.56 | 5.52 | 5.57 | 5.49 | 5.79 | 5.62 | 5.57 |
| 210865_at | 5.13 | 5.25 | 4.92 | 5.08 | 5.06 | 4.81 | 5.02 | 4.99 | 4.64 | 5.23 | 5.19 | 4.97 |
| 211333_s_at | 4.60 | 4.61 | 4.38 | 4.69 | 4.65 | 4.65 | 4.48 | 4.51 | 4.53 | 4.61 | 4.71 | 4.46 |
| 204413_at | 5.65 | 5.94 | 5.50 | 5.78 | 5.93 | 5.58 | 5.49 | 5.84 | 5.49 | 5.46 | 5.73 | 5.28 |
| 206067_s_at | 4.36 | 4.31 | 4.27 | 4.31 | 4.23 | 4.33 | 4.32 | 4.25 | 4.32 | 4.34 | 4.41 | 4.31 |
| 216953_s_at | 5.67 | 5.64 | 5.65 | 5.78 | 5.66 | 5.96 | 5.86 | 5.89 | 5.85 | 5.89 | 5.96 | 5.97 |

TABLE 4B
NFκB-Regulated Genes (See tables reference 1)

| | t-test (two-tailed) | | | | | | Gene |
|---|---|---|---|---|---|---|---|
| Probesets | pAB | pAD | pBC | pCD | Measured | Changed | Symbol |
| FDR Cutoff | 1.0E−02 | 8.2E−03 | 1.2E−02 | 9.4E−03 | AG v. BD | AC v. BD | |
| 205481_at | 9.0E−02 | 1.5E−01 | 1.0E−01 | 1.5E−01 | Yes | No | ADORA1 |
| 216220_s_at | 1.2E−01 | 9.4E−02 | 2.4E−01 | 2.2E−01 | Yes | No | ADORA1 |
| 202834_at | 2.1E−01 | 8.4E−02 | 3.6E−01 | 7.2E−02 | Yes | No | AGT |
| 204151_x_at | 5.8E−01 | 8.6E−01 | 5.1E−01 | 7.5E−01 | Yes | No | AKR1C1 |
| 216594_x_at | 8.4E−01 | 4.0E−01 | 8.3E−01 | 3.0E−01 | Yes | No | AKR1C1 |
| 204445_s_at | 2.5E−02 | 1.7E−01 | 1.4E−01 | 5.8E−01 | Yes | No | ALOX5 |
| 204446_s_at | 1.9E−02 | 2.1E−01 | 1.1E−03 | 1.4E−01 | Yes | No | ALOX5 |
| 214366_s_at | 2.9E−02 | 1.1E−01 | 1.7E−01 | 3.3E−01 | Yes | No | ALOX5 |
| 214953_s_at | 7.0E−01 | 5.2E−01 | 8.2E−01 | 2.6E−01 | Yes | No | APP |
| 211621_at | 7.1E−01 | 6.4E−01 | 2.7E−01 | 2.3E−02 | Yes | No | AR |
| 203174_s_at | 1.6E−01 | 2.5E−01 | 1.1E−01 | 2.7E−01 | Yes | No | ARFRP1 |
| 215984_s_at | 1.2E−01 | 7.5E−01 | 3.7E−02 | 5.3E−01 | Yes | No | ARFRP1 |
| 201891_s_at | 9.5E−01 | 4.6E−01 | 5.4E−01 | 3.1E−01 | Yes | No | B2M |
| 216231_s_at | 7.4E−01 | 9.9E−02 | 9.7E−01 | 8.1E−02 | Yes | No | B2M |
| 203685_at | 1.2E−02 | 3.8E−02 | 1.3E−02 | 7.5E−02 | Yes | No | BCL2 |
| 207005_s_at | 6.0E−02 | 7.0E−01 | 2.1E−02 | 3.0E−01 | Yes | No | BCL2 |
| 205681_at | 1.7E−02 | 1.4E−02 | 8.8E−02 | 1.3E−02 | Yes | No | BCL2A1 |
| 202076_at | 4.8E−01 | 1.1E−01 | 7.3E−01 | 8.4E−02 | Yes | No | BIRC2 |
| 210538_s_at | 4.3E−04 | 6.6E−02 | 7.0E−05 | 1.9E−02 | Yes | No | BIRC3 |
| 205114_s_at | 4.9E−01 | 2.0E−01 | 4.9E−01 | 1.9E−01 | Yes | No | CCL3 |
| 204103_at | 2.2E−02 | 4.3E−01 | 7.5E−02 | 9.2E−01 | Yes | No | CCL4 |
| 201700_at | 3.5E−03 | 3.1E−03 | 4.3E−04 | 3.2E−03 | Yes | No | CCND3 |
| 204118_at | 1.4E−01 | 4.8E−02 | 1.6E−01 | 7.0E−02 | Yes | No | CD48 |
| 209795_at | 2.8E−02 | 4.2E−02 | 1.9E−02 | 4.0E−02 | Yes | No | CD69 |
| 209619_at | 5.7E−01 | 2.4E−02 | 2.3E−01 | 2.2E−02 | Yes | No | CD74 |
| 207176_s_at | 5.0E−01 | 4.9E−02 | 4.5E−01 | 3.8E−02 | Yes | No | CD80 |
| 204440_at | 8.3E−02 | 3.7E−02 | 5.2E−01 | 2.0E−02 | Yes | No | CD83 |
| 202246_s_at | 2.2E−01 | 6.6E−01 | 1.4E−01 | 6.7E−01 | Yes | No | CDK4 |
| 203198_at | 1.0E−01 | 7.3E−03 | 1.9E−02 | 1.0E−02 | Yes | No | CDK9 |
| 202284_s_at | 9.1E−01 | 7.7E−02 | 5.6E−01 | 1.1E−01 | Yes | No | CDKN1A |
| 208485_x_at | 8.3E−01 | 2.4E−01 | 9.3E−02 | 3.0E−01 | Yes | No | CFLAR |
| 209508_x_at | 8.8E−01 | 9.9E−02 | 8.5E−01 | 9.1E−02 | Yes | No | CFLAR |
| 209939_x_at | 1.6E−03 | 5.6E−03 | 3.6E−03 | 3.5E−03 | Yes | No | CFLAR |
| 210563_x_at | 2.5E−03 | 7.5E−02 | 5.7E−03 | 3.8E−02 | Yes | No | CFLAR |
| 211316_x_at | 5.4E−01 | 3.3E−03 | 7.8E−01 | 3.4E−02 | Yes | No | CFLAR |
| 211317_s_at | 3.5E−01 | 1.3E−01 | 4.3E−02 | 5.5E−02 | Yes | No | CFLAR |
| 211862_x_at | 4.3E−01 | 1.5E−02 | 7.0E−02 | 6.5E−02 | Yes | No | CFLAR |
| 214618_at | 6.0E−01 | 1.8E−01 | 9.9E−01 | 1.8E−01 | Yes | No | CFLAR |
| 209716_at | 7.4E−01 | 8.3E−02 | 5.1E−01 | 5.7E−01 | Yes | No | CSF1 |
| 200838_at | 2.4E−02 | 2.3E−01 | 8.1E−03 | 6.1E−02 | Yes | No | CTSB |
| 200839_s_at | 3.7E−03 | 1.7E−02 | 8.6E−03 | 2.5E−02 | Yes | No | CTSB |
| 213274_s_at | 7.8E−01 | 5.5E−01 | 6.2E−01 | 4.3E−01 | Yes | No | CTSB |
| 204470_at | 7.6E−01 | 5.9E−01 | 3.6E−01 | 9.7E−01 | Yes | No | CXCL1 |
| 210163_at | 4.9E−01 | 7.2E−01 | 3.1E−01 | 4.9E−01 | Yes | No | CXCL11 |
| 214974_x_at | 5.9E−01 | 5.7E−01 | 2.4E−01 | 1.2E−01 | Yes | No | CXCL5 |
| 203700_s_at | 8.7E−01 | 2.2E−01 | 9.4E−01 | 3.2E−01 | Yes | No | DIO2 |
| 201041_s_at | 4.7E−03 | 1.0E−02 | 1.3E−03 | 2.7E−03 | Yes | No | DUSP1 |
| 203692_s_at | 3.9E−01 | 3.5E−01 | 4.3E−01 | 2.1E−01 | Yes | No | E2F3 |
| 203693_s_at | 7.7E−01 | 7.1E−01 | 2.9E−01 | 9.8E−01 | Yes | No | E2F3 |
| 211551_at | 8.5E−01 | 8.2E−01 | 3.7E−01 | 2.1E−01 | Yes | No | EGFR |
| 201694_s_at | 9.8E−01 | 2.3E−01 | 8.7E−01 | 2.4E−01 | Yes | No | EGR1 |
| 214766_s_at | 9.4E−02 | 1.9E−02 | 9.6E−02 | 2.1E−02 | Yes | No | ELYS |
| 201313_at | 1.7E−03 | 2.1E−01 | 2.2E−03 | 3.4E−01 | Yes | No | ENO2 |
| 205767_at | 8.1E−02 | 3.8E−01 | 1.5E−01 | 2.7E−01 | Yes | No | EREG |
| 202081_at | 1.1E−02 | 2.6E−03 | 7.1E−03 | 1.8E−03 | Yes | No | ETR101 |
| 205756_s_at | 1.3E−01 | 1.8E−01 | 3.1E−01 | 5.0E−01 | Yes | No | F8 |
| 214701_s_at | 7.2E−01 | 1.3E−01 | 4.7E−01 | 6.4E−01 | Yes | No | FN1 |
| 202275_at | 7.7E−01 | 3.5E−02 | 5.3E−01 | 3.1E−02 | Yes | No | G6PD |
| 207574_s_at | 8.1E−02 | 3.3E−01 | 3.6E−02 | 8.8E−01 | Yes | No | GADD45B |
| 209304_x_at | 1.3E−01 | 2.8E−01 | 6.8E−03 | 2.4E−01 | Yes | No | GADD45B |
| 209305_s_at | 1.2E−01 | 3.4E−01 | 1.2E−01 | 4.1E−01 | Yes | No | GADD45B |
| 202269_x_at | 5.2E−02 | 4.5E−02 | 2.0E−02 | 1.5E−02 | Yes | No | GBP1 |
| 202270_at | 2.9E−02 | 3.8E−02 | 2.9E−02 | 3.6E−02 | Yes | No | GBP1 |
| 200651_at | 3.7E−02 | 3.3E−01 | 8.8E−03 | 1.5E−02 | Yes | No | GNB2L1 |
| 222034_at | 8.6E−02 | 4.0E−01 | 8.0E−02 | 5.0E−01 | Yes | No | GNB2L1 |
| 200824_at | 9.0E−03 | 2.6E−02 | 2.2E−03 | 8.8E−03 | Yes | No | GSTP1 |
| 208729_x_at | 1.7E−03 | 2.2E−05 | 1.2E−04 | 1.8E−04 | Yes | Yes | HLA-B |
| 209140_x_at | 6.0E−04 | 1.4E−04 | 3.6E−04 | 6.9E−05 | Yes | Yes | HLA-B |
| 211911_x_at | 1.3E−05 | 2.4E−06 | 9.6E−05 | 1.3E−05 | Yes | Yes | HLA-B |
| 200943_at | 7.3E−03 | 1.2E−02 | 1.5E−01 | 2.2E−01 | Yes | No | HMGN1 |
| 200944_s_at | 8.2E−02 | 5.5E−02 | 2.8E−01 | 1.9E−01 | Yes | No | HMGN1 |
| 202638_s_at | 1.3E−01 | 3.2E−01 | 1.4E−01 | 7.1E−01 | Yes | No | ICAM1 |
| 215485_s_at | 7.9E−01 | 5.7E−01 | 8.4E−01 | 4.9E−01 | Yes | No | ICAM1 |
| 210354_at | 7.4E−01 | 1.4E−01 | 8.1E−01 | 2.0E−01 | Yes | No | IFNG |
| 202718_at | 7.9E−02 | 8.4E−02 | 7.9E−02 | 8.5E−02 | Yes | No | IGFBP2 |
| 207901_at | 7.8E−01 | 5.2E−01 | 2.3E−01 | 2.1E−01 | Yes | No | IL12B |
| 208200_at | 2.9E−01 | 4.9E−01 | 3.4E−01 | 3.0E−01 | Yes | No | IL1A |
| 205207_at | 7.1E−01 | 6.5E−01 | 8.8E−01 | 8.0E−01 | Yes | No | IL6 |
| 202531_at | 7.7E−03 | 3.6E−03 | 2.3E−02 | 1.2E−02 | Yes | No | IRF1 |
| 203275_at | 3.7E−02 | 1.6E−02 | 2.7E−04 | 2.6E−02 | Yes | No | IRF2 |
| 204562_at | 3.4E−01 | 2.1E−04 | 1.6E−01 | 1.7E−03 | Yes | No | IRF4 |
| 216986_s_at | 3.5E−01 | 4.9E−01 | 3.8E−01 | 5.5E−01 | Yes | No | IRF4 |
| 208436_s_at | 1.5E−01 | 1.0E−01 | 5.4E−02 | 2.7E−02 | Yes | No | IRF7 |
| 213281_at | 1.3E−01 | 4.0E−01 | 4.0E−02 | 2.7E−01 | Yes | No | JUN |
| 201473_at | 3.2E−01 | 8.9E−03 | 5.6E−01 | 3.7E−03 | Yes | No | JUNB |
| 207029_at | 6.7E−01 | 4.8E−01 | 9.6E−01 | 7.9E−01 | Yes | No | KITLG |
| 216264_s_at | 4.1E−01 | 4.6E−01 | 9.0E−02 | 2.4E−01 | Yes | No | LAMB2 |
| 207339_s_at | 8.9E−03 | 1.7E−02 | 3.5E−03 | 2.0E−02 | Yes | No | LTB |
| 213975_s_at | 4.1E−02 | 5.2E−02 | 3.4E−02 | 3.8E−02 | Yes | No | LYZ |
| 208037_s_at | 7.3E−01 | 2.1E−01 | 8.1E−01 | 6.9E−02 | Yes | No | MADCAM1 |
| 206296_x_at | 6.8E−03 | 2.2E−03 | 2.5E−03 | 6.8E−04 | Yes | Yes | MAP4K1 |
| 214219_x_at | 5.2E−03 | 1.0E−03 | 8.4E−03 | 2.7E−03 | Yes | Yes | MAP4K1 |
| 214339_s_at | 1.5E−03 | 6.0E−04 | 1.2E−02 | 3.4E−03 | Yes | Yes | MAP4K1 |
| 201126_s_at | 2.0E−01 | 1.9E−03 | 3.9E−01 | 1.4E−02 | Yes | No | MGAT1 |
| 203936_s_at | 6.7E−01 | 1.3E−01 | 4.5E−01 | 8.5E−02 | Yes | No | MMP9 |
| 202086_at | 2.5E−01 | 3.4E−01 | 9.3E−01 | 8.7E−01 | Yes | No | MX1 |
| 204798_at | 9.2E−02 | 5.9E−02 | 1.0E−01 | 5.5E−02 | Yes | No | MYB |
| 215152_at | 5.3E−01 | 4.8E−01 | 1.9E−01 | 1.4E−01 | Yes | No | MYB |
| 202431_s_at | 1.4E−02 | 1.0E−01 | 1.6E−01 | 4.7E−01 | Yes | No | MYC |
| 209239_at | 6.6E−03 | 1.9E−01 | 6.8E−03 | 4.6E−01 | Yes | No | NFKB1 |
| 207535_s_at | 2.4E−03 | 4.8E−02 | 3.2E−03 | 2.4E−02 | Yes | No | NFKB2 |
| 201502_s_at | 8.8E−01 | 1.3E−01 | 4.7E−01 | 5.5E−02 | Yes | No | NFKBIA |
| 201468_s_at | 2.1E−01 | 1.1E−02 | 3.7E−01 | 1.0E−02 | Yes | No | NQO1 |
| 210519_s_at | 6.2E−02 | 3.2E−03 | 2.7E−02 | 2.7E−04 | Yes | No | NQO1 |
| 201866_s_at | 4.5E−04 | 2.3E−02 | 1.6E−04 | 1.0E−02 | Yes | No | NR3C1 |
| 211671_s_at | 2.8E−04 | 9.0E−02 | 1.1E−04 | 3.0E−02 | Yes | No | NR3C1 |
| 216321_s_at | 4.2E−03 | 3.9E−03 | 3.8E−03 | 1.5E−03 | Yes | Yes | NR3C1 |
| 204622_x_at | 1.6E−01 | 2.8E−01 | 3.3E−01 | 5.8E−01 | Yes | No | NR4A2 |
| 216248_s_at | 8.8E−03 | 1.3E−02 | 1.2E−01 | 1.4E−01 | Yes | No | NR4A2 |
| 210004_at | 9.3E−01 | 4.0E−01 | 6.9E−01 | 7.7E−01 | Yes | No | OLR1 |
| 121_at | 5.9E−01 | 2.9E−01 | 2.6E−01 | 1.1E−01 | Yes | No | PAX8 |
| 203557_s_at | 6.9E−05 | 1.1E−03 | 4.7E−05 | 4.2E−04 | Yes | Yes | PCBD |
| 201202_at | 4.6E−03 | 7.1E−01 | 9.5E−04 | 8.9E−01 | Yes | No | PCNA |
| 213791_at | 7.4E−01 | 5.4E−01 | 4.5E−01 | 9.5E−02 | Yes | No | PENK |
| 200737_at | 3.8E−02 | 2.8E−02 | 3.3E−02 | 2.3E−02 | Yes | No | PGK1 |
| 200738_s_at | 3.6E−02 | 5.7E−02 | 9.5E−03 | 1.7E−02 | Yes | No | PGK1 |
| 217356_s_at | 5.8E−02 | 6.6E−02 | 4.9E−02 | 5.6E−02 | Yes | No | PGK1 |
| 217383_at | 1.1E−01 | 2.1E−01 | 2.1E−01 | 5.1E−01 | Yes | No | PGK1 |
| 209193_at | 7.0E−01 | 3.5E−01 | 5.7E−01 | 4.4E−01 | Yes | No | PIM1 |
| 210145_at | 1.2E−01 | 8.8E−02 | 2.0E−01 | 9.3E−02 | Yes | No | PLA2G4A |
| 201979_s_at | 9.6E−01 | 6.8E−02 | 1.3E−01 | 1.6E−01 | Yes | No | PPP5C |
| 214617_at | 1.9E−02 | 1.9E−01 | 3.7E−02 | 2.7E−01 | Yes | No | PRF1 |
| 202545_at | 9.1E−04 | 6.3E−03 | 5.7E−03 | 8.3E−02 | Yes | No | PRKCD |
| 204279_at | 3.2E−03 | 6.5E−04 | 3.8E−04 | 1.5E−04 | Yes | Yes | PSMB9 |
| 211661_x_at | 1.2E−02 | 7.6E−02 | 5.5E−03 | 4.9E−02 | Yes | No | PTAFR |
| 211892_s_at | 4.9E−01 | 2.8E−01 | 1.9E−01 | 4.1E−01 | Yes | No | PTGIS |
| 206035_at | 3.2E−01 | 7.7E−02 | 1.2E−01 | 6.2E−03 | Yes | No | REL |
| 206036_s_at | 3.4E−02 | 8.9E−02 | 2.8E−02 | 7.4E−02 | Yes | No | REL |
| 205205_at | 4.6E−02 | 5.6E−02 | 4.3E−02 | 5.3E−02 | Yes | No | RELB |
| 209544_at | 5.3E−01 | 2.6E−01 | 9.8E−01 | 9.8E−02 | Yes | No | RIPK2 |
| 209545_s_at | 1.2E−01 | 3.6E−01 | 2.0E−02 | 1.4E−01 | Yes | No | RIPK2 |
| 200872_at | 1.1E−02 | 1.8E−02 | 3.3E−02 | 4.9E−02 | Yes | No | S100A10 |
| 203186_s_at | 4.6E−03 | 5.1E−03 | 4.5E−04 | 1.6E−02 | Yes | No | S100A4 |
| 215223_s_at | 2.2E−01 | 2.2E−02 | 1.6E−01 | 8.0E−03 | Yes | No | SOD2 |
| 216841_s_at | 4.5E−01 | 3.6E−02 | 3.2E−01 | 1.8E−02 | Yes | No | SOD2 |
| 221477_s_at | 1.3E−01 | 1.9E−03 | 1.3E−01 | 2.6E−03 | Yes | No | SOD2 |
| 201471_s_at | 2.1E−01 | 9.6E−02 | 4.6E−01 | 1.9E−01 | Yes | No | SQSTM1 |
| 208048_at | 9.2E−01 | 2.0E−01 | 9.5E−01 | 2.8E−01 | Yes | No | TACR1 |
| 202307_s_at | 1.4E−02 | 6.4E−04 | 1.2E−02 | 5.9E−04 | Yes | No | TAP1 |
| 208829_at | 2.9E−01 | 1.8E−01 | 7.3E−01 | 5.2E−01 | Yes | No | TAPBP |
| 207199_at | 1.8E−02 | 3.9E−02 | 2.4E−03 | 1.4E−01 | Yes | No | TERT |
| 215775_at | 4.0E−01 | 6.1E−01 | 2.9E−01 | 8.4E−02 | Yes | No | THBS1 |
| 216005_at | 2.6E−01 | 5.2E−02 | 2.7E−01 | 6.4E−02 | Yes | No | TNC |
| 202643_s_at | 1.7E−05 | 3.5E−02 | 6.8E−03 | 3.9E−02 | Yes | No | TNFAIP3 |
| 202644_s_at | 7.0E−04 | 5.2E−02 | 5.8E−04 | 1.4E−02 | Yes | No | TNFAIP3 |
| 215346_at | 1.6E−01 | 6.1E−02 | 1.5E−01 | 7.0E−02 | Yes | No | TNFRSF5 |
| 35150_at | 2.5E−01 | 1.3E−01 | 8.9E−02 | 6.3E−02 | Yes | No | TNFRSF5 |
| 207536_s_at | 9.7E−01 | 9.3E−01 | 6.4E−01 | 5.0E−01 | Yes | No | TNFRSF9 |
| 202687_s_at | 2.2E−02 | 3.8E−02 | 1.4E−03 | 6.6E−04 | Yes | No | TNFSF10 |
| 202688_at | 1.7E−04 | 3.6E−04 | 2.5E−02 | 1.8E−02 | Yes | No | TNFSF10 |
| 214329_x_at | 6.8E−02 | 1.1E−01 | 3.2E−02 | 7.6E−02 | Yes | No | TNFSF10 |
| 201746_at | 2.2E−04 | 1.6E−03 | 2.5E−04 | 1.8E−03 | Yes | Yes | TP53 |
| 211300_s_at | 1.6E−02 | 2.3E−02 | 8.0E−03 | 1.2E−02 | Yes | No | TP53 |
| 205599_at | 1.5E−01 | 1.6E−01 | 8.5E−02 | 7.2E−02 | Yes | No | TRAF1 |
| 213191_at | 8.6E−02 | 8.7E−02 | 3.6E−02 | 2.7E−02 | Yes | No | TRIF |
| 203109_at | 9.2E−02 | 1.3E−01 | 4.9E−02 | 1.1E−01 | Yes | No | UBE2M |
| 208997_s_at | 1.7E−02 | 3.2E−01 | 4.6E−02 | 7.4E−01 | Yes | No | UCP2 |
| 208998_at | 2.1E−02 | 7.5E−03 | 1.3E−02 | 4.1E−01 | Yes | No | UCP2 |
| 204881_s_at | 6.4E−02 | 5.4E−02 | 8.9E−02 | 8.3E−02 | Yes | No | UGCG |
| 221765_at | 9.0E−01 | 2.1E−01 | 5.2E−01 | 1.3E−01 | Yes | No | UGCG |
| 203868_s_at | 2.7E−01 | 9.7E−01 | 4.5E−01 | 9.9E−01 | Yes | No | VCAM1 |
| 209946_at | 3.5E−02 | 7.8E−02 | 7.0E−01 | 9.1E−01 | Yes | No | VEGFC |
| 201426_s_at | 3.3E−05 | 1.0E−03 | 2.2E−04 | 5.5E−04 | Yes | Yes | VIM |
| 210301_at | 6.4E−01 | 9.7E−02 | 5.3E−01 | 7.0E−02 | Yes | No | XDH |
| 208544_at | 3.4E−01 | 2.2E−01 | 1.7E−01 | 1.1E−01 | No | No | ADRA2B |
| 210081_at | 6.1E−02 | 3.0E−02 | 5.1E−01 | 1.3E−01 | No | No | AGER |
| 217046_s_at | 5.6E−01 | 5.6E−01 | 1.7E−01 | 2.2E−01 | No | No | AGER |
| 207206_s_at | 7.0E−01 | 3.3E−01 | 7.9E−01 | 2.6E−01 | No | No | ALOX12 |
| 213952_s_at | 8.6E−01 | 1.8E−01 | 7.9E−01 | 1.9E−01 | No | No | ALOX5 |
| 206516_at | 6.1E−01 | 1.4E−01 | 8.8E−01 | 1.9E−01 | No | No | AMH |
| 205820_s_at | 5.3E−01 | 2.8E−02 | 5.0E−01 | 2.1E−01 | No | No | APOC3 |
| 200602_at | 4.8E−01 | 2.1E−02 | 4.5E−01 | 2.2E−02 | No | No | APP |
| 211277_x_at | 1.1E−01 | 8.3E−01 | 3.1E−01 | 2.7E−01 | No | No | APP |
| 211110_s_at | 3.2E−01 | 2.9E−01 | 3.8E−01 | 3.6E−01 | No | No | AR |
| 207367_at | 8.2E−01 | 4.4E−01 | 9.5E−01 | 5.1E−01 | No | No | ATP12A |
| 217904_s_at | 3.3E−01 | 6.1E−01 | 5.1E−01 | 9.4E−01 | No | No | BACE |
| 203684_s_at | 1.2E−01 | 8.2E−01 | 4.8E−02 | 6.0E−01 | No | No | BCL2 |
| 207004_at | 1.9E−01 | 2.6E−01 | 3.3E−01 | 5.3E−01 | No | No | BCL2 |
| 207510_at | 9.6E−01 | 4.3E−01 | 5.3E−01 | 9.2E−02 | No | No | BDKRB1 |
| 202357_s_at | 8.6E−02 | 4.7E−02 | 5.8E−01 | 5.9E−01 | No | No | BF |
| 211920_at | 9.6E−01 | 9.2E−01 | 7.0E−01 | 6.7E−01 | No | No | BF |
| 201261_x_at | 6.7E−01 | 9.1E−01 | 6.5E−01 | 1.0E+00 | No | No | BGN |
| 201262_s_at | 7.9E−01 | 5.1E−01 | 5.5E−01 | 3.8E−01 | No | No | BGN |
| 205289_at | 8.1E−01 | 8.0E−01 | 6.0E−01 | 9.5E−01 | No | No | BMP2 |
| 205290_s_at | 1.9E−01 | 1.0E−01 | 8.3E−01 | 1.7E−01 | No | No | BMP2 |
| 204439_at | 9.1E−02 | 3.7E−02 | 6.7E−02 | 1.0E−01 | No | No | C1orf29 |
| 217767_at | 2.1E−01 | 1.6E−01 | 6.9E−01 | 7.0E−01 | No | No | C3 |
| 220066_at | 1.3E−02 | 6.4E−02 | 5.2E−01 | 1.1E−01 | No | No | CARD15 |
| 203065_s_at | 5.4E−01 | 2.7E−01 | 6.8E−01 | 6.9E−01 | No | No | CAV1 |
| 212097_at | 5.8E−01 | 7.8E−01 | 6.5E−01 | 3.7E−01 | No | No | CAV1 |
| 210133_at | 7.6E−01 | 5.6E−02 | 7.1E−01 | 7.7E−02 | No | No | CCL11 |
| 216598_s_at | 2.6E−01 | 1.7E−01 | 4.1E−02 | 3.3E−02 | No | No | CCL2 |
| 205476_at | 9.2E−01 | 8.2E−01 | 3.8E−01 | 3.3E−01 | No | No | CCL20 |
| 207861_at | 1.4E−01 | 7.1E−01 | 7.8E−01 | 2.9E−01 | No | No | CCL22 |
| 1405_i_at | 3.4E−01 | 3.1E−02 | 8.1E−01 | 1.0E−01 | No | No | CCL5 |
| 204655_at | 6.4E−01 | 5.7E−01 | 6.1E−01 | 8.2E−01 | No | No | CCL5 |
| 208711_s_at | 6.7E−02 | 2.4E−01 | 7.6E−01 | 9.9E−01 | No | No | CCND1 |
| 208712_at | 7.6E−01 | 2.5E−01 | 4.5E−01 | 4.5E−01 | No | No | CCND1 |
| 214019_at | 9.4E−02 | 1.2E−01 | 1.7E−01 | 2.6E−02 | No | No | CCND1 |
| 206991_s_at | 3.2E−01 | 1.6E−01 | 1.9E−01 | 1.1E−01 | No | No | CCR5 |
| 206337_at | 9.8E−01 | 4.6E−01 | 3.0E−01 | 5.5E−01 | No | No | CCR7 |
| 207277_at | 3.9E−01 | 2.6E−02 | 4.3E−01 | 3.5E−02 | No | No | CD209 |
| 207278_s_at | 8.8E−01 | 1.9E−01 | 3.0E−01 | 2.6E−01 | No | No | CD209 |
| 206804_at | 6.5E−01 | 2.0E−01 | 9.7E−01 | 1.3E−01 | No | No | CD3G |
| 210564_x_at | 7.5E−02 | 5.9E−01 | 1.2E−01 | 7.3E−02 | No | No | CFLAR |
| 214486_x_at | 7.2E−01 | 8.1E−02 | 1.4E−01 | 2.5E−01 | No | No | CFLAR |
| 202403_s_at | 1.9E−01 | 1.7E−01 | 2.1E−01 | 1.5E−01 | No | No | COL1A2 |
| 202404_s_at | 1.9E−01 | 9.8E−01 | 2.1E−01 | 4.6E−01 | No | No | COL1A2 |
| 205753_at | 8.9E−01 | 3.6E−01 | 3.5E−01 | 8.0E−01 | No | No | CRP |
| 37020_at | 6.5E−01 | 1.9E−01 | 6.5E−01 | 2.4E−01 | No | No | CRP |
| 207082_at | 4.0E−01 | 3.5E−01 | 9.6E−01 | 9.1E−01 | No | No | CSF1 |
| 210557_x_at | 9.4E−01 | 1.7E−01 | 8.6E−01 | 4.3E−01 | No | No | CSF1 |
| 211839_s_at | 5.4E−01 | 2.7E−01 | 5.9E−01 | 3.0E−01 | No | No | CSF1 |
| 210228_at | 4.9E−01 | 4.9E−01 | 6.8E−01 | 9.2E−01 | No | No | CSF2 |
| 210229_s_at | 6.2E−01 | 1.7E−01 | 4.7E−01 | 1.1E−01 | No | No | CSF2 |
| 207442_at | 6.0E−01 | 5.5E−01 | 3.9E−01 | 4.7E−02 | No | No | CSF3 |
| 213275_x_at | 1.2E−02 | 9.2E−04 | 9.8E−03 | 4.3E−02 | No | No | CTSB |
| 202087_s_at | 8.9E−01 | 5.5E−01 | 6.5E−01 | 7.0E−01 | No | No | CTSL |
| 204533_at | 2.5E−01 | 2.3E−01 | 9.3E−01 | 8.6E−01 | No | No | CXCL10 |
| 211122_s_at | 8.5E−01 | 7.3E−01 | 3.6E−01 | 1.5E−01 | No | No | CXCL11 |
| 209774_x_at | 3.2E−02 | 4.4E−01 | 7.8E−01 | 9.1E−01 | No | No | CXCL2 |
| 207852_at | 6.8E−01 | 8.4E−02 | 7.1E−01 | 1.7E−01 | No | No | CXCL5 |
| 215101_s_at | 9.4E−01 | 4.6E−01 | 5.5E−01 | 8.0E−01 | No | No | CXCL5 |
| 203699_s_at | 2.9E−01 | 2.3E−01 | 5.4E−01 | 9.7E−01 | No | No | DIO2 |
| 210819_x_at | 7.6E−01 | 8.3E−01 | 8.3E−01 | 4.6E−01 | No | No | DIO2 |
| 211215_x_at | 4.8E−01 | 3.1E−01 | 2.6E−01 | 1.5E−01 | No | No | DIO2 |
| 201044_x_at | 8.2E−01 | 7.9E−01 | 8.8E−01 | 8.5E−01 | No | No | DUSP1 |
| 201983_s_at | 5.3E−01 | 1.1E−01 | 3.4E−01 | 7.5E−02 | No | No | EGFR |
| 201984_s_at | 8.1E−01 | 3.0E−01 | 5.0E−01 | 7.6E−01 | No | No | EGFR |
| 210984_x_at | 2.0E−01 | 2.1E−01 | 1.3E−01 | 1.7E−01 | No | No | EGFR |
| 211550_at | 9.8E−01 | 3.4E−01 | 8.3E−01 | 3.0E−01 | No | No | EGFR |
| 211607_x_at | 7.9E−01 | 2.4E−01 | 6.9E−01 | 1.8E−01 | No | No | EGFR |
| 201693_s_at | 3.1E−01 | 2.2E−01 | 5.4E−01 | 6.3E−01 | No | No | EGR1 |
| 201808_s_at | 5.5E−01 | 7.6E−01 | 3.8E−01 | 4.8E−01 | No | No | ENG |
| 201809_s_at | 7.4E−03 | 1.3E−02 | 2.1E−03 | 3.5E−03 | No | No | ENG |
| 207257_at | 3.8E−01 | 3.4E−01 | 5.4E−01 | 5.4E−01 | No | No | EPO |
| 204363_at | 4.4E−01 | 9.9E−01 | 9.9E−01 | 4.1E−01 | No | No | F3 |
| 206759_at | 5.8E−02 | 1.6E−01 | 2.7E−02 | 8.7E−02 | No | No | FCER2 |
| 206760_s_at | 3.8E−02 | 9.5E−02 | 2.0E−02 | 7.0E−02 | No | No | FCER2 |
| 210495_x_at | 7.8E−01 | 3.9E−01 | 3.8E−01 | 6.2E−01 | No | No | FN1 |
| 211719_x_at | 4.7E−01 | 2.6E−01 | 8.8E−01 | 8.0E−01 | No | No | FN1 |
| 212464_s_at | 3.9E−01 | 3.4E−01 | 5.1E−01 | 5.1E−01 | No | No | FN1 |
| 214702_at | 6.2E−02 | 1.4E−01 | 9.8E−01 | 7.3E−01 | No | No | FN1 |
| 216442_x_at | 7.5E−01 | 8.2E−02 | 5.6E−01 | 4.2E−01 | No | No | FN1 |
| 205278_at | 8.7E−02 | 3.8E−03 | 9.0E−01 | 2.6E−01 | No | No | GAD1 |
| 206669_at | 8.4E−01 | 9.4E−01 | 9.3E−01 | 7.7E−01 | No | No | GAD1 |
| 206670_s_at | 1.8E−01 | 4.1E−01 | 1.6E−01 | 5.8E−02 | No | No | GAD1 |
| 213560_at | 4.5E−01 | 3.1E−01 | 9.9E−01 | 9.7E−01 | No | No | GADD45B |
| 220821_at | 5.8E−01 | 3.1E−01 | 6.3E−01 | 6.2E−01 | No | No | GALR1 |
| 205914_s_at | 4.4E−01 | 2.3E−01 | 6.8E−01 | 3.3E−01 | No | No | GRIN1 |
| 205915_x_at | 6.0E−01 | 2.2E−01 | 3.5E−01 | 1.5E−01 | No | No | GRIN1 |
| 210781_x_at | 5.7E−01 | 3.9E−01 | 6.2E−01 | 4.6E−01 | No | No | GRIN1 |
| 210782_x_at | 8.5E−01 | 9.3E−01 | 7.6E−01 | 8.6E−01 | No | No | GRIN1 |
| 211125_x_at | 4.6E−01 | 7.3E−01 | 7.9E−01 | 4.3E−01 | No | No | GRIN1 |
| 206534_at | 6.1E−01 | 1.9E−01 | 4.8E−01 | 1.5E−01 | No | No | GRIN2A |
| 221942_s_at | 7.7E−01 | 1.0E−01 | 5.7E−01 | 5.1E−02 | No | No | GUCY1A3 |
| 207316_at | 1.7E−01 | 1.4E−01 | 3.4E−01 | 2.9E−01 | No | No | HAS1 |
| 205919_at | 2.6E−01 | 9.6E−02 | 8.1E−01 | 2.3E−01 | No | No | HBE1 |
| 217683_at | 5.8E−01 | 6.0E−01 | 1.9E−01 | 3.4E−01 | No | No | HBE1 |
| 203665_at | 2.3E−01 | 7.0E−01 | 2.8E−02 | 1.5E−01 | No | No | HMOX1 |
| 210439_at | 4.6E−02 | 3.1E−01 | 3.7E−01 | 4.2E−01 | No | No | ICOS |
| 201631_s_at | 7.6E−03 | 1.2E−01 | 3.3E−02 | 2.4E−01 | No | No | IER3 |
| 205302_at | 4.8E−01 | 7.3E−02 | 6.6E−01 | 9.5E−01 | No | No | IGFBP1 |
| 207433_at | 9.3E−01 | 3.1E−01 | 5.1E−01 | 9.9E−01 | No | No | IL10 |
| 206924_at | 1.5E−01 | 9.5E−01 | 5.8E−01 | 3.8E−01 | No | No | IL11 |
| 206926_s_at | 4.1E−01 | 4.6E−01 | 2.0E−01 | 2.7E−01 | No | No | IL11 |
| 207844_at | 3.9E−01 | 4.7E−01 | 2.1E−01 | 5.9E−01 | No | No | IL13 |
| 205992_s_at | 4.3E−01 | 4.5E−01 | 3.0E−01 | 3.5E−01 | No | No | IL15 |
| 210118_s_at | 2.8E−01 | 3.9E−01 | 9.6E−01 | 1.6E−01 | No | No | IL1A |
| 205067_at | 2.2E−01 | 9.0E−02 | 4.3E−01 | 1.6E−01 | No | No | IL1B |
| 39402_at | 7.7E−01 | 4.6E−01 | 1.0E−01 | 7.7E−02 | No | No | IL1B |
| 212657_s_at | 3.9E−01 | 8.7E−01 | 9.4E−01 | 4.6E−01 | No | No | IL1RN |
| 212659_s_at | 7.3E−01 | 2.6E−01 | 7.2E−01 | 2.8E−01 | No | No | IL1RN |
| 216243_s_at | 2.0E−01 | 3.6E−01 | 1.2E−01 | 2.5E−01 | No | No | IL1RN |
| 216244_at | 1.0E−01 | 9.4E−02 | 6.2E−01 | 6.1E−01 | No | No | IL1RN |
| 216245_at | 9.3E−01 | 2.6E−01 | 7.9E−01 | 3.0E−01 | No | No | IL1RN |
| 207849_at | 7.5E−01 | 4.9E−01 | 6.1E−01 | 6.8E−01 | No | No | IL2 |
| 206341_at | 6.7E−02 | 5.4E−01 | 2.5E−01 | 6.7E−01 | No | No | IL2RA |
| 211269_s_at | 7.6E−01 | 1.7E−01 | 7.4E−01 | 8.7E−02 | No | No | IL2RA |
| 202859_x_at | 1.1E−01 | 6.5E−01 | 9.1E−01 | 2.6E−01 | No | No | IL8 |
| 208193_at | 7.9E−01 | 9.8E−01 | 2.9E−01 | 3.3E−01 | No | No | IL9 |
| 216987_at | 5.6E−01 | 7.4E−01 | 9.1E−01 | 6.0E−01 | No | No | IRF4 |
| 201464_x_at | 3.4E−01 | 9.4E−01 | 8.4E−02 | 5.0E−01 | No | No | JUN |
| 201465_s_at | 6.0E−01 | 7.9E−02 | 2.6E−01 | 6.3E−01 | No | No | JUN |
| 201466_s_at | 2.8E−01 | 2.9E−01 | 3.5E−01 | 8.9E−01 | No | No | JUN |
| 211124_s_at | 2.3E−01 | 9.1E−01 | 1.6E−01 | 8.7E−01 | No | No | KITLG |
| 204582_s_at | 7.6E−01 | 1.5E−01 | 6.3E−02 | 4.2E−01 | No | No | KLK3 |
| 204583_x_at | 4.7E−01 | 3.6E−01 | 8.9E−01 | 6.9E−01 | No | No | KLK3 |
| 204734_at | 2.8E−01 | 4.5E−01 | 6.8E−01 | 6.7E−01 | No | No | KRT15 |
| 208188_at | 9.6E−02 | 3.4E−02 | 3.0E−02 | 3.0E−02 | No | No | KRT9 |
| 211652_s_at | 7.9E−01 | 1.8E−01 | 2.0E−01 | 1.1E−01 | No | No | LBP |
| 214461_at | 8.8E−01 | 3.5E−01 | 6.7E−01 | 1.7E−01 | No | No | LBP |
| 212531_at | 8.7E−01 | 4.4E−01 | 5.6E−01 | 1.7E−01 | No | No | LCN2 |
| 208949_s_at | 9.1E−01 | 4.9E−02 | 2.2E−01 | 1.4E−01 | No | No | LGALS3 |
| 206975_at | 3.5E−01 | 2.9E−01 | 2.8E−01 | 4.6E−01 | No | No | LTA |
| 204475_at | 2.5E−01 | 2.9E−01 | 1.9E−01 | 4.7E−01 | No | No | MMP1 |
| 205828_at | 8.0E−01 | 2.9E−01 | 7.5E−01 | 2.8E−01 | No | No | MMP3 |
| 205970_at | 6.9E−01 | 9.5E−01 | 3.4E−01 | 6.4E−01 | No | No | MT3 |
| 204673_at | 2.4E−01 | 1.8E−01 | 9.3E−01 | 5.5E−01 | No | No | MUC2 |
| 209636_at | 8.2E−02 | 2.1E−02 | 1.2E−01 | 5.8E−02 | No | No | NFKB2 |
| 211524_at | 7.8E−01 | 4.7E−01 | 5.5E−01 | 5.0E−01 | No | No | NFKB2 |
| 203828_s_at | 7.1E−01 | 4.1E−01 | 5.0E−01 | 3.0E−01 | No | No | NK4 |
| 205440_s_at | 5.0E−01 | 3.3E−01 | 5.3E−01 | 2.5E−01 | No | No | NPY1R |
| 201467_s_at | 2.9E−01 | 5.9E−02 | 3.4E−01 | 5.0E−02 | No | No | NQO1 |
| 204621_s_at | 2.7E−01 | 8.3E−01 | 2.4E−02 | 1.3E−01 | No | No | NR4A2 |
| 205040_at | 3.2E−01 | 1.3E−01 | 7.4E−01 | 7.6E−01 | No | No | ORM1 |
| 205041_s_at | 4.5E−01 | 1.0E−01 | 6.2E−01 | 8.0E−01 | No | No | ORM1 |
| 214465_at | 9.5E−01 | 6.6E−02 | 7.4E−01 | 1.5E−01 | No | No | ORM1 |
| 207921_x_at | 8.1E−01 | 4.4E−01 | 5.5E−01 | 3.2E−01 | No | No | PAX8 |
| 207923_x_at | 3.6E−01 | 5.6E−02 | 1.2E−01 | 3.0E−02 | No | No | PAX8 |
| 207924_x_at | 4.5E−03 | 1.9E−04 | 4.9E−01 | 4.4E−01 | No | No | PAX8 |
| 209552_at | 2.6E−01 | 1.5E−01 | 1.4E−01 | 1.2E−01 | No | No | PAX8 |
| 214528_s_at | 9.7E−01 | 2.0E−01 | 9.6E−01 | 1.9E−01 | No | No | PAX8 |
| 221990_at | 3.0E−01 | 2.5E−01 | 5.4E−01 | 3.3E−01 | No | No | PAX8 |
| 204200_s_at | 1.0E+00 | 8.1E−01 | 4.3E−01 | 6.0E−01 | No | No | PDGFB |
| 216055_at | 3.1E−01 | 1.8E−01 | 8.6E−01 | 3.6E−01 | No | No | PDGFB |
| 216061_x_at | 2.9E−02 | 8.9E−02 | 5.5E−02 | 1.1E−01 | No | No | PDGFB |
| 206803_at | 9.3E−01 | 3.1E−02 | 9.8E−01 | 1.9E−01 | No | No | PDYN |
| 204213_at | 6.0E−01 | 2.7E−01 | 4.0E−01 | 1.8E−01 | No | No | PIGR |
| 203649_s_at | 1.1E−01 | 1.1E−01 | 6.2E−01 | 5.3E−01 | No | No | PLA2G2A |
| 205479_s_at | 2.8E−01 | 2.7E−01 | 5.1E−01 | 5.3E−01 | No | No | PLAU |
| 211668_s_at | 4.3E−01 | 2.8E−01 | 2.9E−01 | 7.3E−01 | No | No | PLAU |
| 205125_at | 4.7E−01 | 8.4E−01 | 1.2E−01 | 8.2E−01 | No | No | PLCD1 |
| 205720_at | 5.2E−01 | 1.6E−01 | 4.9E−01 | 9.2E−01 | No | No | POMC |
| 215705_at | 7.6E−01 | 3.9E−01 | 5.9E−01 | 3.2E−01 | No | No | PPP5C |
| 206278_at | 4.5E−01 | 6.8E−01 | 2.5E−01 | 2.8E−01 | No | No | PTAFR |
| 211663_x_at | 2.1E−01 | 8.9E−02 | 9.4E−01 | 4.6E−01 | No | No | PTGDS |
| 211748_x_at | 8.3E−02 | 2.0E−01 | 4.7E−01 | 5.2E−01 | No | No | PTGDS |
| 212187_x_at | 1.5E−01 | 1.8E−01 | 4.5E−01 | 3.6E−01 | No | No | PTGDS |
| 207388_s_at | 4.8E−01 | 1.3E−01 | 4.1E−01 | 8.3E−02 | No | No | PTGES |
| 210367_s_at | 4.9E−01 | 8.4E−01 | 5.1E−01 | 9.3E−01 | No | No | PTGES |
| 208131_s_at | 3.5E−01 | 2.2E−01 | 1.8E−01 | 1.8E−01 | No | No | PTGIS |
| 210702_s_at | 6.0E−01 | 8.2E−01 | 8.2E−01 | 2.7E−01 | No | No | PTGIS |
| 204748_at | 7.7E−01 | 8.1E−02 | 2.2E−01 | 1.5E−01 | No | No | PTGS2 |
| 204201_s_at | 9.7E−01 | 7.3E−01 | 5.4E−01 | 4.7E−01 | No | No | PTPN13 |
| 206157_at | 7.1E−01 | 6.6E−01 | 5.9E−01 | 6.3E−01 | No | No | PTX3 |
| 216994_s_at | 7.3E−01 | 2.4E−01 | 9.9E−01 | 1.6E−01 | No | No | RUNX2 |
| 221282_x_at | 6.1E−01 | 9.1E−01 | 9.7E−01 | 7.8E−01 | No | No | RUNX2 |
| 221283_at | 9.9E−01 | 3.6E−01 | 2.4E−01 | 3.9E−02 | No | No | RUNX2 |
| 217728_at | 9.2E−01 | 1.3E−01 | 7.9E−01 | 1.1E−01 | No | No | S100A6 |
| 208607_s_at | 6.3E−01 | 2.9E−01 | 5.5E−01 | 2.0E−01 | No | No | SAA2 |
| 214456_x_at | 7.2E−01 | 6.4E−01 | 8.1E−01 | 9.6E−01 | No | No | SAA2 |
| 202071_at | 5.4E−01 | 3.8E−01 | 3.6E−01 | 6.7E−01 | No | No | SDC4 |
| 206211_at | 2.1E−01 | 1.6E−01 | 2.2E−01 | 1.5E−01 | No | No | SELE |
| 206049_at | 6.5E−01 | 9.9E−01 | 5.9E−01 | 7.8E−01 | No | No | SELP |
| 202627_s_at | 9.2E−01 | 3.1E−01 | 5.2E−01 | 8.4E−01 | No | No | SERPINE1 |
| 202628_s_at | 3.5E−01 | 5.6E−01 | 6.8E−01 | 9.0E−01 | No | No | SERPINE1 |
| 210073_at | 1.1E−01 | 9.6E−01 | 8.8E−02 | 2.5E−01 | No | No | SIAT8A |
| 215078_at | 7.8E−01 | 5.6E−01 | 7.0E−01 | 9.6E−01 | No | No | SOD2 |
| 202935_s_at | 5.1E−01 | 5.8E−02 | 7.7E−01 | 1.5E−01 | No | No | SOX9 |
| 202936_s_at | 2.1E−01 | 2.3E−01 | 7.6E−01 | 9.2E−01 | No | No | SOX9 |
| 213112_s_at | 9.7E−01 | 3.4E−01 | 6.6E−01 | 2.2E−01 | No | No | SQSTM1 |
| 203010_at | 4.9E−03 | 1.2E−02 | 1.0E−03 | 6.7E−04 | No | No | STAT5A |
| 208049_s_at | 5.2E−02 | 9.1E−02 | 1.6E−01 | 7.6E−01 | No | No | TACR1 |
| 210637_at | 4.8E−01 | 2.2E−01 | 5.8E−01 | 6.0E−02 | No | No | TACR1 |
| 210294_at | 4.1E−02 | 6.3E−01 | 1.1E−01 | 4.5E−01 | No | No | TAPBP |
| 209277_at | 6.9E−01 | 5.9E−01 | 5.5E−01 | 5.4E−01 | No | No | TFPI2 |
| 209278_s_at | 9.7E−01 | 5.1E−01 | 3.9E−01 | 6.7E−01 | No | No | TFPI2 |
| 211003_x_at | 2.5E−01 | 1.3E−01 | 5.1E−01 | 1.6E−01 | No | No | TGM2 |
| 211573_x_at | 5.1E−01 | 7.7E−02 | 6.7E−01 | 1.6E−01 | No | No | TGM2 |
| 216183_at | 3.7E−01 | 9.8E−01 | 7.2E−01 | 3.9E−01 | No | No | TGM2 |
| 201107_s_at | 9.8E−01 | 1.6E−02 | 6.2E−01 | 1.8E−02 | No | No | THBS1 |
| 201108_s_at | 4.8E−01 | 3.5E−02 | 4.1E−01 | 8.2E−01 | No | No | THBS1 |
| 201109_s_at | 7.6E−01 | 4.5E−01 | 9.8E−01 | 7.3E−01 | No | No | THBS1 |
| 201110_s_at | 1.5E−01 | 3.6E−01 | 9.9E−02 | 2.5E−01 | No | No | THBS1 |
| 203083_at | 7.6E−02 | 4.5E−01 | 3.0E−01 | 7.1E−01 | No | No | THBS2 |
| 201645_at | 6.8E−01 | 9.6E−01 | 9.0E−01 | 5.2E−01 | No | No | TNC |
| 207113_s_at | 8.6E−01 | 2.0E−02 | 9.0E−01 | 2.7E−02 | No | No | TNF |
| 202509_s_at | 3.2E−01 | 7.4E−02 | 3.7E−01 | 1.0E−01 | No | No | TNFAIP2 |
| 202510_s_at | 4.8E−01 | 6.8E−01 | 1.7E−01 | 5.7E−01 | No | No | TNFAIP2 |
| 203508_at | 5.2E−01 | 9.9E−01 | 1.4E−01 | 2.7E−01 | No | No | TNFRSF1B |
| 205153_s_at | 1.0E−01 | 4.9E−02 | 2.3E−02 | 1.1E−02 | No | No | TNFRSF5 |
| 222292_at | 2.1E−01 | 4.4E−01 | 4.1E−01 | 6.6E−02 | No | No | TNFRSF5 |
| 211786_at | 2.9E−01 | 2.0E−02 | 9.3E−01 | 8.0E−02 | No | No | TNFRSF9 |
| 207892_at | 9.8E−01 | 2.2E−01 | 9.7E−01 | 1.8E−01 | No | No | TNFSF5 |
| 210865_at | 4.0E−01 | 8.3E−01 | 5.6E−01 | 1.8E−01 | No | No | TNFSF6 |
| 211333_s_at | 2.2E−01 | 5.8E−01 | 1.7E−03 | 3.2E−01 | No | No | TNFSF6 |
| 204413_at | 7.0E−01 | 3.3E−01 | 3.7E−01 | 5.3E−01 | No | No | TRAF2 |
| 206067_s_at | 4.8E−01 | 4.1E−01 | 8.3E−01 | 2.1E−01 | No | No | WT1 |
| 216953_s_at | 2.4E−01 | 5.2E−03 | 5.0E−01 | 9.6E−02 | No | No | WT1 |

| Probesets | UniGene ID | Gene Title |
|---|---|---|
| FDR Cutoff | | |
| 205481_at | Hs.77867 | adenosine A1 receptor |
| 216220_s_at | Hs.77867 | adenosine A1 receptor |
| 202834_at | Hs.19383 | angiotensinogen |
| 204151_x_at | Hs.295131 | aldo-keto reductase family 1, member C1 |
| 216594_x_at | Hs.295131 | aldo-keto reductase family 1, member C1 |
| 204445_s_at | Hs.89499 | arachidonate 5-lipoxygenase |
| 204446_s_at | Hs.89499 | arachidonate 5-lipoxygenase |
| 214366_s_at | Hs.89499 | arachidonate 5-lipoxygenase |
| 214953_s_at | Hs.177486 | amyloid beta (A4) precursor protein |
| 211621_at | Hs.99915 | androgen receptor |
| 203174_s_at | Hs.389277 | ADP-ribosylation factor related protein 1 |
| 215984_s_at | Hs.389277 | ADP-ribosylation factor related protein 1 |
| 201891_s_at | Hs.48516 | beta-2-microglobulin |
| 216231_s_at | Hs.48516 | beta-2-microglobulin |
| 203685_at | Hs.79241 | B-cell CLL/lymphoma 2 |
| 207005_s_at | Hs.79241 | B-cell CLL/lymphoma 2 |
| 205681_at | Hs.227817 | BCL2-related protein A1 |
| 202076_at | Hs.289107 | baculoviral IAP repeat-containing 2 |
| 210538_s_at | Hs.127799 | baculoviral IAP repeat-containing 3 |
| 205114_s_at | Hs.73817 | chemokine (C-C motif) ligand 3 |
| 204103_at | Hs.75703 | chemokine (C-C motif) ligand 4 |
| 201700_at | Hs.83173 | cyclin D3 |
| 204118_at | Hs.901 | CD48 antigen |
| 209795_at | Hs.82401 | CD69 antigen |
| 209619_at | Hs.446471 | CD74 antigen |
| 207176_s_at | Hs.838 | CD80 antigen |
| 204440_at | Hs.79197 | CD83 antigen |
| 202246_s_at | Hs.95577 | cyclin-dependent kinase 4 |
| 203198_at | Hs.150423 | cyclin-dependent kinase 9 |
| 202284_s_at | Hs.370771 | cyclin-dependent kinase inhibitor 1A (p21, |
| | | Cip1) |
| 208485_x_at | Hs.355724 | CASP8 and FADD-like apoptosis regulator |
| 209508_x_at | Hs.355724 | CASP8 and FADD-like apoptosis regulator |
| 209939_x_at | Hs.355724 | CASP8 and FADD-like apoptosis regulator |
| 210563_x_at | Hs.355724 | CASP8 and FADD-like apoptosis regulator |
| 211316_x_at | Hs.355724 | CASP8 and FADD-like apoptosis regulator |
| 211317_s_at | Hs.355724 | CASP8 and FADD-like apoptosis regulator |
| 211862_x_at | Hs.355724 | CASP8 and FADD-like apoptosis regulator |
| 214618_at | Hs.355724 | CASP8 and FADD-like apoptosis regulator |
| 209716_at | Hs.173894 | colony stimulating factor 1 (macrophage) |
| 200838_at | Hs.135226 | cathepsin B |
| 200839_s_at | Hs.135226 | cathepsin B |
| 213274_s_at | Hs.135226 | cathepsin B |
| 204470_at | Hs.789 | chemokine (C—X—C motif) ligand 1 |
| 210163_at | Hs.103982 | chemokine (C—X—C motif) ligand 11 |
| 214974_x_at | Hs.89714 | chemokine (C—X—C motif) ligand 5 |
| 203700_s_at | Hs.436020 | deiodinase, iodothyronine, type II |
| 201041_s_at | Hs.171695 | dual specificity phosphatase 1 |
| 203692_s_at | Hs.1189 | E2F transcription factor 3 |
| 203693_s_at | Hs.1189 | E2F transcription factor 3 |
| 211551_at | Hs.77432 | epidermal growth factor receptor |
| 201694_s_at | Hs.326035 | early growth response 1 |
| 214766_s_at | Hs.33363 | ELYS transcription factor-like protein |
| | | TMBS62 |
| 201313_at | Hs.511915 | enolase 2, (gamma, neuronal) |
| 205767_at | Hs.115263 | epiregulin |
| 202081_at | Hs.737 | immediate early protein |
| 205756_s_at | Hs.413083 | coagulation factor VIII |
| 214701_s_at | Hs.418138 | fibronectin 1 |
| 202275_at | Hs.80206 | glucose-6-phosphate dehydrogenase |
| 207574_s_at | Hs.110571 | growth arrest and DNA-damage-inducible, |
| | | beta |
| 209304_x_at | Hs.110571 | growth arrest and DNA-damage-inducible, |
| | | beta |
| 209305_s_at | Hs110571 | growth arrest and DNA-damage-inducible, |
| | | beta |
| 202269_x_at | Hs.62661 | guanylate binding protein 1, interferon- |
| | | inducible, 67 kDa |
| 202270_at | Hs.62661 | guanylate binding protein 1, interferon- |
| | | inducible, 67 kDa |
| 200651_at | Hs.5662 | G protein, beta polypeptide 2-like 1 |
| 222034_at | Hs.5662 | G protein, beta polypeptide 2-like 1 |
| 200824_at | Hs.411509 | glutathione S-transferase pi |
| 208729_x_at | Hs.77961 | major histocompatibility complex, class I, B |
| 209140_x_at | Hs.77961 | major histocompatibility complex, class I, B |
| 211911_x_at | Hs.77961 | major histocompatibility complex, class I, B |
| 200943_at | Hs.356285 | high-mobility group nucleosome binding |
| | | domain 1 |
| 200944_s_at | Hs.356285 | high-mobility group nucleosome binding |
| | | domain 1 |
| 202638_s_at | Hs.386467 | intercellular adhesion molecule 1 (CD54) |
| 215485_s_at | Hs.386467 | intercellular adhesion molecule 1 (CD54) |
| 210354_at | Hs.856 | interferon, gamma |
| 202718_at | Hs.433326 | insulin-like growth factor binding protein 2, |
| | | 36 kDa |
| 207901_at | Hs.674 | interleukin 12B |
| 208200_at | Hs.1722 | interleukin 1, alpha |
| 205207_at | Hs.512234 | interleukin 6 (interferon, beta 2) |
| 202531_at | Hs.80645 | interferon regulatory factor 1 |
| 203275_at | Hs.83795 | interferon regulatory factor 2 |
| 204562_at | Hs.127686 | interferon regulatory factor 4 |
| 216986_s_at | Hs.127686 | interferon regulatory factor 4 |
| 208436_s_at | Hs.166120 | interferon regulatory factor 7 |
| 213281_at | Hs.78465 | v-jun sarcoma virus 17 oncogene homolog |
| | | (avian) |
| 201473_at | Hs.400124 | jun B proto-oncogene |
| 207029_at | Hs.1048 | KIT ligand |
| 216264_s_at | Hs.439726 | laminin, beta 2 (laminin S) |
| 207339_s_at | Hs.376208 | lymphotoxin beta |
| 213975_s_at | Hs.234734 | lysozyme |
| 208037_s_at | Hs.102598 | mucosal vascular addressin cell adhesion |
| | | molecule 1 |
| 206296_x_at | Hs.95424 | mitogen-activated protein kinase kinase |
| | | kinase kinase 1 |
| 214219_x_at | Hs.95424 | mitogen-activated protein kinase kinase |
| | | kinase kinase 1 |
| 214339_s_at | Hs.95424 | mitogen-activated protein kinase kinase |
| | | kinase kinase 1 |
| 201126_s_at | Hs.120870 | mannosyl (alpha-1,3-)-glycoprotein beta- |
| | | 1,2-N-acetylglucosaminyltransferase |
| 203936_s_at | Hs.151738 | matrix metalloproteinase 9 |
| 202086_at | Hs.436836 | myxovirus (influenza virus) resistance 1 |
| 204798_at | Hs.407830 | v-myb myeloblastosis viral oncogene |
| | | homolog (avian) |
| 215152_at | Hs.407830 | v-myb myeloblastosis viral oncogene |
| | | homolog (avian) |
| 202431_s_at | Hs.202453 | v-myc myelocytomatosis viral oncogene |
| | | homolog (avian) |
| 209239_at | Hs.160557 | nuclear factor of kappa light polypeptide |
| | | gene enhancer in B-cells 1 (p105) |
| 207535_s_at | Hs.73090 | nuclear factor of kappa light polypeptide |
| | | gene enhancer in B-cells 2 (p49/p100) |
| 201502_s_at | Hs.81328 | nuclear factor of kappa light polypeptide |
| | | gene enhancer in B-cells inhibitor, alpha |
| 201468_s_at | Hs.406515 | NAD(P)H dehydrogenase, quinone 1 |
| 210519_s_at | Hs.406515 | NAD(P)H dehydrogenase, quinone 1 |
| 201866_s_at | Hs.512414 | nuclear receptor subfamily 3, group C, |
| | | member 1 (glucocorticoid receptor) |
| 211671_s_at | Hs.512414 | nuclear receptor subfamily 3, group C, |
| | | member 1 (glucocorticoid receptor) |
| 216321_s_at | Hs.512414 | nuclear receptor subfamily 3, group C, |
| | | member 1 (glucocorticoid receptor) |
| 204622_x_at | Hs.82120 | nuclear receptor subfamily 4, group A, |
| | | member 2 |
| 216248_s_at | Hs.82120 | nuclear receptor subfamily 4, group A, |
| | | member 2 |
| 210004_at | Hs.445299 | oxidised low density lipoprotein (lectin-like) |
| | | receptor 1 |
| 121_at | Hs.308061 | paired box gene 8 |
| 203557_s_at | Hs.3192 | dimerization cofactor of hepatocyte nuclear |
| | | factor 1 alpha (TCF1) |
| 201202_at | Hs.78996 | proliferating cell nuclear antigen |
| 213791_at | Hs.339831 | proenkephalin |
| 200737_at | Hs.78771 | phosphoglycerate kinase 1 |
| 200738_s_at | Hs.78771 | phosphoglycerate kinase 1 |
| 217356_s_at | Hs.78771 | phosphoglycerate kinase 1 |
| 217383_at | Hs.78771 | phosphoglycerate kinase 1 |
| 209193_at | Hs.81170 | pim-1 oncogene |
| 210145_at | Hs.211587 | phospholipase A2, group IVA (cytosolic, |
| | | calcium-dependent) |
| 201979_s_at | Hs.431861 | protein phosphatase 5, catalytic subunit |
| 214617_at | Hs.2200 | perforin 1 (pore forming protein) |
| 202545_at | Hs.155342 | protein kinase C, delta |
| 204279_at | Hs.381081 | proteasome (prosome, macropain) subunit, |
| | | beta type, 9 |
| 211661_x_at | Hs.46 | platelet-activating factor receptor |
| 211892_s_at | Hs.302085 | prostaglandin I2 (prostacyclin) synthase |
| 206035_at | Hs.44313 | v-rel reticuloendotheliosis viral oncogene |
| | | homolog (avian) |
| 206036_s_at | Hs.44313 | v-rel reticuloendotheliosis viral oncogene |
| | | homolog (avian) |
| 205205_at | Hs.307905 | v-rel reticuloendotheliosis viral oncogene |
| | | homolog B |
| 209544_at | Hs.103755 | receptor-interacting serine-threonine kinase 2 |
| 209545_s_at | Hs.103755 | receptor-interacting serine-threonine kinase 2 |
| 200872_at | Hs.143873 | S100 calcium binding protein A10 |
| 203186_s_at | Hs.81256 | S100 calcium binding protein A4 |
| 215223_s_at | Hs.384944 | superoxide dismutase 2, mitochondrial |
| 216841_s_at | Hs.384944 | superoxide dismutase 2, mitochondrial |
| 221477_s_at | Hs.384944 | superoxide dismutase 2, mitochondrial |
| 201471_s_at | Hs.182248 | sequestosome 1 |
| 208048_at | Hs.469066 | tachykinin receptor 1 |
| 202307_s_at | Hs.352018 | transporter 1, ATP-binding cassette, sub- |
| | | family B (MDR/TAP) |
| 208829_at | Hs.370937 | TAP binding protein (tapasin) |
| 207199_at | Hs.439911 | telomerase reverse transcriptase |
| 215775_at | Hs.164226 | thrombospondin 1 |
| 216005_at | Hs.98998 | tenascin C (hexabrachion) |
| 202643_s_at | Hs.211600 | tumor necrosis factor, alpha-induced |
| | | protein 3 |
| 202644_s_at | Hs.211600 | tumor necrosis factor, alpha-induced |
| | | protein 3 |
| 215346_at | Hs.504816 | tumor necrosis factor receptor superfamily, |
| | | member 5 |
| 35150_at | Hs.504816 | tumor necrosis factor receptor superfamily, |
| | | member 5 |
| 207536_s_at | Hs.193418 | tumor necrosis factor receptor superfamily, |
| | | member 9 |
| 202687_s_at | Hs.387871 | tumor necrosis factor (ligand) superfamily, |
| | | member 10 |
| 202688_at | Hs.387871 | tumor necrosis factor (ligand) superfamily, |
| | | member 10 |
| 214329_x_at | Hs.387871 | tumor necrosis factor (ligand) superfamily, |
| | | member 10 |
| 201746_at | Hs.408312 | tumor protein p53 (Li-Fraumeni syndrome) |
| 211300_s_at | Hs.408312 | tumor protein p53 (Li-Fraumeni syndrome) |
| 205599_at | Hs.438253 | TNF receptor-associated factor 1 |
| 213191_at | Hs.29344 | TIR domain containing adaptor inducing |
| | | interferon-beta |
| 203109_at | Hs.406068 | ubiquitin-conjugating enzyme E2M (UBC12 |
| | | homolog, yeast) |
| 208997_s_at | Hs.80658 | uncoupling protein 2 (mitochondrial, proton |
| | | carrier) |
| 208998_at | Hs.80658 | uncoupling protein 2 (mitochondrial, proton |
| | | carrier) |
| 204881_s_at | Hs.432605 | UDP-glucose ceramide glucosyltransferase |
| 221765_at | Hs.432605 | UDP-glucose ceramide glucosyltransferase |
| 203868_s_at | Hs.109225 | vascular cell adhesion molecule 1 |
| 209946_at | Hs.79141 | vascular endothelial growth factor C |
| 201426_s_at | Hs.435800 | vimentin |
| 210301_at | Hs.250 | xanthine dehydrogenase |
| 208544_at | Hs.247686 | adrenergic, alpha-2B-, receptor |
| 210081_at | Hs.184 | advanced glycosylation end product- |
| | | specific receptor |
| 217046_s_at | Hs.184 | advanced glycosylation end product- |
| | | specific receptor |
| 207206_s_at | Hs.1200 | arachidonate 12-lipoxygenase |
| 213952_s_at | Hs.89499 | arachidonate 5-lipoxygenase |
| 206516_at | Hs.112432 | anti-Mullerian hormone |
| 205820_s_at | Hs.73849 | apolipoprotein C-III |
| 200602_at | Hs.177486 | amyloid beta (A4) precursor protein |
| 211277_x_at | Hs.177486 | amyloid beta (A4) precursor protein |
| 211110_s_at | Hs.99915 | androgen receptor |
| 207367_at | Hs.147111 | ATPase, H+/K+ transporting, nongastric, |
| | | alpha polypeptide |
| 217904_s_at | Hs.49349 | beta-site APP-cleaving enzyme |
| 203684_s_at | Hs.79241 | B-cell CLL/lymphoma 2 |
| 207004_at | Hs.79241 | B-cell CLL/lymphoma 2 |
| 207510_at | Hs.46348 | bradykinin receptor B1 |
| 202357_s_at | Hs.69771 | B-factor, properdin |
| 211920_at | Hs.69771 | B-factor, properdin |
| 201261_x_at | Hs.821 | biglycan |
| 201262_s_at | Hs.821 | biglycan |
| 205289_at | Hs.73853 | bone morphogenetic protein 2 |
| 205290_s_at | Hs.73853 | bone morphogenetic protein 2 |
| 204439_at | Hs.389724 | chromosome 1 open reading frame 29 |
| 217767_at | Hs.284394 | complement component 3 |
| 220066_at | Hs.135201 | caspase recruitment domain family, |
| | | member 15 |
| 203065_s_at | Hs.74034 | caveolin 1, caveolae protein, 22 kDa |
| 212097_at | Hs.74034 | caveolin 1, caveolae protein, 22 kDa |
| 210133_at | Hs.54460 | chemokine (C-C motif) ligand 11 |
| 216598_s_at | Hs.303649 | chemokine (C-C motif) ligand 2 |
| 205476_at | Hs.75498 | chemokine (C-C motif) ligand 20 |
| 207861_at | Hs.97203 | chemokine (C-C motif) ligand 22 |
| 1405_i_at | Hs.489044 | chemokine (C-C motif) ligand 5 |
| 204655_at | Hs.489044 | chemokine (C-C motif) ligand 5 |
| 208711_s_at | Hs.371468 | cyclin D1 |
| 208712_at | Hs.371468 | cyclin D1 |
| 214019_at | Hs.371468 | cyclin D1 |
| 206991_s_at | Hs.511796 | chemokine (C-C motif) receptor 5 |
| 206337_at | Hs.1652 | chemokine (C-C motif) receptor 7 |
| 207277_at | Hs.278694 | CD209 antigen |
| 207278_s_at | Hs.278694 | CD209 antigen |
| 206804_at | Hs.2259 | CD3G antigen, gamma polypeptide (TiT3 |
| | | complex) |
| 210564_x_at | Hs.355724 | CASP8 and FADD-like apoptosis regulator |
| 214486_x_at | Hs.355724 | CASP8 and FADD-like apoptosis regulator |
| 202403_s_at | Hs.232115 | collagen, type I, alpha 2 |
| 202404_s_at | Hs.232115 | collagen, type I, alpha 2 |
| 205753_at | Hs.76452 | C-reactive protein, pentraxin-related |
| 37020_at | Hs.76452 | C-reactive protein, pentraxin-related |
| 207082_at | Hs.173894 | colony stimulating factor 1 (macrophage) |
| 210557_x_at | Hs.173894 | colony stimulating factor 1 (macrophage) |
| 211839_s_at | Hs.173894 | colony stimulating factor 1 (macrophage) |
| 210228_at | Hs.1349 | colony stimulating factor 2 (granulocyte- |
| | | macrophage) |
| 210229_s_at | Hs.1349 | colony stimulating factor 2 (granulocyte- |
| | | macrophage) |
| 207442_at | Hs.2233 | colony stimulating factor 3 (granulocyte) |
| 213275_x_at | Hs.135226 | cathepsin B |
| 202087_s_at | Hs.418123 | cathepsin L |
| 204533_at | Hs.413924 | chemokine (C—X—C motif) ligand 10 |
| 211122_s_at | Hs.103982 | chemokine (C—X—C motif) ligand 11 |
| 209774_x_at | Hs.75765 | chemokine (C—X—C motif) ligand 2 |
| 207852_at | Hs.89714 | chemokine (C—X—C motif) ligand 5 |
| 215101_s_at | Hs.89714 | chemokine (C—X—C motif) ligand 5 |
| 203699_s_at | Hs.436020 | deiodinase, iodothyronine, type II |
| 210819_x_at | Hs.436020 | deiodinase, iodothyronine, type II |
| 211215_x_at | Hs.436020 | deiodinase, iodothyronine, type II |
| 201044_x_at | Hs.171695 | dual specificity phosphatase 1 |
| 201983_s_at | Hs.77432 | epidermal growth factor receptor |
| 201984_s_at | Hs.77432 | epidermal growth factor receptor |
| 210984_x_at | Hs.77432 | epidermal growth factor receptor |
| 211550_at | Hs.77432 | epidermal growth factor receptor |
| 211607_x_at | Hs.77432 | epidermal growth factor receptor |
| 201693_s_at | Hs.326035 | early growth response 1 |
| 201808_s_at | Hs.76753 | endoglin (Osler-Rendu-Weber syndrome 1) |
| 201809_s_at | Hs.76753 | endoglin (Osler-Rendu-Weber syndrome 1) |
| 207257_at | Hs.2303 | erythropoietin |
| 204363_at | Hs.62192 | coagulation factor III (thromboplastin, |
| | | tissue factor) |
| 206759_at | Hs.1416 | Fc fragment of IgE, low affinity II, receptor |
| | | for (CD23A) |
| 206760_s_at | Hs.1416 | Fc fragment of IgE, low affinity II, receptor |
| | | for (CD23A) |
| 210495_x_at | Hs.418138 | fibronectin 1 |
| 211719_x_at | Hs.418138 | fibronectin 1 |
| 212464_s_at | Hs.418138 | fibronectin 1 |
| 214702_at | Hs.418138 | fibronectin 1 |
| 216442_x_at | Hs.418138 | fibronectin 1 |
| 205278_at | Hs.420036 | glutamate decarboxylase 1 (brain, 67 kDa) |
| 206669_at | Hs.420036 | glutamate decarboxylase 1 (brain, 67 kDa) |
| 206670_s_at | Hs.420036 | glutamate decarboxylase 1 (brain, 67 kDa) |
| 213560_at | Hs.110571 | growth arrest and DNA-damage-inducible, |
| | | beta |
| 220821_at | Hs.272191 | galanin receptor 1 |
| 205914_s_at | Hs.105 | glutamate receptor, ionotropic, N-methyl D- |
| | | aspartate 1 |
| 205915_x_at | Hs.105 | glutamate receptor, ionotropic, N-methyl D- |
| | | aspartate 1 |
| 210781_x_at | Hs.105 | glutamate receptor, ionotropic, N-methyl D- |
| | | aspartate 1 |
| 210782_x_at | Hs.105 | glutamate receptor, ionotropic, N-methyl D- |
| | | aspartate 1 |
| 211125_x_at | Hs.105 | glutamate receptor, ionotropic, N-methyl D- |
| | | aspartate 1 |
| 206534_at | Hs.125338 | glutamate receptor, ionotropic, N-methyl D- |
| | | aspartate 2A |
| 221942_s_at | Hs.433488 | guanylate cyclase 1, soluble, alpha 3 |
| 207316_at | Hs.57697 | hyaluronan synthase 1 |
| 205919_at | Hs.117848 | hemoglobin, epsilon 1 |
| 217683_at | Hs.117848 | hemoglobin, epsilon 1 |
| 203665_at | Hs.202833 | heme oxygenase (decycling) 1 |
| 210439_at | Hs.56247 | inducible T-cell co-stimulator |
| 201631_s_at | Hs.76095 | immediate early response 3 |
| 205302_at | Hs.401316 | insulin-like growth factor binding protein 1 |
| 207433_at | Hs.193717 | interleukin 10 |
| 206924_at | Hs.1721 | interleukin 11 |
| 206926_s_at | Hs.1721 | interleukin 11 |
| 207844_at | Hs.845 | interleukin 13 |
| 205992_s_at | Hs.168132 | interleukin 15 |
| 210118_s_at | Hs.1722 | interleukin 1, alpha |
| 205067_at | Hs.126256 | interleukin 1, beta |
| 39402_at | Hs.126256 | interleukin 1, beta |
| 212657_s_at | Hs.81134 | interleukin 1 receptor antagonist |
| 212659_s_at | Hs.81134 | interleukin 1 receptor antagonist |
| 216243_s_at | Hs.81134 | interleukin 1 receptor antagonist |
| 216244_at | Hs.81134 | interleukin 1 receptor antagonist |
| 216245_at | Hs.81134 | interleukin 1 receptor antagonist |
| 207849_at | Hs.89679 | interleukin 2 |
| 206341_at | Hs.130058 | interleukin 2 receptor, alpha |
| 211269_s_at | Hs.130058 | interleukin 2 receptor, alpha |
| 202859_x_at | Hs.624 | interleukin 8 |
| 208193_at | Hs.960 | interleukin 9 |
| 216987_at | Hs.127686 | interferon regulatory factor 4 |
| 201464_x_at | Hs.78465 | v-jun sarcoma virus 17 oncogene homolog |
| | | (avian) |
| 201465_s_at | Hs.78465 | v-jun sarcoma virus 17 oncogene homolog |
| | | (avian) |
| 201466_s_at | Hs.78465 | v-jun sarcoma virus 17 oncogene homolog |
| | | (avian) |
| 211124_s_at | Hs.1048 | KIT ligand |
| 204582_s_at | Hs.171995 | kallikrein 3, (prostate specific antigen) |
| 204583_x_at | Hs.171995 | kallikrein 3, (prostate specific antigen) |
| 204734_at | Hs.80342 | keratin 15 |
| 208188_at | Hs.2783 | keratin 9 (epidermolytic palmoplantar |
| | | keratoderma) |
| 211652_s_at | Hs.154078 | lipopolysaccharide binding protein |
| 214461_at | Hs.154078 | lipopolysaccharide binding protein |
| 212531_at | Hs.204238 | lipocalin 2 (oncogene 24p3) |
| 208949_s_at | Hs.411701 | lectin, galactoside-binding, soluble, 3 |
| | | (galectin 3) |
| 206975_at | Hs.36 | lymphotoxin alpha (TNF superfamily, |
| | | member 1) |
| 204475_at | Hs.83169 | matrix metalloproteinase 1 (interstitial |
| | | collagenase) |
| 205828_at | Hs.375129 | matrix metalloproteinase 3 (stromelysin 1, |
| | | progelatinase) |
| 205970_at | Hs.73133 | metallothionein 3 (growth inhibitory factor |
| | | (neurotrophic)) |
| 204673_at | Hs.315 | mucin 2, intestinal/tracheal |
| 209636_at | Hs.73090 | nuclear factor of kappa light polypeptide |
| | | gene enhancer in B-cells 2 (p49/p100) |
| 211524_at | Hs.73090 | nuclear factor of kappa light polypeptide |
| | | gene enhancer in B-cells 2 (p49/p100) |
| 203828_s_at | Hs.943 | natural killer cell transcript 4 |
| 205440_s_at | Hs.169266 | neuropeptide Y receptor Y1 |
| 201467_s_at | Hs.406515 | NAD(P)H dehydrogenase, quinone 1 |
| 204621_s_at | Hs.82120 | nuclear receptor subfamily 4, group A, |
| | | member 2 |
| 205040_at | Hs.278388 | orosomucoid 1 |
| 205041_s_at | Hs.278388 | orosomucoid 1 |
| 214465_at | Hs.278388 | orosomucoid 1 |
| 207921_x_at | Hs.308061 | paired box gene 8 |
| 207923_x_at | Hs.308061 | paired box gene 8 |
| 207924_x_at | Hs.308061 | paired box gene 8 |
| 209552_at | Hs.308061 | paired box gene 8 |
| 214528_s_at | Hs.308061 | paired box gene 8 |
| 221990_at | Hs.308061 | paired box gene 8 |
| 204200_s_at | Hs.1976 | platelet-derived growth factor beta |
| | | polypeptide |
| 216055_at | Hs.1976 | platelet-derived growth factor beta |
| | | polypeptide |
| 216061_x_at | Hs.1976 | platelet-derived growth factor beta |
| | | polypeptide |
| 206803_at | Hs.22584 | prodynorphin |
| 204213_at | Hs.205126 | polymeric immunoglobulin receptor |
| 203649_s_at | Hs.76422 | phospholipase A2, group IIA |
| 205479_s_at | Hs.77274 | plasminogen activator, urokinase |
| 211668_s_at | Hs.77274 | plasminogen activator, urokinase |
| 205125_at | Hs.80776 | phospholipase C, delta 1 |
| 205720_at | Hs.1897 | proopiomelanocortin (beta-endorphin) |
| 215705_at | Hs.431861 | protein phosphatase 5, catalytic subunit |
| 206278_at | Hs.46 | platelet-activating factor receptor |
| 211663_x_at | Hs.446429 | prostaglandin D2 synthase 21 kDa (brain) |
| 211748_x_at | Hs.446429 | prostaglandin D2 synthase 21 kDa (brain) |
| 212187_x_at | Hs.446429 | prostaglandin D2 synthase 21 kDa (brain) |
| 207388_s_at | Hs.146688 | prostaglandin E synthase |
| 210367_s_at | Hs.146688 | prostaglandin E synthase |
| 208131_s_at | Hs.302085 | prostaglandin I2 (prostacyclin) synthase |
| 210702_s_at | Hs.302085 | prostaglandin I2 (prostacyclin) synthase |
| 204748_at | Hs.196384 | prostaglandin-endoperoxide synthase 2 |
| 204201_s_at | Hs.387553 | APO-1/CD95 (Fas)-associated |
| | | phosphatase |
| 206157_at | Hs.2050 | pentaxin-related gene, rapidly induced by |
| | | IL-1 beta |
| 216994_s_at | Hs.122116 | runt-related transcription factor 2 |
| 221282_x_at | Hs.122116 | runt-related transcription factor 2 |
| 221283_at | Hs.122116 | runt-related transcription factor 2 |
| 217728_at | Hs.275243 | S100 calcium binding protein A6 (calcyclin) |
| 208607_s_at | Hs.1955 | serum amyloid A2 |
| 214456_x_at | Hs.1955 | serum amyloid A2 |
| 202071_at | Hs.252189 | syndecan 4 |
| 206211_at | Hs.89546 | selectin E |
| 206049_at | Hs.73800 | selectin P |
| 202627_s_at | Hs.414795 | serine (or cysteine) proteinase inhibitor, |
| | | clade E, member 1 |
| 202628_s_at | Hs.414795 | serine (or cysteine) proteinase inhibitor, |
| | | clade E, member 1 |
| 210073_at | Hs.408614 | sialyltransferase 8A |
| 215078_at | Hs.384944 | superoxide dismutase 2, mitochondrial |
| 202935_s_at | Hs.2316 | SRY (sex determining region Y)-box 9 |
| 202936_s_at | Hs.2316 | SRY (sex determining region Y)-box 9 |
| 213112_s_at | Hs.182248 | sequestosome 1 |
| 203010_at | Hs.437058 | signal transducer and activator of |
| | | transcription 5A |
| 208049_s_at | Hs.469066 | tachykinin receptor 1 |
| 210637_at | Hs.469066 | tachykinin receptor 1 |
| 210294_at | Hs.370937 | TAP binding protein (tapasin) |
| 209277_at | Hs.438231 | tissue factor pathway inhibitor 2 |
| 209278_s_at | Hs.438231 | tissue factor pathway inhibitor 2 |
| 211003_x_at | Hs.512708 | transglutaminase 2 |
| 211573_x_at | Hs.512708 | transglutaminase 2 |
| 216183_at | Hs.512708 | transglutaminase 2 |
| 201107_s_at | Hs.164226 | thrombospondin 1 |
| 201108_s_at | Hs.164226 | thrombospondin 1 |
| 201109_s_at | Hs.164226 | thrombospondin 1 |
| 201110_s_at | Hs.164226 | thrombospondin 1 |
| 203083_at | Hs.458354 | thrombospondin 2 |
| 201645_at | Hs.98998 | tenascin C (hexabrachion) |
| 207113_s_at | Hs.241570 | tumor necrosis factor (TNF superfamily, |
| | | member 2) |
| 202509_s_at | Hs.101382 | tumor necrosis factor, alpha-induced |
| | | protein 2 |
| 202510_s_at | Hs.101382 | tumor necrosis factor, alpha-induced |
| | | protein 2 |
| 203508_at | Hs.256278 | tumor necrosis factor receptor superfamily, |
| | | member 1B |
| 205153_s_at | Hs.504816 | tumor necrosis factor receptor superfamily, |
| | | member 5 |
| 222292_at | Hs.504816 | tumor necrosis factor receptor superfamily, |
| | | member 5 |
| 211786_at | Hs.193418 | tumor necrosis factor receptor superfamily, |
| | | member 9 |
| 207892_at | Hs.652 | tumor necrosis factor (ligand) superfamily, |
| | | member 5 |
| 210865_at | Hs.2007 | tumor necrosis factor (ligand) superfamily, |
| | | member 6 |
| 211333_s_at | Hs.2007 | tumor necrosis factor (ligand) superfamily, |
| | | member 6 |
| 204413_at | Hs.437575 | TNF receptor-associated factor 2 |
| 206067_s_at | Hs.1145 | Wilms tumor 1 |
| 216953_s_at | Hs.1145 | Wilms tumor 1 |

TABLE 5A
Apoptosis Genes (See tables reference 2)

| | Log2 fluorescence intensity | | | | | | | | | | | |
|---|---|---|---|---|---|---|---|---|---|---|---|---|
| | | | | | | | | | | | | D4 |
| Probesets | Ly1 (A1) | Ly1 (A2) | Ly1 (A3) | Ly7 (B1) | Ly7 (B2) | Ly7 (B3) | Ly8 (C1) | Ly8 (C2) | Ly8 (C3) | D4 (D1) | D4 (D2) | (D3) |
| FDR Cutoff | | | | | | | | | | | | |
| 201715_s_at | 7.49 | 7.34 | 7.38 | 7.81 | 7.43 | 7.20 | 7.58 | 7.31 | 7.42 | 7.52 | 7.26 | 7.31 |
| 205013_s_at | 6.80 | 6.61 | 7.16 | 8.05 | 8.54 | 7.91 | 6.83 | 6.96 | 6.09 | 8.08 | 8.97 | 8.50 |
| 204833_at | 7.13 | 6.79 | 6.97 | 6.46 | 6.26 | 6.45 | 7.08 | 6.86 | 7.26 | 6.87 | 6.70 | 7.01 |
| 213026_at | 7.90 | 7.23 | 7.61 | 7.16 | 6.45 | 6.79 | 7.93 | 7.41 | 7.72 | 7.15 | 6.91 | 7.43 |
| 213930_at | 4.96 | 4.73 | 4.68 | 4.51 | 4.47 | 4.51 | 4.97 | 4.54 | 4.72 | 4.64 | 4.52 | 4.68 |
| 221666_s_at | 8.11 | 8.25 | 8.15 | 5.95 | 5.54 | 5.61 | 8.25 | 7.98 | 8.13 | 5.49 | 5.41 | 5.20 |
| 1861_at | 5.38 | 5.57 | 5.56 | 5.28 | 5.68 | 5.61 | 5.46 | 5.50 | 5.71 | 5.31 | 5.75 | 5.86 |
| 209406_at | 7.02 | 7.41 | 7.77 | 7.40 | 7.25 | 7.40 | 6.73 | 7.35 | 7.41 | 7.52 | 7.54 | 7.65 |
| 217911_s_at | 7.04 | 7.17 | 7.41 | 5.54 | 4.84 | 5.01 | 8.02 | 7.90 | 7.53 | 4.87 | 4.90 | 4.80 |
| 202984_s_at | 5.59 | 5.40 | 6.34 | 5.66 | 5.60 | 5.70 | 6.03 | 6.16 | 6.77 | 5.35 | 5.41 | 5.70 |
| 202985_s_at | 9.47 | 9.41 | 9.60 | 9.16 | 8.96 | 9.19 | 9.85 | 9.78 | 9.77 | 8.03 | 8.59 | 8.47 |
| 203728_at | 7.34 | 7.40 | 7.41 | 7.30 | 7.36 | 7.43 | 7.31 | 7.43 | 7.33 | 7.11 | 7.04 | 7.36 |
| 208478_s_at | 6.11 | 5.37 | 5.76 | 5.84 | 6.27 | 6.23 | 5.76 | 5.61 | 5.71 | 4.88 | 5.72 | 6.59 |
| 211833_s_at | 6.77 | 6.22 | 6.94 | 6.58 | 6.69 | 6.92 | 6.43 | 6.32 | 6.74 | 5.76 | 6.11 | 7.50 |
| 205263_at | 7.40 | 7.11 | 7.48 | 7.15 | 7.15 | 7.30 | 7.21 | 7.26 | 7.75 | 6.52 | 6.52 | 7.01 |
| 208536_s_at | 4.89 | 4.82 | 4.66 | 5.00 | 4.35 | 4.82 | 4.94 | 4.81 | 5.00 | 4.97 | 5.22 | 4.93 |
| 222343_at | 5.34 | 5.63 | 5.16 | 5.01 | 4.99 | 4.84 | 5.36 | 5.50 | 5.35 | 5.19 | 5.88 | 5.32 |
| 205780_at | 9.40 | 9.19 | 9.01 | 10.02 | 9.47 | 9.65 | 9.34 | 9.02 | 9.14 | 8.50 | 8.84 | 9.07 |
| 220451_s_at | 5.05 | 4.97 | 4.89 | 5.15 | 5.11 | 5.03 | 4.92 | 5.26 | 4.92 | 5.41 | 5.18 | 5.20 |
| 221478_at | 7.13 | 5.29 | 6.73 | 6.19 | 6.58 | 6.54 | 7.14 | 5.77 | 6.78 | 6.13 | 6.07 | 6.41 |
| 221479_s_at | 7.97 | 6.69 | 7.53 | 7.13 | 7.46 | 7.51 | 8.20 | 7.14 | 7.63 | 7.07 | 7.32 | 7.38 |
| 204950_at | 6.57 | 6.49 | 6.83 | 6.05 | 6.19 | 6.28 | 6.23 | 6.37 | 6.49 | 6.03 | 6.01 | 5.88 |
| 220162_s_at | 5.13 | 4.96 | 4.95 | 4.88 | 4.87 | 4.92 | 5.03 | 4.91 | 4.91 | 4.92 | 4.92 | 4.88 |
| 209970_x_at | 7.75 | 8.08 | 7.53 | 5.51 | 5.56 | 5.28 | 7.44 | 7.98 | 7.85 | 5.44 | 5.42 | 5.20 |
| 211366_x_at | 8.01 | 8.27 | 8.09 | 5.91 | 6.04 | 5.89 | 8.06 | 8.05 | 8.33 | 5.83 | 6.13 | 5.67 |
| 211367_s_at | 5.99 | 6.23 | 6.16 | 4.32 | 4.02 | 4.13 | 6.12 | 6.25 | 6.63 | 4.38 | 4.41 | 4.14 |
| 211368_s_at | 7.23 | 7.48 | 7.16 | 4.88 | 4.89 | 4.78 | 7.07 | 7.48 | 7.78 | 4.84 | 4.75 | 4.56 |
| 202763_at | 6.85 | 6.55 | 6.50 | 7.21 | 6.89 | 6.88 | 6.97 | 6.71 | 6.80 | 6.87 | 6.51 | 7.04 |
| 209310_s_at | 7.06 | 6.66 | 7.29 | 5.21 | 4.80 | 4.95 | 6.61 | 6.44 | 6.84 | 5.08 | 4.93 | 4.97 |
| 213596_at | 4.50 | 4.30 | 4.47 | 4.34 | 4.21 | 4.33 | 4.34 | 4.33 | 4.38 | 4.38 | 4.40 | 4.25 |
| 209790_s_at | 6.67 | 6.85 | 6.71 | 5.30 | 5.23 | 5.19 | 6.99 | 7.04 | 7.33 | 6.29 | 6.19 | 6.40 |
| 211464_x_at | 4.88 | 4.96 | 5.17 | 4.33 | 4.56 | 4.44 | 4.97 | 5.27 | 5.39 | 4.68 | 4.76 | 4.83 |
| 222201_s_at | 6.69 | 6.56 | 6.88 | 6.72 | 6.61 | 6.88 | 6.70 | 6.56 | 7.01 | 6.56 | 6.20 | 6.69 |
| 221188_s_at | 7.36 | 7.50 | 7.39 | 6.91 | 7.14 | 6.76 | 7.30 | 7.63 | 7.28 | 7.12 | 7.15 | 7.09 |
| 209833_at | 6.99 | 7.04 | 6.86 | 6.48 | 6.52 | 6.73 | 6.98 | 6.78 | 6.98 | 7.25 | 7.26 | 7.29 |
| 201111_at | 8.18 | 8.84 | 9.16 | 9.71 | 9.66 | 9.89 | 8.35 | 8.76 | 8.92 | 8.57 | 8.54 | 9.09 |
| 201112_s_at | 10.07 | 10.29 | 10.14 | 10.54 | 10.37 | 10.61 | 10.04 | 10.31 | 10.40 | 10.12 | 9.88 | 10.12 |
| 210766_s_at | 9.10 | 9.59 | 9.33 | 9.77 | 9.60 | 9.70 | 9.21 | 9.53 | 9.58 | 9.06 | 8.99 | 9.27 |
| 202468_s_at | 7.92 | 8.28 | 8.13 | 7.32 | 7.29 | 7.53 | 8.25 | 8.48 | 8.64 | 8.02 | 7.87 | 8.47 |
| 208905_at | 12.04 | 12.19 | 12.14 | 11.95 | 11.77 | 11.92 | 12.05 | 12.17 | 12.17 | 12.30 | 12.14 | 12.28 |
| 201095_at | 8.80 | 8.75 | 8.51 | 8.92 | 8.84 | 8.66 | 8.57 | 8.36 | 8.37 | 8.29 | 8.50 | 8.47 |
| 208822_s_at | 9.68 | 9.55 | 9.62 | 9.78 | 9.66 | 9.79 | 9.78 | 9.47 | 9.69 | 9.53 | 9.43 | 9.57 |
| 203890_s_at | 6.39 | 6.33 | 6.45 | 6.65 | 6.41 | 6.62 | 6.69 | 6.37 | 6.70 | 6.24 | 6.65 | 6.41 |
| 203891_s_at | 5.49 | 5.10 | 5.25 | 5.76 | 5.23 | 5.39 | 5.54 | 5.41 | 5.39 | 5.72 | 5.14 | 5.37 |
| 217840_at | 9.40 | 8.97 | 8.91 | 9.08 | 9.05 | 8.71 | 9.40 | 8.91 | 8.84 | 8.80 | 8.83 | 8.81 |
| 202480_s_at | 5.82 | 5.86 | 5.76 | 6.44 | 6.46 | 6.39 | 5.82 | 5.91 | 5.73 | 5.99 | 6.05 | 5.96 |
| 211255_x_at | 5.35 | 5.38 | 5.42 | 5.76 | 6.23 | 6.15 | 5.24 | 5.22 | 5.06 | 5.60 | 5.69 | 5.68 |
| 215158_s_at | 7.59 | 7.63 | 7.64 | 8.25 | 8.40 | 8.16 | 7.64 | 7.46 | 7.50 | 7.96 | 8.06 | 7.98 |
| 203277_at | 8.42 | 8.50 | 8.15 | 8.90 | 8.84 | 8.48 | 8.54 | 8.51 | 8.30 | 8.43 | 8.64 | 8.18 |
| 219350_s_at | 9.44 | 9.31 | 9.26 | 9.37 | 9.40 | 9.41 | 9.41 | 9.26 | 9.36 | 9.07 | 9.11 | 9.18 |
| 205554_s_at | 5.34 | 5.35 | 5.26 | 5.52 | 5.25 | 5.43 | 5.65 | 5.39 | 5.60 | 5.54 | 5.44 | 5.47 |
| 209831_x_at | 8.53 | 8.39 | 7.95 | 8.03 | 8.51 | 8.17 | 8.44 | 8.25 | 8.04 | 7.80 | 7.84 | 7.76 |
| 214992_s_at | 6.95 | 6.24 | 5.85 | 6.30 | 6.84 | 6.29 | 6.62 | 6.12 | 6.25 | 5.51 | 5.56 | 5.30 |
| 208289_s_at | 7.21 | 7.13 | 7.21 | 7.42 | 7.20 | 7.49 | 7.15 | 7.23 | 7.31 | 7.26 | 6.89 | 7.31 |
| 216396_s_at | 8.60 | 8.55 | 8.70 | 8.69 | 9.03 | 9.03 | 8.61 | 8.70 | 8.54 | 8.55 | 8.96 | 8.80 |
| 202535_at | 8.37 | 8.43 | 8.45 | 8.53 | 8.37 | 8.38 | 8.39 | 8.22 | 8.28 | 7.87 | 8.20 | 8.09 |
| 218080_x_at | 8.63 | 8.78 | 8.39 | 9.31 | 9.52 | 9.52 | 8.65 | 8.56 | 8.87 | 9.13 | 9.17 | 9.24 |
| 202723_s_at | 7.81 | 8.03 | 7.97 | 6.62 | 6.75 | 6.77 | 7.66 | 8.26 | 7.59 | 6.91 | 7.66 | 7.16 |
| 202724_s_at | 8.53 | 8.70 | 9.02 | 7.33 | 7.44 | 7.24 | 8.63 | 8.85 | 8.29 | 8.29 | 8.70 | 8.15 |
| 204132_s_at | 6.11 | 5.80 | 5.85 | 5.93 | 5.30 | 5.76 | 6.51 | 5.84 | 6.21 | 6.06 | 6.05 | 6.16 |
| 201635_s_at | 9.05 | 8.88 | 9.03 | 8.94 | 8.89 | 9.14 | 9.12 | 8.85 | 9.26 | 8.91 | 9.10 | 9.34 |
| 201636_at | 6.26 | 6.13 | 6.67 | 6.25 | 5.94 | 6.10 | 6.02 | 6.39 | 6.54 | 6.18 | 5.96 | 6.71 |
| 201637_s_at | 9.55 | 9.26 | 9.58 | 9.54 | 9.45 | 9.72 | 9.68 | 9.40 | 9.71 | 9.50 | 9.55 | 9.96 |
| 203725_at | 9.09 | 8.62 | 9.19 | 8.20 | 8.11 | 8.37 | 9.59 | 8.95 | 9.20 | 7.99 | 7.32 | 8.24 |
| 207574_s_at | 7.55 | 6.78 | 6.81 | 6.37 | 6.11 | 6.33 | 7.73 | 6.90 | 7.36 | 7.45 | 7.12 | 7.56 |
| 209304_x_at | 7.93 | 7.32 | 6.99 | 6.93 | 6.71 | 6.64 | 7.80 | 7.38 | 7.66 | 7.92 | 7.70 | 7.83 |
| 209305_s_at | 7.57 | 6.94 | 6.82 | 6.59 | 6.37 | 6.71 | 7.51 | 6.69 | 7.25 | 7.40 | 7.40 | 7.39 |
| 214467_at | 6.83 | 6.46 | 6.61 | 6.38 | 6.17 | 6.15 | 6.21 | 6.42 | 6.51 | 6.44 | 6.53 | 6.57 |
| 220864_s_at | 10.48 | 10.34 | 10.40 | 10.40 | 10.41 | 10.16 | 10.51 | 10.15 | 10.37 | 10.90 | 11.11 | 11.03 |
| 206864_s_at | 5.75 | 5.74 | 6.60 | 4.93 | 4.99 | 4.90 | 6.34 | 5.97 | 5.98 | 4.47 | 4.58 | 4.49 |
| 206865_at | 6.88 | 6.92 | 7.33 | 6.02 | 6.27 | 6.14 | 7.20 | 7.19 | 6.48 | 6.10 | 6.03 | 6.02 |
| 207180_s_at | 7.89 | 7.83 | 7.60 | 6.13 | 5.54 | 6.22 | 7.98 | 7.64 | 8.01 | 7.50 | 7.50 | 7.57 |
| 209448_at | 7.29 | 7.26 | 7.43 | 4.67 | 4.94 | 4.74 | 7.46 | 7.46 | 7.85 | 7.41 | 6.82 | 7.46 |
| 210253_at | 5.84 | 6.14 | 5.94 | 5.06 | 5.07 | 5.11 | 5.89 | 5.97 | 6.30 | 5.83 | 5.64 | 5.88 |
| 209929_s_at | 6.93 | 6.93 | 7.00 | 6.99 | 7.00 | 6.90 | 6.88 | 6.85 | 6.74 | 6.85 | 6.83 | 6.92 |
| 36004_at | 8.59 | 8.64 | 8.38 | 8.44 | 8.66 | 8.44 | 8.53 | 8.56 | 8.33 | 8.60 | 8.66 | 8.42 |
| 203836_s_at | 4.75 | 5.26 | 5.39 | 5.40 | 6.05 | 5.84 | 5.05 | 5.34 | 5.09 | 4.84 | 5.10 | 5.07 |
| 203837_at | 4.91 | 4.95 | 5.12 | 5.20 | 5.51 | 5.26 | 4.69 | 5.02 | 4.77 | 4.82 | 4.89 | 4.86 |
| 202086_at | 5.28 | 5.17 | 5.31 | 5.47 | 5.32 | 5.27 | 5.42 | 5.37 | 5.29 | 5.49 | 5.29 | 5.26 |
| 207738_s_at | 4.51 | 4.58 | 4.63 | 4.20 | 4.44 | 4.20 | 4.71 | 4.74 | 4.58 | 4.59 | 5.48 | 5.16 |
| 217465_at | 4.24 | 4.29 | 4.34 | 4.27 | 4.24 | 4.39 | 4.20 | 4.35 | 4.39 | 4.30 | 4.45 | 4.37 |
| 201502_s_at | 9.28 | 8.98 | 8.75 | 9.09 | 9.14 | 8.67 | 9.01 | 8.78 | 8.68 | 9.09 | 9.69 | 9.61 |
| 204862_s_at | 7.59 | 7.26 | 7.36 | 6.59 | 6.48 | 6.59 | 7.69 | 7.40 | 7.43 | 6.66 | 6.18 | 6.54 |
| 205851_at | 5.87 | 6.41 | 6.30 | 5.74 | 5.71 | 5.79 | 5.99 | 6.13 | 5.95 | 5.60 | 5.61 | 5.54 |
| 204004_at | 8.69 | 9.02 | 8.97 | 8.19 | 7.92 | 7.96 | 8.50 | 9.06 | 8.58 | 8.97 | 9.12 | 8.98 |
| 204005_s_at | 5.37 | 5.63 | 5.98 | 5.03 | 4.80 | 5.12 | 5.24 | 5.26 | 5.64 | 5.80 | 5.60 | 5.89 |
| 204025_s_at | 6.56 | 6.31 | 6.59 | 6.70 | 6.58 | 6.63 | 6.57 | 6.38 | 6.54 | 6.54 | 6.48 | 6.79 |
| 213581_at | 8.25 | 8.61 | 8.56 | 7.88 | 7.79 | 7.68 | 8.32 | 8.65 | 8.59 | 8.05 | 7.83 | 8.06 |
| 202730_s_at | 7.12 | 6.85 | 7.10 | 6.78 | 6.84 | 6.93 | 7.01 | 6.86 | 6.91 | 6.47 | 6.94 | 6.99 |
| 202731_at | 7.40 | 7.15 | 7.83 | 7.14 | 7.06 | 7.28 | 7.44 | 7.18 | 7.55 | 6.96 | 7.20 | 7.38 |
| 212593_s_at | 9.63 | 9.46 | 9.83 | 9.42 | 9.42 | 9.50 | 9.57 | 9.59 | 9.50 | 9.29 | 9.44 | 9.53 |
| 212594_at | 5.65 | 5.16 | 5.88 | 5.13 | 4.93 | 4.84 | 5.59 | 5.38 | 5.46 | 5.55 | 5.32 | 6.08 |
| 219275_at | 7.57 | 7.74 | 7.77 | 7.70 | 7.70 | 7.83 | 7.55 | 7.63 | 8.08 | 7.38 | 7.50 | 7.95 |
| 203415_at | 8.96 | 8.79 | 8.98 | 9.00 | 8.85 | 8.99 | 8.70 | 8.53 | 8.72 | 8.84 | 8.85 | 9.02 |
| 217746_s_at | 9.70 | 9.61 | 9.88 | 9.75 | 9.67 | 9.79 | 10.13 | 9.74 | 10.03 | 9.48 | 9.25 | 9.64 |
| 205512_s_at | 8.95 | 9.24 | 9.21 | 8.81 | 8.81 | 8.87 | 9.20 | 9.17 | 9.46 | 8.95 | 9.31 | 9.37 |
| 207002_s_at | 3.75 | 3.82 | 3.74 | 3.65 | 3.73 | 3.66 | 3.68 | 3.71 | 3.69 | 3.63 | 3.70 | 3.70 |
| 209318_x_at | 3.93 | 3.92 | 3.94 | 3.83 | 3.92 | 3.96 | 3.82 | 3.98 | 3.97 | 3.89 | 3.90 | 3.93 |
| 216347_s_at | 6.07 | 5.66 | 5.70 | 5.95 | 5.57 | 5.69 | 6.09 | 5.79 | 5.94 | 6.20 | 5.64 | 5.91 |
| 203089_s_at | 7.78 | 7.72 | 7.44 | 8.21 | 8.02 | 8.08 | 7.79 | 7.50 | 7.61 | 7.97 | 8.07 | 8.11 |
| 211152_s_at | 6.93 | 6.92 | 6.73 | 7.15 | 7.15 | 7.07 | 6.78 | 6.82 | 6.67 | 7.14 | 7.11 | 7.09 |
| 209941_at | 6.82 | 6.04 | 6.28 | 6.48 | 6.24 | 6.40 | 6.70 | 6.41 | 6.52 | 7.03 | 7.10 | 7.13 |
| 209544_at | 4.32 | 4.30 | 4.29 | 4.30 | 4.15 | 4.33 | 4.31 | 4.25 | 4.23 | 4.29 | 4.38 | 4.41 |
| 209545_s_at | 8.73 | 8.32 | 8.08 | 7.95 | 7.82 | 7.89 | 8.39 | 8.18 | 8.47 | 8.14 | 8.21 | 8.11 |
| 218286_s_at | 8.23 | 7.95 | 8.38 | 8.06 | 7.78 | 8.02 | 8.22 | 8.01 | 8.10 | 8.14 | 8.20 | 8.43 |
| 203489_at | 9.28 | 9.15 | 9.05 | 8.68 | 8.61 | 8.57 | 9.58 | 9.52 | 9.74 | 8.63 | 8.95 | 8.81 |
| 210792_x_at | 9.72 | 9.83 | 9.46 | 9.22 | 9.18 | 9.13 | 9.98 | 10.02 | 10.05 | 9.08 | 9.58 | 9.37 |
| 222030_at | 5.94 | 6.06 | 6.03 | 5.98 | 6.15 | 5.96 | 6.01 | 6.15 | 6.02 | 5.80 | 5.95 | 5.86 |
| 202693_s_at | 8.34 | 7.38 | 7.69 | 7.26 | 6.34 | 6.83 | 8.48 | 7.77 | 7.87 | 9.78 | 9.51 | 9.42 |
| 202695_s_at | 5.70 | 5.56 | 5.72 | 5.70 | 5.51 | 5.74 | 5.86 | 5.58 | 5.71 | 7.20 | 7.48 | 7.39 |
| 205214_at | 5.14 | 5.18 | 5.77 | 6.19 | 6.35 | 6.38 | 5.29 | 5.51 | 5.50 | 6.35 | 6.94 | 6.59 |
| 200976_s_at | 8.89 | 8.51 | 8.65 | 8.22 | 8.05 | 8.18 | 9.08 | 8.77 | 9.01 | 8.45 | 8.63 | 8.76 |
| 200977_s_at | 9.06 | 8.77 | 8.90 | 8.36 | 8.20 | 8.44 | 9.10 | 8.82 | 9.47 | 8.87 | 8.55 | 9.19 |
| 213786_at | 4.27 | 4.16 | 4.42 | 4.14 | 4.26 | 4.21 | 4.23 | 4.32 | 4.39 | 4.20 | 4.28 | 4.62 |
| 201446_s_at | 7.75 | 7.62 | 7.84 | 7.68 | 7.50 | 7.74 | 7.78 | 7.49 | 7.89 | 7.45 | 7.25 | 7.42 |
| 201447_at | 6.71 | 6.33 | 6.53 | 6.12 | 6.28 | 6.38 | 6.35 | 6.12 | 6.28 | 6.09 | 5.86 | 6.34 |
| 201448_at | 6.94 | 6.75 | 7.43 | 6.77 | 6.48 | 6.80 | 6.59 | 6.52 | 6.99 | 6.57 | 6.15 | 6.92 |
| 201449_at | 5.61 | 5.51 | 5.90 | 5.66 | 5.52 | 5.60 | 5.26 | 5.56 | 5.75 | 5.05 | 4.98 | 5.41 |
| 201450_s_at | 5.42 | 5.40 | 5.83 | 5.43 | 5.42 | 5.41 | 5.42 | 5.43 | 5.54 | 5.29 | 4.93 | 5.38 |
| 217052_x_at | 5.83 | 5.69 | 5.88 | 5.92 | 5.92 | 5.81 | 5.79 | 5.80 | 5.75 | 5.98 | 5.86 | 5.87 |
| 217164_at | 4.44 | 4.24 | 4.17 | 4.17 | 4.08 | 4.09 | 4.37 | 4.13 | 4.21 | 4.18 | 4.08 | 4.18 |
| 202406_s_at | 9.80 | 9.75 | 9.65 | 9.81 | 9.67 | 9.80 | 9.81 | 9.73 | 9.92 | 9.25 | 9.26 | 9.67 |
| 217500_at | 5.26 | 5.19 | 4.95 | 5.34 | 5.06 | 5.24 | 5.18 | 5.04 | 5.19 | 5.21 | 5.17 | 5.24 |
| 207643_s_at | 6.10 | 5.91 | 5.76 | 6.19 | 6.29 | 5.86 | 5.93 | 5.93 | 5.72 | 5.09 | 5.22 | 5.32 |
| 215346_at | 6.64 | 6.15 | 6.16 | 6.82 | 6.86 | 6.41 | 6.44 | 6.49 | 5.92 | 6.97 | 6.90 | 6.83 |
| 35150_at | 8.56 | 8.35 | 8.22 | 8.42 | 8.72 | 8.53 | 8.28 | 8.41 | 8.06 | 8.67 | 8.99 | 8.50 |
| 204780_s_at | 4.90 | 4.78 | 4.86 | 4.90 | 4.93 | 5.00 | 4.59 | 4.62 | 4.82 | 4.71 | 4.65 | 4.78 |
| 215719_x_at | 4.38 | 4.20 | 4.35 | 4.43 | 4.35 | 4.40 | 4.00 | 4.26 | 4.28 | 4.07 | 4.27 | 4.15 |
| 216252_x_at | 4.83 | 4.65 | 4.67 | 4.91 | 4.91 | 4.79 | 4.55 | 4.61 | 4.66 | 4.53 | 4.65 | 4.47 |
| 206150_at | 9.36 | 8.87 | 9.24 | 7.95 | 7.97 | 7.90 | 9.42 | 9.00 | 8.86 | 9.66 | 9.62 | 9.70 |
| 207536_s_at | 5.11 | 5.46 | 5.06 | 5.16 | 5.17 | 5.31 | 5.18 | 5.22 | 5.17 | 5.17 | 5.31 | 5.20 |
| 202687_s_at | 4.47 | 4.44 | 4.24 | 3.95 | 4.07 | 4.00 | 4.33 | 4.39 | 4.41 | 4.06 | 4.09 | 4.01 |
| 202688_at | 4.95 | 4.86 | 4.95 | 4.28 | 4.36 | 4.31 | 5.09 | 4.80 | 5.22 | 4.24 | 4.25 | 4.37 |
| 214329_x_at | 3.74 | 3.82 | 3.96 | 3.58 | 3.66 | 3.63 | 3.75 | 3.91 | 3.88 | 3.58 | 3.77 | 3.63 |
| 206508_at | 5.59 | 5.65 | 5.60 | 7.85 | 8.89 | 7.93 | 5.59 | 5.81 | 5.45 | 5.69 | 5.96 | 5.81 |
| 207216_at | 6.14 | 6.11 | 6.05 | 6.68 | 6.95 | 6.86 | 6.21 | 6.28 | 5.95 | 6.27 | 6.32 | 6.03 |
| 206907_at | 5.15 | 5.09 | 5.10 | 5.76 | 6.73 | 6.15 | 5.08 | 5.15 | 4.94 | 5.49 | 6.14 | 5.81 |
| 203120_at | 6.09 | 6.27 | 6.23 | 5.60 | 5.57 | 5.66 | 5.93 | 6.21 | 6.14 | 5.28 | 5.32 | 5.09 |
| 209863_s_at | 4.89 | 4.89 | 5.04 | 5.14 | 5.61 | 5.49 | 4.74 | 5.00 | 4.92 | 4.93 | 5.06 | 4.99 |
| 211193_at | 4.37 | 4.60 | 4.38 | 4.35 | 4.52 | 4.50 | 4.33 | 4.43 | 4.42 | 4.50 | 4.45 | 4.47 |
| 1729_at | 6.37 | 6.32 | 6.39 | 6.26 | 6.25 | 6.33 | 6.32 | 6.39 | 6.33 | 6.28 | 6.36 | 6.30 |
| 205599_at | 5.00 | 5.55 | 5.30 | 5.47 | 6.03 | 5.60 | 5.07 | 5.44 | 5.16 | 5.51 | 5.90 | 5.51 |
| 208315_x_at | 5.55 | 5.65 | 5.56 | 5.33 | 5.31 | 4.93 | 5.74 | 5.57 | 5.51 | 5.75 | 5.81 | 5.80 |
| 221571_at | 7.09 | 6.94 | 7.35 | 5.83 | 5.46 | 5.85 | 6.77 | 6.73 | 6.93 | 7.26 | 7.41 | 7.60 |
| 202871_at | 7.06 | 6.86 | 6.80 | 7.40 | 7.42 | 7.28 | 7.23 | 7.06 | 6.68 | 7.14 | 7.65 | 7.39 |
| 211899_s_at | 7.63 | 7.52 | 7.46 | 7.86 | 8.18 | 8.14 | 7.56 | 7.39 | 7.10 | 7.55 | 8.36 | 7.84 |
| 204352_at | 5.72 | 5.67 | 6.02 | 6.04 | 6.16 | 5.88 | 5.54 | 5.54 | 5.40 | 5.16 | 5.87 | 5.35 |
| 207953_at | 4.94 | 5.25 | 5.11 | 5.09 | 5.12 | 4.98 | 5.05 | 5.30 | 4.88 | 5.01 | 4.92 | 5.02 |
| 208014_x_at | 5.09 | 5.22 | 5.26 | 4.84 | 5.72 | 5.46 | 5.20 | 5.35 | 5.09 | 5.60 | 5.38 | 5.21 |
| 209364_at | 6.32 | 6.16 | 6.06 | 6.06 | 6.05 | 6.11 | 6.35 | 6.16 | 6.40 | 6.32 | 6.61 | 6.60 |
| 221454_at | 5.31 | 5.26 | 5.13 | 5.34 | 5.23 | 5.15 | 5.32 | 5.31 | 5.29 | 5.56 | 5.38 | 5.19 |
| 210025_s_at | 6.03 | 6.03 | 5.71 | 6.03 | 5.83 | 5.87 | 5.88 | 5.90 | 5.94 | 6.24 | 5.90 | 5.90 |
| 210026_s_at | 5.08 | 5.17 | 5.07 | 4.97 | 5.23 | 5.10 | 5.01 | 5.07 | 5.03 | 5.02 | 5.03 | 5.02 |
| 214207_s_at | 4.85 | 4.79 | 4.74 | 4.86 | 4.83 | 4.79 | 4.96 | 4.90 | 4.71 | 4.94 | 4.99 | 4.89 |
| 220598_at | 5.89 | 5.75 | 5.50 | 5.84 | 5.79 | 5.73 | 5.83 | 5.68 | 5.71 | 6.01 | 5.79 | 5.70 |
| 220599_s_at | 5.41 | 5.36 | 5.47 | 5.54 | 5.44 | 5.40 | 5.30 | 5.52 | 5.40 | 5.37 | 5.57 | 5.58 |
| 220066_at | 4.51 | 4.50 | 4.51 | 4.55 | 4.57 | 4.59 | 4.54 | 4.61 | 4.47 | 4.73 | 4.60 | 4.62 |
| 221073_s_at | 7.60 | 7.63 | 7.40 | 7.24 | 7.41 | 7.30 | 7.53 | 7.47 | 7.47 | 7.45 | 7.24 | 7.07 |
| 206011_at | 5.71 | 5.71 | 5.64 | 4.95 | 4.89 | 5.07 | 5.44 | 5.83 | 5.76 | 5.12 | 5.10 | 5.17 |
| 207500_at | 4.76 | 4.79 | 4.81 | 4.72 | 4.79 | 4.63 | 4.81 | 4.81 | 4.79 | 4.78 | 4.97 | 4.73 |
| 208791_at | 4.89 | 4.94 | 4.95 | 4.93 | 5.07 | 5.05 | 4.79 | 4.85 | 4.88 | 5.14 | 4.94 | 4.90 |
| 208792_s_at | 5.08 | 4.98 | 4.94 | 5.13 | 4.87 | 5.05 | 5.00 | 5.05 | 5.05 | 5.26 | 5.05 | 5.00 |
| 222043_at | 4.35 | 4.37 | 4.38 | 4.29 | 4.50 | 4.37 | 4.26 | 4.33 | 4.23 | 4.41 | 4.43 | 4.34 |
| 210765_at | 5.49 | 5.46 | 5.50 | 5.64 | 5.55 | 5.80 | 5.40 | 5.56 | 5.40 | 5.55 | 5.51 | 5.54 |
| 213712_at | 4.21 | 4.07 | 3.99 | 4.09 | 4.15 | 3.99 | 4.09 | 3.95 | 4.10 | 4.23 | 4.06 | 4.15 |
| 203139_at | 4.74 | 5.00 | 5.17 | 4.68 | 4.99 | 4.93 | 4.77 | 4.96 | 4.67 | 4.95 | 4.98 | 4.85 |
| 218325_s_at | 6.13 | 6.75 | 6.70 | 6.35 | 6.75 | 6.66 | 6.06 | 6.79 | 6.49 | 5.79 | 6.45 | 5.83 |
| 206939_at | 4.39 | 4.49 | 4.67 | 4.48 | 4.65 | 4.55 | 4.47 | 4.74 | 4.47 | 4.67 | 4.74 | 4.60 |
| 210165_at | 5.44 | 5.45 | 5.33 | 5.44 | 5.62 | 5.35 | 5.39 | 5.41 | 5.05 | 5.58 | 5.73 | 5.43 |
| 210655_s_at | 6.86 | 6.41 | 6.06 | 6.12 | 5.92 | 6.05 | 6.66 | 6.41 | 6.48 | 6.36 | 6.57 | 6.39 |
| 213560_at | 5.74 | 5.81 | 5.58 | 5.53 | 5.79 | 5.52 | 5.60 | 5.68 | 5.57 | 5.63 | 5.63 | 5.58 |
| 204121_at | 4.46 | 4.63 | 4.42 | 4.67 | 4.61 | 4.67 | 4.67 | 4.68 | 4.64 | 4.79 | 4.89 | 4.70 |
| 205848_at | 3.88 | 4.02 | 3.90 | 3.86 | 3.88 | 3.90 | 3.84 | 3.93 | 3.89 | 3.82 | 4.02 | 3.88 |
| 208000_at | 5.69 | 5.89 | 5.89 | 5.78 | 5.72 | 6.05 | 5.71 | 5.78 | 5.92 | 6.06 | 6.05 | 5.88 |
| 205488_at | 4.57 | 4.45 | 4.44 | 4.51 | 4.40 | 4.49 | 4.51 | 4.49 | 4.46 | 4.70 | 4.57 | 4.53 |
| 210164_at | 4.43 | 4.47 | 4.35 | 4.39 | 4.33 | 4.37 | 4.40 | 4.27 | 4.27 | 4.49 | 4.41 | 4.37 |
| 210321_at | 4.93 | 5.23 | 4.77 | 5.03 | 5.03 | 4.95 | 5.12 | 5.04 | 5.00 | 5.13 | 5.21 | 5.07 |
| 206863_x_at | 4.78 | 4.58 | 4.46 | 4.74 | 4.76 | 4.46 | 4.61 | 4.60 | 4.50 | 4.66 | 4.68 | 4.50 |
| 207062_at | 4.27 | 4.35 | 4.43 | 4.28 | 4.49 | 4.53 | 4.10 | 4.41 | 4.27 | 4.32 | 4.38 | 4.36 |
| 206975_at | 5.18 | 5.31 | 5.23 | 5.18 | 5.25 | 5.13 | 5.49 | 5.45 | 5.12 | 5.38 | 6.09 | 5.30 |
| 203005_at | 5.39 | 5.35 | 5.25 | 5.47 | 5.27 | 5.12 | 5.62 | 5.42 | 5.10 | 5.66 | 5.38 | 5.30 |
| 205858_at | 4.54 | 4.55 | 4.46 | 4.63 | 4.43 | 4.61 | 4.57 | 4.64 | 4.36 | 4.62 | 4.50 | 4.53 |
| 220402_at | 4.06 | 4.01 | 4.15 | 4.01 | 4.21 | 4.05 | 3.98 | 4.09 | 4.08 | 4.06 | 4.09 | 4.08 |
| 220403_s_at | 4.71 | 4.63 | 4.63 | 4.73 | 4.70 | 4.64 | 4.86 | 4.69 | 4.68 | 4.86 | 4.74 | 4.71 |
| 214090_at | 3.92 | 4.10 | 4.18 | 4.02 | 4.13 | 4.08 | 3.95 | 3.96 | 4.02 | 4.02 | 4.25 | 4.17 |
| 214237_x_at | 5.58 | 5.52 | 5.46 | 5.71 | 5.41 | 5.50 | 5.50 | 5.64 | 5.57 | 5.83 | 5.75 | 5.59 |
| 207634_at | 5.73 | 5.79 | 5.41 | 5.66 | 5.80 | 5.69 | 5.59 | 6.06 | 5.52 | 5.86 | 5.68 | 5.60 |
| 213585_s_at | 6.04 | 6.08 | 6.08 | 6.01 | 6.08 | 6.14 | 5.89 | 6.12 | 5.93 | 6.03 | 6.17 | 5.97 |
| 222152_at | 5.25 | 5.20 | 5.19 | 5.17 | 5.29 | 5.21 | 5.15 | 5.21 | 5.13 | 5.52 | 5.16 | 5.30 |
| 207943_x_at | 3.89 | 4.05 | 3.99 | 3.87 | 4.00 | 4.01 | 3.92 | 3.96 | 4.01 | 3.93 | 3.94 | 3.95 |
| 218849_s_at | 5.97 | 5.82 | 5.83 | 5.89 | 5.97 | 6.07 | 5.74 | 5.93 | 5.91 | 5.91 | 6.02 | 5.97 |
| 207468_s_at | 4.50 | 4.48 | 4.34 | 4.62 | 4.50 | 4.52 | 4.57 | 4.52 | 4.40 | 4.57 | 4.55 | 4.48 |
| 202694_at | 4.38 | 4.55 | 4.63 | 4.23 | 4.52 | 4.41 | 4.41 | 4.47 | 4.43 | 4.45 | 4.60 | 4.63 |
| 206222_at | 4.61 | 4.55 | 4.45 | 4.59 | 4.42 | 4.54 | 4.51 | 4.54 | 4.62 | 4.78 | 4.55 | 4.54 |
| 211163_s_at | 4.67 | 4.57 | 4.53 | 4.71 | 4.47 | 4.65 | 4.65 | 4.54 | 4.66 | 4.85 | 4.68 | 4.68 |
| 210654_at | 5.17 | 4.86 | 4.83 | 4.94 | 4.89 | 4.96 | 5.28 | 4.89 | 4.93 | 5.01 | 5.03 | 5.07 |
| 210847_x_at | 4.91 | 5.00 | 4.85 | 5.02 | 5.04 | 4.82 | 5.03 | 5.11 | 4.92 | 5.23 | 5.23 | 4.89 |
| 211282_x_at | 5.86 | 6.00 | 5.84 | 5.97 | 5.96 | 5.93 | 5.90 | 6.08 | 5.88 | 6.36 | 6.28 | 5.89 |
| 211841_s_at | 5.74 | 5.87 | 5.59 | 5.80 | 5.82 | 5.81 | 5.88 | 5.81 | 5.80 | 6.08 | 5.85 | 5.76 |
| 216042_at | 6.20 | 6.41 | 6.49 | 6.45 | 6.50 | 6.47 | 6.43 | 6.48 | 6.39 | 6.54 | 6.51 | 6.40 |
| 219422_at | 4.87 | 4.97 | 4.78 | 5.02 | 5.07 | 4.91 | 5.02 | 5.00 | 4.89 | 5.22 | 4.93 | 4.85 |
| 219423_x_at | 5.37 | 5.36 | 5.32 | 5.35 | 5.20 | 5.38 | 5.27 | 5.35 | 5.26 | 5.45 | 5.37 | 5.30 |
| 205153_s_at | 5.94 | 5.42 | 5.24 | 6.04 | 6.24 | 6.03 | 5.75 | 5.47 | 5.33 | 6.25 | 6.50 | 6.10 |
| 222292_at | 4.59 | 4.65 | 4.73 | 4.57 | 4.65 | 4.52 | 4.51 | 4.47 | 4.60 | 4.66 | 4.67 | 4.85 |
| 204781_s_at | 5.53 | 5.72 | 5.57 | 5.90 | 5.74 | 5.89 | 5.33 | 5.36 | 5.67 | 5.53 | 5.76 | 5.52 |
| 211786_at | 4.61 | 4.53 | 4.49 | 4.64 | 4.53 | 4.64 | 4.52 | 4.65 | 4.62 | 4.71 | 4.76 | 4.67 |
| 207907_at | 4.98 | 4.62 | 4.72 | 5.11 | 4.72 | 4.74 | 5.00 | 4.87 | 4.78 | 5.11 | 5.00 | 4.92 |
| 207892_at | 5.42 | 5.61 | 5.56 | 5.39 | 5.63 | 5.56 | 5.52 | 5.57 | 5.49 | 5.79 | 5.62 | 5.57 |
| 210865_at | 5.13 | 5.25 | 4.92 | 5.08 | 5.06 | 4.81 | 5.02 | 4.99 | 4.64 | 5.23 | 5.19 | 4.97 |
| 211333_s_at | 4.60 | 4.61 | 4.38 | 4.69 | 4.65 | 4.65 | 4.48 | 4.51 | 4.53 | 4.61 | 4.71 | 4.46 |
| 220804_s_at | 4.87 | 4.91 | 4.80 | 5.16 | 4.87 | 5.00 | 4.99 | 4.91 | 5.03 | 5.34 | 5.06 | 5.12 |
| 207382_at | 5.47 | 5.47 | 5.56 | 5.45 | 5.39 | 5.49 | 5.01 | 5.50 | 5.43 | 5.52 | 5.44 | 5.54 |
| 211194_s_at | 4.44 | 4.51 | 4.48 | 4.46 | 4.65 | 4.65 | 4.41 | 4.42 | 4.35 | 4.42 | 4.67 | 4.49 |
| 211195_s_at | 5.10 | 5.42 | 5.38 | 4.98 | 5.35 | 5.36 | 5.08 | 5.46 | 5.19 | 5.27 | 5.34 | 5.40 |
| 211834_s_at | 4.82 | 4.91 | 4.73 | 4.96 | 4.84 | 4.82 | 4.95 | 4.97 | 4.79 | 5.01 | 4.88 | 4.63 |
| 205641_s_at | 6.94 | 6.99 | 6.88 | 6.96 | 6.83 | 6.62 | 7.00 | 7.03 | 6.98 | 7.00 | 7.02 | 6.90 |
| 213443_at | 5.19 | 5.29 | 5.31 | 5.18 | 5.28 | 5.19 | 5.33 | 5.23 | 5.26 | 5.43 | 5.33 | 5.21 |

TABLE 5B
Apoptosis Genes (See tables reference 2)

| | t-test (two-tailed) | | | | | | |
|---|---|---|---|---|---|---|---|
| Probesets | pAB | pAD | pBC | pCD | Measured | Changed | Gene Symbol |
| FDR Cutoff | 1.0E−02 | 8.2E−03 | 1.2E−02 | 9.4E−03 | AC v. BD | AC v. BD | |
| 201715_s_at | 7.2E−01 | 6.7E−01 | 8.4E−01 | 5.4E−01 | Yes | No | ACINUS |
| 205013_s_at | 6.6E−03 | 9.0E−03 | 1.2E−02 | 7.2E−03 | Yes | No | ADORA2A |
| 204833_at | 1.1E−02 | 5.0E−01 | 1.3E−02 | 2.4E−01 | Yes | No | APG12L |
| 213026_at | 5.1E−02 | 1.7E−01 | 2.9E−02 | 6.9E−02 | Yes | No | APG12L |
| 213930_at | 7.4E−02 | 1.7E−01 | 1.8E−01 | 4.1E−01 | Yes | No | APG12L |
| 221666_s_at | 9.7E−04 | 1.0E−04 | 2.5E−04 | 2.1E−05 | Yes | Yes | ASC |
| 1861_at | 9.1E−01 | 5.2E−01 | 8.4E−01 | 6.8E−01 | Yes | No | BAD |
| 209406_at | 8.6E−01 | 5.1E−01 | 4.8E−01 | 2.0E−01 | Yes | No | BAG2 |
| 217911_s_at | 3.2E−03 | 1.1E−03 | 8.4E−04 | 1.7E−03 | Yes | Yes | BAG3 |
| 202984_s_at | 7.3E−01 | 4.3E−01 | 9.9E−02 | 4.9E−02 | Yes | No | BAG5 |
| 202985_s_at | 1.5E−02 | 1.5E−02 | 5.9E−03 | 1.2E−02 | Yes | No | BAG5 |
| 203728_at | 6.6E−01 | 1.5E−01 | 8.7E−01 | 1.9E−01 | Yes | No | BAK1 |
| 208478_s_at | 2.4E−01 | 9.8E−01 | 8.1E−02 | 9.5E−01 | Yes | No | BAX |
| 211833_s_at | 7.5E−01 | 7.7E−01 | 2.2E−01 | 9.5E−01 | Yes | No | BAX |
| 205263_at | 3.9E−01 | 3.7E−02 | 3.6E−01 | 3.9E−02 | Yes | No | BCL10 |
| 208536_s_at | 7.7E−01 | 9.9E−02 | 4.2E−01 | 3.4E−01 | Yes | No | BCL2L11 |
| 222343_at | 7.3E−02 | 7.5E−01 | 3.1E−03 | 8.1E−01 | Yes | No | BCL2L11 |
| 205780_at | 6.5E−02 | 1.3E−01 | 5.4E−02 | 1.5E−01 | Yes | No | BIK |
| 220451_s_at | 9.6E−02 | 3.6E−02 | 6.6E−01 | 1.8E−01 | Yes | No | BIRC7 |
| 221478_at | 9.4E−01 | 7.8E−01 | 7.8E−01 | 4.7E−01 | Yes | No | BNIP3L |
| 221479_s_at | 9.4E−01 | 7.4E−01 | 4.5E−01 | 3.2E−01 | Yes | No | BNIP3L |
| 204950_at | 2.7E−02 | 1.3E−02 | 1.4E−01 | 1.8E−02 | Yes | No | CARD8 |
| 220162_s_at | 1.7E−01 | 2.2E−01 | 2.8E−01 | 3.9E−01 | Yes | No | CARD9 |
| 209970_x_at | 8.9E−04 | 1.0E−03 | 1.0E−03 | 1.2E−03 | Yes | Yes | CASP1 |
| 211366_x_at | 8.1E−05 | 5.4E−04 | 2.4E−04 | 3.5E−04 | Yes | Yes | CASP1 |
| 211367_s_at | 8.7E−05 | 1.1E−04 | 8.7E−04 | 1.1E−03 | Yes | Yes | CASP1 |
| 211368_s_at | 4.8E−04 | 3.8E−05 | 5.3E−03 | 2.2E−03 | Yes | Yes | CASP1 |
| 202763_at | 8.2E−02 | 4.2E−01 | 2.9E−01 | 9.1E−01 | Yes | No | CASP3 |
| 209310_s_at | 1.5E−03 | 6.0E−03 | 6.1E−04 | 2.0E−03 | Yes | Yes | CASP4 |
| 213596_at | 1.7E−01 | 3.7E−01 | 3.1E−01 | 9.1E−01 | Yes | No | CASP4 |
| 209790_s_at | 1.1E−04 | 5.7E−03 | 1.7E−03 | 5.6E−03 | Yes | Yes | CASP6 |
| 211464_x_at | 7.9E−03 | 8.8E−02 | 1.2E−02 | 5.8E−02 | Yes | No | CASP6 |
| 222201_s_at | 8.5E−01 | 2.8E−01 | 8.9E−01 | 2.4E−01 | Yes | No | CASP8AP2 |
| 221188_s_at | 3.6E−02 | 9.0E−03 | 4.3E−02 | 1.3E−01 | Yes | No | CIDEB |
| 209833_at | 2.0E−02 | 2.6E−02 | 3.2E−02 | 2.9E−02 | Yes | No | CRADD |
| 201111_at | 6.3E−02 | 9.8E−01 | 1.4E−02 | 8.2E−01 | Yes | No | CSE1L |
| 201112_s_at | 2.5E−02 | 2.8E−01 | 1.3E−01 | 2.0E−01 | Yes | No | CSE1L |
| 210766_s_at | 1.2E−01 | 2.5E−01 | 1.5E−01 | 8.4E−02 | Yes | No | CSE1L |
| 202468_s_at | 6.5E−03 | 9.5E−01 | 2.4E−03 | 2.1E−01 | Yes | No | CTNNAL1 |
| 208905_at | 3.2E−02 | 1.6E−01 | 2.7E−02 | 1.6E−01 | Yes | No | CYCS |
| 201095_at | 3.7E−01 | 8.5E−02 | 2.3E−02 | 8.9E−01 | Yes | No | DAP |
| 208822_s_at | 9.5E−02 | 1.4E−01 | 4.2E−01 | 2.8E−01 | Yes | No | DAP3 |
| 203890_s_at | 1.5E−01 | 7.6E−01 | 8.4E−01 | 3.9E−01 | Yes | No | DAPK3 |
| 203891_s_at | 4.3E−01 | 5.7E−01 | 9.5E−01 | 8.6E−01 | Yes | No | DAPK3 |
| 217840_at | 5.0E−01 | 2.2E−01 | 6.6E−01 | 3.1E−01 | Yes | No | DDX41 |
| 202480_s_at | 1.0E−04 | 8.0E−03 | 2.7E−03 | 5.3E−02 | Yes | No | DEDD |
| 211255_x_at | 4.3E−02 | 2.5E−03 | 1.7E−02 | 4.4E−03 | Yes | No | DEDD |
| 215158_s_at | 8.7E−03 | 1.8E−03 | 1.3E−03 | 4.2E−03 | Yes | Yes | DEDD |
| 203277_at | 8.8E−02 | 7.4E−01 | 1.4E−01 | 8.5E−01 | Yes | No | DFFA |
| 219350_s_at | 4.2E−01 | 3.8E−02 | 3.8E−01 | 1.9E−02 | Yes | No | DIABLO |
| 205554_s_at | 4.0E−01 | 1.5E−02 | 2.7E−01 | 5.2E−01 | Yes | No | DNASE1L3 |
| 209831_x_at | 8.3E−01 | 1.1E−01 | 9.7E−01 | 5.6E−02 | Yes | No | DNASE2 |
| 214992_s_at | 7.4E−01 | 1.0E−01 | 5.6E−01 | 1.3E−02 | Yes | No | DNASE2 |
| 208289_s_at | 1.6E−01 | 8.6E−01 | 2.5E−01 | 6.5E−01 | Yes | No | EI24 |
| 216396_s_at | 1.0E−01 | 3.3E−01 | 1.0E−01 | 3.3E−01 | Yes | No | EI24 |
| 202535_at | 8.6E−01 | 5.8E−02 | 1.4E−01 | 1.1E−01 | Yes | No | FADD |
| 218080_x_at | 5.5E−03 | 2.7E−02 | 3.3E−03 | 2.2E−02 | Yes | No | FAF1 |
| 202723_s_at | 1.8E−04 | 7.7E−02 | 2.8E−02 | 1.2E−01 | Yes | No | FOXO1A |
| 202724_s_at | 4.8E−03 | 1.7E−01 | 1.0E−02 | 4.2E−01 | Yes | No | FOXO1A |
| 204132_s_at | 3.2E−01 | 2.0E−01 | 1.3E−01 | 6.7E−01 | Yes | No | FOXO3A |
| 201635_s_at | 9.6E−01 | 4.2E−01 | 5.9E−01 | 8.3E−01 | Yes | No | FXR1 |
| 201636_at | 2.6E−01 | 8.2E−01 | 3.1E−01 | 9.1E−01 | Yes | No | FXR1 |
| 201637_s_at | 4.4E−01 | 3.0E−01 | 8.6E−01 | 6.9E−01 | Yes | No | FXR1 |
| 203725_at | 3.6E−02 | 3.5E−02 | 2.0E−02 | 1.8E−02 | Yes | No | GADD45A |
| 207574_s_at | 8.1E−02 | 3.3E−01 | 3.6E−02 | 8.8E−01 | Yes | No | GADD45B |
| 209304_x_at | 1.3E−01 | 2.8E−01 | 6.8E−03 | 2.4E−01 | Yes | No | GADD45B |
| 209305_s_at | 1.2E−01 | 3.4E−01 | 1.2E−01 | 4.1E−01 | Yes | No | GADD45B |
| 214467_at | 4.4E−02 | 3.9E−01 | 2.8E−01 | 2.8E−01 | Yes | No | GPR65 |
| 220864_s_at | 4.4E−01 | 1.8E−03 | 9.0E−01 | 1.0E−02 | Yes | No | GRIM19 |
| 206864_s_at | 6.1E−02 | 3.2E−02 | 8.5E−03 | 3.5E−03 | Yes | No | HRK |
| 206865_at | 1.1E−02 | 1.8E−02 | 6.5E−02 | 6.1E−02 | Yes | No | HRK |
| 207180_s_at | 6.7E−03 | 9.8E−02 | 3.9E−03 | 9.0E−02 | Yes | No | HTATIP2 |
| 209448_at | 5.1E−05 | 6.9E−01 | 1.9E−04 | 2.3E−01 | Yes | No | HTATIP2 |
| 210253_at | 7.9E−03 | 1.7E−01 | 1.5E−02 | 1.6E−01 | Yes | No | HTATIP2 |
| 209929_s_at | 7.6E−01 | 7.5E−02 | 5.9E−02 | 4.6E−01 | Yes | No | IKBKG |
| 36004_at | 8.4E−01 | 8.5E−01 | 7.1E−01 | 4.5E−01 | Yes | No | IKBKG |
| 203836_s_at | 8.2E−02 | 5.9E−01 | 6.9E−02 | 2.7E−01 | Yes | No | MAP3K5 |
| 203837_at | 5.5E−02 | 1.6E−01 | 2.3E−02 | 8.1E−01 | Yes | No | MAP3K5 |
| 202086_at | 2.5E−01 | 3.4E−01 | 9.3E−01 | 8.7E−01 | Yes | No | MX1 |
| 207738_s_at | 5.3E−02 | 1.9E−01 | 2.1E−02 | 2.7E−01 | Yes | No | NCKAP1 |
| 217465_at | 9.1E−01 | 2.0E−01 | 8.5E−01 | 4.4E−01 | Yes | No | NCKAP1 |
| 201502_s_at | 8.8E−01 | 1.3E−01 | 4.7E−01 | 5.5E−02 | Yes | No | NFKBIA |
| 204862_s_at | 7.1E−03 | 8.4E−03 | 4.2E−03 | 6.2E−03 | Yes | No | NME3 |
| 205851_at | 1.1E−01 | 6.4E−02 | 2.4E−02 | 8.1E−03 | Yes | No | NME6 |
| 204004_at | 3.4E−03 | 3.4E−01 | 4.2E−02 | 2.1E−01 | Yes | No | PAWR |
| 204005_s_at | 4.2E−02 | 6.1E−01 | 7.6E−02 | 8.0E−02 | Yes | No | PAWR |
| 204025_s_at | 2.3E−01 | 4.3E−01 | 1.2E−01 | 4.1E−01 | Yes | No | PDCD2 |
| 213581_at | 1.1E−02 | 2.7E−02 | 6.1E−03 | 1.5E−02 | Yes | No | PDCD2 |
| 202730_s_at | 1.8E−01 | 3.2E−01 | 3.0E−01 | 5.3E−01 | Yes | No | PDCD4 |
| 202731_at | 2.7E−01 | 3.1E−01 | 1.6E−01 | 2.7E−01 | Yes | No | PDCD4 |
| 212593_s_at | 2.1E−01 | 1.7E−01 | 5.5E−02 | 2.0E−01 | Yes | No | PDCD4 |
| 212594_at | 9.0E−02 | 8.0E−01 | 1.0E−02 | 5.3E−01 | Yes | No | PDCD4 |
| 219275_at | 5.6E−01 | 6.9E−01 | 9.5E−01 | 5.8E−01 | Yes | No | PDCD5 |
| 203415_at | 6.7E−01 | 9.4E−01 | 2.2E−02 | 4.3E−02 | Yes | No | PDCD6 |
| 217746_s_at | 9.2E−01 | 1.3E−01 | 1.8E−01 | 3.4E−02 | Yes | No | PDCD6IP |
| 205512_s_at | 7.9E−02 | 6.7E−01 | 3.7E−02 | 7.0E−01 | Yes | No | PDCD8 |
| 207002_s_at | 6.9E−02 | 5.4E−02 | 6.4E−01 | 5.8E−01 | Yes | No | PLAGL1 |
| 209318_x_at | 5.6E−01 | 1.8E−01 | 7.6E−01 | 7.9E−01 | Yes | No | PLAGL1 |
| 216347_s_at | 6.8E−01 | 6.5E−01 | 2.3E−01 | 8.9E−01 | Yes | No | PPP1R13B |
| 203089_s_at | 3.0E−02 | 4.5E−02 | 1.3E−02 | 2.1E−02 | Yes | No | PRSS25 |
| 211152_s_at | 4.0E−02 | 5.4E−02 | 5.6E−03 | 1.2E−02 | Yes | No | PRSS25 |
| 209941_at | 9.7E−01 | 9.0E−02 | 2.0E−01 | 1.6E−02 | Yes | No | RIPK1 |
| 209544_at | 5.3E−01 | 2.6E−01 | 9.8E−01 | 9.8E−02 | Yes | No | RIPK2 |
| 209545_s_at | 1.2E−01 | 3.6E−01 | 2.0E−02 | 1.4E−01 | Yes | No | RIPK2 |
| 218286_s_at | 2.1E−01 | 6.7E−01 | 2.2E−01 | 2.4E−01 | Yes | No | RNF7 |
| 203489_at | 5.8E−03 | 4.1E−02 | 9.7E−04 | 3.2E−03 | Yes | No | SIVA |
| 210792_x_at | 3.7E−02 | 1.5E−01 | 2.4E−05 | 4.0E−02 | Yes | No | SIVA |
| 222030_at | 8.1E−01 | 6.7E−02 | 7.1E−01 | 3.7E−02 | Yes | No | SIVA |
| 202693_s_at | 6.2E−02 | 1.5E−02 | 2.5E−02 | 9.4E−03 | Yes | No | STK17A |
| 202695_s_at | 9.0E−01 | 2.1E−04 | 5.6E−01 | 1.5E−04 | Yes | No | STK17A |
| 205214_at | 3.5E−02 | 9.7E−03 | 9.1E−04 | 1.1E−02 | Yes | No | STK17B |
| 200976_s_at | 2.7E−02 | 6.4E−01 | 4.8E−03 | 5.9E−02 | Yes | No | TAX1BP1 |
| 200977_s_at | 6.7E−03 | 8.6E−01 | 3.8E−02 | 3.8E−01 | Yes | No | TAX1BP1 |
| 213786_at | 4.0E−01 | 6.1E−01 | 1.3E−01 | 7.4E−01 | Yes | No | TAX1BP1 |
| 201446_s_at | 3.7E−01 | 1.5E−02 | 6.1E−01 | 8.0E−02 | Yes | No | TIA1 |
| 201447_at | 1.2E−01 | 7.7E−02 | 9.2E−01 | 4.0E−01 | Yes | No | TIA1 |
| 201448_at | 2.2E−01 | 1.8E−01 | 9.4E−01 | 6.0E−01 | Yes | No | TIA1 |
| 201449_at | 5.9E−01 | 4.3E−02 | 6.7E−01 | 1.3E−01 | Yes | No | TIA1 |
| 201450_s_at | 4.5E−01 | 1.5E−01 | 3.5E−01 | 1.8E−01 | Yes | No | TIA1 |
| 217052_x_at | 3.0E−01 | 2.1E−01 | 9.7E−02 | 7.7E−02 | Yes | No | TIA1 |
| 217164_at | 1.6E−01 | 2.3E−01 | 2.1E−01 | 3.3E−01 | Yes | No | TIA1 |
| 202406_s_at | 6.7E−01 | 1.2E−01 | 4.8E−01 | 7.8E−02 | Yes | No | TIAL1 |
| 217500_at | 5.6E−01 | 5.1E−01 | 4.9E−01 | 3.0E−01 | Yes | No | TIAL1 |
| 207643_s_at | 3.2E−01 | 5.6E−03 | 1.8E−01 | 2.6E−03 | Yes | No | TNFRSF1A |
| 215346_at | 1.6E−01 | 6.1E−02 | 1.5E−01 | 7.0E−02 | Yes | No | TNFRSF5 |
| 35150_at | 2.5E−01 | 1.3E−01 | 8.9E−02 | 6.3E−02 | Yes | No | TNFRSF5 |
| 204780_s_at | 1.0E−01 | 5.7E−02 | 5.1E−02 | 6.6E−01 | Yes | No | TNFRSF6 |
| 215719_x_at | 2.9E−01 | 1.4E−01 | 1.3E−01 | 8.9E−01 | Yes | No | TNFRSF6 |
| 216252_x_at | 1.1E−01 | 1.0E−01 | 8.6E−03 | 4.0E−01 | Yes | No | TNFRSF6 |
| 206150_at | 1.3E−02 | 7.4E−02 | 1.9E−02 | 7.5E−02 | Yes | No | TNFRSF7 |
| 207536_s_at | 9.7E−01 | 9.3E−01 | 6.4E−01 | 5.0E−01 | Yes | No | TNFRSF9 |
| 202687_s_at | 2.2E−02 | 3.8E−02 | 1.4E−03 | 6.6E−04 | Yes | No | TNFSF10 |
| 202688_at | 1.7E−04 | 3.6E−04 | 2.5E−02 | 1.8E−02 | Yes | No | TNFSF10 |
| 214329_x_at | 6.8E−02 | 1.1E−01 | 3.2E−02 | 7.6E−02 | Yes | No | TNFSF10 |
| 206508_at | 1.6E−02 | 1.2E−01 | 1.0E−02 | 2.1E−01 | Yes | No | TNFSF7 |
| 207216_at | 7.1E−03 | 3.6E−01 | 6.7E−03 | 6.7E−01 | Yes | No | TNFSF8 |
| 206907_at | 5.9E−02 | 6.2E−02 | 4.9E−02 | 4.4E−02 | Yes | No | TNFSF9 |
| 203120_at | 2.8E−03 | 6.3E−04 | 2.1E−02 | 1.5E−03 | Yes | No | TP53BP2 |
| 209863_s_at | 6.4E−02 | 4.2E−01 | 4.3E−02 | 2.9E−01 | Yes | No | TP73L |
| 211193_at | 9.3E−01 | 7.6E−01 | 3.8E−01 | 1.0E−01 | Yes | No | TP73L |
| 1729_at | 8.2E−02 | 2.1E−01 | 1.3E−01 | 3.6E−01 | Yes | No | TRADD |
| 205599_at | 1.5E−01 | 1.6E−01 | 8.5E−02 | 7.2E−02 | Yes | No | TRAF1 |
| 208315_x_at | 8.4E−02 | 1.4E−02 | 6.4E−02 | 1.2E−01 | Yes | No | TRAF3 |
| 221571_at | 1.3E−03 | 1.3E−01 | 5.2E−03 | 9.5E−03 | Yes | No | TRAF3 |
| 202871_at | 1.2E−02 | 6.0E−02 | 1.3E−01 | 1.4E−01 | Yes | No | TRAF4 |
| 211899_s_at | 2.0E−02 | 2.5E−01 | 1.6E−02 | 1.2E−01 | Yes | No | TRAF4 |
| 204352_at | 1.8E−01 | 2.5E−01 | 9.0E−03 | 8.9E−01 | Yes | No | TRAF5 |
| 207953_at | 7.8E−01 | 3.4E−01 | 9.5E−01 | 5.4E−01 | No | No | AD7C-NTP |
| 208014_x_at | 6.3E−01 | 2.0E−01 | 6.8E−01 | 2.6E−01 | No | No | AD7C-NTP |
| 209364_at | 2.9E−01 | 5.6E−02 | 8.0E−02 | 1.6E−01 | No | No | BAD |
| 221454_at | 9.5E−01 | 3.2E−01 | 3.3E−01 | 5.7E−01 | No | No | BOK |
| 210025_s_at | 9.1E−01 | 5.9E−01 | 9.9E−01 | 4.5E−01 | No | No | CARD10 |
| 210026_s_at | 9.4E−01 | 1.1E−01 | 5.0E−01 | 4.1E−01 | No | No | CARD10 |
| 214207_s_at | 4.1E−01 | 2.5E−02 | 7.8E−01 | 3.8E−01 | No | No | CARD10 |
| 220598_at | 6.1E−01 | 4.6E−01 | 4.7E−01 | 4.2E−01 | No | No | CARD14 |
| 220599_s_at | 4.5E−01 | 3.3E−01 | 5.4E−01 | 3.6E−01 | No | No | CARD14 |
| 220066_at | 1.3E−02 | 6.4E−02 | 5.2E−01 | 1.1E−01 | No | No | CARD15 |
| 221073_s_at | 7.3E−02 | 1.1E−01 | 6.1E−02 | 1.7E−01 | No | No | CARD4 |
| 206011_at | 2.0E−03 | 5.4E−05 | 1.4E−02 | 4.0E−02 | No | No | CASP1 |
| 207500_at | 2.6E−01 | 6.3E−01 | 1.9E−01 | 7.7E−01 | No | No | CASP5 |
| 208791_at | 1.7E−01 | 4.6E−01 | 3.9E−02 | 1.7E−01 | No | No | CLU |
| 208792_s_at | 8.5E−01 | 3.4E−01 | 8.5E−01 | 4.9E−01 | No | No | CLU |
| 222043_at | 7.9E−01 | 4.7E−01 | 1.9E−01 | 4.2E−02 | No | No | CLU |
| 210765_at | 1.4E−01 | 3.4E−02 | 9.5E−02 | 2.8E−01 | No | No | CSE1L |
| 213712_at | 8.7E−01 | 5.2E−01 | 6.5E−01 | 2.0E−01 | No | No | CTNNAL1 |
| 203139_at | 5.5E−01 | 7.5E−01 | 6.3E−01 | 2.9E−01 | No | No | DAPK1 |
| 218325_s_at | 8.0E−01 | 1.6E−01 | 6.0E−01 | 2.3E−01 | No | No | DATF1 |
| 206939_at | 6.7E−01 | 1.8E−01 | 9.9E−01 | 3.4E−01 | No | No | DCC |
| 210165_at | 5.3E−01 | 1.7E−01 | 2.7E−01 | 1.2E−01 | No | No | DNASE1 |
| 210655_s_at | 2.1E−01 | 9.8E−01 | 7.2E−03 | 4.5E−01 | No | No | FOXO3A |
| 213560_at | 4.5E−01 | 3.1E−01 | 9.9E−01 | 9.7E−01 | No | No | GADD45B |
| 204121_at | 1.4E−01 | 2.8E−02 | 5.7E−01 | 1.4E−01 | No | No | GADD45G |
| 205848_at | 3.4E−01 | 7.6E−01 | 8.0E−01 | 7.6E−01 | No | No | GAS2 |
| 208000_at | 8.4E−01 | 1.3E−01 | 6.9E−01 | 8.2E−02 | No | No | GML |
| 205488_at | 7.6E−01 | 1.6E−01 | 6.1E−01 | 1.5E−01 | No | No | GZMA |
| 210164_at | 2.8E−01 | 8.9E−01 | 3.5E−01 | 1.1E−01 | No | No | GZMB |
| 210321_at | 8.6E−01 | 3.6E−01 | 3.2E−01 | 2.2E−01 | No | No | GZMH |
| 206863_x_at | 7.6E−01 | 9.5E−01 | 4.9E−01 | 5.4E−01 | No | No | HRK |
| 207062_at | 4.2E−01 | 9.6E−01 | 2.2E−01 | 4.0E−01 | No | No | IAPP |
| 206975_at | 3.5E−01 | 2.9E−01 | 2.8E−01 | 4.6E−01 | No | No | LTA |
| 203005_at | 7.3E−01 | 4.0E−01 | 6.4E−01 | 7.5E−01 | No | No | LTBR |
| 205858_at | 6.3E−01 | 5.5E−01 | 7.7E−01 | 7.9E−01 | No | No | NGFR |
| 220402_at | 8.4E−01 | 9.2E−01 | 6.3E−01 | 5.4E−01 | No | No | P53AIP1 |
| 220403_s_at | 4.4E−01 | 1.3E−01 | 4.7E−01 | 7.7E−01 | No | No | P53AIP1 |
| 214090_at | 9.1E−01 | 4.7E−01 | 7.2E−02 | 1.0E−01 | No | No | PAWR |
| 214237_x_at | 8.2E−01 | 7.7E−02 | 7.8E−01 | 1.5E−01 | No | No | PAWR |
| 207634_at | 6.2E−01 | 6.6E−01 | 9.7E−01 | 9.6E−01 | No | No | PDCD1 |
| 213585_s_at | 8.5E−01 | 8.7E−01 | 3.0E−01 | 4.3E−01 | No | No | PDCD2 |
| 222152_at | 8.3E−01 | 4.0E−01 | 2.2E−01 | 2.5E−01 | No | No | PDCD6 |
| 207943_x_at | 7.6E−01 | 5.0E−01 | 9.0E−01 | 4.5E−01 | No | No | PLAGL1 |
| 218849_s_at | 2.3E−01 | 1.9E−01 | 2.1E−01 | 1.9E−01 | No | No | RAI |
| 207468_s_at | 1.6E−01 | 1.9E−01 | 4.8E−01 | 5.7E−01 | No | No | SFRP5 |
| 202694_at | 2.9E−01 | 6.7E−01 | 6.1E−01 | 1.5E−01 | No | No | STK17A |
| 206222_at | 7.5E−01 | 4.2E−01 | 5.5E−01 | 5.0E−01 | No | No | TNFRSF10C |
| 211163_s_at | 8.2E−01 | 1.1E−01 | 9.2E−01 | 1.7E−01 | No | No | TNFRSF10C |
| 210654_at | 8.5E−01 | 5.2E−01 | 4.9E−01 | 9.8E−01 | No | No | TNFRSF10D |
| 210847_x_at | 6.5E−01 | 2.2E−01 | 5.3E−01 | 5.1E−01 | No | No | TNFRSF25 |
| 211282_x_at | 4.2E−01 | 1.9E−01 | 9.9E−01 | 2.6E−01 | No | No | TNFRSF25 |
| 211841_s_at | 4.3E−01 | 2.5E−01 | 4.7E−01 | 5.6E−01 | No | No | TNFRSF25 |
| 216042_at | 3.3E−01 | 3.1E−01 | 2.5E−01 | 3.5E−01 | No | No | TNFRSF25 |
| 219422_at | 1.5E−01 | 3.7E−01 | 6.1E−01 | 8.0E−01 | No | No | TNFRSF25 |
| 219423_x_at | 5.8E−01 | 6.7E−01 | 7.7E−01 | 2.0E−01 | No | No | TNFRSF25 |
| 205153_s_at | 1.0E−01 | 4.9E−02 | 2.3E−02 | 1.1E−02 | No | No | TNFRSF5 |
| 222292_at | 2.1E−01 | 4.4E−01 | 4.1E−01 | 6.6E−02 | No | No | TNFRSF5 |
| 204781_s_at | 4.1E−02 | 9.5E−01 | 5.2E−02 | 3.3E−01 | No | No | TNFRSF6 |
| 211786_at | 2.9E−01 | 2.0E−02 | 9.3E−01 | 8.0E−02 | No | No | TNFRSF9 |
| 207907_at | 6.4E−01 | 1.4E−01 | 8.7E−01 | 2.1E−01 | No | No | TNFSF14 |
| 207892_at | 9.8E−01 | 2.2E−01 | 9.7E−01 | 1.8E−01 | No | No | TNFSF5 |
| 210865_at | 4.0E−01 | 8.3E−01 | 5.6E−01 | 1.8E−01 | No | No | TNFSF6 |
| 211333_s_at | 2.2E−01 | 5.8E−01 | 1.7E−03 | 3.2E−01 | No | No | TNFSF6 |
| 220804_s_at | 2.1E−01 | 5.2E−02 | 7.5E−01 | 1.4E−01 | No | No | TP73 |
| 207382_at | 2.6E−01 | 9.7E−01 | 4.9E−01 | 3.5E−01 | No | No | TP73L |
| 211194_s_at | 2.1E−01 | 62E−01 | 7.4E−02 | 2.2E−01 | No | No | TP73L |
| 211195_s_at | 6.7E−01 | 7.9E−01 | 9.3E−01 | 5.3E−01 | No | No | TP73L |
| 211834_s_at | 4.7E−01 | 8.6E−01 | 6.9E−01 | 6.5E−01 | No | No | TP73L |
| 205641_s_at | 3.3E−01 | 4.4E−01 | 1.8E−01 | 5.2E−01 | No | No | TRADD |
| 213443_at | 4.0E−01 | 4.3E−01 | 2.5E−01 | 4.9E−01 | No | No | TRADD |

| Probesets | UniGene ID | Gene Title |
|---|---|---|
| FDR Cutoff | | |
| 201715_s_at | Hs.227133 | apoptotic chromatin condensation inducer |
| | | in the nucleus |
| 205013_s_at | Hs.197029 | adenosine A2a receptor |
| 204833_at | Hs.264482 | APG12 autophagy 12-like (S. cerevisiae) |
| 213026_at | Hs.264482 | APG12 autophagy 12-like (S. cerevisiae) |
| 213930_at | Hs.264482 | APG12 autophagy 12-like (S. cerevisiae) |
| 221666_s_at | Hs.197875 | apoptosis-associated speck-like protein |
| | | containing a CARD |
| 1861_at | Hs.76366 | BCL2-antagonist of cell death |
| 209406_at | Hs.55220 | BCL2-associated athanogene 2 |
| 217911_s_at | Hs.15259 | BCL2-associated athanogene 3 |
| 202984_s_at | Hs.5443 | BCL2-associated athanogene 5 |
| 202985_s_at | Hs.5443 | BCL2-associated athanogene 5 |
| 203728_at | Hs.93213 | BCL2-antagonist/killer 1 |
| 208478_s_at | Hs.159428 | BCL2-associated X protein |
| 211833_s_at | Hs.159428 | BCL2-associated X protein |
| 205263_at | Hs.193516 | B-cell CLL/lymphoma 10 |
| 208536_s_at | Hs.84063 | BCL2-like 11 (apoptosis facilitator) |
| 222343_at | Hs.84063 | BCL2-like 11 (apoptosis facilitator) |
| 205780_at | Hs.155419 | BCL2-interacting killer (apoptosis-inducing) |
| 220451_s_at | Hs.256126 | baculoviral IAP repeat-containing 7 (livin) |
| 221478_at | Hs.132955 | BCL2/adenovirus E1B 19 kDa interacting |
| | | protein 3-like |
| 221479_s_at | Hs.132955 | BCL2/adenovirus E1B 19 kDa interacting |
| | | protein 3-like |
| 204950_at | Hs.446146 | caspase recruitment domain family, |
| | | member 8 |
| 220162_s_at | Hs.271815 | caspase recruitment domain family, |
| | | member 9 |
| 209970_x_at | Hs.2490 | caspase 1 |
| 211366_x_at | Hs.2490 | caspase 1 |
| 211367_s_at | Hs.2490 | caspase 1 |
| 211368_s_at | Hs.2490 | caspase 1 |
| 202763_at | Hs.141125 | caspase 3 |
| 209310_s_at | Hs.74122 | caspase 4 |
| 213596_at | Hs.74122 | caspase 4 |
| 209790_s_at | Hs.3280 | caspase 6 |
| 211464_x_at | Hs.3280 | caspase 6 |
| 222201_s_at | Hs.122843 | CASP8 associated protein 2 |
| 221188_s_at | Hs.448590 | cell death-inducing DFFA-like effector b |
| 209833_at | Hs.155566 | CASP2 and RIPK1 domain containing |
| | | adaptor with death domain |
| 201111_at | Hs.90073 | CSE1 chromosome segregation 1-like |
| | | (yeast) |
| 201112_s_at | Hs.90073 | CSE1 chromosome segregation 1-like |
| | | (yeast) |
| 210766_s_at | Hs.90073 | CSE1 chromosome segregation 1-like |
| | | (yeast) |
| 202468_s_at | Hs.58488 | catenin (cadherin-associated protein), |
| | | alpha-like 1 |
| 208905_at | Hs.437060 | cytochrome c, somatic |
| 201095_at | Hs.75189 | death-associated protein |
| 208822_s_at | Hs.270920 | death associated protein 3 |
| 203890_s_at | Hs.153908 | death-associated protein kinase 3 |
| 203891_s_at | Hs.153908 | death-associated protein kinase 3 |
| 217840_at | Hs.274317 | DEAD (Asp-Glu-Ala-Asp) box polypeptide |
| | | 41 |
| 202480_s_at | Hs.169681 | death effector domain containing |
| 211255_x_at | Hs.169681 | death effector domain containing |
| 215158_s_at | Hs.169681 | death effector domain containing |
| 203277_at | Hs.484782 | DNA fragmentation factor, 45 kDa, alpha |
| | | polypeptide |
| 219350_s_at | Hs.169611 | diablo homolog (Drosophila) |
| 205554_s_at | Hs.88646 | deoxyribonuclease I-like 3 |
| 209831_x_at | Hs.118243 | deoxyribonuclease II, lysosomal |
| 214992_s_at | Hs.118243 | deoxyribonuclease II, lysosomal |
| 208289_s_at | Hs.343911 | etoposide induced 2.4 mRNA |
| 216396_s_at | Hs.343911 | etoposide induced 2.4 mRNA |
| 202535_at | Hs.86131 | Fas (TNFRSF6)-associated via death |
| | | domain |
| 218080_x_at | Hs.12899 | Fas (TNFRSF6) associated factor 1 |
| 202723_s_at | Hs.170133 | forkhead box O1A (rhabdomyosarcoma) |
| 202724_s_at | Hs.170133 | forkhead box O1A (rhabdomyosarcoma) |
| 204132_s_at | Hs.14845 | forkhead box O3A |
| 201635_s_at | Hs.408096 | fragile X mental retardation, autosomal |
| | | homolog 1 |
| 201636_at | Hs.408096 | fragile X mental retardation, autosomal |
| | | homolog 1 |
| 201637_s_at | Hs.408096 | fragile X mental retardation, autosomal |
| | | homolog 1 |
| 203725_at | Hs.80409 | growth arrest and DNA-damage-inducible, |
| | | alpha |
| 207574_s_at | Hs.110571 | growth arrest and DNA-damage-inducible, |
| | | beta |
| 209304_x_at | Hs.110571 | growth arrest and DNA-damage-inducible, |
| | | beta |
| 209305_s_at | Hs.110571 | growth arrest and DNA-damage-inducible, |
| | | beta |
| 214467_at | Hs.131924 | G protein-coupled receptor 65 |
| 220864_s_at | Hs.279574 | cell death-regulatory protein GRIM19 |
| 206864_s_at | Hs.87247 | harakiri, BCL2 interacting protein (contains |
| | | only BH3 domain) |
| 206865_at | Hs.87247 | harakiri, BCL2 interacting protein (contains |
| | | only BH3 domain) |
| 207180_s_at | Hs.90753 | HIV-1 Tat interactive protein 2, 30 kDa |
| 209448_at | Hs.90753 | HIV-1 Tat interactive protein 2, 30 kDa |
| 210253_at | Hs.90753 | HIV-1 Tat interactive protein 2, 30 kDa |
| 209929_s_at | Hs.43505 | inhibitor of kappa light polypeptide gene |
| | | enhancer in B-cells, kinase gamma |
| 36004_at | Hs.43505 | inhibitor of kappa light polypeptide gene |
| | | enhancer in B-cells, kinase gamma |
| 203836_s_at | Hs.151988 | mitogen-activated protein kinase kinase |
| | | kinase 5 |
| 203837_at | Hs.151988 | mitogen-activated protein kinase kinase |
| | | kinase 5 |
| 202086_at | Hs.436836 | myxovirus (influenza virus) resistance 1 |
| 207738_s_at | Hs.278411 | NCK-associated protein 1 |
| 217465_at | Hs.278411 | NCK-associated protein 1 |
| 201502_s_at | Hs.81328 | nuclear factor of kappa light polypeptide |
| | | gene enhancer in B-cells inhibitor, alpha |
| 204862_s_at | Hs.81687 | non-metastatic cells 3, protein expressed in |
| 205851_at | Hs.465558 | non-metastatic cells 6, protein expressed in |
| | | (nucleoside-diphosphate kinase) |
| 204004_at | Hs.406074 | PRKC, apoptosis, WT1, regulator |
| 204005_s_at | Hs.406074 | PRKC, apoptosis, WT1, regulator |
| 204025_s_at | Hs.367900 | programmed cell death 2 |
| 213581_at | Hs.367900 | programmed cell death 2 |
| 202730_s_at | Hs.257697 | programmed cell death 4 (neoplastic |
| | | transformation inhibitor) |
| 202731_at | Hs.257697 | programmed cell death 4 (neoplastic |
| | | transformation inhibitor) |
| 212593_s_at | Hs.257697 | programmed cell death 4 (neoplastic |
| | | transformation inhibitor) |
| 212594_at | Hs.257697 | programmed cell death 4 (neoplastic |
| | | transformation inhibitor) |
| 219275_at | Hs.443831 | programmed cell death 5 |
| 203415_at | Hs.24087 | programmed cell death 6 |
| 217746_s_at | Hs.9663 | programmed cell death 6 interacting protein |
| 205512_s_at | Hs.18720 | programmed cell death 8 (apoptosis- |
| | | inducing factor) |
| 207002_s_at | Hs.132911 | pleiomorphic adenoma gene-like 1 |
| 209318_x_at | Hs.132911 | pleiomorphic adenoma gene-like 1 |
| 216347_s_at | Hs.371546 | protein phosphatase 1, regulatory |
| | | (inhibitor) subunit 13B |
| 203089_s_at | Hs.115721 | protease, serine, 25 |
| 211152_s_at | Hs.115721 | protease, serine, 25 |
| 209941_at | Hs.390758 | receptor (TNFRSF)-interacting serine- |
| | | threonine kinase 1 |
| 209544_at | Hs.103755 | receptor-interacting serine-threonine kinase 2 |
| 209545_s_at | Hs.103755 | receptor-interacting serine-threonine kinase 2 |
| 218286_s_at | Hs.512849 | ring finger protein 7 |
| 203489_at | Hs.112058 | CD27-binding (Siva) protein |
| 210792_x_at | Hs.112058 | CD27-binding (Siva) protein |
| 222030_at | Hs.112058 | CD27-binding (Siva) protein |
| 202693_s_at | Hs.9075 | serine/threonine kinase 17a (apoptosis- |
| | | inducing) |
| 202695_s_at | Hs.9075 | serine/threonine kinase 17a (apoptosis- |
| | | inducing) |
| 205214_at | Hs.88297 | serine/threonine kinase 17b (apoptosis- |
| | | inducing) |
| 200976_s_at | Hs.5437 | Tax1 (human T-cell leukemia virus type I) |
| | | binding protein 1 |
| 200977_s_at | Hs.5437 | Tax1 (human T-cell leukemia virus type I) |
| | | binding protein 1 |
| 213786_at | Hs.5437 | Tax1 (human T-cell leukemia virus type I) |
| | | binding protein 1 |
| 201446_s_at | Hs.391858 | TIA1 cytotoxic granule-associated RNA |
| | | binding protein |
| 201447_at | Hs.391858 | TIA1 cytotoxic granule-associated RNA |
| | | binding protein |
| 201448_at | Hs.391858 | TIA1 cytotoxic granule-associated RNA |
| | | binding protein |
| 201449_at | Hs.391858 | TIA1 cytotoxic granule-associated RNA |
| | | binding protein |
| 201450_s_at | Hs.391858 | TIA1 cytotoxic granule-associated RNA |
| | | binding protein |
| 217052_x_at | Hs.391858 | TIA1 cytotoxic granule-associated RNA |
| | | binding protein |
| 217164_at | Hs.391858 | TIA1 cytotoxic granule-associated RNA |
| | | binding protein |
| 202406_s_at | Hs.335786 | TIA1 cytotoxic granule-associated RNA |
| | | binding protein-like 1 |
| 217500_at | Hs.335786 | TIA1 cytotoxic granule-associated RNA |
| | | binding protein-like 1 |
| 207643_s_at | Hs.159 | tumor necrosis factor receptor superfamily, |
| | | member 1A |
| 215346_at | Hs.504816 | tumor necrosis factor receptor superfamily, |
| | | member 5 |
| 35150_at | Hs.504816 | tumor necrosis factor receptor superfamily, |
| | | member 5 |
| 204780_s_at | Hs.82359 | tumor necrosis factor receptor superfamily, |
| | | member 6 |
| 215719_x_at | Hs.82359 | tumor necrosis factor receptor superfamily, |
| | | member 6 |
| 216252_x_at | Hs.82359 | tumor necrosis factor receptor superfamily, |
| | | member 6 |
| 206150_at | Hs.355307 | tumor necrosis factor receptor superfamily, |
| | | member 7 |
| 207536_s_at | Hs.193418 | tumor necrosis factor receptor superfamily, |
| | | member 9 |
| 202687_s_at | Hs.387871 | tumor necrosis factor (ligand) superfamily, |
| | | member 10 |
| 202688_at | Hs.387871 | tumor necrosis factor (ligand) superfamily, |
| | | member 10 |
| 214329_x_at | Hs.387871 | tumor necrosis factor (ligand) superfamily, |
| | | member 10 |
| 206508_at | Hs.99899 | tumor necrosis factor (ligand) superfamily, |
| | | member 7 |
| 207216_at | Hs.177136 | tumor necrosis factor (ligand) superfamily, |
| | | member 8 |
| 206907_at | Hs.1524 | tumor necrosis factor (ligand) superfamily, |
| | | member 9 |
| 203120_at | Hs.44585 | tumor protein p53 binding protein, 2 |
| 209863_s_at | Hs.137569 | tumor protein p73-like |
| 211193_at | Hs.137569 | tumor protein p73-like |
| 1729_at | Hs.89862 | TNFRSF1A-associated via death domain |
| 205599_at | Hs.438253 | TNF receptor-associated factor 1 |
| 208315_x_at | Hs.297660 | TNF receptor-associated factor 3 |
| 221571_at | Hs.297660 | TNF receptor-associated factor 3 |
| 202871_at | Hs.8375 | TNF receptor-associated factor 4 |
| 211899_s_at | Hs.8375 | TNF receptor-associated factor 4 |
| 204352_at | Hs.385685 | TNF receptor-associated factor 5 |
| 207953_at | Hs.129735 | neuronal thread protein |
| 208014_x_at | Hs.129735 | neuronal thread protein |
| 209364_at | Hs.76366 | BCL2-antagonist of cell death |
| 221454_at | Hs.293753 /// | BCL2-related ovarian killer /// BCL2-related |
| | Hs.293753 | ovarian killer |
| 210025_s_at | Hs.57973 | caspase recruitment domain family, |
| | | member 10 |
| 210026_s_at | Hs.57973 | caspase recruitment domain family, |
| | | member 10 |
| 214207_s_at | Hs.57973 | caspase recruitment domain family, |
| | | member 10 |
| 220598_at | Hs.306227 | caspase recruitment domain family, |
| | | member 14 |
| 220599_s_at | Hs.306227 | caspase recruitment domain family, |
| | | member 14 |
| 220066_at | Hs.135201 | caspase recruitment domain family, |
| | | member 15 |
| 221073_s_at | Hs.19405 | caspase recruitment domain family, |
| | | member 4 |
| 206011_at | Hs.2490 | caspase 1 |
| 207500_at | Hs.213327 | caspase 5, apoptosis-related cysteine |
| | | protease |
| 208791_at | Hs.436657 | clusterin |
| 208792_s_at | Hs.436657 | clusterin |
| 222043_at | Hs.436657 | clusterin |
| 210765_at | Hs.90073 | CSE1 chromosome segregation 1-like |
| | | (yeast) |
| 213712_at | Hs.58488 | catenin (cadherin-associated protein), |
| | | alpha-like 1 |
| 203139_at | Hs.244318 | death-associated protein kinase 1 |
| 218325_s_at | Hs.438300 | death associated transcription factor 1 |
| 206939_at | Hs.172562 | deleted in colorectal carcinoma |
| 210165_at | Hs.436928 | deoxyribonuclease I |
| 210655_s_at | Hs.14845 | forkhead box O3A |
| 213560_at | Hs.110571 | growth arrest and DNA-damage-inducible, |
| | | beta |
| 204121_at | Hs.9701 | growth arrest and DNA-damage-inducible, |
| | | gamma |
| 205848_at | Hs.135665 | growth arrest-specific 2 |
| 208000_at | Hs.86161 | GPI anchored molecule like protein |
| 205488_at | Hs.90708 | granzyme A |
| 210164_at | Hs.1051 | granzyme B |
| 210321_at | Hs.348264 | granzyme H |
| 206863_x_at | Hs.87247 | harakiri |
| 207062_at | Hs.142255 | islet amyloid polypeptide |
| 206975_at | Hs.36 | lymphotoxin alpha (TNF superfamily, |
| | | member 1) |
| 203005_at | Hs.1116 | lymphotoxin beta receptor (TNFR |
| | | superfamily, member 3) |
| 205858_at | Hs.415768 | nerve growth factor receptor (TNFR |
| | | superfamily, member 16) |
| 220402_at | Hs.160953 | p53-regulated apoptosis-inducing protein 1 |
| 220403_s_at | Hs.160953 | p53-regulated apoptosis-inducing protein 1 |
| 214090_at | Hs.406074 | PRKC, apoptosis, WT1, regulator |
| 214237_x_at | Hs.406074 | PRKC, apoptosis, WT1, regulator |
| 207634_at | Hs.158297 | programmed cell death 1 |
| 213585_s_at | Hs.367900 | programmed cell death 2 |
| 222152_at | Hs.24087 | programmed cell death 6 |
| 207943_x_at | Hs.132911 | pleiomorphic adenoma gene-like 1 |
| 218849_s_at | Hs.324051 | ReIA-associated inhibitor |
| 207468_s_at | Hs.279565 | secreted frizzled-related protein 5 |
| 202694_at | Hs.9075 | serine/threonine kinase 17a (apoptosis- |
| | | inducing) |
| 206222_at | Hs.119684 | tumor necrosis factor receptor superfamily, |
| | | member 10c, decoy without an intracellular |
| | | domain |
| 211163_s_at | Hs.119684 | tumor necrosis factor receptor superfamily, |
| | | member 10c, decoy without an intracellular |
| | | domain |
| 210654_at | Hs.129844 | tumor necrosis factor receptor superfamily, |
| | | member 10d, decoy with truncated death |
| | | domain |
| 210847_x_at | Hs.299558 | tumor necrosis factor receptor superfamily, |
| | | member 25 |
| 211282_x_at | Hs.299558 | tumor necrosis factor receptor superfamily, |
| | | member 25 |
| 211841_s_at | Hs.299558 | tumor necrosis factor receptor superfamily, |
| | | member 25 |
| 216042_at | Hs.299558 | tumor necrosis factor receptor superfamily, |
| | | member 25 |
| 219422_at | Hs.299558 | tumor necrosis factor receptor superfamily, |
| | | member 25 |
| 219423_x_at | Hs.299558 | tumor necrosis factor receptor superfamily, |
| | | member 25 |
| 205153_s_at | Hs.504816 | tumor necrosis factor receptor superfamily, |
| | | member 5 |
| 222292_at | Hs.504816 | tumor necrosis factor receptor superfamily, |
| | | member 5 |
| 204781_s_at | Hs.82359 | tumor necrosis factor receptor superfamily, |
| | | member 6 |
| 211786_at | Hs.193418 /// | tumor necrosis factor receptor superfamily, |
| | Hs.193418 | member 9 |
| 207907_at | Hs.129708 | tumor necrosis factor (ligand) superfamily, |
| | | member 14 |
| 207892_at | Hs.652 | tumor necrosis factor (ligand) superfamily, |
| | | member 5 (hyper-IgM syndrome) |
| 210865_at | Hs.2007 | tumor necrosis factor (ligand) superfamily, |
| | | member 6 |
| 211333_s_at | Hs.2007 | tumor necrosis factor (ligand) superfamily, |
| | | member 6 |
| 220804_s_at | Hs.192132 | tumor protein p73 |
| 207382_at | Hs.137569 | tumor protein p73-like |
| 211194_s_at | Hs.137569 | tumor protein p73-like |
| 211195_s_at | Hs.137569 | tumor protein p73-like |
| 211834_s_at | Hs.137569 | tumor protein p73-like |
| 205641_s_at | Hs.89862 | TNFRSF1A-associated via death domain |
| 213443_at | Hs.89862 | TNFRSF1A-associated via death domain |

TABLE 6A
Anti-apoptosis Genes (See tables reference 2)

| | Log2 fluorescence intensity | | | | | | | | | | | |
|---|---|---|---|---|---|---|---|---|---|---|---|---|
| | Ly1 | | Ly1 | | Ly7 | | | | | | | |
| Probesets | (A1) | Ly1 (A2) | (A3) | Ly7 (B1) | (B2) | Ly7 (B3) | Ly8 (C1) | Ly8 (C2) | Ly8 (C3) | D4 (D1) | D4 (D2) | D4 (D3) |
| 209165_at | 8.62 | 8.82 | 8.67 | 9.02 | 9.04 | 9.05 | 8.63 | 8.75 | 8.87 | 8.79 | 8.83 | 8.88 |
| 207163_s_at | 8.37 | 8.44 | 8.22 | 8.02 | 7.99 | 7.73 | 8.91 | 8.88 | 9.02 | 7.82 | 8.07 | 8.05 |
| 219366_at | 6.89 | 7.05 | 7.18 | 7.20 | 7.26 | 7.11 | 6.72 | 6.91 | 7.16 | 7.39 | 7.24 | 7.57 |
| 202387_at | 8.52 | 8.61 | 8.78 | 8.20 | 8.16 | 8.25 | 8.52 | 8.55 | 8.67 | 8.88 | 9.19 | 9.28 |
| 211475_s_at | 8.87 | 9.19 | 9.17 | 8.66 | 8.62 | 8.74 | 8.86 | 9.00 | 9.14 | 9.48 | 9.71 | 9.88 |
| 219624_at | 4.35 | 4.34 | 4.42 | 4.54 | 4.47 | 4.64 | 4.35 | 4.41 | 4.63 | 4.53 | 4.44 | 4.65 |
| 203685_at | 4.75 | 4.61 | 4.79 | 4.33 | 4.46 | 4.42 | 4.62 | 4.65 | 4.68 | 4.48 | 4.58 | 4.41 |
| 207005_s_at | 6.62 | 6.60 | 6.62 | 6.35 | 6.12 | 5.92 | 7.06 | 6.73 | 6.62 | 6.53 | 6.85 | 6.23 |
| 205681_at | 4.31 | 4.32 | 4.29 | 4.59 | 4.61 | 4.49 | 4.45 | 4.48 | 4.30 | 6.84 | 8.14 | 7.47 |
| 206665_s_at | 4.49 | 4.59 | 4.55 | 4.82 | 4.99 | 4.84 | 4.70 | 4.49 | 4.54 | 4.63 | 4.57 | 4.51 |
| 212312_at | 5.94 | 6.29 | 6.48 | 6.61 | 6.93 | 6.42 | 6.13 | 6.05 | 6.30 | 6.07 | 6.30 | 6.33 |
| 215037_s_at | 6.26 | 6.14 | 6.06 | 6.32 | 6.32 | 6.15 | 6.21 | 6.00 | 5.84 | 5.72 | 6.04 | 5.80 |
| 209311_at | 5.34 | 5.42 | 5.37 | 5.36 | 5.30 | 5.48 | 5.26 | 5.52 | 5.31 | 5.13 | 5.13 | 5.11 |
| 208945_s_at | 7.70 | 7.53 | 7.75 | 7.37 | 7.22 | 7.31 | 7.78 | 7.50 | 7.93 | 7.41 | 7.17 | 7.54 |
| 208946_s_at | 8.10 | 7.84 | 8.04 | 7.39 | 7.20 | 7.16 | 8.03 | 8.06 | 8.09 | 7.74 | 7.43 | 7.93 |
| 202076_at | 8.63 | 8.09 | 8.59 | 8.79 | 8.32 | 8.73 | 8.67 | 8.13 | 8.76 | 7.56 | 7.54 | 8.28 |
| 210538_s_at | 4.64 | 4.75 | 4.80 | 6.89 | 6.65 | 6.57 | 4.49 | 4.35 | 4.62 | 4.95 | 5.43 | 5.21 |
| 206536_s_at | 4.28 | 4.41 | 4.82 | 4.26 | 4.40 | 4.42 | 4.42 | 4.48 | 4.62 | 4.25 | 4.44 | 4.33 |
| 202094_at | 8.05 | 8.01 | 7.73 | 8.69 | 8.38 | 8.37 | 7.94 | 8.10 | 8.08 | 8.54 | 8.31 | 8.45 |
| 202095_s_at | 9.08 | 9.07 | 8.69 | 8.95 | 8.59 | 8.79 | 8.92 | 8.79 | 9.01 | 9.52 | 9.38 | 9.59 |
| 210334_x_at | 8.24 | 8.19 | 7.87 | 8.34 | 8.21 | 8.27 | 8.13 | 7.97 | 8.29 | 8.75 | 8.54 | 8.49 |
| 220451_s_at | 5.05 | 4.97 | 4.89 | 5.15 | 5.11 | 5.03 | 4.92 | 5.26 | 4.92 | 5.41 | 5.18 | 5.20 |
| 204930_s_at | 6.45 | 6.22 | 6.30 | 6.33 | 6.24 | 6.40 | 6.46 | 6.44 | 6.50 | 6.61 | 6.40 | 6.44 |
| 207829_s_at | 6.53 | 6.20 | 6.04 | 6.06 | 5.97 | 5.91 | 6.29 | 5.82 | 6.14 | 6.09 | 6.25 | 6.11 |
| 37226_at | 6.07 | 5.86 | 5.75 | 5.62 | 5.75 | 5.71 | 5.90 | 5.81 | 5.90 | 5.82 | 5.76 | 5.83 |
| 209308_s_at | 7.78 | 7.94 | 8.01 | 8.21 | 8.48 | 8.42 | 7.82 | 7.98 | 8.09 | 7.73 | 7.51 | 7.99 |
| 201848_s_at | 8.63 | 6.93 | 7.89 | 7.57 | 7.62 | 7.52 | 9.02 | 7.71 | 8.66 | 6.81 | 6.87 | 7.16 |
| 201849_at | 9.01 | 7.37 | 8.80 | 8.63 | 8.71 | 8.80 | 9.58 | 8.66 | 9.43 | 7.94 | 7.51 | 8.25 |
| 206044_s_at | 5.45 | 5.87 | 5.68 | 5.76 | 5.94 | 5.90 | 5.59 | 5.79 | 5.66 | 5.82 | 5.86 | 5.71 |
| 208485_x_at | 5.64 | 5.22 | 5.33 | 5.39 | 5.51 | 5.40 | 5.30 | 5.36 | 5.30 | 5.31 | 5.25 | 4.96 |
| 209508_x_at | 6.13 | 6.12 | 6.08 | 6.12 | 6.31 | 5.96 | 6.16 | 6.13 | 6.03 | 5.98 | 5.95 | 5.74 |
| 209939_x_at | 6.86 | 6.88 | 7.01 | 7.80 | 7.75 | 7.78 | 6.85 | 7.01 | 7.04 | 6.40 | 6.16 | 6.11 |
| 210563_x_at | 6.61 | 6.84 | 6.71 | 7.62 | 7.36 | 7.39 | 6.74 | 6.73 | 6.85 | 6.59 | 6.34 | 6.51 |
| 211316_x_at | 6.52 | 6.42 | 6.35 | 6.52 | 6.68 | 6.33 | 6.48 | 6.64 | 6.28 | 6.06 | 5.97 | 5.95 |
| 211317_s_at | 4.50 | 4.60 | 4.70 | 4.66 | 4.65 | 4.71 | 4.51 | 4.60 | 4.59 | 4.42 | 4.51 | 4.47 |
| 211862_x_at | 5.34 | 5.18 | 5.07 | 5.44 | 5.23 | 5.21 | 5.02 | 5.04 | 5.05 | 4.80 | 4.82 | 4.56 |
| 214618_at | 4.41 | 4.69 | 4.77 | 4.70 | 4.55 | 4.85 | 4.68 | 4.69 | 4.73 | 4.81 | 4.98 | 4.74 |
| 200046_at | 9.67 | 9.39 | 9.48 | 9.81 | 9.69 | 9.87 | 9.64 | 9.29 | 9.63 | 10.02 | 10.12 | 10.22 |
| 208309_s_at | 6.42 | 6.54 | 6.55 | 6.69 | 6.71 | 6.79 | 6.49 | 6.49 | 6.56 | 7.10 | 7.29 | 7.23 |
| 210017_at | 4.77 | 4.61 | 4.96 | 5.15 | 5.09 | 5.35 | 4.57 | 4.64 | 4.90 | 5.35 | 5.34 | 5.91 |
| 210018_x_at | 6.59 | 6.48 | 6.63 | 6.63 | 6.76 | 6.77 | 6.25 | 6.20 | 6.68 | 6.95 | 7.11 | 7.28 |
| 209239_at | 8.35 | 8.59 | 8.52 | 7.22 | 7.63 | 7.16 | 8.37 | 8.51 | 8.19 | 8.15 | 8.49 | 8.00 |
| 201739_at | 5.51 | 5.60 | 5.45 | 7.04 | 6.57 | 6.94 | 5.46 | 5.43 | 5.43 | 6.23 | 6.92 | 6.69 |
| 218878_s_at | 7.86 | 7.41 | 7.82 | 8.01 | 7.75 | 8.14 | 7.74 | 7.38 | 7.89 | 7.59 | 7.24 | 7.67 |
| 207827_x_at | 4.96 | 5.09 | 5.07 | 5.42 | 5.36 | 5.17 | 4.94 | 5.48 | 5.04 | 5.37 | 5.08 | 5.18 |
| 211546_x_at | 4.85 | 5.05 | 4.98 | 5.20 | 5.08 | 4.95 | 5.09 | 5.24 | 4.98 | 5.35 | 5.11 | 5.12 |
| 201085_s_at | 7.33 | 7.39 | 8.10 | 7.28 | 7.32 | 7.53 | 7.50 | 7.44 | 7.87 | 6.48 | 7.17 | 7.28 |
| 201086_x_at | 9.82 | 9.89 | 9.88 | 9.81 | 9.67 | 9.54 | 9.87 | 9.88 | 9.69 | 9.29 | 9.29 | 9.40 |
| 213538_at | 7.15 | 7.26 | 7.53 | 7.58 | 7.73 | 7.69 | 7.04 | 7.29 | 7.30 | 6.94 | 7.14 | 7.32 |
| 214988_s_at | 9.49 | 9.56 | 9.84 | 9.65 | 9.46 | 9.44 | 9.60 | 9.67 | 9.60 | 9.02 | 9.09 | 9.32 |
| 200803_s_at | 9.79 | 9.57 | 9.21 | 9.70 | 9.80 | 9.80 | 9.59 | 9.50 | 9.51 | 10.10 | 10.30 | 10.17 |
| 200804_at | 9.84 | 9.29 | 9.26 | 9.40 | 9.38 | 9.24 | 9.62 | 9.39 | 9.29 | 9.75 | 10.02 | 10.00 |
| 202643_s_at | 5.55 | 5.58 | 5.49 | 6.45 | 6.41 | 6.37 | 5.45 | 5.43 | 5.12 | 5.70 | 5.71 | 5.85 |
| 202644_s_at | 5.97 | 6.21 | 6.10 | 7.05 | 7.32 | 7.10 | 5.88 | 6.03 | 5.70 | 6.31 | 6.63 | 6.37 |
| 206092_x_at | 6.40 | 6.62 | 6.47 | 6.78 | 6.77 | 6.53 | 6.47 | 6.53 | 6.28 | 6.44 | 6.53 | 6.29 |
| 206467_x_at | 5.20 | 5.29 | 5.20 | 5.60 | 5.61 | 5.43 | 4.97 | 5.54 | 5.21 | 5.31 | 5.45 | 5.40 |
| 213829_x_at | 6.99 | 7.06 | 7.06 | 6.75 | 7.16 | 6.99 | 7.16 | 7.21 | 6.91 | 7.00 | 7.19 | 6.73 |
| 203684_s_at | 5.83 | 5.85 | 5.80 | 5.67 | 5.80 | 5.73 | 5.90 | 5.99 | 5.83 | 5.84 | 6.00 | 5.70 |
| 207004_at | 5.45 | 5.49 | 5.32 | 5.60 | 5.63 | 5.42 | 5.40 | 5.56 | 5.41 | 5.52 | 5.47 | 5.50 |
| 221320_at | 5.08 | 5.13 | 4.93 | 5.10 | 5.14 | 5.07 | 5.07 | 5.34 | 5.00 | 5.29 | 5.13 | 5.09 |
| 204861_s_at | 4.72 | 4.63 | 4.66 | 4.41 | 4.56 | 4.38 | 4.38 | 4.34 | 4.36 | 4.44 | 4.32 | 4.27 |
| 206537_at | 4.52 | 4.59 | 4.74 | 4.53 | 4.72 | 4.61 | 4.63 | 4.56 | 4.61 | 4.70 | 4.65 | 4.59 |
| 210564_x_at | 5.67 | 5.47 | 5.43 | 5.94 | 5.68 | 5.73 | 5.60 | 5.55 | 5.62 | 5.40 | 5.54 | 5.48 |
| 214486_x_at | 6.39 | 6.54 | 6.38 | 6.43 | 6.58 | 6.39 | 6.30 | 6.43 | 6.28 | 6.22 | 6.34 | 6.06 |
| 201631_s_at | 5.33 | 5.28 | 5.26 | 5.58 | 5.57 | 5.71 | 5.37 | 5.46 | 5.24 | 5.68 | 5.40 | 5.44 |
| 221566_s_at | 5.07 | 4.97 | 4.76 | 5.18 | 4.75 | 5.03 | 5.16 | 5.01 | 5.19 | 5.37 | 5.10 | 5.06 |
| 221567_at | 5.33 | 5.41 | 5.25 | 5.18 | 5.11 | 5.18 | 5.46 | 5.47 | 5.35 | 5.41 | 5.46 | 5.28 |
| 206248_at | 5.94 | 5.88 | 5.69 | 5.78 | 5.87 | 5.91 | 5.80 | 5.83 | 5.83 | 6.09 | 5.93 | 5.70 |
| 204466_s_at | 5.34 | 5.61 | 5.50 | 5.34 | 5.68 | 5.51 | 5.33 | 5.50 | 5.37 | 5.46 | 5.68 | 5.39 |
| 204467_s_at | 5.23 | 5.24 | 5.43 | 5.27 | 5.16 | 5.36 | 5.23 | 5.12 | 5.35 | 5.42 | 5.27 | 5.29 |
| 221371_at | 5.35 | 5.52 | 5.46 | 5.20 | 5.25 | 5.47 | 5.23 | 5.45 | 5.34 | 5.36 | 5.58 | 5.53 |
| 221601_s_at | 6.63 | 6.71 | 6.50 | 5.84 | 5.82 | 6.09 | 6.48 | 6.45 | 6.36 | 6.18 | 6.28 | 5.85 |
| 221602_s_at | 7.02 | 6.91 | 6.92 | 6.78 | 6.73 | 6.61 | 7.12 | 7.15 | 6.83 | 7.25 | 6.88 | 6.78 |

TABLE 6B
Anti-apoptosis Genes (See tables reference 2)

| | t-test (two-tailed) | | | | Meas- | | Gene | | |
|---|---|---|---|---|---|---|---|---|---|
| Probesets | pAB | pAD | pBC | pCD | ured | Changed | Symbol | UniGene ID | Gene Title |
| 209165_at | 2.6E−02 | 1.4E−01 | 5.0E−02 | 3.4E−01 | Yes | No | AATF | Hs.311079 | apoptosis antagonizing |
| | | | | | | | | | transcription factor |
| 207163_s_at | 2.3E−02 | 2.5E−02 | 2.6E−03 | 1.5E−03 | Yes | No | AKT1 | Hs.368861 | v-akt murine thymoma viral |
| | | | | | | | | | oncogene homolog 1 |
| 219366_at | 2.2E−01 | 4.9E−02 | 1.7E−01 | 4.7E−02 | Yes | No | AVEN | Hs.63168 | apoptosis, caspase activation |
| | | | | | | | | | inhibitor |
| 202387_at | 2.2E−02 | 3.8E−02 | 4.9E−03 | 3.6E−02 | Yes | No | BAG1 | Hs.377484 | BCL2-associated athanogene |
| 211475_s_at | 4.7E−02 | 1.7E−02 | 3.9E−02 | 1.1E−02 | Yes | No | BAG1 | Hs.377484 | BCL2-associated athanogene |
| 219624_at | 4.6E−02 | 9.1E−02 | 4.4E−01 | 5.0E−01 | Yes | No | BAG4 | Hs.194726 | BCL2-associated athanogene 4 |
| 203685_at | 1.2E−02 | 3.8E−02 | 1.3E−02 | 7.5E−02 | Yes | No | BCL2 | Hs.79241 | B-cell CLL/lymphoma 2 |
| 207005_s_at | 6.0E−02 | 7.0E−01 | 2.1E−02 | 3.0E−01 | Yes | No | BCL2 | Hs.79241 | B-cell CLL/lymphoma 2 |
| 205681_at | 1.7E−02 | 1.4E−02 | 8.8E−02 | 1.3E−02 | Yes | No | BCL2A1 | Hs.227817 | BCL2-related protein A1 |
| 206665_s_at | 1.2E−02 | 5.8E−01 | 2.2E−02 | 9.5E−01 | Yes | No | BCL2L1 | Hs.305890 | BCL2-like 1 |
| 212312_at | 1.3E−01 | 9.8E−01 | 6.1E−02 | 5.7E−01 | Yes | No | BCL2L1 | Hs.305890 | BCL2-like 1 |
| 215037_s_at | 2.4E−01 | 6.7E−02 | 1.3E−01 | 3.3E−01 | Yes | No | BCL2L1 | Hs.305890 | BCL2-like 1 |
| 209311_at | 9.4E−01 | 4.8E−03 | 8.8E−01 | 9.5E−02 | Yes | No | BCL2L2 | Hs.410026 | BCL2-like 2 |
| 208945_s_at | 1.5E−02 | 1.1E−01 | 6.2E−02 | 9.8E−02 | Yes | No | BECN1 | Hs.12272 | beclin 1 (coiled-coil, myosin- |
| | | | | | | | | | like BCL2 interacting protein) |
| 208946_s_at | 2.4E−03 | 1.7E−01 | 5.6E−03 | 1.3E−01 | Yes | No | BECN1 | Hs.12272 | beclin 1 (coiled-coil, myosin- |
| | | | | | | | | | like BCL2 interacting protein) |
| 202076_at | 4.8E−01 | 1.1E−01 | 7.3E−01 | 8.4E−02 | Yes | No | BIRC2 | Hs.289107 | baculoviral IAP repeat- |
| | | | | | | | | | containing 2 |
| 210538_s_at | 4.3E−04 | 6.6E−02 | 7.0E−05 | 1.9E−02 | Yes | No | BIRC3 | Hs.127799 /// | baculoviral IAP repeat- |
| | | | | | | | | Hs.127799 | containing 3 |
| 206536_s_at | 5.0E−01 | 4.3E−01 | 1.5E−01 | 1.1E−01 | Yes | No | BIRC4 | Hs.356076 | baculoviral IAP repeat- |
| | | | | | | | | | containing 4 |
| 202094_at | 1.9E−02 | 1.9E−02 | 3.6E−02 | 1.1E−02 | Yes | No | BIRC5 | Hs.1578 | baculoviral IAP repeat- |
| | | | | | | | | | containing 5 (survivin) |
| 202095_s_at | 3.6E−01 | 3.2E−02 | 3.6E−01 | 2.7E−03 | Yes | No | BIRC5 | Hs.1578 | baculoviral IAP repeat- |
| | | | | | | | | | containing 5 (survivin) |
| 210334_x_at | 2.6E−01 | 3.0E−02 | 2.5E−01 | 2.1E−02 | Yes | No | BIRC5 | Hs.1578 | baculoviral IAP repeat- |
| | | | | | | | | | containing 5 (survivin) |
| 220451_s_at | 9.6E−02 | 3.6E−02 | 6.6E−01 | 1.8E−01 | Yes | No | BIRC7 | Hs.256126 | baculoviral IAP repeat- |
| | | | | | | | | | containing 7 (livin) |
| 204930_s_at | 9.6E−01 | 1.6E−01 | 7.1E−02 | 8.6E−01 | Yes | No | BNIP1 | Hs.145726 | BCL2/adenovirus E1B 19 kDa |
| | | | | | | | | | interacting protein 1 |
| 207829_s_at | 1.9E−01 | 5.5E−01 | 5.3E−01 | 6.9E−01 | Yes | No | BNIP1 | Hs.145726 | BCL2/adenovirus E1B 19 kDa |
| | | | | | | | | | interacting protein 1 |
| 37226_at | 1.6E−01 | 4.4E−01 | 2.5E−02 | 1.6E−01 | Yes | No | BNIP1 | Hs.145726 | BCL2/adenovirus E1B 19 kDa |
| | | | | | | | | | interacting protein 1 |
| 209308_s_at | 1.3E−02 | 3.7E−01 | 2.3E−02 | 2.6E−01 | Yes | No | BNIP2 | Hs.204539 | BCL2/adenovirus E1B 19 kDa |
| | | | | | | | | | interacting protein 2 |
| 201848_s_at | 6.7E−01 | 2.2E−01 | 1.5E−01 | 5.1E−02 | Yes | No | BNIP3 | Hs.79428 | BCL2/adenovirus E1B 19 kDa |
| | | | | | | | | | interacting protein 3 |
| 201849_at | 6.0E−01 | 4.5E−01 | 2.1E−01 | 2.4E−02 | Yes | No | BNIP3 | Hs.79428 | BCL2/adenovirus E1B 19 kDa |
| | | | | | | | | | interacting protein 3 |
| 206044_s_at | 2.2E−01 | 3.9E−01 | 7.9E−02 | 2.0E−01 | Yes | No | BRAF | Hs.162967 | v-raf murine sarcoma viral |
| | | | | | | | | | oncogene homolog B1 |
| 208485_x_at | 8.3E−01 | 2.4E−01 | 9.3E−02 | 3.0E−01 | Yes | No | CFLAR | Hs.355724 | CASP8 and FADD-like |
| | | | | | | | | | apoptosis regulator |
| 209508_x_at | 8.8E−01 | 9.9E−02 | 8.5E−01 | 9.1E−02 | Yes | No | CFLAR | Hs.355724 | CASP8 and FADD-like |
| | | | | | | | | | apoptosis regulator |
| 209939_x_at | 1.6E−03 | 5.6E−03 | 3.6E−03 | 3.5E−03 | Yes | No | CFLAR | Hs.355724 | CASP8 and FADD-like |
| | | | | | | | | | apoptosis regulator |
| 210563_x_at | 2.5E−03 | 7.5E−02 | 5.7E−03 | 3.8E−02 | Yes | No | CFLAR | Hs.355724 | CASP8 and FADD-like |
| | | | | | | | | | apoptosis regulator |
| 211316_x_at | 5.4E−01 | 3.3E−03 | 7.8E−01 | 3.4E−02 | Yes | No | CFLAR | Hs.355724 | CASP8 and FADD-like |
| | | | | | | | | | apoptosis regulator |
| 211317_s_at | 3.5E−01 | 1.3E−01 | 4.3E−02 | 5.5E−02 | Yes | No | CFLAR | Hs.355724 | CASP8 and FADD-like |
| | | | | | | | | | apoptosis regulator |
| 211862_x_at | 4.3E−01 | 1.5E−02 | 7.0E−02 | 6.5E−02 | Yes | No | CFLAR | Hs.355724 | CASP8 and FADD-like |
| | | | | | | | | | apoptosis regulator |
| 214618_at | 6.0E−01 | 1.8E−01 | 9.9E−01 | 1.8E−01 | Yes | No | CFLAR | Hs.355724 | CASP8 and FADD-like |
| | | | | | | | | | apoptosis regulator |
| 200046_at | 5.5E−02 | 5.4E−03 | 1.3E−01 | 2.0E−02 | Yes | No | DAD1 | Hs.82890 | defender against cell death 1 |
| 208309_s_at | 1.5E−02 | 7.2E−04 | 5.9E−03 | 2.4E−03 | Yes | No | MALT1 | Hs.180566 | mucosa associated lymphoid |
| | | | | | | | | | tissue lymphoma translocation |
| | | | | | | | | | gene 1 |
| 210017_at | 3.6E−02 | 3.7E−02 | 2.0E−02 | 2.9E−02 | Yes | No | MALT1 | Hs.180566 | mucosa associated lymphoid |
| | | | | | | | | | tissue lymphoma translocation |
| | | | | | | | | | gene 1 |
| 210018_x_at | 7.4E−02 | 1.6E−02 | 1.4E−01 | 2.0E−02 | Yes | No | MALT1 | Hs.180566 | mucosa associated lymphoid |
| | | | | | | | | | tissue lymphoma translocation |
| | | | | | | | | | gene 1 |
| 209239_at | 6.6E−03 | 1.9E−01 | 6.8E−03 | 4.6E−01 | Yes | No | NFKB1 | Hs.160557 | nuclear factor of kappa light |
| | | | | | | | | | polypeptide gene enhancer in B- |
| | | | | | | | | | cells 1 (p105) |
| 201739_at | 7.2E−03 | 2.8E−02 | 9.8E−03 | 2.9E−02 | Yes | No | SGK | Hs.296323 | serum/glucocorticoid regulated |
| | | | | | | | | | kinase |
| 218878_s_at | 2.1E−01 | 3.7E−01 | 2.0E−01 | 4.4E−01 | Yes | No | SIRT1 | Hs.31176 | sirtuin (silent mating type |
| | | | | | | | | | information regulation 2 |
| | | | | | | | | | homolog) 1 (S. cerevisiae) |
| 207827_x_at | 4.4E−02 | 1.8E−01 | 4.4E−01 | 7.9E−01 | Yes | No | SNCA | Hs.76930 | synuclein, alpha (non A4 |
| | | | | | | | | | component of amyloid |
| | | | | | | | | | precursor) |
| 211546_x_at | 2.7E−01 | 8.2E−02 | 8.1E−01 | 4.5E−01 | Yes | No | SNCA | Hs.76930 | synuclein, alpha (non A4 |
| | | | | | | | | | component of amyloid |
| | | | | | | | | | precursor) |
| 201085_s_at | 4.6E−01 | 1.5E−01 | 2.3E−01 | 1.1E−01 | Yes | No | SON | Hs.430541 | SON DNA binding protein |
| 201086_x_at | 1.3E−01 | 5.3E−04 | 2.3E−01 | 5.1E−03 | Yes | No | SON | Hs.430541 | SON DNA binding protein |
| 213538_at | 7.5E−02 | 3.1E−01 | 1.7E−02 | 6.0E−01 | Yes | No | SON | Hs.430541 | SON DNA binding protein |
| 214988_s_at | 4.3E−01 | 2.6E−02 | 2.6E−01 | 2.6E−02 | Yes | No | SON | Hs.430541 | SON DNA binding protein |
| 200803_s_at | 2.8E−01 | 4.6E−02 | 6.2E−03 | 2.3E−03 | Yes | No | TEGT | Hs.35052 | testis enhanced gene transcript |
| | | | | | | | | | (BAX inhibitor 1) |
| 200804_at | 5.9E−01 | 1.2E−01 | 4.6E−01 | 2.0E−02 | Yes | No | TEGT | Hs.35052 | testis enhanced gene transcript |
| | | | | | | | | | (BAX inhibitor 1) |
| 202643_s_at | 1.7E−05 | 3.5E−02 | 6.8E−03 | 3.9E−02 | Yes | No | TNFAIP3 | Hs.211600 | tumor necrosis factor, |
| | | | | | | | | | alpha-induced protein 3 |
| 202644_s_at | 7.0E−04 | 5.2E−02 | 5.8E−04 | 1.4E−02 | Yes | No | TNFAIP3 | Hs.211600 | tumor necrosis factor, |
| | | | | | | | | | alpha-induced protein 3 |
| 206092_x_at | 1.3E−01 | 4.6E−01 | 7.0E−02 | 9.4E−01 | Yes | No | TNFRSF6B | Hs.348183 | tumor necrosis factor receptor |
| | | | | | | | | | superfamily, member 6b, decoy |
| 206467_x_at | 1.6E−02 | 3.7E−02 | 2.0E−01 | 4.6E−01 | Yes | No | TNFRSF6B | Hs.348183 | tumor necrosis factor receptor |
| | | | | | | | | | superfamily, member 6b, decoy |
| 213829_x_at | 6.2E−01 | 6.9E−01 | 4.5E−01 | 5.1E−01 | Yes | No | TNFRSF6B | Hs.348183 | tumor necrosis factor receptor |
| | | | | | | | | | superfamily, member 6b, decoy |
| 203684_s_at | 1.2E−01 | 8.2E−01 | 4.8E−02 | 6.0E−01 | No | No | BCL2 | Hs.79241 | B-cell CLL/lymphoma 2 |
| 207004_at | 1.9E−01 | 2.6E−01 | 3.3E−01 | 5.3E−01 | No | No | BCL2 | Hs.79241 | B-cell CLL/lymphoma 2 |
| 221320_at | 4.3E−01 | 2.2E−01 | 7.9E−01 | 7.9E−01 | No | No | BCL2L10 | Hs.283672 | BCL2-like 10 (apoptosis |
| | | | | | | | | | facilitator) |
| 204861_s_at | 4.6E−02 | 9.8E−03 | 2.7E−01 | 7.8E−01 | No | No | BIRC1 | Hs.79019 | baculoviral IAP repeat- |
| | | | | | | | | | containing 1 |
| 206537_at | 9.6E−01 | 6.9E−01 | 7.6E−01 | 3.0E−01 | No | No | BIRC4 | Hs.356076 | baculoviral IAP repeat- |
| | | | | | | | | | containing 4 |
| 210564_x_at | 7.5E−02 | 5.9E−01 | 1.2E−01 | 7.3E−02 | No | No | CFLAR | Hs.355724 | CASP8 and FADD-like |
| | | | | | | | | | apoptosis regulator |
| 214486_x_at | 7.2E−01 | 8.1E−02 | 1.4E−01 | 2.5E−01 | No | No | CFLAR | Hs.355724 | CASP8 and FADD-like |
| | | | | | | | | | apoptosis regulator |
| 201631_s_at | 7.6E−03 | 1.2E−01 | 3.3E−02 | 2.4E−01 | No | No | IER3 | Hs.76095 | immediate early response 3 |
| 221566_s_at | 7.3E−01 | 1.4E−01 | 4.1E−01 | 6.4E−01 | No | No | NOL3 | Hs.462911 | nucleolar protein 3 |
| | | | | | | | | | (apoptosis repressor with CARD |
| | | | | | | | | | domain) |
| 221567_at | 4.1E−02 | 5.0E−01 | 7.0E−03 | 5.5E−01 | No | No | NOL3 | Hs.462911 | nucleolar protein 3 |
| | | | | | | | | | (apoptosis repressor with CARD |
| | | | | | | | | | domain) |
| 206248_at | 8.9E−01 | 6.5E−01 | 4.7E−01 | 5.1E−01 | No | No | PRKCE | Hs.155281 | protein kinase C, epsilon |
| 204466_s_at | 8.3E−01 | 8.2E−01 | 4.0E−01 | 3.6E−01 | No | No | SNCA | Hs.76930 | synuclein, alpha (non A4 |
| | | | | | | | | | component of amyloid |
| | | | | | | | | | precursor) |
| 204467_s_at | 6.9E−01 | 7.3E−01 | 7.6E−01 | 3.2E−01 | No | No | SNCA | Hs.76930 | synuclein, alpha (non A4 |
| | | | | | | | | | component of amyloid |
| | | | | | | | | | precursor) |
| 221371_at | 2.7E−01 | 5.9E−01 | 7.6E−01 | 1.9E−01 | No | No | TNFSF18 | Hs.248197 | tumor necrosis factor (ligand) |
| | | | | | | | | | superfamily, member 18 |
| 221601_s_at | 4.0E−03 | 4.2E−02 | 1.7E−02 | 1.2E−01 | No | No | TOSO | Hs.58831 | regulator of Fas-induced |
| | | | | | | | | | apoptosis |
| 221602_s_at | 2.1E−02 | 9.1E−01 | 6.5E−02 | 7.2E−01 | No | No | TOSO | Hs.58831 | regulator of Fas-induced |
| | | | | | | | | | apoptosis |

TABLE 7A
B-Cell Maturation Specific Genes

| | Log2 fluorescence intensity | | | | | | | | | | | |
|---|---|---|---|---|---|---|---|---|---|---|---|---|
| | Ly1 | | Ly1 | | Ly7 | | | | | | | |
| Probesets | (A1) | Ly1 (A2) | (A3) | Ly7 (B1) | (B2) | Ly7 (B3) | Ly8 (C1) | Ly8 (C2) | Ly8 (C3) | D4 (D1) | D4 (D2) | D4 (D3) |
| 202094_at | 8.05 | 8.01 | 7.73 | 8.69 | 8.38 | 8.37 | 7.94 | 8.10 | 8.08 | 8.54 | 8.31 | 8.45 |
| 202095_s_at | 9.08 | 9.07 | 8.69 | 8.95 | 8.59 | 8.79 | 8.92 | 8.79 | 9.01 | 9.52 | 9.38 | 9.59 |
| 210334_x_at | 8.24 | 8.19 | 7.87 | 8.34 | 8.21 | 8.27 | 8.13 | 7.97 | 8.29 | 8.75 | 8.54 | 8.49 |
| 204581_at | 7.31 | 6.65 | 6.97 | 9.29 | 9.12 | 9.07 | 7.64 | 6.88 | 7.08 | 9.45 | 9.76 | 9.57 |
| 217422_s_at | 6.48 | 6.12 | 6.21 | 8.47 | 8.56 | 8.33 | 6.65 | 5.98 | 5.96 | 8.61 | 9.38 | 8.76 |
| 38521_at | 8.75 | 8.60 | 8.51 | 9.72 | 9.86 | 9.76 | 8.71 | 8.54 | 8.42 | 9.89 | 10.19 | 10.05 |
| 206545_at | 4.02 | 3.98 | 4.01 | 6.13 | 6.64 | 5.89 | 3.90 | 3.92 | 3.96 | 4.05 | 3.99 | 3.95 |
| 211856_x_at | 5.39 | 5.68 | 5.60 | 5.93 | 6.48 | 6.52 | 5.47 | 5.51 | 5.52 | 5.61 | 5.69 | 5.51 |
| 211861_x_at | 4.01 | 4.10 | 3.93 | 4.35 | 4.88 | 4.82 | 3.89 | 3.97 | 4.11 | 4.17 | 4.14 | 4.07 |
| 204192_at | 10.20 | 8.83 | 9.62 | 9.58 | 9.67 | 9.21 | 9.74 | 8.62 | 8.54 | 9.85 | 9.94 | 9.72 |
| 205692_s_at | 6.94 | 7.34 | 7.45 | 9.32 | 9.34 | 9.36 | 6.89 | 7.41 | 7.26 | 9.01 | 9.14 | 8.98 |
| 209835_x_at | 4.51 | 4.41 | 4.43 | 4.50 | 4.51 | 4.58 | 4.52 | 4.56 | 4.51 | 4.57 | 4.59 | 4.57 |
| 209795_at | 4.56 | 4.42 | 4.80 | 4.00 | 4.14 | 4.03 | 4.36 | 4.48 | 4.27 | 4.07 | 4.06 | 4.06 |
| 200675_at | 11.42 | 11.58 | 11.30 | 10.93 | 10.69 | 10.90 | 11.18 | 11.33 | 11.20 | 11.17 | 11.01 | 11.17 |
| 202910_s_at | 6.46 | 6.62 | 6.38 | 6.06 | 6.19 | 5.99 | 6.51 | 6.78 | 6.67 | 6.22 | 6.45 | 6.40 |
| 207691_x_at | 5.60 | 5.66 | 5.54 | 5.00 | 5.03 | 4.97 | 5.40 | 5.14 | 5.33 | 4.95 | 4.99 | 4.73 |
| 209473_at | 6.69 | 6.41 | 6.61 | 5.49 | 6.05 | 6.08 | 6.38 | 6.44 | 6.45 | 5.77 | 5.45 | 5.61 |
| 209474_s_at | 4.91 | 4.79 | 4.92 | 4.40 | 4.63 | 4.46 | 4.79 | 4.71 | 4.87 | 4.27 | 4.51 | 4.34 |
| 219731_at | 6.64 | 7.01 | 6.91 | 6.84 | 6.91 | 6.64 | 6.59 | 6.86 | 6.69 | 6.28 | 6.68 | 6.18 |
| 211430_s_at | 10.70 | 10.74 | 10.73 | 5.10 | 5.78 | 5.96 | 11.52 | 11.37 | 10.89 | 10.06 | 10.59 | 9.59 |
| 211641_x_at | 5.33 | 5.52 | 5.28 | 5.10 | 4.90 | 4.92 | 5.28 | 5.34 | 5.36 | 4.95 | 5.09 | 4.95 |
| 214973_x_at | 6.67 | 6.76 | 6.76 | 6.37 | 6.65 | 6.31 | 6.67 | 7.06 | 6.47 | 6.48 | 6.50 | 6.49 |
| 217281_x_at | 5.93 | 5.68 | 5.35 | 5.19 | 4.81 | 5.11 | 5.87 | 5.69 | 5.41 | 5.41 | 5.27 | 5.05 |
| 203233_at | 6.56 | 6.42 | 6.48 | 7.84 | 7.90 | 7.58 | 6.86 | 6.55 | 6.39 | 7.02 | 7.41 | 6.91 |
| 204864_s_at | 4.96 | 4.97 | 4.82 | 4.84 | 4.96 | 5.03 | 5.02 | 5.02 | 4.99 | 5.10 | 4.94 | 4.98 |
| 212195_at | 4.36 | 4.05 | 4.17 | 4.19 | 4.22 | 4.08 | 5.14 | 4.49 | 4.80 | 3.83 | 3.92 | 3.88 |
| 204562_at | 8.22 | 8.60 | 8.59 | 8.69 | 9.38 | 8.42 | 8.35 | 8.44 | 7.86 | 6.36 | 6.28 | 6.01 |
| 216986_s_at | 6.51 | 6.37 | 6.35 | 6.45 | 6.80 | 6.42 | 6.55 | 6.43 | 6.23 | 6.43 | 6.42 | 6.01 |
| 201286_at | 4.49 | 4.58 | 4.38 | 4.48 | 4.61 | 4.54 | 4.48 | 4.46 | 4.55 | 4.70 | 4.60 | 4.43 |
| 206641_at | 4.61 | 4.74 | 4.44 | 7.47 | 8.13 | 7.40 | 4.55 | 4.51 | 4.66 | 6.82 | 7.58 | 7.39 |
| 206729_at | 5.68 | 5.98 | 5.69 | 5.51 | 5.49 | 5.75 | 5.68 | 5.76 | 5.65 | 5.70 | 5.76 | 5.59 |
| 206508_at | 5.59 | 5.65 | 5.60 | 7.85 | 8.89 | 7.93 | 5.59 | 5.81 | 5.45 | 5.69 | 5.96 | 5.81 |
| 220674_at | 4.69 | 4.54 | 4.34 | 4.92 | 4.75 | 4.68 | 4.72 | 4.52 | 4.54 | 4.81 | 4.87 | 4.68 |
| 204489_s_at | 5.37 | 5.38 | 5.30 | 5.34 | 5.37 | 5.26 | 5.34 | 5.40 | 5.22 | 5.33 | 5.32 | 5.20 |
| 204490_s_at | 4.32 | 4.47 | 4.55 | 4.72 | 4.51 | 4.47 | 4.44 | 4.35 | 4.38 | 4.72 | 4.51 | 4.44 |
| 210916_s_at | 4.67 | 4.58 | 4.52 | 4.58 | 4.54 | 4.57 | 4.65 | 4.59 | 4.61 | 4.93 | 4.81 | 4.67 |
| 212014_x_at | 4.45 | 4.45 | 4.32 | 4.42 | 4.48 | 4.45 | 4.44 | 4.44 | 4.43 | 4.57 | 4.51 | 4.48 |
| 212063_at | 3.94 | 4.15 | 4.11 | 4.02 | 4.17 | 4.06 | 3.95 | 4.08 | 3.97 | 4.04 | 4.06 | 4.14 |
| 216056_at | 4.69 | 4.92 | 5.00 | 4.77 | 5.03 | 5.04 | 4.71 | 4.79 | 4.79 | 4.86 | 4.86 | 4.78 |
| 216062_at | 5.78 | 5.65 | 5.73 | 5.81 | 5.57 | 5.76 | 5.77 | 5.72 | 5.76 | 6.13 | 5.76 | 5.89 |
| 217523_at | 3.76 | 3.80 | 3.80 | 3.79 | 3.78 | 3.86 | 3.79 | 3.76 | 3.82 | 3.84 | 3.81 | 3.77 |
| 205544_s_at | 4.95 | 4.95 | 4.88 | 4.82 | 4.97 | 4.88 | 4.88 | 4.83 | 5.04 | 4.90 | 4.95 | 4.83 |
| 203716_s_at | 4.97 | 5.02 | 5.03 | 5.20 | 4.75 | 5.08 | 5.16 | 4.95 | 5.07 | 5.69 | 5.14 | 5.22 |
| 203717_at | 4.07 | 3.92 | 4.05 | 4.07 | 4.07 | 4.05 | 3.97 | 4.06 | 4.03 | 4.16 | 4.12 | 4.07 |
| 211478_s_at | 3.98 | 3.98 | 3.95 | 3.98 | 4.09 | 4.02 | 4.10 | 4.03 | 3.85 | 4.03 | 4.09 | 4.02 |
| 206759_at | 4.75 | 4.58 | 4.59 | 5.06 | 4.88 | 4.78 | 4.63 | 4.51 | 4.49 | 5.09 | 4.88 | 4.68 |
| 206760_s_at | 5.79 | 5.92 | 5.84 | 6.05 | 5.96 | 5.97 | 5.89 | 5.79 | 5.82 | 6.00 | 6.09 | 5.90 |
| 211636_at | 4.17 | 4.18 | 3.93 | 4.13 | 4.09 | 4.16 | 4.32 | 4.18 | 4.14 | 4.20 | 4.24 | 4.09 |
| 211642_at | 4.35 | 4.44 | 4.33 | 4.41 | 4.10 | 4.36 | 4.57 | 4.24 | 4.37 | 4.55 | 4.23 | 4.29 |
| 211878_at | 4.06 | 3.90 | 3.82 | 4.00 | 3.92 | 4.01 | 3.99 | 3.94 | 3.89 | 3.96 | 3.99 | 3.98 |
| 215721_at | 5.21 | 5.04 | 5.02 | 4.84 | 4.64 | 4.81 | 5.15 | 5.06 | 5.00 | 4.87 | 4.86 | 4.69 |
| 217260_x_at | 5.40 | 5.36 | 5.29 | 4.82 | 5.15 | 5.13 | 4.98 | 5.47 | 5.21 | 5.15 | 5.21 | 4.82 |
| 206341_at | 4.41 | 4.38 | 4.35 | 4.32 | 4.23 | 4.32 | 4.47 | 4.27 | 4.43 | 4.50 | 4.52 | 4.29 |
| 211269_s_at | 5.99 | 5.92 | 6.07 | 5.93 | 6.13 | 5.99 | 6.01 | 6.15 | 5.98 | 5.84 | 5.90 | 5.96 |
| 205945_at | 4.07 | 4.13 | 4.08 | 4.02 | 4.16 | 4.17 | 3.99 | 4.08 | 4.05 | 4.06 | 4.25 | 4.06 |
| 217489_s_at | 4.53 | 4.54 | 4.44 | 4.51 | 4.47 | 4.54 | 4.59 | 4.44 | 4.51 | 4.56 | 4.89 | 4.61 |
| 204863_s_at | 4.10 | 4.16 | 4.08 | 4.11 | 4.19 | 4.16 | 4.12 | 4.10 | 4.18 | 4.19 | 4.12 | 4.14 |
| 211000_s_at | 5.05 | 5.01 | 4.88 | 4.98 | 4.95 | 5.02 | 5.03 | 5.17 | 5.05 | 5.14 | 5.09 | 4.90 |
| 212196_at | 4.13 | 4.06 | 4.04 | 4.04 | 4.07 | 4.13 | 4.26 | 4.25 | 4.22 | 4.02 | 4.15 | 4.09 |
| 216987_at | 3.88 | 3.74 | 3.80 | 3.79 | 3.74 | 3.79 | 3.78 | 3.72 | 3.82 | 3.80 | 3.77 | 3.80 |
| 201287_s_at | 6.37 | 6.41 | 6.38 | 6.27 | 6.36 | 6.24 | 6.32 | 6.44 | 6.29 | 6.43 | 6.24 | 6.28 |

TABLE 7B
B-Cell Maturation Specific Genes

| | t-test (two-tailed) | | | | | | Gene | | | |
|---|---|---|---|---|---|---|---|---|---|---|
| Probesets | pAB | pAD | pBC | pCD | Measured | Changed | Symbol | UniGene ID | Gene Title | Ref. |
| FDR Cutoff | 1.0E−02 | 8.2E−03 | 1.2E−02 | 9.4E−03 | AC v. BD | AC v. BD | | | | |
| 202094_at | 1.9E−02 | 1.9E−02 | 3.6E−02 | 1.1E−02 | Yes | No | BIRC5 | Hs.1578 | baculoviral IAP repeat- | 3 |
| | | | | | | | | | containing 5 (survivin) | |
| 202095_s_at | 3.6E−01 | 3.2E−02 | 3.6E−01 | 2.7E−03 | Yes | No | BIRC5 | Hs.1578 | baculoviral IAP repeat- | 3 |
| | | | | | | | | | containing 5 (survivin) | |
| 210334_x_at | 2.6E−01 | 3.0E−02 | 2.5E−01 | 2.1E−02 | Yes | No | BIRC5 | Hs.1578 | baculoviral IAP repeat- | 3 |
| | | | | | | | | | containing 5 (survivin) | |
| 204581_at | 3.8E−03 | 1.4E−03 | 8.7E−03 | 3.9E−03 | Yes | Yes | CD22 | Hs.262150 | CD22 antigen | 4 |
| 217422_s_at | 2.4E−04 | 2.7E−03 | 6.2E−03 | 1.2E−03 | Yes | Yes | CD22 | Hs.262150 | CD22 antigen | 4 |
| 38521_at | 4.5E−04 | 3.0E−04 | 1.3E−03 | 2.6E−04 | Yes | Yes | CD22 | Hs.262150 | CD22 antigen | 4 |
| 206545_at | 9.6E−03 | 8.7E−01 | 8.8E−03 | 1.3E−01 | Yes | No | CD28 | Hs.1987 | CD28 antigen (Tp44) | 4 |
| 211856_x_at | 4.2E−02 | 6.6E−01 | 5.0E−02 | 1.7E−01 | Yes | No | CD28 | Hs.1987 | CD28 antigen (Tp44) | 4 |
| 211861_x_at | 4.7E−02 | 1.2E−01 | 3.9E−02 | 1.6E−01 | Yes | No | CD28 | Hs.1987 | CD28 antigen (Tp44) | 4 |
| 204192_at | 8.9E−01 | 5.5E−01 | 3.1E−01 | 1.5E−01 | Yes | No | CD37 | Hs.153053 | CD37 antigen | 4 |
| 205692_s_at | 5.2E−03 | 4.2E−03 | 4.9E−03 | 3.9E−03 | Yes | Yes | CD38 | Hs.174944 | CD38 antigen (p45) | 4 |
| 209835_x_at | 1.1E−01 | 4.6E−02 | 9.6E−01 | 8.9E−02 | Yes | No | CD44 | Hs.306278 | CD44 antigen | 5 |
| 209795_at | 2.8E−02 | 4.2E−02 | 1.9E−02 | 4.0E−02 | Yes | No | CD69 | Hs.82401 | CD69 antigen (p60, early | 4 |
| | | | | | | | | | T-cell activation antigen) | |
| 200675_at | 5.9E−03 | 3.7E−02 | 1.7E−02 | 1.7E−01 | Yes | No | CD81 | Hs.54457 | CD81 antigen (target of | 4 |
| | | | | | | | | | antiproliferative antibody | |
| | | | | | | | | | 1) | |
| 202910_s_at | 1.2E−02 | 2.5E−01 | 5.7E−03 | 4.9E−02 | Yes | No | CD97 | Hs.3107 | CD97 antigen | 4 |
| 207691_x_at | 1.0E−03 | 5.2E−03 | 6.0E−02 | 2.3E−02 | Yes | No | ENTPD1 | Hs.444105 | CD39 antigen | 4 |
| 209473_at | 5.1E−02 | 1.6E−03 | 1.0E−01 | 9.3E−03 | Yes | No | ENTPD1 | Hs.444105 | CD39 antigen | 4 |
| 209474_s_at | 1.5E−02 | 6.4E−03 | 3.0E−02 | 1.1E−02 | Yes | No | ENTPD1 | Hs.444105 | CD39 antigen | 4 |
| 219731_at | 7.1E−01 | 7.4E−02 | 5.0E−01 | 1.5E−01 | Yes | No | ENTPD1 | Hs.444105 | CD39 antigen | 4 |
| 211430_s_at | 2.6E−03 | 1.6E−01 | 1.2E−04 | 3.4E−02 | Yes | No | IGHG3 | Hs.413826 | immunoglobulin heavy | 4 |
| | | | | | | | | | constant gamma 3 (G3m | |
| | | | | | | | | | marker) | |
| 211641_x_at | 1.5E−02 | 1.7E−02 | 1.9E−02 | 8.8E−03 | Yes | No | IGHG3 | Hs.413826 | immunoglobulin heavy | 4 |
| | | | | | | | | | constant gamma 3 (G3m | |
| | | | | | | | | | marker) | |
| 214973_x_at | 1.0E−01 | 1.2E−02 | 2.3E−01 | 2.9E−01 | Yes | No | IGHG3 | Hs.413826 | immunoglobulin heavy | 4 |
| | | | | | | | | | constant gamma 3 (G3m | |
| | | | | | | | | | marker) | |
| 217281_x_at | 4.6E−02 | 1.2E−01 | 2.7E−02 | 7.7E−02 | Yes | No | IGHG3 | Hs.413826 | immunoglobulin heavy | 4 |
| | | | | | | | | | constant gamma 3 (G3m | |
| | | | | | | | | | marker) | |
| 203233_at | 2.2E−03 | 4.6E−02 | 3.2E−03 | 6.6E−02 | Yes | No | IL4R | Hs.75545 | CD124 antigen | 4 |
| 204864_s_at | 7.5E−01 | 2.4E−01 | 3.4E−01 | 9.8E−01 | Yes | No | IL6ST | Hs.71968 | gp130 | 4 |
| 212195_at | 8.0E−01 | 6.6E−02 | 6.8E−02 | 3.6E−02 | Yes | No | IL6ST | Hs.71968 | gp130 | 4 |
| 204562_at | 3.4E−01 | 2.1E−04 | 1.6E−01 | 1.7E−03 | Yes | No | IRF4 | Hs.127686 | interferon regulatory | 6 |
| | | | | | | | | | factor 4 | |
| 216986_s_at | 3.5E−01 | 4.9E−01 | 3.8E−01 | 5.5E−01 | Yes | No | IRF4 | Hs.127686 | interferon regulatory | 6 |
| | | | | | | | | | factor 4 | |
| 201286_at | 4.8E−01 | 4.0E−01 | 4.0E−01 | 4.2E−01 | Yes | No | SDC1 | Hs.82109 | CD138 antigen | 4 |
| 206641_at | 2.4E−03 | 3.1E−03 | 4.3E−03 | 5.5E−03 | Yes | Yes | TNFRSF17 | Hs.2556 | B-cell maturation factor | 7 |
| 206729_at | 2.0E−01 | 4.4E−01 | 3.2E−01 | 8.6E−01 | Yes | No | TNFRSF8 | Hs.1314 | CD30 antigen | 4 |
| 206508_at | 1.6E−02 | 1.2E−01 | 1.0E−02 | 2.1E−01 | Yes | No | TNFSF7 | Hs.99899 | CD70 antigen | 4 |
| 220674_at | 1.1E−01 | 1.1E−01 | 1.1E−01 | 8.5E−02 | No | No | CD22 | Hs.262150 | CD22 antigen | 4 |
| 204489_s_at | 5.3E−01 | 2.4E−01 | 9.7E−01 | 6.0E−01 | No | No | CD44 | Hs.306278 | CD44 antigen | 5 |
| 204490_s_at | 3.1E−01 | 3.6E−01 | 1.5E−01 | 1.8E−01 | No | No | CD44 | Hs.306278 | CD44 antigen | 5 |
| 210916_s_at | 6.4E−01 | 8.4E−02 | 8.2E−02 | 1.2E−01 | No | No | CD44 | Hs.306278 | CD44 antigen | 5 |
| 212014_x_at | 4.2E−01 | 1.1E−01 | 4.5E−01 | 9.0E−02 | No | No | CD44 | Hs.306278 | CD44 antigen | 5 |
| 212063_at | 8.0E−01 | 8.1E−0.1 | 2.2E−01 | 1.8E−01 | No | No | CD44 | Hs.306278 | CD44 antigen | 5 |
| 216056_at | 5.8E−01 | 7.4E−01 | 1.6E−01 | 1.3E−01 | No | No | CD44 | Hs.306278 | CD44 antigen | 5 |
| 216062_at | 9.5E−01 | 1.9E−01 | 6.7E−01 | 2.4E−01 | No | No | CD44 | Hs.306278 | CD44 antigen | 5 |
| 217523_at | 4.2E−01 | 4.0E−01 | 4.5E−01 | 4.5E−01 | No | No | CD44 | Hs.306278 | CD44 antigen | 5 |
| 205544_s_at | 5.5E−01 | 5.1E−01 | 8.1E−01 | 8.3E−01 | No | No | CR2 | Hs.73792 | CD21 antigen | 4 |
| 203716_s_at | 9.9E−01 | 1.8E−01 | 7.5E−01 | 2.2E−01 | No | No | DPP4 | Hs.44926 | CD26 antigen | 4 |
| 203717_at | 3.7E−01 | 1.4E−01 | 2.4E−01 | 6.3E−02 | No | No | DPP4 | Hs.44926 | CD26 antigen | 4 |
| 211478_s_at | 2.0E−01 | 4.2E−02 | 7.3E−01 | 5.7E−01 | No | No | DPP4 | Hs.44926 | CD26 antigen | 4 |
| 206759_at | 5.8E−02 | 1.6E−01 | 2.7E−02 | 8.7E−02 | No | No | FCER2 | Hs.1416 | CD23 antigen | 4 |
| 206760_s_at | 3.8E−02 | 9.5E−02 | 2.0E−02 | 7.0E−02 | No | No | FCER2 | Hs.1416 | CD23 antigen | 4 |
| 211636_at | 7.2E−01 | 4.3E−01 | 2.3E−01 | 6.1E−01 | No | No | IGHG3 | Hs.413826 | immunoglobulin heavy | 4 |
| | | | | | | | | | constant gamma 3 (G3m | |
| | | | | | | | | | marker) | |
| 211642_at | 5.0E−01 | 9.0E−01 | 4.8E−01 | 7.9E−01 | No | No | IGHG3 | Hs.413826 | immunoglobulin heavy | 4 |
| | | | | | | | | | constant gamma 3 (G3m | |
| | | | | | | | | | marker) | |
| 211878_at | 5.5E−01 | 5.7E−01 | 3.8E−01 | 3.3E−01 | No | No | IGHG3 | Hs.413826 | immunoglobulin heavy | 4 |
| | | | | | | | | | constant gamma 3 (G3m | |
| | | | | | | | | | marker) | |
| 215721_at | 1.9E−02 | 2.7E−02 | 2.0E−02 | 2.5E−02 | No | No | IGHG3 | Hs.413826 | immunoglobulin heavy | 4 |
| | | | | | | | | | constant gamma 3 (G3m | |
| | | | | | | | | | marker) | |
| 217260_x_at | 8.6E−02 | 1.3E−01 | 3.6E−01 | 4.4E−01 | No | No | IGHG3 | Hs.413826 | immunoglobulin heavy | 4 |
| | | | | | | | | | constant gamma 3 (G3m | |
| | | | | | | | | | marker) | |
| 206341_at | 6.7E−02 | 5.4E−01 | 2.5E−01 | 6.7E−01 | No | No | IL2RA | Hs.130058 | CD25 antigen | 4 |
| 211269_s_at | 7.6E−01 | 1.7E−01 | 7.4E−01 | 8.7E−02 | No | No | IL2RA | Hs.130058 | CD25 antigen | 4 |
| 205945_at | 7.1E−01 | 7.1E−01 | 2.5E−01 | 3.1E−01 | No | No | IL6R | Hs.193400 | CD126 antigen | 4 |
| 217489_s_at | 8.6E−01 | 2.1E−01 | 9.6E−01 | 2.3E−01 | No | No | IL6R | Hs.193400 | CD126 antigen | 4 |
| 204863_s_at | 2.5E−01 | 2.9E−01 | 5.7E−01 | 6.4E−01 | No | No | IL6ST | Hs.71968 | CD126 antigen | 4 |
| 211000_s_at | 9.8E−01 | 5.4E−01 | 1.2E−01 | 6.4E−01 | No | No | IL6ST | Hs.71968 | CD126 antigen | 4 |
| 212196_at | 9.6E−01 | 8.8E−01 | 1.3E−02 | 4.1E−02 | No | No | IL6ST | Hs.71968 | CD126 antigen | 4 |
| 216987_at | 5.6E−01 | 7.4E−01 | 9.1E−01 | 6.0E−01 | No | No | IRF4 | Hs.127686 | interferon regulatory | 6 |
| | | | | | | | | | factor 4 | |
| 201287_s_at | 1.1E−01 | 3.6E−01 | 3.6E−01 | 6.6E−01 | No | No | SDC1 | Hs.82109 | CD138 antigen | 4 |

TABLE 8A
Pan-B-Cell Genes

| | Log2 fluorescence intensity | | | | | | | | | | | |
|---|---|---|---|---|---|---|---|---|---|---|---|---|
| | Ly1 | | Ly1 | | Ly7 | | | | | | | |
| Probesets | (A1) | Ly1 (A2) | (A3) | Ly7 (B1) | (B2) | Ly7 (B3) | Ly8 (C1) | Ly8 (C2) | Ly8 (C3) | D4 (D1) | D4 (D2) | D4 (D3) |
| 206398_s_at | 10.29 | 10.05 | 10.02 | 9.37 | 9.43 | 9.01 | 10.05 | 9.89 | 9.62 | 8.35 | 8.93 | 8.50 |
| 215925_s_at | 7.46 | 6.84 | 7.74 | 6.66 | 7.11 | 6.71 | 7.15 | 6.81 | 6.93 | 7.20 | 7.36 | 7.17 |
| 209619_at | 12.37 | 11.96 | 12.10 | 12.07 | 12.17 | 11.94 | 12.11 | 11.63 | 11.56 | 12.67 | 12.83 | 12.63 |
| 205049_s_at | 10.15 | 9.84 | 9.81 | 10.50 | 10.49 | 10.12 | 9.82 | 9.54 | 9.51 | 10.19 | 10.41 | 10.05 |
| 205297_s_at | 10.93 | 10.97 | 10.91 | 9.82 | 9.84 | 9.68 | 10.91 | 10.80 | 10.64 | 9.51 | 9.79 | 9.71 |
| 207176_s_at | 6.11 | 6.33 | 6.32 | 6.31 | 6.25 | 6.39 | 6.13 | 6.37 | 6.25 | 6.58 | 6.49 | 6.50 |
| 210356_x_at | 10.06 | 9.78 | 10.31 | 10.94 | 11.02 | 10.94 | 9.54 | 9.82 | 9.63 | 11.02 | 11.32 | 10.99 |
| 217418_x_at | 10.10 | 97.6 | 10.14 | 10.89 | 10.94 | 10.87 | 9.45 | 9.75 | 9.59 | 10.96 | 11.22 | 10.85 |
| 221969_at | 8.41 | 8.70 | 8.84 | 7.69 | 7.68 | 7.55 | 8.46 | 8.75 | 8.41 | 8.01 | 8.06 | 7.96 |
| 215346_at | 6.64 | 6.15 | 6.16 | 6.82 | 6.86 | 6.41 | 6.44 | 6.49 | 5.92 | 6.97 | 6.90 | 6.83 |
| 35150_at | 8.56 | 8.35 | 8.22 | 8.42 | 8.72 | 8.53 | 8.28 | 8.41 | 8.06 | 8.67 | 8.99 | 8.50 |
| 220068_at | 10.38 | 10.38 | 10.30 | 10.12 | 10.15 | 10.03 | 9.95 | 10.02 | 10.07 | 9.85 | 10.05 | 10.03 |
| 206802_at | 6.05 | 6.11 | 5.94 | 6.12 | 6.03 | 5.87 | 6.18 | 6.05 | 5.78 | 6.25 | 6.05 | 5.91 |
| 205153_s_at | 5.94 | 5.42 | 5.24 | 6.04 | 6.24 | 6.03 | 5.75 | 5.47 | 5.33 | 6.25 | 6.50 | 6.10 |
| 222292_at | 4.59 | 4.65 | 4.73 | 4.57 | 4.65 | 4.52 | 4.51 | 4.47 | 4.60 | 4.66 | 4.67 | 4.85 |

TABLE 8B
Pan-B-Cell Genes

| | t-test (two-tailed) | | | | | | Gene | | | |
|---|---|---|---|---|---|---|---|---|---|---|
| Probesets | pAB | pAD | pBC | pCD | Measured | Changed | Symbol | UniGene ID | Gene Title | Ref. |
| 206398_s_at | 8.4E−03 | 4.9E−03 | 3.3E−02 | 5.8E−03 | Yes | No | CD19 | Hs.96023 | CD19 antigen | 4 |
| 215925_s_at | 1.8E−01 | 7.4E−01 | 4.8E−01 | 8.8E−02 | Yes | No | CD72 | Hs.116481 | CD72 antigen | 4 |
| 209619_at | 5.7E−01 | 2.4E−02 | 2.3E−01 | 2.2E−02 | Yes | No | CD74 | Hs.446471 | CD74 antigen | 4 |
| 205049_s_at | 5.9E−02 | 1.4E−01 | 1.0E−02 | 1.5E−02 | Yes | No | CD79A | Hs.79630 | CD79A antigen | 4 |
| | | | | | | | | | (immunoglobulin- | |
| | | | | | | | | | associated alpha) | |
| 205297_s_at | 7.0E−04 | 3.1E−03 | 1.1E−03 | 6.3E−04 | Yes | Yes | CD79B | Hs.89575 | CD79B antigen | 4 |
| | | | | | | | | | (immunoglobulin- | |
| | | | | | | | | | associated beta) | |
| 207176_s_at | 5.0E−01 | 4.9E−02 | 4.5E−01 | 3.8E−02 | Yes | No | CD80 | Hs.838 | CD80 antigen (CD28 | 4 |
| | | | | | | | | | antigen ligand 1, B7-1 | |
| | | | | | | | | | antigen) | |
| 210356_x_at | 2.4E−02 | 6.6E−03 | 1.9E−03 | 5.9E−04 | Yes | No | MS4A1 | Hs.438040 | CD20 antigen | 4 |
| 217418_x_at | 1.5E−02 | 3.5E−03 | 3.0E−03 | 7.2E−04 | Yes | No | MS4A1 | Hs.438040 | CD20 antigen | 4 |
| 221969_at | 8.5E−03 | 3.2E−02 | 6.1E−03 | 3.1E−02 | Yes | No | PAX5 | Hs.22030 | paired box gene 5 (B-cell | 7 |
| | | | | | | | | | lineage specific activator | |
| | | | | | | | | | protein) | |
| 215346_at | 1.6E−01 | 6.1E−02 | 1.5E−01 | 7.0E−02 | Yes | No | TNFRSF5 | Hs.504816 | CD40 antigen | 4 |
| 35150_at | 2.5E−01 | 1.3E−01 | 8.9E−02 | 6.3E−02 | Yes | No | TNFRSF5 | Hs.504816 | CD40 antigen | 4 |
| 220068_at | 6.7E−03 | 1.5E−02 | 1.7E−01 | 6.4E−01 | Yes | No | VPREB3 | Hs.136713 | pre-B lymphocyte gene 3 | 8 |
| 206802_at | 8.0E−01 | 7.5E−01 | 9.8E−01 | 6.8E−01 | No | No | PAX5 | Hs.22030 | paired box gene 5 (B-cell | 7 |
| | | | | | | | | | lineage specific activator | |
| | | | | | | | | | protein) | |
| 205153_s_at | 1.0E−01 | 4.9E−02 | 2.3E−02 | 1.1E−02 | No | No | TNFRSF5 | Hs.504816 | CD40 antigen | 4 |
| 222292_at | 2.1E−01 | 4.4E−01 | 4.1E−01 | 6.6E−02 | No | No | TNFRSF5 | Hs.504816 | CD40 antigen | 4 |

TABLE 9A
Pre-B/GC Marker Genes

| | Log2 fluorescence intensity | | | | | | | | | | | |
|---|---|---|---|---|---|---|---|---|---|---|---|---|
| | Ly1 | | Ly1 | | Ly7 | | | | | | | |
| Probesets | (A1) | Ly1 (A2) | (A3) | Ly7 (B1) | (B2) | Ly7 (B3) | Ly8 (C1) | Ly8 (C2) | Ly8 (C3) | D4 (D1) | D4 (D2) | D4 (D3) |
| 219488_at | 5.20 | 5.39 | 5.13 | 5.80 | 5.94 | 5.60 | 5.40 | 5.34 | 5.33 | 5.84 | 5.77 | 5.52 |
| 204639_at | 8.75 | 8.80 | 8.72 | 9.11 | 9.15 | 8.83 | 8.78 | 8.89 | 8.83 | 5.93 | 6.35 | 5.98 |
| 203140_at | 8.23 | 7.80 | 9.55 | 9.61 | 9.72 | 9.33 | 8.39 | 8.31 | 7.70 | 9.82 | 10.45 | 9.94 |
| 215990_s_at | 7.37 | 6.79 | 7.81 | 8.18 | 8.00 | 7.78 | 7.47 | 7.01 | 6.83 | 8.45 | 8.89 | 8.25 |
| 208650_s_at | 8.87 | 8.94 | 9.42 | 8.48 | 8.33 | 8.51 | 9.08 | 9.14 | 9.02 | 3.88 | 3.93 | 4.12 |
| 208651_x_at | 9.18 | 9.18 | 9.57 | 8.92 | 8.82 | 8.87 | 9.26 | 9.36 | 9.01 | 6.22 | 6.34 | 6.23 |
| 209771_x_at | 11.06 | 11.00 | 11.43 | 10.63 | 10.45 | 10.50 | 11.13 | 11.13 | 10.86 | 6.02 | 6.69 | 6.70 |
| 209772_s_at | 9.04 | 8.92 | 9.10 | 8.21 | 8.10 | 8.25 | 9.20 | 8.71 | 8.61 | 7.01 | 6.93 | 6.84 |
| 266_s_at | 8.55 | 8.65 | 9.22 | 8.30 | 8.13 | 8.35 | 8.60 | 8.66 | 8.61 | 5.50 | 4.92 | 5.35 |
| 201005_at | 10.54 | 10.55 | 10.64 | 4.88 | 4.53 | 4.83 | 10.91 | 10.82 | 10.60 | 5.25 | 4.95 | 4.80 |
| 203066_at | 8.28 | 9.29 | 9.15 | 5.61 | 6.06 | 5.98 | 8.64 | 9.25 | 8.62 | 6.19 | 6.26 | 6.02 |
| 209374_s_at | 12.45 | 12.38 | 12.47 | 12.36 | 12.36 | 12.26 | 12.49 | 12.46 | 12.41 | 6.35 | 5.73 | 5.78 |
| 212827_at | 12.25 | 12.13 | 12.18 | 11.99 | 12.17 | 11.86 | 12.24 | 12.01 | 11.91 | 6.42 | 6.55 | 6.25 |
| 213674_x_at | 9.33 | 7.92 | 9.07 | 7.08 | 7.36 | 7.05 | 9.34 | 8.15 | 8.32 | 6.30 | 6.00 | 5.70 |
| 222285_at | 6.09 | 5.74 | 6.62 | 5.59 | 5.99 | 5.48 | 5.90 | 5.86 | 5.69 | 5.61 | 5.54 | 5.43 |
| 206660_at | 10.68 | 11.35 | 10.55 | 5.41 | 4.82 | 4.94 | 9.07 | 10.28 | 10.32 | 5.10 | 4.93 | 5.30 |
| 203434_s_at | 7.60 | 7.86 | 8.01 | 7.56 | 7.39 | 7.64 | 8.20 | 8.29 | 8.55 | 8.97 | 8.79 | 9.05 |
| 203435_s_at | 8.64 | 8.83 | 8.81 | 8.37 | 7.97 | 8.43 | 8.75 | 8.99 | 9.11 | 8.96 | 8.86 | 8.97 |
| 206591_at | 7.55 | 8.15 | 8.76 | 4.39 | 4.15 | 4.25 | 8.34 | 8.84 | 8.68 | 4.23 | 4.00 | 4.32 |
| 215117_at | 4.08 | 4.38 | 4.81 | 3.72 | 3.60 | 3.69 | 4.16 | 4.87 | 4.38 | 3.78 | 3.78 | 3.72 |
| 213435_at | 6.68 | 6.84 | 7.27 | 5.62 | 5.52 | 5.94 | 7.01 | 7.31 | 7.28 | 5.24 | 4.63 | 5.11 |
| 215591_at | 3.86 | 3.90 | 3.70 | 3.83 | 3.66 | 3.76 | 3.85 | 3.77 | 3.84 | 3.85 | 3.75 | 3.77 |
| 209995_s_at | 11.95 | 12.19 | 12.19 | 10.79 | 11.03 | 10.72 | 12.13 | 12.27 | 12.15 | 8.59 | 8.80 | 8.85 |
| 39318_at | 12.14 | 12.32 | 12.26 | 11.06 | 11.18 | 10.93 | 12.27 | 12.49 | 12.25 | 8.75 | 8.94 | 8.48 |
| 221349_at | 7.42 | 7.98 | 8.39 | 3.97 | 4.10 | 3.83 | 7.19 | 7.15 | 7.10 | 4.75 | 4.85 | 4.35 |
| 210487_at | 4.94 | 4.60 | 4.78 | 5.05 | 4.70 | 4.78 | 4.91 | 4.79 | 4.89 | 5.12 | 5.05 | 4.87 |
| 203939_at | 4.68 | 4.72 | 4.66 | 4.71 | 4.61 | 4.67 | 4.68 | 4.62 | 4.78 | 4.66 | 4.73 | 4.73 |

TABLE 9B
Pre-B/GC Marker Genes

| | t-test (two-tailed) | | | | | | Gene | | | |
|---|---|---|---|---|---|---|---|---|---|---|
| Probesets | pAB | pAD | pBC | pCD | Measured | Changed | Symbol | UniGene ID | Gene Title | Ref. |
| 219488_at | 1.4E−02 | 2.2E−02 | 4.3E−02 | 6.3E−02 | Yes | No | A4GALT | Hs.105956 | alpha 1,4- | 9 |
| | | | | | | | | | galactosyltransferase | |
| 204639_at | 1.0E−01 | 1.8E−03 | 1.8E−01 | 1.4E−03 | Yes | No | ADA | Hs.407135 | adenosine deaminase | 10 |
| 203140_at | 1.9E−01 | 8.5E−02 | 9.9E−03 | 2.8E−03 | Yes | No | BCL6 | Hs.155024 | B-cell CLL/lymphoma | 6 |
| | | | | | | | | | 6 (zinc finger protein | |
| | | | | | | | | | 51) | |
| 215990_s_at | 1.4E−01 | 3.4E−02 | 2.5E−02 | 6.1E−03 | Yes | No | BCL6 | Hs.155024 | B-cell CLL/lymphoma | 6 |
| | | | | | | | | | 6 (zinc finger protein | |
| | | | | | | | | | 51) | |
| 208650_s_at | 5.4E−02 | 2.1E−04 | 1.3E−03 | 1.2E−05 | Yes | No | CD24 | Hs.375108 | CD24 antigen | 4 |
| 208651_x_at | 7.0E−02 | 8.9E−04 | 7.2E−02 | 3.9E−04 | Yes | No | CD24 | Hs.375108 | CD24 antigen | 4 |
| 209771_x_at | 2.8E−02 | 2.2E−04 | 1.2E−02 | 7.2E−04 | Yes | No | CD24 | Hs.375108 | CD24 antigen | 4 |
| 209772_s_at | 3.4E−04 | 9.5E−06 | 6.2E−02 | 6.0E−03 | Yes | No | CD24 | Hs.375108 | CD24 antigen | 4 |
| 266_s_at | 1.1E−01 | 2.5E−04 | 2.5E−02 | 2.4E−03 | Yes | No | CD24 | Hs.375108 | CD24 antigen | 4 |
| 201005_at | 1.1E−04 | 2.7E−04 | 2.4E−06 | 1.0E−05 | Yes | Yes | CD9 | Hs.387579 | CD9 antigen (p24) | 4 |
| 203066_at | 4.3E−03 | 9.8E−03 | 5.9E−04 | 2.7E−03 | Yes | No | GALNAC4S- | Hs.523379 | B cell RAG associated | 4 |
| | | | | | | | | | protein 6ST | |
| 209374_s_at | 8.0E−02 | 7.5E−04 | 5.0E−02 | 7.6E−04 | Yes | No | IGHM | Hs.439852 | immunoglobulin heavy | 4 |
| | | | | | | | | | constant mu | |
| 212827_at | 1.7E−01 | 3.8E−05 | 7.4E−01 | 2.0E−06 | Yes | No | IGHM | Hs.439852 | immunoglobulin heavy | 4 |
| | | | | | | | | | constant mu | |
| 213674_x_at | 5.9E−02 | 1.4E−02 | 5.3E−02 | 9.4E−03 | Yes | No | IGHM | Hs.439852 | immunoglobulin heavy | 4 |
| | | | | | | | | | constant mu | |
| 222285_at | 2.1E−01 | 1.3E−01 | 5.1E−01 | 3.0E−02 | Yes | No | IGHM | Hs.439852 | immunoglobulin heavy | 4 |
| | | | | | | | | | constant mu | |
| 206660_at | 8.7E−05 | 4.3E−04 | 2.5E−03 | 4.9E−03 | Yes | Yes | IGLL1 | Hs.348935 | immunoglobulin | 11 |
| | | | | | | | | | lambda-like | |
| | | | | | | | | | polypeptide 1 | |
| 203434_s_at | 1.2E−01 | 2.8E−03 | 4.7E−03 | 1.4E−02 | Yes | No | MME | Hs.307734 | CALLA (CD10) | 4 |
| 203435_s_at | 5.9E−02 | 8.0E−02 | 2.2E−02 | 8.8E−01 | Yes | No | MME | Hs.307734 | CALLA (CD10) | 4 |
| 206591_at | 6.2E−03 | 4.9E−03 | 1.5E−04 | 5.3E−05 | Yes | Yes | RAG1 | Hs.73958 | recombination | 4 |
| | | | | | | | | | activating gene 1 | |
| 215117_at | 6.6E−02 | 8.5E−02 | 5.8E−02 | 7.4E−02 | Yes | No | RAG2 | Hs.159376 | recombination | 4 |
| | | | | | | | | | activating gene 2 | |
| 213435_at | 6.2E−03 | 1.6E−03 | 1.0E−03 | 1.8E−03 | Yes | Yes | SATB2 | Hs.412327 | SATB family member | 12 |
| | | | | | | | | | 2 | |
| 215591_at | 4.0E−01 | 6.7E−01 | 2.9E−01 | 5.1E−01 | Yes | No | SATB2 | Hs.412327 | SATB family member | 12 |
| | | | | | | | | | 2 | |
| 209995_s_at | 6.0E−04 | 7.8E−06 | 1.4E−03 | 2.9E−05 | Yes | Yes | TCL1A | Hs.2484 | T-cell leukemia/ | 13 |
| | | | | | | | | | lymphoma 1A | |
| 39318_at | 3.2E−04 | 3.8E−04 | 2.7E−04 | 1.2E−04 | Yes | Yes | TCL1A | Hs.2484 | T-cell leukemia/ | 13 |
| | | | | | | | | | lymphoma 1A | |
| 221349_at | 3.1E−03 | 1.7E−03 | 1.5E−04 | 3.0E−03 | Yes | Yes | VPREB1 | Hs.247979 | pre-B lymphocyte | 4 |
| | | | | | | | | | gene 1 | |
| 210487_at | 6.5E−01 | 1.3E−01 | 8.6E−01 | 1.7E−01 | No | No | DNTT | Hs.397294 | deoxynucleotidyl- | 4 |
| | | | | | | | | | transferase, terminal | |
| 203939_at | 5.7E−01 | 5.2E−01 | 6.4E−01 | 8.1E−01 | No | No | NT5E | Hs.153952 | 5′-nucleotidase, ecto | 4 |
| | | | | | | | | | (CD73) | |

TABLE 10A
CD40/BCR Signaling Genes

| | Log2 fluorescence intensity | | | | | | | | | | | |
|---|---|---|---|---|---|---|---|---|---|---|---|---|
| | Ly1 | | Ly1 | | Ly7 | | | | | | | |
| Probesets | (A1) | Ly1 (A2) | (A3) | Ly7 (B1) | (B2) | Ly7 (B3) | Ly8 (C1) | Ly8 (C2) | Ly8 (C3) | D4 (D1) | D4 (D2) | D4 (D3) |
| 207163_s_at | 8.37 | 8.44 | 8.22 | 8.02 | 7.99 | 7.73 | 8.91 | 8.88 | 9.02 | 7.82 | 8.07 | 8.05 |
| 205367_at | 7.63 | 7.61 | 7.50 | 10.59 | 10.54 | 10.27 | 7.14 | 7.60 | 7.42 | 10.20 | 10.20 | 10.16 |
| 205263_at | 7.40 | 7.11 | 7.48 | 7.15 | 7.15 | 7.30 | 7.21 | 7.26 | 7.75 | 6.52 | 6.52 | 7.01 |
| 207655_s_at | 8.48 | 8.44 | 9.00 | 9.52 | 9.42 | 9.15 | 8.32 | 8.53 | 8.60 | 7.89 | 7.90 | 8.20 |
| 220059_at | 8.32 | 8.08 | 8.17 | 10.14 | 10.19 | 10.19 | 8.09 | 7.95 | 7.95 | 9.89 | 9.72 | 9.93 |
| 205504_at | 5.84 | 5.90 | 6.11 | 8.32 | 8.16 | 7.96 | 6.01 | 5.67 | 5.89 | 8.53 | 8.41 | 8.56 |
| 202987_at | 3.92 | 4.01 | 3.98 | 3.91 | 3.98 | 3.98 | 3.98 | 3.98 | 3.91 | 4.05 | 4.00 | 3.99 |
| 215411_s_at | 8.69 | 8.39 | 8.38 | 7.63 | 7.89 | 7.72 | 8.67 | 8.42 | 8.46 | 7.63 | 8.29 | 7.97 |
| 202514_at | 5.84 | 5.68 | 6.02 | 6.28 | 5.89 | 6.29 | 5.72 | 5.37 | 5.83 | 7.48 | 7.16 | 7.52 |
| 202515_at | 7.80 | 7.90 | 8.22 | 8.34 | 8.38 | 8.41 | 7.32 | 7.66 | 7.95 | 9.27 | 9.16 | 9.57 |
| 202516_s_at | 4.11 | 4.09 | 4.21 | 4.25 | 4.27 | 4.29 | 4.08 | 4.01 | 4.00 | 4.35 | 4.38 | 4.65 |
| 210105_s_at | 7.34 | 6.89 | 7.31 | 9.23 | 9.39 | 9.09 | 8.35 | 7.31 | 7.86 | 6.33 | 6.57 | 6.74 |
| 212486_s_at | 4.25 | 4.27 | 4.29 | 4.44 | 4.47 | 4.50 | 4.50 | 4.35 | 4.41 | 4.34 | 4.45 | 4.27 |
| 216033_s_at | 5.83 | 5.47 | 5.45 | 7.46 | 7.51 | 7.15 | 6.70 | 5.65 | 5.99 | 5.34 | 5.24 | 5.18 |
| 215075_s_at | 8.22 | 8.28 | 8.29 | 9.36 | 8.96 | 9.22 | 8.40 | 8.16 | 8.52 | 8.88 | 8.84 | 8.95 |
| 209341_s_at | 6.54 | 6.37 | 6.60 | 6.94 | 6.59 | 6.67 | 6.40 | 6.25 | 6.63 | 6.68 | 6.61 | 7.33 |
| 209342_s_at | 5.13 | 5.16 | 5.24 | 5.28 | 5.44 | 5.22 | 5.11 | 4.98 | 5.01 | 5.21 | 5.56 | 5.47 |
| 204890_s_at | 6.02 | 5.83 | 5.85 | 8.82 | 8.88 | 8.99 | 6.16 | 6.07 | 5.98 | 8.20 | 8.66 | 8.47 |
| 204891_s_at | 6.82 | 6.23 | 6.68 | 10.30 | 10.26 | 10.18 | 6.71 | 6.30 | 6.10 | 9.51 | 9.88 | 9.69 |
| 202625_at | 7.52 | 7.05 | 6.96 | 8.05 | 8.27 | 7.82 | 7.75 | 7.24 | 7.63 | 8.01 | 8.07 | 8.29 |
| 202626_s_at | 8.72 | 8.09 | 8.04 | 9.19 | 9.51 | 8.74 | 9.08 | 8.55 | 8.65 | 9.02 | 9.22 | 9.30 |
| 210754_s_at | 8.53 | 7.97 | 8.02 | 9.13 | 9.22 | 8.82 | 8.96 | 8.19 | 8.62 | 9.01 | 9.00 | 9.13 |
| 202670_at | 8.89 | 8.59 | 8.73 | 9.68 | 9.64 | 9.73 | 8.99 | 8.45 | 8.93 | 9.89 | 9.95 | 10.12 |
| 202424_at | 9.87 | 9.65 | 9.69 | 9.89 | 9.91 | 9.76 | 9.95 | 9.69 | 9.59 | 10.40 | 10.75 | 10.68 |
| 213490_s_at | 7.94 | 7.58 | 7.44 | 7.80 | 7.95 | 7.56 | 7.97 | 7.51 | 7.38 | 8.42 | 8.90 | 8.51 |
| 205192_at | 5.77 | 5.64 | 5.85 | 6.24 | 6.35 | 6.41 | 5.69 | 6.02 | 5.69 | 5.95 | 5.90 | 5.78 |
| 204725_s_at | 7.15 | 6.75 | 7.14 | 7.83 | 7.71 | 7.78 | 7.05 | 6.66 | 6.99 | 7.45 | 7.60 | 7.70 |
| 211063_s_at | 6.89 | 6.73 | 7.12 | 7.76 | 7.33 | 7.66 | 6.79 | 6.67 | 6.99 | 7.56 | 7.21 | 7.74 |
| 209239_at | 8.35 | 8.59 | 8.52 | 7.22 | 7.63 | 7.16 | 8.37 | 8.51 | 8.19 | 8.15 | 8.49 | 8.00 |
| 207535_sat | 5.46 | 5.26 | 5.22 | 6.18 | 6.01 | 5.92 | 5.38 | 5.08 | 5.09 | 5.51 | 5.90 | 5.73 |
| 201502_s_at | 9.28 | 8.98 | 8.75 | 9.09 | 9.14 | 8.67 | 9.01 | 8.78 | 8.68 | 9.09 | 9.69 | 9.61 |
| 203879_at | 8.35 | 8.58 | 8.42 | 7.09 | 6.98 | 6.98 | 7.93 | 8.47 | 8.19 | 7.56 | 7.75 | 7.61 |
| 211230_s_at | 6.07 | 5.96 | 5.42 | 5.58 | 5.82 | 5.30 | 5.95 | 5.81 | 5.37 | 5.58 | 5.98 | 5.40 |
| 204613_at | 9.30 | 9.43 | 9.38 | 9.25 | 9.63 | 9.25 | 9.37 | 9.37 | 9.34 | 9.19 | 9.44 | 9.34 |
| 207957_s_at | 5.70 | 5.09 | 5.70 | 5.41 | 5.87 | 5.58 | 8.16 | 7.82 | 7.67 | 4.39 | 4.59 | 4.42 |
| 209685_s_at | 7.37 | 6.44 | 6.92 | 7.33 | 7.47 | 7.24 | 9.65 | 9.18 | 8.97 | 5.98 | 5.98 | 5.71 |
| 201244_s_at | 8.29 | 8.30 | 8.19 | 8.62 | 8.29 | 8.43 | 8.63 | 8.44 | 8.49 | 8.06 | 8.28 | 8.38 |
| 201783_s_at | 8.92 | 8.75 | 8.73 | 9.02 | 8.78 | 8.74 | 8.90 | 8.78 | 8.67 | 8.52 | 8.39 | 8.45 |
| 209878_s_at | 8.04 | 7.74 | 7.64 | 7.97 | 8.00 | 7.87 | 8.09 | 7.67 | 7.73 | 7.33 | 7.82 | 7.42 |
| 205205_at | 7.31 | 7.02 | 6.65 | 7.70 | 7.99 | 7.46 | 7.25 | 6.85 | 6.45 | 7.51 | 7.92 | 7.51 |
| 212777_at | 5.76 | 5.73 | 5.67 | 5.92 | 6.09 | 6.00 | 5.78 | 5.68 | 5.72 | 6.03 | 5.78 | 5.82 |
| 212780_at | 6.36 | 5.94 | 5.97 | 6.28 | 5.81 | 6.13 | 6.25 | 5.69 | 6.41 | 5.82 | 5.79 | 6.27 |
| 208991_at | 8.05 | 7.95 | 7.88 | 7.14 | 7.51 | 7.43 | 8.24 | 8.17 | 7.94 | 6.72 | 6.86 | 7.39 |
| 207540_s_at | 9.18 | 9.26 | 9.23 | 8.25 | 8.11 | 8.30 | 9.16 | 9.14 | 9.44 | 8.20 | 8.38 | 8.56 |
| 209269_s_at | 6.75 | 6.81 | 7.10 | 6.33 | 6.52 | 6.35 | 6.94 | 6.74 | 7.14 | 6.22 | 6.22 | 6.25 |
| 207616_s_at | 8.31 | 7.68 | 7.48 | 8.12 | 8.06 | 8.05 | 8.29 | 7.58 | 8.20 | 8.65 | 8.61 | 8.53 |
| 209451_at | 5.19 | 4.51 | 5.20 | 5.32 | 5.25 | 5.66 | 5.18 | 4.88 | 5.39 | 5.59 | 5.65 | 5.70 |
| 210458_s_at | 3.97 | 3.83 | 3.98 | 3.91 | 3.91 | 3.99 | 3.93 | 3.84 | 3.98 | 4.14 | 4.12 | 4.11 |
| 205599_at | 5.00 | 5.55 | 5.30 | 5.47 | 6.03 | 5.60 | 5.07 | 5.44 | 5.16 | 5.51 | 5.90 | 5.51 |
| 208315_x_at | 5.55 | 5.65 | 5.56 | 5.33 | 5.31 | 4.93 | 5.74 | 5.57 | 5.51 | 5.75 | 5.81 | 5.80 |
| 221571_at | 7.09 | 6.94 | 7.35 | 5.83 | 5.46 | 5.85 | 6.77 | 6.73 | 6.93 | 7.26 | 7.41 | 7.60 |
| 204352_at | 5.72 | 5.67 | 6.02 | 6.04 | 6.16 | 5.88 | 5.54 | 5.54 | 5.40 | 5.16 | 5.87 | 5.35 |
| 206219_s_at | 6.09 | 6.19 | 5.90 | 8.23 | 8.18 | 8.01 | 6.40 | 6.15 | 6.04 | 8.44 | 8.86 | 9.03 |
| 215988_s_at | 4.45 | 4.32 | 4.29 | 4.34 | 4.32 | 4.23 | 4.34 | 4.39 | 4.26 | 4.33 | 4.15 | 4.36 |
| 211027_s_at | 4.96 | 4.64 | 4.82 | 4.93 | 4.98 | 4.92 | 4.82 | 4.63 | 4.92 | 4.93 | 4.95 | 4.99 |
| 207187_at | 7.17 | 7.41 | 7.19 | 7.19 | 7.37 | 7.31 | 7.41 | 7.34 | 7.08 | 7.13 | 7.30 | 6.98 |
| 211108_s_at | 4.89 | 4.86 | 4.80 | 4.87 | 4.90 | 4.75 | 5.10 | 4.77 | 4.81 | 4.91 | 4.75 | 4.65 |
| 211109_at | 4.61 | 4.70 | 4.63 | 4.77 | 4.65 | 4.64 | 4.70 | 4.67 | 4.62 | 4.61 | 4.74 | 4.63 |
| 213487_at | 6.08 | 6.09 | 5.75 | 6.04 | 5.97 | 6.03 | 5.79 | 5.95 | 6.00 | 6.17 | 6.23 | 5.99 |
| 209636_at | 5.26 | 5.15 | 4.96 | 6.02 | 5.62 | 5.38 | 5.43 | 5.18 | 5.00 | 5.55 | 5.84 | 5.51 |
| 211524_at | 4.38 | 4.48 | 4.55 | 4.30 | 4.55 | 4.48 | 4.31 | 4.47 | 4.37 | 4.40 | 4.44 | 4.43 |
| 216995_x_at | 5.36 | 5.54 | 5.37 | 5.45 | 5.58 | 5.39 | 5.35 | 5.51 | 5.41 | 5.41 | 5.42 | 5.27 |
| 208992_s_at | 7.88 | 7.90 | 7.71 | 7.45 | 7.52 | 7.51 | 7.95 | 7.75 | 7.98 | 7.61 | 7.33 | 7.52 |
| 204413_at | 5.65 | 5.94 | 5.50 | 5.78 | 5.93 | 5.58 | 5.49 | 5.84 | 5.49 | 5.46 | 5.73 | 5.28 |
| 205558_at | 6.49 | 6.36 | 6.72 | 6.31 | 6.41 | 6.41 | 6.30 | 6.50 | 6.44 | 6.00 | 6.45 | 6.20 |

TABLE 10B
CD40/BCR Signaling Genes

| | t-test (two-tailed) | | | | | | Gene | | | |
|---|---|---|---|---|---|---|---|---|---|---|
| Probesets | pAB | pAD | pBC | pCD | Measured | Changed | Symbol | UniGene ID | Gene Title | Ref. |
| 207163_s_at | 2.3E−02 | 2.5E−02 | 2.6E−03 | 1.5E−03 | Yes | No | AKT1 | Hs.368861 | v-akt murine thymoma viral | 14 |
| | | | | | | | | | oncogene homolog 1 | |
| 205367_at | 2.9E−04 | 7.2E−05 | 8.6E−05 | 2.1E−03 | Yes | Yes | APS | Hs.371366 | adaptor protein with | 15 |
| | | | | | | | | | pleckstrin homology and | |
| | | | | | | | | | src homology 2 domains | |
| 205263_at | 3.9E−01 | 3.7E−02 | 3.6E−01 | 3.9E−02 | Yes | No | BCL10 | Hs.193516 | B-cell CLL/lymphoma 10 | 16 |
| 207655_s_at | 3.6E−02 | 5.0E−02 | 3.8E−03 | 2.2E−02 | Yes | No | BLNK | Hs.167746 | B-cell linker | 17 |
| 220059_at | 7.0E−04 | 6.3E−05 | 1.2E−04 | 3.9E−05 | Yes | Yes | BRDG1 | Hs.121128 | BCR downstream signaling | 18 |
| | | | | | | | | | 1 | |
| 205504_at | 1.2E−04 | 7.0E−05 | 9.4E−05 | 2.2E−04 | Yes | Yes | BTK | Hs.159494 | Bruton | 19 |
| | | | | | | | | | agammaglobulinemia | |
| | | | | | | | | | tyrosine kinase | |
| 202987_at | 7.1E−01 | 2.8E−01 | 9.9E−01 | 1.3E−01 | Yes | No | ACT1 | Hs.437508 | Nuclear factor kappa B | 20 |
| | | | | | | | | | activator 1 | |
| 215411_s_at | 5.2E−03 | 9.2E−02 | 2.2E−03 | 8.5E−02 | Yes | No | ACT1 | Hs.437508 | Nuclear factor kappa B | 20 |
| | | | | | | | | | activator 1 | |
| 202514_at | 1.4E−01 | 5.5E−04 | 5.5E−02 | 7.3E−04 | Yes | No | DLG1 | Hs.389893 | discs, large homolog 1 | 21 |
| | | | | | | | | | (Drosophila) | |
| 202515_at | 8.1E−02 | 1.5E−03 | 5.6E−02 | 2.7E−03 | Yes | No | DLG1 | Hs.389893 | discs, large homolog 1 | 21 |
| | | | | | | | | | (Drosophila) | |
| 202516_s_at | 5.6E−02 | 6.4E−02 | 2.6E−03 | 4.0E−02 | Yes | No | DLG1 | Hs389893 | discs, large homolog 1 | 22 |
| | | | | | | | | | (Drosophila) | |
| 210105_s_at | 8.1E−04 | 3.1E−02 | 3.5E−02 | 3.6E−02 | Yes | No | FYN | Hs.390567 | FYN oncogene related to | 22 |
| | | | | | | | | | SRC, FGR, YES | |
| 212486_s_at | 1.0E−03 | 2.6E−01 | 3.8E−01 | 3.7E−01 | Yes | No | FYN | Hs.390567 | FYN oncogene related to | 22 |
| | | | | | | | | | SRC, FGR, YES | |
| 216033_s_at | 4.4E−04 | 9.9E−02 | 4.3E−02 | 1.0E−01 | Yes | No | FYN | Hs.390567 | FYN oncogene related to | 22 |
| | | | | | | | | | SRC, FGR, YES | |
| 215075_s_at | 1.4E−02 | 1.9E−04 | 6.8E−03 | 2.9E−02 | Yes | No | GRB2 | Hs.411366 | growth factor receptor- | 23 |
| | | | | | | | | | bound protein 2 | |
| 209341_s_at | 1.6E−01 | 2.5E−01 | 1.2E−01 | 1.8E−01 | Yes | No | IKBKB | Hs.413513 | inhibitor of kappa light | 24 |
| | | | | | | | | | polypeptide gene enhancer | |
| | | | | | | | | | in B-cells, kinase beta | |
| 209342_s_at | 1.5E−01 | 1.4E−01 | 2.8E−02 | 5.5E−02 | Yes | No | IKBKB | Hs.413513 | inhibitor of kappa light | 24 |
| | | | | | | | | | polypeptide gene enhancer | |
| | | | | | | | | | in B-cells, kinase beta | |
| 204890_s_at | 4.0E−06 | 6.5E−04 | 2.6E−06 | 1.0E−03 | Yes | Yes | LCK | Hs.1765 | lymphocyte-specific protein | 25 |
| | | | | | | | | | tyrosine kinase | |
| 204891_s_at | 1.6E−03 | 3.6E−04 | 1.5E−03 | 3.3E−04 | Yes | Yes | LCK | Hs.1765 | lymphocyte-specific protein | 25 |
| | | | | | | | | | tyrosine kinase | |
| 202625_at | 1.9E−02 | 1.8E−02 | 6.7E−02 | 4.2E−02 | Yes | No | LYN | Hs.80887 | v-yes-1 Yamaguchi | 26 |
| | | | | | | | | | sarcoma viral related | |
| | | | | | | | | | oncogene homolog | |
| 202626_s_at | 5.1E−02 | 4.1E−02 | 2.4E−01 | 1.1E−01 | Yes | No | LYN | Hs.80887 | v-yes-1 Yamaguchi | 26 |
| | | | | | | | | | sarcoma viral related | |
| | | | | | | | | | oncogene homolog | |
| 210754_s_at | 2.0E−02 | 3.4E−02 | 1.6E−01 | 1.7E−01 | Yes | No | LYN | Hs.80887 | v-yes-1 Yamaguchi | 26 |
| | | | | | | | | | sarcoma viral related | |
| | | | | | | | | | oncogene homolog | |
| 202670_at | 4.6E−03 | 4.5E−04 | 3.2E−02 | 1.1E−02 | Yes | No | MAP2K1 | Hs.132311 | mitogen-activated protein | 27 |
| | | | | | | | | | kinase kinase 1 | |
| 202424_at | 2.4E−01 | 4.2E−03 | 4.2E−01 | 4.6E−03 | Yes | No | MAP2K2 | Hs.366546 | mitogen-activated protein | 27 |
| | | | | | | | | | kinase kinase 2 | |
| 213490_s_at | 5.6E−01 | 1.0E−02 | 5.1E−01 | 1.4E−02 | Yes | No | MAP2K2 | Hs.366546 | mitogen-activated protein | 27 |
| | | | | | | | | | kinase kinase 2 | |
| 205192_at | 2.2E−03 | 1.9E−01 | 2.5E−02 | 5.8E−01 | Yes | No | MAP3K14 | Hs.440315 | NIK | 28 |
| 204725_s_at | 2.2E−02 | 2.9E−02 | 1.3E−02 | 1.4E−02 | Yes | No | NCK1 | Hs.54589 | NOK adaptor protein 1 | 29 |
| 211063_s_at | 1.8E−02 | 4.3E−02 | 1.1E−02 | 2.8E−02 | Yes | No | NCK1 | Hs.54589 | NCK adaptor protein 1 | 29 |
| 209239_at | 6.6E−03 | 1.9E−01 | 6.8E−03 | 4.6E−01 | Yes | No | NFKB1 | Hs.160557 | nuclear factor of kappa | 30 |
| | | | | | | | | | light polypeptide gene | |
| | | | | | | | | | enhancer in B-cells 1 | |
| | | | | | | | | | (p105) | |
| 207535_s_at | 2.4E−03 | 4.8E−02 | 3.2E−03 | 2.4E−02 | Yes | No | NFKB2 | Hs.73090 | nuclear factor of kappa | 31 |
| | | | | | | | | | light polypeptide gene | |
| | | | | | | | | | enhancer in B-cells 2 | |
| | | | | | | | | | (p49/p100) | |
| 201502_s_at | 8.8E−01 | 1.3E−01 | 4.7E−01 | 5.5E−02 | Yes | No | NFKBIA | Hs.81328 | nuclear factor of kappa | 30 |
| | | | | | | | | | light polypeptide gene | |
| | | | | | | | | | enhancer in B-cells | |
| | | | | | | | | | inhibitor, alpha | |
| 203879_at | 2.5E−04 | 8.6E−04 | 1.3E−02 | 5.7E−02 | Yes | No | PIK3CD | Hs.426967 | phosphoinositide-3-kinase, | 26 |
| | | | | | | | | | catalytic, delta polypeptide | |
| 211230_s_at | 3.8E−01 | 5.7E−01 | 5.7E−01 | 8.2E−01 | Yes | No | PIK3CD | Hs.426967 | phosphoinositide-3-kinase, | 26 |
| | | | | | | | | | catalytic, delta polypeptide | |
| 204613_at | 9.7E−01 | 5.9E−01 | 9.1E−01 | 6.5E−01 | Yes | No | PLCG2 | Hs.512298 | phospholipase C, gamma 2 | 26 |
| | | | | | | | | | (phosphatidylinositol- | |
| | | | | | | | | | Specific) | |
| 207957_s_at | 6.4E−01 | 2.8E−02 | 3.5E−04 | 4.2E−04 | Yes | No | PRKCB1 | Hs.349845 | protein kinase C, beta 1 | 32 |
| 209685_s_at | 2.4E−01 | 5.2E−02 | 6.1E−03 | 9.2E−04 | Yes | No | PRKCB1 | Hs.349845 | protein kinase C, beta 1 | 32 |
| 201244_s_at | 1.8E−01 | 8.7E−01 | 5.7E−01 | 8.4E−02 | Yes | No | RAF1 | Hs.257266 | v-raf-1 murine leukemia | 26 |
| | | | | | | | | | viral oncogene homolog 1 | |
| 201783_s_at | 7.2E−01 | 1.4E−02 | 6.1E−01 | 1.9E−02 | Yes | No | RELA | Hs.132594 | v-rel reticuloendotheliosis | 30 |
| | | | | | | | | | viral oncogene homolog A | |
| 209878_s_at | 3.8E−01 | 2.2E−01 | 4.8E−01 | 2.0E−01 | Yes | No | RELA | Hs.132594 | v-rel reticuloendotheliosis | 30 |
| | | | | | | | | | viral oncogene homolog A | |
| 205205_at | 4.6E−02 | 5.6E−02 | 4.3E−02 | 5.3E−02 | Yes | No | RELB | Hs.307905 | v-rel reticuloendotheliosis | 31 |
| | | | | | | | | | viral oncogene homolog B | |
| 212777_at | 1.4E−02 | 1.7E−01 | 1.4E−02 | 1.9E−01 | Yes | No | SOS1 | Hs.326392 | son of sevenless homolog 1 | 23 |
| | | | | | | | | | (Drosophila) | |
| 212780_at | 9.4E−01 | 5.6E−01 | 8.8E−01 | 6.0E−01 | Yes | No | SOS1 | Hs.326392 | son of sevenless homolog 1 | 23 |
| | | | | | | | | | (Drosophila) | |
| 208991_at | 1.9E−02 | 3.5E−02 | 6.8E−03 | 1.9E−02 | Yes | No | STAT3 | Hs.421342 | signal transducer and | 26 |
| | | | | | | | | | activator of transcription 3 | |
| 207540_s_at | 7.9E−04 | 1.1E−02 | 2.0E−03 | 3.7E−03 | Yes | No | SYK | Hs.192182 | spleen tyrosine kinase | 26 |
| 209269_s_at | 2.7E−02 | 2.6E−02 | 2.6E−02 | 2.6E−02 | Yes | No | SYK | Hs.192182 | spleen tyrosine kinase | 26 |
| 207616_s_at | 4.2E−01 | 8.7E−02 | 8.2E−01 | 1.2E−01 | Yes | No | TANK | Hs.146847 | TRAF family member- | 33 |
| | | | | | | | | | associated NFKB activator | |
| 209451_at | 1.9E−01 | 9.5E−02 | 2.6E−01 | 7.4E−02 | Yes | No | TANK | Hs.146847 | TRAF family member- | 33 |
| | | | | | | | | | associated NFKB activator | |
| 210458_s_at | 9.1E−01 | 4.8E−02 | 7.3E−01 | 3.3E−02 | Yes | No | TANK | Hs.146847 | TRAF family member- | 33 |
| | | | | | | | | | associated NFKB activator | |
| 205599_at | 1.5E−01 | 1.6E−01 | 8.5E−02 | 7.2E−02 | Yes | No | TRAF1 | Hs.438253 | TNF receptor-associated | 26 |
| | | | | | | | | | factor 1 | |
| 208315_x_at | 8.4E−02 | 1.4E−02 | 6.4E−02 | 1.2E−01 | Yes | No | TRAF3 | Hs.297660 | TNF receptor-associated | 26 |
| | | | | | | | | factor 3 | | |
| 221571_at | 1.3E−03 | 1.3E−01 | 5.2E−03 | 9.5E−03 | Yes | No | TRAF3 | Hs.297660 | TNF receptor-associated | 26 |
| | | | | | | | | factor 3 | | |
| 204352_at | 1.8E−01 | 2.5E−01 | 9.0E−03 | 8.9E−01 | Yes | No | TRAF5 | Hs.385685 | TNF receptor-associated | 26 |
| | | | | | | | | factor 5 | | |
| 206219_s_at | 6.2E−05 | 9.8E−04 | 2.9E−04 | 6.7E−04 | Yes | Yes | VAV1 | Hs.116237 | vav 1 oncogene | 23 |
| 215988_s_at | 4.0E−01 | 4.3E−01 | 5.7E−01 | 5.8E−01 | No | No | DLG1 | Hs.389893 | discs, large homolog 1 | 21 |
| | | | | | | | | (Drosophila) | | |
| 211027_s_at | 2.8E−01 | 2.4E−01 | 2.2E−01 | 1.8E−01 | No | No | IKBKB | Hs.413513 | inhibitor of kappa light | 24 |
| | | | | | | | | | polypeptide gene enhancer | |
| | | | | | | | | | in B-cells, kinase bete | |
| 207187_at | 7.5E−01 | 3.8E−01 | 9.1E−01 | 3.7E−01 | No | No | JAK3 | Hs.210387 | Janus kinase 3 (a protein | 26 |
| | | | | | | | | | tyrosine kinase, leukocyte) | |
| 211108_s_at | 9.1E−01 | 4.3E−01 | 6.7E−01 | 4.0E−01 | No | No | JAK3 | Hs.210387 | Janus kinase 3 (a protein | 26 |
| | | | | | | | | | tyrosine kinase, leukocyte) | |
| 211109_at | 4.6E−01 | 8.1E−01 | 6.2E−01 | 9.5E−01 | No | No | JAK3 | Hs.210387 | Janus kinase 3 (a protein | 26 |
| | | | | | | | | | tyrosine kinase, leukocyte) | |
| 213487_at | 7.4E−01 | 3.1E−01 | 2.4E−01 | 8.2E−02 | No | No | MAP2K2 | Hs.366546 | mitogen-activated protein | 27 |
| | | | | | | | | | kinase kinase 2 | |
| 209636_at | 8.2E−02 | 2.1E−02 | 1.2E−01 | 5.8E−02 | No | No | NFKB2 | Hs.73090 | nuclear factor of kappa | 14 |
| | | | | | | | | | light polypeptide gene | |
| | | | | | | | | | enhancer in B-cells 2 | |
| | | | | | | | | | (p49/p100) | |
| 211524_at | 7.8E−01 | 4.7E−01 | 5.5E−01 | 5.0E−01 | No | No | NFKB2 | Hs.73090 | nuclear factor of kappa | 14 |
| | | | | | | | | | light polypeptide gene | |
| | | | | | | | | | enhancer in B-cells 2 | |
| | | | | | | | | | (p49/p100) | |
| 216995_x_at | 5.8E−01 | 4.8E−01 | 5.4E−01 | 4.4E−01 | No | No | RAF1 | Hs.257266 | v-raf-1 murine leukemia | 26 |
| | | | | | | | | | viral oncogene homolog 1 | |
| 208992_s_at | 2.0E−02 | 3.1E−02 | 2.4E−02 | 2.1E−02 | No | No | STAT3 | Hs.421342 | signal transducer and | 26 |
| | | | | | | | | | activator of transcription 3 | |
| 204413_at | 7.0E−01 | 3.3E−01 | 3.7E−01 | 5.3E−01 | No | No | TRAF2 | Hs.437575 | TNF receptor-associated | 26 |
| | | | | | | | | | factor 2 | |
| 205558_at | 3.0E−01 | 1.4E−01 | 6.5E−01 | 2.7E−01 | No | No | TRAF6 | Hs.444172 | TNF receptor-associated | 26 |
| | | | | | | | | | factor 6 | |

FDR (false discovery rate) cutoffs are the same for Tables 5B and 6B. FDR cutoffs are the same for Tables 7B, 8B, 9B and 10B.

### TABLE REFERENCES

1. Donze O, Deng J, Curran J, Sladek R, Picard D, Sonenberg N. The protein kinase PKR: a molecular clock that sequentially activates survival and death programs. Embo J. 2004; 23:564-571.
2. Affymetrix, Santa Ana, Calif., USA. Gene Ontology Classification. 2004.
3. Granziero L, Ghia P, Circosta P, Gottardi D, Strola G, Geuna M, Montagna L, Piccoli P, Chilosi M, Caligaris-Cappio F. Survivin is expressed on CD40 stimulation and interfaces proliferation and apoptosis in B-cell chronic lymphocytic leukemia. Blood. 2001; 97:2777-2783.
4. Janeway C A, Travers P, Walport M, Shlomchik M. Immunobiology (ed 5th): Garland Publishing; 2001.
5. Collado A, de Andres A, Canadas E, Ruiz-Cabello F, Gomez O, Pedrinaci S, Gamido F. Characterization of CD44 antigen during lymphoid ontogeny. Immunobiology. 1991; 183:1-11.
6. Colomo L, Lopez-Guillermo A, Perales M, Rives S, Martinez A, Bosch F, Colomer D, Falini B, Montserrat E, Campo E. Clinical impact of the differentiation profile assessed by immunophenotyping in patients with diffuse large B-cell lymphoma. Blood. 2003; 101:78-84.
7. Hamada T, Yonetani N, Ueda C, Maesako Y, Akasaka H, Akasaka T, Ohno H, Kawakami K, Amakawa R, Okuma M. Expression of the PAX5/BSAP transcription factor in haematological tumour cells and further molecular characterization of the t(9; 14)(p13; q32) translocation in B-cell non-Hodgkin's lymphoma. Br J Haematol. 1998; 102:691-700.
8. Rosnet O, Mattei M G, Delattre O, Schiff C. VPREB3: cDNA characterization and expression in human and chromosome mapping in human and mouse. Cytogenet Cell Genet. 1999; 87:205-208.
9. Taga S, Tetaud C, Mangeney M, Tursz T, Wiels J. Sequential changes in glycolipid expression during human B cell differentiation: enzymatic bases. Biochim Biophys Acta. 1995; 1254:56-65.
10. Pieters R, Huismans D R, Loonen A H, Peters G J, Hahlen K, van der Does-van den Berg A, van Wering E R, Veerman A J. Adenosine deaminase and purine nucleoside phosphorylase in childhood lymphoblastic leukemia: relation with differentiation stage, in vitro drug resistance and clinical prognosis. Leukemia. 1992; 6:375-380.
11. Wang Y H, Nomura J, Faye-Petersen O M, Cooper M D. Surrogate light chain production during B cell differentiation: differential intracellular versus cell surface expression. J Immunol. 1998; 161:1132-1139.
12. Dobreva G, Dambacher J, Grosschedl R. SUMO modification of a novel MAR-binding protein, SATB2, modulates immunoglobulin mu gene expression. Genes Dev. 2003; 17:3048-3061.
13. Narducci M G, Pescarmona E, Lazzeri C, Signoretti S, Lavinia A M, Remotti D, Scala E, Baroni C D, Stoppacciaro A, Croce C M, Russo G. Regulation of TCL1 expression in B- and T-cell lymphomas and reactive lymphoid tissues. Cancer Res. 2000; 60:2095-2100.
14. Andjelic S, Hsia C, Suzuki H, Kadowaki T, Koyasu S, Liou H C. Phosphatidylinositol 3-kinase and NF-kappa B/Rel are at the divergence of CD40-mediated proliferation and survival pathways. J Immunol. 2000; 165:3860-3867.
15. Yokouchi M, Suzuki R, Masuhara M, Komiya S, Inoue A, Yoshimura A. Cloning and characterization of APS, an adaptor molecule containing PH and SH2 domains that is tyrosine phosphorylated upon B-cell receptor stimulation. Oncogene. 1997; 15:7-15.
16. Ruland J, Mak T W. Transducing signals from antigen receptors to nuclear factor kappaB. Immunol Rev. 2003; 193:93-100.
17. Tsuji S, Okamoto M, Yamada K, Okamoto N, Goitsuka R, Arnold R, Kiefer F, Kitamura D. B cell adaptor containing src homology 2 domain (BASH) links B cell receptor signaling to the activation of hematopoietic progenitor kinase 1. J Exp Med. 2001; 194:529-539.
18. Ohya K, Kajigaya S, Kitanaka A, Yoshida K, Miyazato A, Yamashita Y, Yamanaka T, Ikeda U, Shimada K, Ozawa K, Mano H. Molecular cloning of a docking protein, BRDG1, that acts downstream of the Tec tyrosine kinase. Proc Natl Acad Sci USA. 1999; 96:11976-11981.
19. Brunner C, Avots A, Kreth H W, Serfling E, Schuster V. Bruton's tyrosine kinase is activated upon CD40 stimulation in human B lymphocytes. Immunobiology. 2002; 206:432-440.
20. Qian Y, Qin J, Cui G, Naramura M, Snow E C, Ware C F, Fairchild R L, Omori S A, Rickert R C, Scott M, Kotzin B L, Li X. Act1, a Negative Regulator in CD40- and BAFF-Mediated B Cell Survival. Immunity. 2004; 21:575-587.
21. Xavier R, Rabizadeh S, Ishiguro K, Andre N, Ortiz J B, Wachtel H, Morris D G, Lopez-Ilasaca M, Shaw A C, Swat W, Seed B. Discs large (Dlg1) complexes in lymphocyte activation. J Cell Biol. 2004; 166:173-178.
22. Pleiman C M, Clark M R, Gauen L K, Winitz S, Coggeshall K M, Johnson G L, Shaw A S, Cambier J C. Mapping of sites on the Src family protein tyrosine kinases p55blk, p59fyn, and p56lyn which interact with the effector molecules phospholipase C-gamma 2, microtubule-associated protein kinase, GTPase-activating protein, and phosphatidylinositol 3-kinase. Mol Cell Biol. 1993; 13:5877-5887.
23. Smit L, van der Horst G, Borst J. Sos, Vav, and C3G participate in B cell receptor-induced signaling pathways and differentially associate with Shc-Grb2, Crk, and Crk-L adaptors. J Biol Chem. 1996; 271:8564-8569.
24. Ren H, Schmalstieg A, Yuan D, Gaynor R B. I-kappa B kinase beta is critical for B cell proliferation and antibody response. J Immunol. 2002; 168:577-587.
25. Dal Porto J M, Burke K, Cambier J C. Regulation of BCR signal transduction in B-1 cells requires the expression of the Src family kinase Lck. Immunity. 2004; 21:443-453.
26. van Kooten C, Banchereau J. CD40-CD40 ligand. J Leukoc Biol. 2000; 67:2-17.
27. Gulbins E, Brenner B, Schlottmann K, Koppenhoefer U, Linderkamp O, Coggeshall K M, Lang F. Activation of the Ras signaling pathway by the CD40 receptor. J Immunol. 1996; 157:2844-2850.
28. Ramakrishnan P, Wang W, Wallach D. Receptor-Specific Signaling for Both the Alternative and the Canonical NF-kappaB Activation Pathways by NF-kappaB-Inducing Kinase. Immunity. 2004; 21:477-489.
29. Fu C, Turck C W, Kurosaki T, Chan A C. BLNK: a central linker protein in B cell activation. Immunity. 1998; 9:93-103.
30. Berberich I, Shu G L, Clark E A. Cross-linking CD40 on B cells rapidly activates nuclear factor-kappa B. J Immunol. 1994; 153:4357-4366.
31. Beinke S, Ley S C. Functions of NF-kappaB1 and NF-kappaB2 in immune cell biology. Biochem J. 2004; Pt.
32. Sakata N, Kawasome H, Terada N, Gerwins P, Johnson G L, Gelfand E W. Differential activation and regulation of mitogen-activated protein kinases through the antigen receptor and CD40 in human B cells. Eur J Immunol. 1999; 29:2999-3008.
33. Cheng G, Baltimore D. TANK, a co-inducer with TRAF2 of TNF- and CD 40L-mediated NF-kappaB activation. Genes Dev. 1996; 10:963-973.

### Example 3

### Signaling in the LCK-VAV-MAPK Pathway Differs in CD40-Sensitive and CD40-Resistant Cell Lines

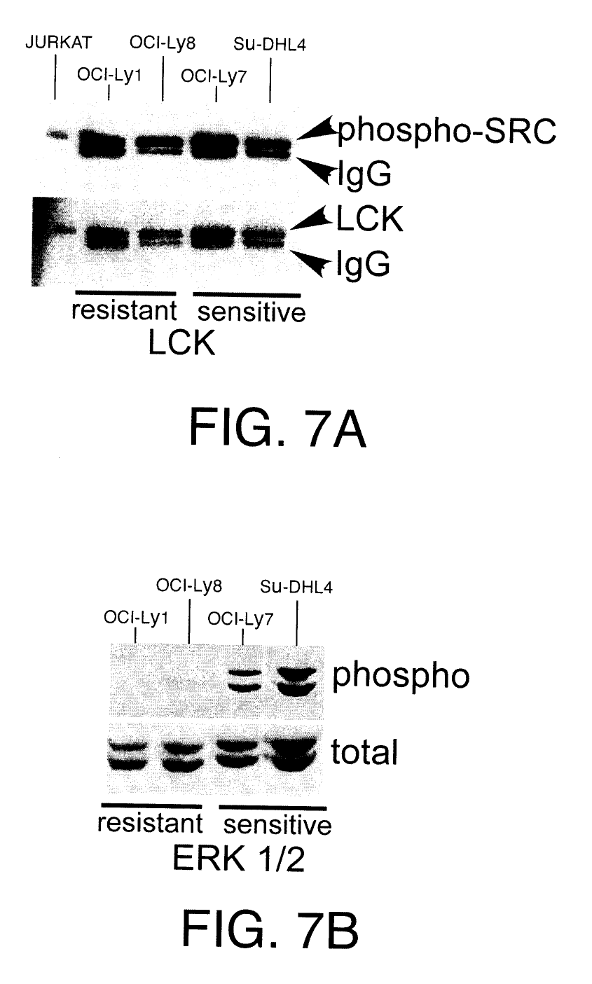

In lymphoma cells, CD40 signaling is mediated by three interacting pathways. Current evidence suggests that the NFκB pathway is the most important of these pathways for controlling cell survival. Microarray studies showed that CD40 sensitivity was not associated with differences in expression of transcripts encoding members of this pathway, but rather with changes in the expression level of LCK and VAV1, which activate the RAS-RAF-ERK pathway. To further characterize the association between the MAPK pathway and CD40 sensitivity, we investigated activation of LCK (which activates VAV1) and MAP kinases (which are activated by VAV1). LCK is a member of the src protein family of kinases, which can be phosphorylated at two sites, one of which activates the kinase, whereas the other is inhibitory. Western blots showed that activated LCK was present in all four cell lines in the absence of CD40 stimulation (FIG. 7A). This is consistent with previous results that have identified constitutively active LCK in the majority of B-cell malignancies (27). In contrast, only CD40-sensitive OCI-Ly7 and Su-DHL4 (but not CD40-resistant OCI-Ly1 or OCI-Ly8) contained constitutively phosphorylated ERK p42/p44 (FIG. 7B). As p38 and jnk have also been shown to be activated by CD40 (28), and are known to be downstream targets of VAV (29, 30), we investigated the phosphorylation state of these MAPK family proteins. p38 was constitutively phosphorylated in all cell lines and JNK was present but not phosphorylated in any of the cell lines.

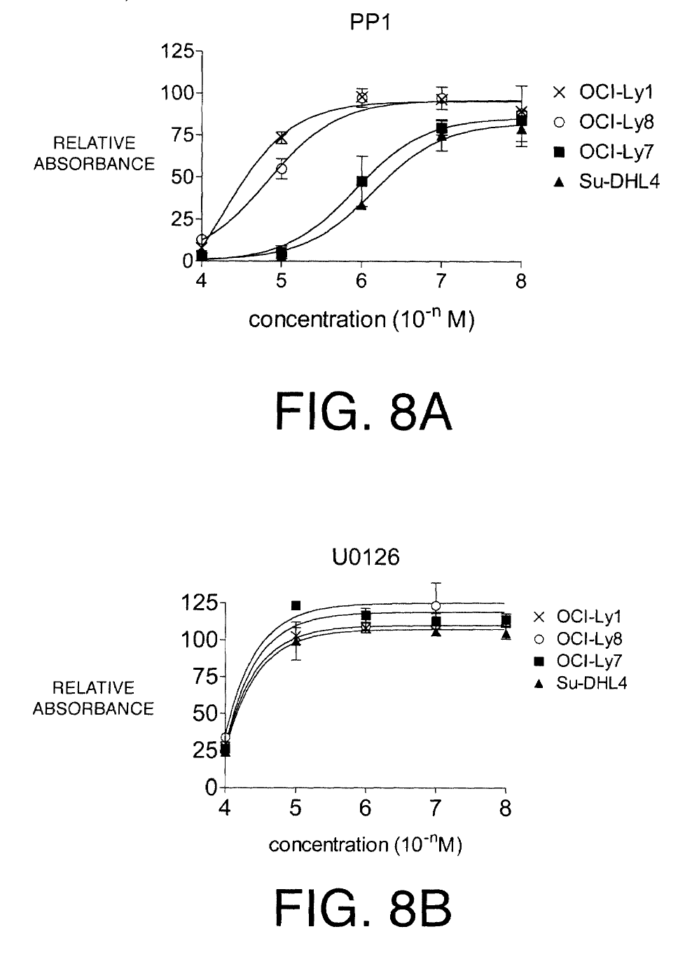

LCK transgenic mice have been shown to develop thymic lymphomas containing constitutively activated VAV and ERK, and cell lines derived from such tumors were dependent on tyrosine phosphorylation and raf-dependent ERK activation for survival (31). CD40-sensitive but not CD40-resistant cell lines express VAV, a central part of the LCK-ERK signal transduction pathway; therefore, we would expect growth of CD40-sensitive but not CD40-resistant cells would be expected to be inhibited by blocking LCK or ERK activity. Cells were exposed to a range of concentrations of the src family kinase inhibitor PP1 (32). As predicted, growth of CD40-sensitive OCI-Ly7 and Su-DHL4 cells was inhibited by PP1 more efficiently than growth of CD40-resistant OCI-Ly1 and OCI-Ly8 cells (FIG. 8A). In contrast, similar experiments with the MEK 1/2 inhibitor U0126, which prevents phosphorylation of ERK (33), did not show differential effects on growth of CD40-sensitive and CD40-resistant DLCBL lines (FIG. 8B). These observations suggest that LCK but not ERK is involved in maintaining growth of CD40-sensitive DLCBL cell lines.

### Example 4

### Phosphorylated ERK is Required for CD40-Mediated Cell Death

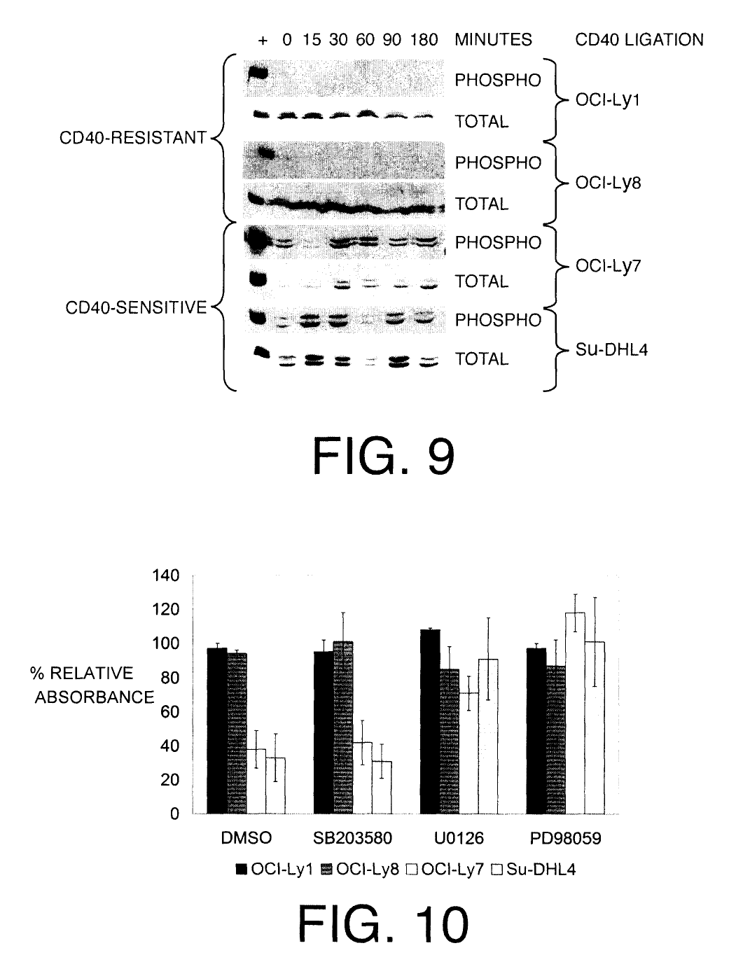

CD40 stimulation has been shown to activate both ERK and p38; however, inhibition of ERK, but not p38 phosphorylation, has been reported to enhance CD40-mediated apoptosis in a carcinoma cell line (34). We therefore determined the effect of CD40 stimulation on ERK activation by Western blotting for phosphorylated and total ERK. ERK was constitutively phosphorylated in OCI-Ly7 and Su-DHL4 cells, and not phosphorylated before or after CD40 ligation in OCI-1 and OCI-Ly8 cells (FIG. 9). This suggests that unlike carcinoma cell lines, DLCBL cell lines do not upregulate ERK activity upon CD40 signaling; however, constitutively active ERK may influence the outcome of CD40 signaling. For example, if ERK protects DLCBL against CD40-mediated apoptosis, inhibition of ERK prior to CD40 stimulation should enhance cell death in OCI-Ly7 and Su-DHL4 cells by reducing constitutive ERK activity, whereas OCI-Ly1 and OCI-Ly8 cells which lack active ERK should be unaffected by an ERK inhibitor. This hypothesis was tested using pharmacologic inhibitors of MEK. Su-DHL4 cells were incubated in the presence of 10 μM U0126 for 48 hours, permeabilized and stained with an anti-phospho-ERK antibody. Phosphorylation of ERK was reduced after addition of the MEK inhibitor U0126 as shown by flow cytometry. See Materials and Methods section of Examples. To determine if ERK is involved in CD40-mediated cell death, cells were treated with the MEK1/2 inhibitors U0126 or PD98058 prior to CD40 stimulation. The p38 inhibitor SB230580, which has been shown previously to have no effect on activation-induced cell death (11) was used as a negative control. Viable cell counts were measured 96 hours after CD40 ligation. The MEK1/2 inhibitors U0126 and PD98058, but not the p38 inhibitor SB230580, reduced CD40-mediated cell death in OCI-Ly7 and Su-DHL4 cells (FIG. 10), suggesting that ERK promotes rather than opposes activation-induced cell death in DLCBL cell lines.

### Example 5

### Expression of p-SRC, VAV, and p-ERK in DLCBL Tumor Samples

To determine if variations in ERK signaling exist in human tumors, immunohistochemical staining for phosphorylated SRC family kinases (p-SRC), VAV, and p-ERK was performed. An initial set of 25 blocks was pre-screened for expression of the B-cell markers CD20 and CD79a. Twenty samples were found to be positive and stained with antibodies to p-SRC, VAV, and p-ERK. Staining intensity was scored using the H-score system, which combines the percentage of positive cells with an intensity grade to yield a final range of 0 (negative staining) to 300 (all cells strongly positive).

TABLE 11

| Intensity | p-SRC | VAV | p-ERK |
|---|---|---|---|
| 0 | 10 | 0 | 6 |
| 1-99 | 7 | 5 | 5 |
| 100-199 | 1 | 3 | 3 |
| 200-300 | 2 | 12 | 6 |

The following correlations were observed. All six samples lacking phosphorylated ERK were also p-SRC negative. The four p-ERK positive samples lacking p-SRC had very high levels of VAV expression, suggesting that constitutive activation of VAV may abrogate the need for active SRC family kinases. Where VAV expression was moderate, staining intensity of p-ERK correlated with intensity of p-SRC.

The tumor samples were from biopsies performed at the Montreal General and Royal Victoria Hospitals in Montreal between 1991 and 1993. All samples had been classified as diffuse large B-cell lymphoma at diagnosis. This was confirmed by staining for expression of CD20 with an antibody from DAKO. CD20 is the standard marker for B-cells. Dr. Rene Michel of McGill University is kindly thanked for his assistance in these immunohistochemical studies on tumor samples.

Antibodies for staining of VAV, phospho-ERK, and phospho-SRC family were from Cell Signaling Technology, Danvers, Mass., USA (www.cellsignal.com). The VAV antibody was #2502. The presence of VAV has been associated with increased NFκB activity. The phospho-ERK antibody #4376 detects specifically the active form of ERK, the form that is required for CD40-mediated cell death. The phospho-SRC family #2101 detects SRC as well as the related kinases Lyn, Fyn, Lck, Yes and Hck in the active form. These kinases are part of a signaling pathway that can activate ERK.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of determining whether first cells respond to CD40 stimulation by undergoing apoptosis, said method comprising testing said first cells for their profile of gene expression, and comparing said profile with a second profile of gene expression of second cells known to respond to CD40 stimulation by undergoing apoptosis; wherein, if said profile of gene expression shows expression levels of genes characteristic of the second profile, said cells respond to CD40 stimulation by undergoing apoptosis.

2. The method of claim 1 wherein testing is by analysis of RNA from said first cells and said second cells on one or more arrays.

3. A method of determining whether first cells respond to CD40 stimulation by undergoing apoptosis, said method comprising testing said first cells for levels of expression of one or more genes, and comparing a first set of levels of expression of said one or more genes to a second set of levels of expression of said one or more genes in second cells known to respond to CD40 stimulation by undergoing apoptosis; wherein, if the first set of levels of expression of said one or more genes in said first cells is characteristic of said second set of levels of the second cells, said first cells respond to CD40 stimulation by undergoing apoptosis.

4. The method of claim 3 wherein testing is by analysis of RNA from said first cells and said second cells on one or more arrays.

5. A method for determining whether a population of cells is CD40-sensitive, said method comprising quantitating expression of one or more genes in a sample of cells from the population, wherein said one or more genes in diffuse large-cell B-lymphoma (DLCBL) cell lines are differentially regulated between CD40-sensitive DLCBL cell lines and CD40-resistant DLCBL cell lines, and comparing quantities of expression of said one or more genes in said sample to quantities of expression of said one or more genes in CD40-resistant DLCBL cell lines, wherein if said one or more genes are differentially regulated between the cells in the sample and the CD40-resistant DLCBL cell lines, then the population of cells is CD40-sensitive.

6. The method of claim 5 wherein the one or more genes are selected from B-cell maturation specific genes.

7. The method of claim 5 wherein the one or more genes are selected from members of the CD40 signaling pathway.

8. The method of claim 5 wherein the one or more genes are selected from the group consisting of: RAG1, RAG2, IGLL1, CD9, VPREB1, CD22, CD38, Bruton's tyrosine kinase, VAV1, LYN, LCK and MEK1/MAP2K1.

9. The method of claim 5 wherein the gene is RAG1.

10. The method of claim 5 wherein the gene is VAV1.

11. The method of claim 5 wherein quantitating expression is by reverse transcription polymerase chain reaction (RT-PCR).

12. The method of claim 5 wherein quantitating expression and comparing quantities is by analysis on arrays.

13. The method of claim 5 wherein quantitating expression and comparing quantities is by immunohistochemistry.

14. A method for determining whether a population of cells is CD40-sensitive or CD40-resistant, said method comprising testing a sample of cells from said population for the presence or absence of phosphorylated ERK, whereby, if phosphorylated ERK is present, the population of cells is CD40-sensitive, and if phosphorylated ERK is absent, the population of cells is CD40-resistant.

15. The method of claim 14 wherein the sample of cells is tested by immunoblot of non-denatured lysates from the sample of cells using anti-phospho-ERK antibodies.

16. The method of claim 14 wherein the sample of cells is tested by immunostaining.

17. The method of claim 14 wherein the immunostaining is by immunofluorescent labeled anti-phospho-ERK antibodies

18. The method of claim 14 wherein the immunostaining is by immunohistochemistry using anti-phospho-ERK antibodies conjugated to a reactive label.

19. The method of claim 14 wherein the sample of cells is a tissue biopsy or a fluid sample from a human or animal.

20. The method of claim 14 wherein the population of cells is a primary culture of cells from a human or animal.

21. The method of claim 14 wherein the population of cells is a cell line.

22. The method of claim 14 wherein the population of cells comprises B-cell lymphoma cells.

23. The method of claim 14 wherein the population of cells comprises diffuse large-cell B-lymphoma cells.

24. The method of claim 14 wherein the population of cells comprises cancer cells.

25. The method of claim 14 wherein the cancer cells are derived from endothelial cells.

26. The method of claim 14 wherein the cancer cells are derived from epithelial cells.

27. The method of claim 14 wherein the cancer cells are derived from fibroblasts.

28. The method of claim 14 wherein the cancer cells are breast cancer cells.

29. The method of claim 14 wherein the cancer cells are prostate cancer cells.

30. The method of claim 14 wherein the cancer cells are lung cancer cells.

31. The method of claim 14 wherein the cancer cells are colon cancer cells.

32. A method for determining whether a population of cells is CD40-sensitive or CD40-resistant, said method comprising testing a sample of cells from said population for the presence or absence of p-SRC, whereby, if p-SRC is absent, the population of cells is CD40-resistant.

33. A kit for determining whether a population of cells is CD40-sensitive or CD40-resistant, comprising anti-phospho-ERK antibody, anti-VAV antibody, and anti-RAG1 antibody.


SEQUENCE LISTING (12 sequences)

SEQ ID NO: 1 - DNA, 20 bp, Artificial Sequence
  PCR primer
tgatgaagggcctctctacg

SEQ ID NO: 2 - DNA, 20 bp, Artificial Sequence
  PCR primer
tttggaccctcaagagtggc

SEQ ID NO: 3 - DNA, 20 bp, Artificial Sequence
  PCR primer
gactatggctccgattcgac

SEQ ID NO: 4 - DNA, 20 bp, Artificial Sequence
  PCR primer
cctgacaagaagccgaagga

SEQ ID NO: 5 - DNA, 20 bp, Artificial Sequence
  PCR primer
gaaggtgaaggtcggagtca

SEQ ID NO: 6 - DNA, 20 bp, Artificial Sequence
  PCR primer
gactcttgcccttcgaacag

SEQ ID NO: 7 - DNA, 20 bp, Artificial Sequence
  PCR primer
atgaactggtccgccattac

SEQ ID NO: 8 - DNA, 20 bp, Artificial Sequence
  PCR primer
cctaccccatgatgttgccc

SEQ ID NO: 9 - DNA, 23 bp, Artificial Sequence
  PCR primer
agagagcagagaacacactttgc

SEQ ID NO: 10 - DNA, 20 bp, Artificial Sequence
  PCR primer
ttcgacaaggcccactctag

SEQ ID NO: 11 - DNA, 20 bp, Artificial Sequence
  PCR primer
cacctgctgtgagaagttcg

SEQ ID NO: 12 - DNA, 21 bp, Artificial Sequence
  PCR primer
tagatgtcaccggacaggctg